(19) United States
(12) Patent Application Publication
(10) Pub. No.: US 2016/0346240 A1
(43) Pub. Date: Dec. 1, 2016

(54) METHODS OF CHEMOTHERAPY USING CHEMOTHERAPEUTIC AGENTS BASED ON BETA-SUBSTITUTED BETA-AMINO ACIDS AND ANALOGS

(71) Applicant: Quadriga Biosciences, Inc., Los Altos, CA (US)

(72) Inventors: Bernd Jandeleit, Menlo Park, CA (US); Wolf-Nicolas Fischer, Sunnyvale, CA (US); Kerry J. Koller, San Francisco, CA (US); Gordon Ringold, Los Altos Hills, CA (US)

(21) Appl. No.: 15/233,566

(22) Filed: Aug. 10, 2016

Related U.S. Application Data
(63) Continuation-in-part of application No. 15/063,171, filed on Mar. 7, 2016.
Application No. 15/063,171 is a continuation of application No. 14/613,143, filed on Feb. 3, 2015, now Pat. No. 9,394,237.
(60) Provisional application No. 61/935,246, filed on Feb. 3, 2014.

(51) Int. Cl. A61K 31/197; A61K 31/27; A61K 31/255; A61K 45/06; A61K 31/662

(52) U.S. Cl. CPC A61K 31/197; A61K 45/06; A61K 31/662; A61K 31/255; A61K 31/27

(57) ABSTRACT

Compositions and methods for protecting normal, healthy cells during chemotherapy are disclosed. The methods include administering one or more compounds that inhibit the cell cycle of rapidly regenerating normal cells and a chemotherapeutic agent. The cell cycle inhibitors can be administered prior to administration of a chemotherapeutic agent. The chemotherapeutic agents can be β-substituted β-amino acids, β-substituted β-amino acid derivatives, and β-substituted β-amino acid analogs. Pharmaceutical compositions comprising the β-substituted β-amino acid derivatives and β-substituted β-amino acid analogs and uses thereof are also disclosed. The β-substituted β-amino acid derivatives and β-substituted β-amino acid analogs can be administered in conjunction with one or more compounds that inhibit the cell cycle of rapidly proliferating normal healthy cells. The cell cycle inhibitors can ameliorate the adverse effects of the β-substituted β-amino acid derivatives and β-substituted β-amino acid analogs.

This application is a continuation-in-part of U.S. application Ser. No. 15/063,171 filed on Mar. 7, 2016, which is a continuation of U.S. application Ser. No. 14/613,143, on Feb. 3, 2015, issued as U.S. Pat. No. 9,394,237, which claims the benefit under 35 U.S.C. §119(e) of U.S. Provisional Application No. 61/935,246 filed on Feb. 3, 2014, each of which is incorporated by reference in its entirety.

### FIELD

The present invention relates to compositions and methods for protecting normal, healthy cells during chemotherapy. The methods include administering one or more compounds that inhibit the cell cycle of rapidly regenerating normal cells and a chemotherapeutic agent. The cell cycle inhibitors can be administered prior to administration of a chemotherapeutic agent. The chemotherapeutic agents can be β-substituted β-amino acids, β-substituted β-amino acid derivatives, and β-substituted β-amino acid analogs. The β-substituted β-amino acid derivatives and β-substituted β-amino acid analogs are selective substrates for LAT1/4F2hc and exhibit rapid uptake and retention in tissue such as tumors expressing the LAT1/4F2hc transporter. Pharmaceutical compositions comprising the β-substituted β-amino acid derivatives and β-substituted β-amino acid analogs and uses thereof are also disclosed. The β-substituted β-amino acid derivatives and β-substituted β-amino acid analogs can be administered in conjunction with one or more compounds that inhibit the cell cycle of rapidly proliferating normal healthy cells. The cell cycle inhibitors can ameliorate the adverse effects of the β-substituted β-amino acid derivatives and β-substituted β-amino acid analogs.

### BACKGROUND

The ability to selectively target chemotherapy has immense value in clinical practice. Cancer is a leading cause of death in the developed world, with one in every three people developing cancer during his or her lifetime. There are many treatment options for cancer including surgery, chemotherapy, radiation therapy, immunotherapy, and monoclonal antibody treatment. Unfortunately, for many patients cancer treatment options are limited and response rates remain low.

Surgery is the oldest effective form of tumor therapy and can often result in a complete cure, depending of the type and nature of the tumor. Many tumors, however, occur in locations and/or number that make surgery impossible or impractical. Also, surgical debulking is not guaranteed to remove all abnormal cells, particularly in the case of tumors located in the brain where maximum preservation of normal tissue is desired. Residual abnormal cells pose an increased risk of tumor re-growth and/or metastasis.

Radiation therapy is often used as an adjunct to surgery. Various types of radiation, both from external and implanted sources, have been used with some success. Low linear-energy-transfer (LET) sources, such as β-particles and γ-rays, require repeated treatments over extended periods of time to produce any significant reduction in tumor cells. High LET sources, such as neutrons, protons or α-particles, do not require oxygen to enhance their biological effectiveness. External beam therapy has been available for decades, however, significant radiation damage occurs to normal tissues, and patients often succumb to widespread radiation-induced necrosis (Laramore, et al., Cancer, 1978, 42(1), 96-103).

Chemotherapy is used in attempts to cure or palliate cancer. Small molecule chemotherapeutics target rapidly dividing cells, halting cell proliferation by interfering with DNA replication, cytoskeletal rearrangements and/or signaling pathways that promote cell growth. Disruption of cell division slows the growth of malignant cells and may also kill tumor cells by triggering apoptosis. Alkylating agents, such as bis(2-chloroethyl)amine derivatives, act by covalent interaction with nucleophilic heteroatoms in DNA or proteins. It is believed that these difunctional agents are able to crosslink a DNA chain within a double helix in an intrastrand or interstrand fashion, or to crosslink between DNA, proteins or other vital macromolecules. The crosslinking results in inhibitory effects on DNA replication and transcription with subsequent cell death. Since these drugs also indiscriminately kill normal populations of rapidly proliferating cells, such as those found in the immune system and in the gastrointestinal tract, side effects that limit tolerated doses, are common.

The harsh side effects and the ultimate failure of most chemotherapy regimens have motivated investigation of alternatives, including drugs that target specifically tumor cells. Normal cells and tumor cells differ markedly in nutrient and energy metabolism, a phenomenon known as the Warburg effect (Ganapathy, et al., Pharmacol Ther, 2009, 121(1), 29-40; and Vander Heiden, et al., Science, 2009, 324(5930), 1029-1033). Enhanced proliferation in tumor cells places increased demand for nutrients to serve as building blocks for the biosynthesis of macromolecules and as sources of energy. Tumor-selective nutrient accumulation is most clearly evident in imaging studies of human tumors using positron emission tomography (PET) and [¹⁸F]-fluorodeoxyglucose (FDG). FDG accumulates at high levels in many kinds of solid tumors and is thought to be taken up into tumor cells by sugar transporters. Amino acids are the primary source of cellular nitrogen, used for nucleotide, glutathione, amino sugar, and protein synthesis. In addition, tumors often utilize the carbon skeletons of amino acids as an oxidative fuel source for ATP generation in addition to glucose and fatty acids (Baggetto, Biochimie, 1992, 74(11), 959-974; Mazurek and Eigenbrodt, 2003, Anticancer Res, 2003, 23(2A), 1149-1154; and DeBerardinis, et al., Proc Natl Acad Sci USA, 2007, 104(49), 19345-19350). Therefore, tumor cells must express select specific transporters to satisfy maintenance and growth requirements for nutritional amino acids. To compete with surrounding tissue for nutrients, tumor cells upregulate levels of certain transporters to allow for more efficient extraction of nutrients than that of the host tissue.

Amino acid transport across the plasma membrane in mammalian cells is mediated by different transport “systems” such as the sodium-dependent systems A, ASC and N, and sodium-independent system L (Christensen, Phys Rev, 1990, 70, 43-77). System L is a ubiquitous plasma membrane amino acid transport system that is characterized by the sodium-independent uptake of bulky, hydrophobic amino acids and its high affinity interaction with 2-amino-bicyclo[2,2,1]heptane-2-carboxylic acid (BCH). System L activity is presently attributed to four sodium-independent transporters (LAT1-4). However, most cancers over-express only one member, the large amino acid transporter 1 (LAT1/4F2hc). This transporter is a heterodimer consisting of a light chain (LAT1) that constitutes the transporter and a heavy chain 4F2hc (also known as CD98, or Tumor Antigene TA1) that is required for proper targeting of the light chain to the plasma membrane. The expression and activity of LAT1/4F2hc correlates with cell proliferation and cancer growth; and up-regulation of LAT1/4F2hc has been observed, for example, in cancers of brain, colon, lung, liver, pancreas, and skin (Jager, et al., J Nucl Med, 1998, 39(10), 1736-1743; Ohkame, et al., J Surg Oncol, 2001,78(4), 265-267; Tamai, et al., Cancer Detect Prev, 2001, 25(5), 439-445; Kim, et al., Anticancer Res, 2004, 24(3a),1671-1675; Kobayashi, et al., Neurosurgery, 2008, 62(2), 493-503; Imai, et al., Histopathology, 2009, 54(7), 804-813; and Kaira, et al., 2009, Lung Cancer, 66(1), 120-126). Furthermore, the expression of LAT1/4F2hc has been used as an independent factor to predict poor prognoses in patients with astrocytic brain tumors, lung cancer, and prostate cancer (Nawashiro, et al., Int J Canc, 2006, 119(3), 484-492; Kaira, et al., Lung Cancer, 2009, 66(1), 120-126; Kaira, et al., Cancer Sci, 2008, 99(12), 2380-2386; and Sakata, et al., Pathol Int, 2009, 59(1), 7-18). Inhibition of LAT1/4F2hc-mediated transport with non-metabolizable amino acids such as BCH can reduce growth and induce apoptosis in cancer cells in vitro (Kim, et al., Biol Pharm Bull, 2008, 31(6), 1096-1100; Shennan and Thomson, Oncol Rep, 2008, 20(4), 885-889; and Kaji, et al., Int J Gynecol Cancer, 2010, 20(3), 329-336). Clinical studies have shown that the specificity and positive predictive value of L-[3-¹⁸F]-α-methyltyrosine ([¹⁸F]-FAMT) PET is superior to [¹⁸F]-FDG PET. The uptake of [¹⁸F]-FAMT in tumors has been closely correlated with LAT1 expression (Haase, et al., J Nucl Med, 2007, 48(12), 2063-2071; Kaira, et al., Clin Cancer Res, 2007, 13(21), 6369-6378; and Urakami, et al., Nucl Med Biol, 2009, 36(3), 295-303).

In particular, melphalan is an effective chemotherapy drug used in treating multiple myeloma, ovarian cancer, retinoblastoma, and other hematopoietic tumors. However, substrates such as gabapentin are reported to be transported much more rapidly than melphalan (Uchino, et al., Mol Pharmacol 2002, 61(4), 729-737). It is widely believed that uptake of melphalan (Alkeran®, otherwise known as L-Phenylalanine Mustard, or L-PAM) into cells is mediated by amino acid transporters. Melphalan is an alkylating agent linked to the essential amino acid phenylalanine. Because normal cells and tumor cells differ markedly in nutrient and energy metabolism (Warburg effect) (Vander Heiden, et al., Science, 2009, 324(5930), 1029-1033), melphalan was introduced into clinical practice with the expectation that it would preferentially accumulate in rapidly dividing tumor cells compared to normal cells, thereby increasing its overall therapeutic index. Surprisingly, melphalan caused many of the same side effects as other conventional alkylation agents, including myelosuppression. In a series of publications, Vistica et al. examined melphalan transport in different cell types and identified two independent transport systems for melphalan. One system, presumed to be System L, is characterized by the sodium-independent uptake of bulky, hydrophobic amino acids and its sensitivity toward inhibition with 2-amino-bicyclo[2,2,1]heptane-2-carboxylic acid (BCH) (Vistica, Biochim Biophys Acta, 1979, 550(2), 309-317). A second transport system is sodium-dependent, exhibits its highest affinity for leucine, but is insensitive to both BCH and the system A-specific inhibitor α-amino-isobutyric acid (A1B) (Vistica, Biochim Biophys Acta, 1979, 550(2), 309-317). Although LAT1 is overexpressed on the cell surface of almost all tumor cells regardless of the tissue of origin, response rates to melphalan are low for most cancer types, and the drug is only approved for the treatment of multiple myeloma and ovarian cancer. Melphalan is a poor substrate for LAT1 compared to other large amino acids such as phenylalanine or leucine (Uchino, et al., Mol Pharmacol 2002, 61(4), 729-737; and Hosoya, et al., Biol Pharm Bull, 2008, 31(11), 2126-2130). Nitrogen mustard derivatives with higher selectivity toward the LAT1/4F2hc system could reduce side effects associated with nitrogen mustard therapy, allow for an increase in dose, and extend the use into other areas of cancer treatment.

Although the potential for active transport strategies for increasing drug uptake into tumor cells is known and generally accepted, chemotherapeutics and tumor imaging agents have in general not been optimized for transporters known to be over-expressed in tumor cells. While the general concept of using LAT1/2Fhc-selective compounds to deliver therapeutic agents to tumors is appreciated, the existing art gives no guidance as to how one prepares a composition that exploits LAT1/4F2hc selective compounds. Thus, there is a need for new therapeutic agents that are more selective toward LAT1/4F2hc.

Several amino acid-related drugs that are substrates of the LAT1/4F2hc transporter are known including L-Dopa, 3-O-methyldopa, droxidopa, carbidopa, 3,3′,5′-triiodothyronine, thyroxine, gabapentin, and melphalan (Uchino, et al., Mol Pharm 2002, 61(4), 729-737; and del Amo et al., Eur J Pharm Sci, 2008, 35(3), 161-174).

In chemotherapy, cytotoxic agents are used to treat proliferative disorders or autoimmune diseases such as cancer, psoriasis, arthritis, lupus and multiple sclerosis. Cytotoxic agents for treating the proliferative disorder can also be toxic to normal, healthy cells. This can lead to a variety of side effects such as bone marrow suppression that can limit the dose and thereby the therapeutic efficacy of a chemotherapeutic regimen.

Bone marrow suppression is characterized by both myelosuppression (anemia, neutropenia, agranulocytosis, and thrombocytopenia) and lymphopenia. Anemia is characterized by a reduction in the number of red blood cells or erythrocytes, the quantity of hemoglobin, or the volume of packed red blood cells. Neutropenia is characterized by a selective decrease in the number of circulating neutrophils and an enhanced susceptibility to bacterial infections. Thrombocytopenia is characterized by a reduction in platelet number with increased susceptibility to bleeding. Lymphopenia is characterized by a reduction in the number of circulating lymphocytes such as T-cells and B-cells. Lymphopenic patients are predisposed to infections. Adjusting chemotherapy doses and dose regimens to minimize the effects of bone marrow suppression can reduce therapeutic efficacy and compromise disease control and survival.

In addition to bone marrow suppression, chemotherapeutic agents can adversely affect other healthy cells such as renal epithelial cells. Damage caused to renal tubular epithelia can lead to chronic kidney disease, multi-organ failure, sepsis, and death.

Chemoprotective compounds and therapies useful in reducing the side effects of certain chemotherapeutic agents are known. Small molecules have been used to reduce some of the side effects of certain chemotherapeutic compounds. For example, leukovorin has been used to mitigate the effects of methotrexate on bone marrow cells and on gastrointestinal mucosa cells. Amifostine has been used to reduce the incidence of neutropenia-related fever and mucositis in patients receiving alkylating or platinum-containing chemotherapeutics. Also, dexrazoxane has been used to provide cardioprotection from anthracycline anti-cancer compounds. Unfortunately, there is concern that many chemoprotectants, such as dexrazoxane and amifostine, can decrease the efficacy of chemotherapy.

Additional chemoprotectant therapies include the use of growth factors. Hematopoietic growth factors such as recombinant proteins include granulocyte colony stimulating factor (G-CSF) and granulocyte-macrophage colony stimulating factor (GM-CSF) and their derivatives for the treatment of neutropenia, and erythropoietin (EPO) and its derivatives for the treatment of anemia. EPO has significant toxicity in cancer patients, leading to increased thrombosis, relapse and death in several large randomized trials. G-CSF and GM-CSF may increase the late risk of secondary bone marrow disorders such as leukemia and myelodysplasia. Although growth factors can speed recovery of some blood cell lineages, no therapy exists to treat suppression of platelets, macrophages, T-cells or B-cells.

Recently, CDK4/6 inhibitors are being investigated for use in chemoprotective therapies. Hematopoietic stem cells (HSPCs) give rise to progenitor cells which in turn give rise to differentiated blood components including lymphocytes, erythrocytes, platelets, granulocytes, monocytes. HSPCs require the activity of CDK4/6 for proliferation. A number of CDK 4/6 inhibitors have been identified, including pyrido[2,3-d]pyrimidines, 2-anilinopyrimidines, diaryl ureas, benzoyl-2,4-diaminothiazoles, indolo[6,7-a]pyrrolo[3,4-c]carbazoles, and oxindoles (Sharma et al., Curr. Cancer Drug Targets 8 (2008) 53-75). U.S. Application Publication No. 2011/0224227 describes the use of certain CDK4/6 inhibitors, such as PD0332991 and 2BrIC (see Zhu, et al., J. Med. Chem., 46 (11) 2027-2030 (2003); PCT/US2009/059281) to reduce or prevent the effects of cytotoxic compounds on HSPCs in a subject undergoing chemotherapeutic treatments. See also U.S. Application Publication No. 2012/0100100.

Improved methods of mitigating adverse effects of chemotherapy, and in particular chemotherapy based on the administration of chemotherapeutic agents based on β-substituted β-amino acid derivatives and β-substituted β-amino acid analogs disclosed herein are desired.

### SUMMARY

Differentiation of malignant cancer tissue from neighboring nonmalignant tissue can be accomplished by exploiting changes in biochemical fluxes that occur in response to metabolic, genetic, and/or microstructural changes in the malignant cells. Compounds provided by the present disclosure substantially improve chemotherapy of tissue expressing the LAT1/4F2hc transporter including malignant tumors. The β-substituted β-amino acid derivatives and β-substituted β-amino acid analogs provided by the present disclosure provide greater uptake selectivity for the target tissue or cells expressing the LAT1/4F2hc transporter with low non-specific uptake for non-target tissues or cells.

Embodiments provided by the present disclosure provide novel β-substituted β-amino acid derivatives and β-substituted β-amino acid analogs, and methods of using such derivatives, for example, as chemotherapeutic agents. Certain embodiments further relate to methods of synthesizing β-substituted β-amino acid derivatives and β-substituted β-amino acid analogs and to pharmaceutical compositions comprising such derivatives. The β-substituted β-amino acid derivatives and β-substituted β-amino acid analogs the present disclosure exhibit selectivity for LAT1/4F2hc and therefore accumulate in cancerous cells when administered to a subject in vivo. Advantages provided by compounds of the present disclosure reflect the properties of LAT1/4F2hc substrates, namely, blood brain-barrier (BBB) permeability, rapid uptake, and prolonged retention in tumors expressing the LAT1/4F2hc transporter, and further serve as chemotherapeutic agents.

According to the present invention, methods of reducing the effects of chemotherapy on normal/healthy cells in a patient being treated for cancer or abnormal cell proliferation are disclosed, comprising administering to the patient a therapeutically effective amount of a cell cycle inhibitor; and administering to the patient a therapeutically effective amount of a compound of Formula (1):

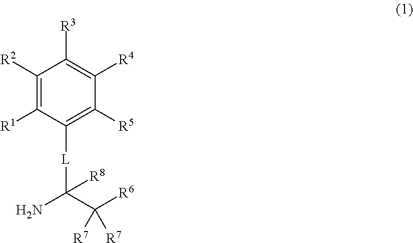

or a pharmaceutically acceptable salt thereof, wherein:
at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅-C₁₀ heteroaryl, C₆₋₁₆ heteroarylalkyl, and substituted C₆₋₁₆ heteroarylalkyl;

one of R¹, R², R³, R⁴, and R⁵ comprises a chemotherapeutic moiety;

each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;

R⁶ is selected from a carboxylic acid (—COOH), a carboxylic acid analog, and a carboxylic acid (bio)isostere;

each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;

R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

According to the present invention, methods of promoting recovery from the effects of a chemotherapeutic regimen for treating cancer in a patient are disclosed comprising: administering to the patient a therapeutically effective amount of a cell cycle inhibitor to inhibit the proliferation of normal, healthy cells; and administering a therapeutically effective about of a compound of Formula (1).

According to the present invention, methods of treating cancer in a patient are disclosed, comprising administering to a patient in need of such treatment a therapeutically effective amount of a cell cycle inhibitor; and a therapeutically effective amount of a compound of Formula (1).

### BRIEF DESCRIPTION OF THE DRAWINGS

Those skilled in the art will understand that the drawings described herein are for illustration purposes only. The drawings are not intended to limit the scope of the present disclosure.

FIGS. 1A-1C show the effect of melphalan and compound (5) at concentrations of 0.3 μM, 1 μM and 3 μM on erythroid and myelid hematopoietic colonies.

FIG. 2 shows tumor volume in mice during dosing of compound (5).

FIG. 3 shows tumor volume in mice during dosing of melphalan.

FIGS. 4A-4D shows the survival, body weight, white cell count, and granulocyte count, respectively, following IP administration of various doses of compound (5) to mice.

FIGS. 5A-5C show the tumor volume in a triple negative breast cancer xenograft mouse model with administration of vehicle or regimens of compound (5).

FIGS. 5D-5G show the body weight change, white blood cell count, and granulocyte count during administration of vehicle or regimens of compound (5) for the triple negative breast cancer xenograft model in FIGS. 5A-5C.

FIGS. 6A-6C show the tumor volume in a prostate cancer xenograft mouse model with administration of vehicle or regimens of compound (5).

FIGS. 6D-6G show the body weight change, white blood cell count, and granulocyte count during administration of vehicle or regimens of compound (5) for the prostate cancer xenograft model in FIGS. 6A-6C.

FIG. 7 shows the tumor volume for large prostate tumors in the PC3 xenograft model following IP administration of a regimen of compound (5) at a dose of 5 mg/kg.

FIGS. 8A-8D show the tumor volume in the PC3 xenograft mouse model following escalation in the dose of compound (5) to 7.5 mg/kg, three times per week for three weeks.

FIGS. 9A-9D show the tumor volume in the PC3 xenograft mouse model following escalation in the dose of compound (5) to 10 mg/kg, three times per week for three weeks.

FIGS. 10A-10C show the change in body weight, white blood cell count, and granulocyte count, respectively, for the animals subjected to the escalated dosing as presented in FIGS. 8A-8D and FIGS. 9A-9D.

FIG. 11 shows the change in tumor volume in a PC3 xenograft mouse model during and following weekly intravenous (IV) dosing of compound (5).

FIG. 12 shows the change in tumor volume in a PC3 xenograft mouse model during and following weekly intravenous (IV) dosing of compound (7).

FIG. 13 shows the change in tumor volume in a PC3 xenograft mouse model during and following weekly intravenous (IV) dosing of compound (9).

FIG. 14 shows the change in tumor volume in a triple negative breast cancer (MDA-MB-231) xenograft mouse model during and following weekly intravenous (IV) dosing of compound (5) or (7).

FIG. 15 shows the change in tumor volume in a glioblastoma (U251) mouse orthotopic xenograft model during and following weekly dosing of compound (5) or temozolomide.

FIG. 16 shows the change in tumor volume in a glioblastoma (U251) mouse orthotopic xenograft model during and following dosing regimens of compound (5) or temozolomide.

FIG. 17 shows the change in tumor volume in an orthotopic multiple myeloma (U266) mouse xenograft model during and following dosing regimens of compound (5) or bortezomib.

FIG. 18 shows the change in body weight for the orthotopic multiple myeloma (U266) mouse xenograft model shown in FIG. 17 during and following dosing regimens of compound (5) or bortezomib.

FIG. 19 shows the percent change in body weight of rats dosed with methotrexate, compound (5), or a combination of methotrexate and compound (5).

FIG. 20 shows the white blood cell count of rats dosed with methotrexate, compound (5), or a combination of methotrexate and compound (5).

FIG. 21 shows the granulocyte cell count of rats dosed with methotrexate, compound (5), or a combination of methotrexate and compound (5).

FIG. 22 shows the lymphocyte cell count of rats dosed with methotrexate, compound (5), or a combination of methotrexate and compound (5).

FIG. 23 shows the platelet count of rats dosed with methotrexate, compound (5), or a combination of methotrexate and compound (5).

Reference is now made to certain compounds and methods. The disclosed embodiments are not intended to be limiting of the claims. To the contrary, the claims are intended to cover all alternatives, modifications, and equivalents.

### DETAILED DESCRIPTION

A dash (“—”) that is not between two letters or symbols is used to indicate a point of attachment for a moiety or substituent. For example, —CONH₂ is attached through the carbon atom.

“Alkyl” refers to a saturated or unsaturated, branched, or straight-chain, monovalent hydrocarbon radical derived by the removal of one hydrogen atom from a single carbon atom of a parent alkane, alkene, or alkyne. Examples of alkyl groups include methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like. The term “alkyl” is specifically intended to include groups having any degree or level of saturation, i.e., groups having exclusively carbon-carbon single bonds, groups having one or more carbon-carbon double bonds, groups having one or more carbon-carbon triple bonds, and groups having combinations of carbon-carbon single, double, and triple bonds. Where a specific level of saturation is intended, the terms alkanyl, alkenyl, and alkynyl are used. In certain embodiments, an alkyl group is C₁₋₆ alkyl, C₁₋₅ alkyl, C₁₋₄ alkyl, C₁₋₃ alkyl, and in certain embodiments, ethyl or methyl.

“Alkylsulfanyl” also referred to as “alkylthio”, refers to a radical —SR where R is alkyl or cycloalkyl as defined herein. Examples of alkylsulfanyl groups include methylsulfanyl, ethylsulfanyl, propylsulfanyl, isopropylsulfanyl, butylsulfanyl, and cyclohexylsulfanyl. In certain embodiments, an alkylsulfanyl group is C₁₋₆ alkylsulfanyl, in certain embodiments, C₁₋₅ alkylsulfanyl, in certain embodiments, C₁₋₄ alkylsulfanyl, in certain embodiments, C₁₋₃ alkylsulfanyl, in certain embodiments, ethylsulfanyl (ethylthio), and in certain embodiments, methylsulfanyl (methylthio).

“Alkylsulfinyl” refers to a radical —S(O)R where R is alkyl or cycloalkyl as defined herein. Examples of alkylsulfinyl groups include methylsulfinyl, ethylsulfinyl, propylsulfinyl, isopropylsulfinyl, butylsulfinyl, and cyclohexylsulfinyl. In certain embodiments, an alkylsulfinyl group is C₁₋₆ alkylsulfinyl, in certain embodiments, C₁₋₅ alkylsulfinyl, in certain embodiments, C₁₋₄ alkylsulfinyl, in certain embodiments, C₁₋₃ alkylsulfinyl, in certain embodiments, ethylsulfinyl, and in certain embodiments, methylsulfinyl.

“Alkylsulfonyl” refers to a radical —S(O)₂R where R is alkyl or cycloalkyl as defined herein. Examples of alkylsulfonyl groups include methylsulfonyl, ethylsulfonyl, propylsulfonyl, isopropylsulfonyl, butylsulfonyl, and cyclohexylsulfonyl. In certain embodiments, an alkylsulfonyl group is C₁₋₆ alkylsulfonyl, in certain embodiments, C₁₋₅ alkylsulfonyl, in certain embodiments, C₁₋₄ alkylsulfonyl, in certain embodiments, C₁₋₃ alkylsulfonyl, in certain embodiments, ethylsulfonyl, and in certain embodiments, methylsulfonyl.

“Alkoxy” refers to a radical —OR where R is alkyl as defined herein. Examples of alkoxy groups include methoxy, ethoxy, propoxy, and butoxy. In certain embodiments, an alkoxy group is C₁₋₆ alkoxy, in certain embodiments, C₁₋₅ alkoxy, in certain embodiments, C₁₋₄ alkoxy, in certain embodiments, C₁₋₃ alkoxy, and in certain embodiments, ethoxy or methoxy.

“Aryl” by itself or as part of another substituent refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes a phenyl ring fused to a 5- to 7-membered heterocycloalkyl ring containing one or more heteroatoms selected from N, O, and S. For such fused, bicyclic ring systems wherein only one of the rings is a carbocyclic aromatic ring, the radical carbon atom may be at the carbocyclic aromatic ring or at the heterocycloalkyl ring. Examples of aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group is C₆₋₁₀ aryl, C₆₋₉ aryl, C₆₋₈ aryl, and in certain embodiments, phenyl. Aryl, however, does not encompass or overlap in any way with heteroaryl, separately defined herein.

“Arylalkyl” refers to an acyclic alkyl radical in which one of the hydrogen atoms bonded to a carbon atom is replaced with an aryl group. Examples of arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₁₆ arylalkyl, e.g., the alkanyl, alkenyl or alkynyl moiety of the arylalkyl group is C₁₋₆ and the aryl moiety is C₆₋₁₀, in certain embodiments, an arylalkyl group is C₇₋₁₆ arylalkyl, e.g., the alkanyl, alkenyl or alkynyl moiety of the arylalkyl group is C₁₋₆ and the aryl moiety is C₆₋₁₀. In certain embodiments, an arylalkyl group is C₇₋₉ arylalkyl, wherein the alkyl moiety is C₁₋₃ alkyl and the aryl moiety is phenyl. In certain embodiments, an arylalkyl group is C₇₋₁₆ arylalkyl, C₇₋₁₄ arylalkyl, C₇₋₁₂ arylalkyl, C₇₋₁₀ arylalkyl, C₇₋₈ arylalkyl, and in certain embodiments, benzyl.

Bioisosteres are atoms or molecules that fit the broadest definition for isosteres. The concept of bioisosterism is based on the notion that single atom, groups, moieties, or whole molecules, which have chemical and physical similarities, produce similar biological effects. A bioisostere of a parent compound can still be recognized and accepted by its appropriate target, but its functions will be altered as compared to the parent molecule. Parameters affected with bioisosteric replacements include, for example, size, conformation, inductive and mesomeric effects, polarizability, capacity for electrostatic interactions, charge distribution, H-bond formation capacity, pKa (acidity), solubility, hydrophobicity, lipophilicity, hydrophilicity, polarity, potency, selectivity, reactivity, or chemical and metabolic stability, ADME (absorption, distribution, metabolism, and excretion). Although common in pharmaceuticals, carboxyl groups or carboxylic acid functional groups (—CO₂H) in a parent molecule may be replaced with a suitable surrogate or (bio)isostere to overcome chemical or biological shortcomings while retaining the desired attributes of the parent molecule bearing one or more carboxyl groups or carboxylic acid functional groups (—CO₂H). Examples of suitable surrogates or (bio)isosteres of carboxyl groups or carboxylic acid functional groups (—CO₂H) include hydroxamic acids (—CONR¹²OH); boronic acids (—B(OH)(OR¹²), phosphinic acids or derivatives thereof (—PO(OH)R¹²), phosphonic acid or derivatives thereof (—PO(OH)(OR¹²), sulfinic acid (—SOOH), sulfonic acid (—SO₂OH), sulfonamide (—SO₂NHR¹² or —NHSO₂R¹²), sulfonimide or acyl sulfonimide (—SO₂NHCOR¹² or —CONHSO₂R¹²), sulfonylureas (—SO₂NHCONHR¹² or —NHCONHSO₂R¹²), amide (—CONHR¹² or —NHCOR¹²), wherein R¹² in any of the foregoing is selected from hydrogen, C₁₋₆ alkyl, C₁₋₄ fluoroalkyl, C₃₋₆ cycloalkyl, and C₆₋₁₀ aryl, acylcyanamide (—CONHCN); 2,2,2-trifluoroethan-1-ols (—CH(CF₃)OH), 2,2,2-trifluoromethyl ketones and hydrates thereof (—COCF₃ and —C(OH)₂CF₃), acidic heterocycles and their annular tautomers such as, for example, tetrazole, 5-oxo-1,2,4-oxadiazole, 5-oxo-1,2,4-thiadiazole, 5-thioxo-1,2,4-oxadiazole, thiazolidinedione, oxazolidinedione, oxadiazolidinedione, 3-hydroxyisoxazole, 3-hydroxyisothiazole, 1-hydroxy-imidazole, 1-hydroxy-pyrazole, 1-hydroxy-triazole, 1H-imidazol-2-ol, tetrazole-5-thiol, 3-hydroxyquinolin-2-ones, 4-hydroxyquinolin-2-ones, tetronic acid, tetramic acid, mercaptoazoles such as sulfanyl-1H-imidazole, sulfinyl-1H-imidazole, sulfonyl-1H-imidazole, sulfanyl-1H-triazole, sulfinyl-1H-triazole, sulfonyl-1H-triazole, sulfanyl-1H-1,2,4-triazole, sulfinyl-1H-1,2,4-triazole, sulfonyl-1H-1,2,4-triazole, sulfanyl-1,4-dihydro-1,2,4-triazol-5-one, sulfinyl-1,4-dihydro-1,2,4-triazol-5-one, sulfonyl-1,4-dihydro-1,2,4-triazol-5-one, sulfanyl 1H-tetrazole, sulfanyl 2H-tetrazole, sulfinyl 1H-tetrazole, sulfinyl 2H-tetrazole, sulfonyl 1H-tetrazole, sulfonyl 2H-tetrazole, or sulfonimidamides; and; acidic oxocarbocycles or cyclic polyones and their resonance forms such as, for example, cyclopentane-1,3-diones, squaric acids, squareamides, mixed squaramates, or 2,6-difluorophenols.

“Compounds” of Formula (1) and moieties of Formula (2) disclosed herein include any specific compounds within these formulae. Compounds may be identified either by their chemical structure and/or chemical name. Compounds are named using the ChemDraw Ultra 12.0 (CambridgeSoft, Cambridge, Mass.) nomenclature program. When the chemical structure and chemical name conflict the chemical structure is determinative of the identity of the compound. The compounds described herein may comprise one or more stereogenic centers and/or double bonds and therefore may exist as stereoisomers such as double-bond isomers (i.e., geometric isomers), enantiomers, diastereomers, or atropisomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan.

Compounds of Formula (1) and moieties of Formula (2) include optical isomers of compounds of Formula (1) and moieties of Formula (2), racemates thereof, and other mixtures thereof. In such embodiments, the single enantiomers or diastereomers may be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column with chiral stationary phases. In addition, compounds of Formula (1) include (Z)- and (E)-forms (or cis- and trans-forms) of compounds with double bonds either as single geometric isomers or mixtures thereof.

Compounds of Formula (1) and moieties of Formula (2) may also exist in several tautomeric forms including the enol form, the keto form, and mixtures thereof. Accordingly, the chemical structures depicted herein encompass all possible tautomeric forms of the illustrated compounds. Compounds may exist in unsolvated forms as well as solvated forms, including hydrated forms. Certain compounds may exist in multiple crystalline, co-crystalline, or amorphous forms. Compounds of Formula (1) include pharmaceutically acceptable salts thereof, or pharmaceutically acceptable solvates of the free acid form of any of the foregoing, as well as crystalline forms of any of the foregoing

Compounds of Formula (1) are also referred to herein as β-substituted β-amino acid derivatives and/or as β-substituted β-amino acid analogs.

“Chemotherapeutic moiety” refers to a moiety effective in treating cancer including, any of those disclosed herein. In certain embodiments, a chemotherapeutic moiety may be any suitable chemotherapeutic moiety of a chemotherapeutic drugs known in the art that retains cytotoxic activity when bonded either directly or indirectly through a suitable spacing moiety to a β-amino acid derivative, β-amino acid analog, or β-amino acid carboxylic acid (bio)isostere as a LAT1 recognition element provided by the present disclosure. The conjugate or fusion product of the chemotherapeutic moiety with the β-amino acid derivative, β-amino acid analog, or β-amino acid carboxylic acid (bio)isostere is simultaneous a selective substrate for the LAT1/4F2hc transporter.

In certain embodiments, the chemotherapeutic moiety, is selected from a nitrogen mustard (—N(—CR₂—CR₂—X)₂), a N-monoalkyl or N,N-dialkyl triazene (—N═N—NR₂), a haloacetamide (—NR—CO—CH₂—X), an epoxide (—CROCR—R), an aziridine (—NC₂H₄), a Michael acceptor (—CR═CR-EWG-), a sulfonate or a bissulfonate ester (—OSO₂R or ROSO₂—), an N-nitrosourea (—NR—CO—N(NO)R), a bissulfonyl hydrazine (R″SO₂—NR—N(−)-SO₂R′″, —SO₂—NR—NR′—SO₂R′″, or R″SO₂—NR—NR′—SO₂—), a phosphoramidate (—O—P(═O)(N(R)—CH₂—CH₂—X)₂ or —O—P(═O)(N(—CH₂—CH₂—X)₂)₂, and a radionuclide such as, for example, 131-iodine (¹³¹[I]—) or 211-astatine (²¹¹[At]—).

In certain embodiments of a compound of Formula (1), the chemotherapeutic moiety is a moiety Formula (2a):

-A-NQ(-Z—C(R¹¹)₂—C(R¹¹)₂—R⁹)(—C(R¹¹)₂—C(R¹¹)₂—R⁹) (2a)

wherein,

A is selected from a bond (“—”), oxygen (—O—), sulfur (—S—), amino (—NR¹⁰—), methylene (—CH₂—), methyleneoxy (—CH₂—O—), oxycarbonyl (—O—C(═O)—), thiocarbonyl (—S—C(═O)—), aminocarbonyl (—NR¹⁰—C(═O)—), oxythiocarbonyl (—O—C(═S)—), thiothiocarbonyl (—S—C(═S)—), aminothiocarbonyl (—NR¹⁰—C(═S)—), methyleneoxycarbonyl (—CH₂—O—C(═O)—), methylenethiocarbonyl (—CH₂—S—C(═O)—), methyleneaminocarbonyl (—CH₂—NR¹⁰—C(═O)—), methyleneoxythiocarbonyl (—CH₂—O—C(═S)—), methylenethiothiocarbonyl (—CH₂—S—C(═S)—), methyleneaminothiocarbonyl (—CH₂—NR¹⁰—C(═S)—), carbonyl (—C(═O)—), methylencarbonyl (—CH₂—C(═O)—), thiocarbonyl (—C(═S)—), and methylenthiocarbonyl (—CH₂—C(═S)—);

Z is selected from a bond (“—”) and oxygen (—O—);

Q is selected from —O⁻ (a negatively charged oxygen atom) that is bound to a positively charged nitrogen atom) and a free electron pair (:), with the proviso that when Q is —O⁻ (a negatively charged oxygen atom that is bound to a positively charged nitrogen atom), A is selected from a bond (“—”) and methylene (—CH₂—), Z is a bond (“—”), and the chemotherapeutic moiety of Formula (2) is an N-oxide (-A-N⁺(—O⁻)(—C(R¹¹)₂—C(R¹¹)₂—R⁹)₂); and

each R¹¹ is independently selected from hydrogen, deuterio, and C₁₋₃ alkyl; and

each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

“Cycloalkyl” refers to a saturated or partially unsaturated cyclic alkyl radical. In certain embodiments, a cycloalkyl group is C₃₋₆ cycloalkyl, C₃₋₅ cycloalkyl, C₅₋₆ cycloalkyl, cyclopropyl, cyclopentyl, and in certain embodiments, cyclohexyl. In certain embodiments, cycloalkyl is selected from cyclopropyl, cyclobutyl, cyclopentyl, and cyclohexyl.

“Cycloalkylalkyl” refers to an acyclic alkyl radical in which one of the hydrogen atoms bonded to a carbon atom is replaced with a cycloalkyl group as defined herein. Where specific alkyl moieties are intended, the nomenclature cycloalkylalkyl, cycloalkylalkenyl, or cycloalkylalkynyl is used. In certain embodiments, a cycloalkylalkyl group is C₄₋₃₀ cycloalkylalkyl, e.g., the alkanyl, alkenyl, or alkynyl moiety of the cycloalkylalkyl group is C₁₋₁₀ and the cycloalkyl moiety of the cycloalkylalkyl moiety is C₃₋₂₀, and in certain embodiments, an cycloalkylalkyl group is C₄₋₂₀ cycloalkylalkyl, e.g., the alkanyl, alkenyl, or alkynyl moiety of the cycloalkylalkyl group is C₁₋₈ and the cycloalkyl moiety of the cycloalkylalkyl group is C₃₋₁₂. In certain embodiments, cycloalkylalkyl is C₄₋₉ cycloalkylalkyl, wherein the alkyl moiety of the cycloalkylalkyl group is C₁₋₃ alkyl, and the cycloalkyl moiety of the cycloalkylalkyl group is C₃₋₆ cycloalkyl. In certain embodiments, a cycloalkylalkyl group is C₄₋₁₂ cycloalkylalkyl, C₄₋₁₀ cycloalkylalkyl, C₄₋₈ cycloalkylalkyl, and C₄₋₆ cycloalkylalkyl. In certain embodiments a cycloalkylalkyl group is cyclopropylmethyl (—CH₂-cyclo-C₃H₅), cyclopentylmethyl (—CH₂-cyclo-C₅H₉), or cyclohexylmethyl (—CH₂-cyclo-C₆H₁₁). In certain embodiments a cycloalkylalkyl group is cyclopropylethenyl (—CH═CH-cyclo-C₃H₅), cyclopentylethynyl (—C≡C-cyclo-C₅H₉), or the like.

“Cycloalkylheteroalkyl” by itself or as part of another substituent refers to a heteroalkyl group in which one or more of the carbon atoms (and certain associated hydrogen atoms) of an alkyl group are independently replaced with the same or different heteroatomic group or groups and in which one of the hydrogen atoms bonded to a carbon atom is replaced with a cycloalkyl group. Where specific alkyl moieties are intended, the nomenclature cycloalkylheteroalkanyl, cycloalkylheteroalkenyl, and cycloalkylheteroalkynyl is used. In certain embodiments of cycloalkylheteroalkyl, the heteroatomic group is selected from —O—, —S—, —NH—, —N(—CH₃)—, —SO—, and —SO₂—, in certain embodiments, the heteroatomic group is selected from —O— and —NH—, and in certain embodiments the heteroatomic group is —O— or —NH—.

“Cycloalkyloxy” refers to a radical —OR where R is cycloalkyl as defined herein. Examples of cycloalkyloxy groups include cyclopropyloxy, cyclobutyloxy, cyclopentyloxy, and cyclohexyloxy. In certain embodiments, a cycloalkyloxy group is C₃₋₆ cycloalkyloxy, in certain embodiments, C₃₋₅ cycloalkyloxy, in certain embodiments, C₅₋₆ cycloalkyloxy, and in certain embodiments, cyclopropyloxy, cyclobutyloxy, cyclopentyloxy, or cyclohexyloxy.

“Disease” refers to a disease, disorder, condition, or symptom of any of the foregoing.

“Fluoroalkyl” refers to an alkyl group as defined herein in which one or more of the hydrogen atoms is replaced with a fluoro. In certain embodiments, a fluoroalkyl group is C₁₋₆ fluoroalkyl, C₁₋₅ fluoroalkyl, C₁₋₄ fluoroalkyl, and C₁₋₃ fluoroalkyl. In certain embodiments, the fluoroalkyl group is pentafluoroethyl (—CF₂CF₃), and in certain embodiments, trifluoromethyl (—CF₃).

“Fluoroalkoxy” refers to an alkoxy group as defined herein in which one or more of the hydrogen atoms is replaced with a fluoro. In certain embodiments, a fluoroalkoxy group is C₁₋₆ fluoroalkoxy, C₁₋₅ fluoroalkoxy, C₁₋₄ fluoroalkoxy C₁₋₃, or fluoroalkoxy, and in certain embodiments, —OCF₂CF₃ or —OCF₃.

“β-Substituted β-amino acid derivative” refers to β-substituted β-amino acid derivatives having a carboxyl group, e.g., β-substituted β-amino acid.

“β-Substituted β-amino acid analog” refers to β-substituted β-amino acid derivatives in which the carboxyl group is replaced with a phosphinic acid group, a sulfinic acid group, or others, e.g., 3-aminopropylphosphinic acids, 3-aminopropylsulfinic acids, and others.

“Halogen” refers to a fluoro, chloro, bromo, or iodo group.

“Heteroalkoxy” refers to an alkoxy group in which one or more of the carbon atoms are replaced with a heteroatom. In certain embodiments, the heteroalkoxy group is C₁₋₆ heteroalkoxy, in certain embodiments, C₁₋₅ heteroalkoxy, in certain embodiments, C₁₋₄ heteroalkoxy, and in certain embodiments, C₁₋₃ heteroalkoxy. In certain embodiments of heteroalkoxy, the heteroatomic group is selected from —O—, —S—, —NH—, —NR—, —SO₂—, and —SO₂—, in certain embodiments, the heteroatomic group is selected from —O— and —NH—, and in certain embodiments the heteroatomic group is —O— and —NH—. In certain embodiments, a heteroalkoxy group is C₁₋₆ heteroalkoxy, C₁₋₅ heteroalkoxy, C₁₋₄ heteroalkoxy, and in certain embodiments C₁₋₃ heteroalkoxy.

“Heteroalkyl” by itself or as part of another substituent refer to an alkyl group in which one or more of the carbon atoms (and certain associated hydrogen atoms) are independently replaced with the same or different heteroatomic group or groups. Examples of heteroatomic groups include —O—, —S—, —NH—, —NR—, —O—O—, —S—S—, ═N—N═, —N═N—, —N═N—NR—, —PR—, —P(O)OR—, —P(O)R—, —POR—, —SO—, —SO₂—, —Sn(R)₂—, and the like, where each R is independently selected from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, and substituted C₇₋₁₈ heteroarylalkyl. In certain embodiments, each R is independently selected from hydrogen and C₁₋₃ alkyl. Reference to, for example, a C₁₋₆ heteroalkyl, means a C₁₋₆ alkyl group in which at least one of the carbon atoms (and certain associated hydrogen atoms) is replaced with a heteroatom. For example, C₁₋₆ heteroalkyl includes groups having five carbon atoms and one heteroatom, groups having four carbon atoms and two heteroatoms, etc. In certain embodiments of heteroalkyl, the heteroatomic group is selected from —O—, —S—, —NH—, —N(—CH₃)—, —SO—, and —SO₂—, in certain embodiments, the heteroatomic group is selected from —O— and —NH—, and in certain embodiments, the heteroatomic group is —O— or —NH—. In certain embodiments, a heteroalkyl group is C₁₋₆ heteroalkyl, C₁₋₅ heteroalkyl, or C₁₋₄ heteroalkyl, and in certain embodiments, C₁₋₃ heteroalkyl.

“Heteroaryl” by itself or as part of another substituent refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which may be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. In certain embodiments, when the total number of N, S, and O atoms in the heteroaryl group exceeds one, the heteroatoms may or may not be adjacent to one another. In certain embodiments, the total number of heteroatoms in the heteroaryl group is not more than two. In certain embodiments of heteroaryl, the heteroatomic group is selected from —O—, —S—, —NH—, —N(—CH₃)—, —SO—, and —SO₂—, in certain embodiments, the heteroatomic group is selected from —O— and —NH—, and in certain embodiments the heteroatomic group is —O— or —NH—. In certain embodiments, a heteroaryl group is selected from C₅₋₁₀ heteroaryl, C₅₋₉ heteroaryl, C₅₋₈ heteroaryl, C₅₋₇ heteroaryl, C₅₋₆ heteroaryl, and in certain embodiments, is C₅ heteroaryl and C₆ heteroaryl.

Examples of heteroaryl groups include groups derived from acridine, arsindole, carbazole, α-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like. In certain embodiments, heteroaryl groups are those derived from thiophene, pyrrole, benzothiophene, benzofuran, indole, pyridine, quinoline, imidazole, oxazole, or pyrazine. For example, in certain embodiments, heteroaryl is C₅ heteroaryl and is selected from furyl, thienyl, pyrrolyl, imidazolyl, pyrazolyl, isothiazolyl, or isoxazolyl. In certain embodiments, heteroaryl is C₆ heteroaryl, and is selected from pyridinyl, pyrazinyl, pyrimidinyl, and pyridazinyl.

“Heteroarylalkyl” refers to an arylalkyl group in which one of the carbon atoms (and certain associated hydrogen atoms) is replaced with a heteroatom. In certain embodiments, a heteroarylalkyl group is C₆₋₁₆ heteroarylalkyl, C₆₋₁₄ heteroarylalkyl, C₆₋₁₂ heteroarylalkyl, C₆₋₁₀ heteroarylalkyl, C₆₋₈ heteroarylalkyl, or C₇ heteroarylalkyl, and in certain embodiments, C₆ heteroarylalkyl. In certain embodiments of heteroarylalkyl, the heteroatomic group is selected from —O—, —S—, —NH—, —N(—CH₃)—, —SO—, and —SO₂—, in certain embodiments, the heteroatomic group is selected from —O— and —NH—, and in certain embodiments the heteroatomic group is —O— or —NH—.

“Heterocycloalkyl” by itself or as part of another substituent refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system violates the Hückel-rule. Examples of heteroatoms to replace the carbon atom(s) include N, P, O, S, and Si. Examples of heterocycloalkyl groups include groups derived from epoxides, azirines, thiiranes, imidazolidine, morpholine, piperazine, piperidine, pyrazolidine, pyrrolidine, quinuclidine, and the like. In certain embodiments, heterocycloalkyl is C₅ heterocycloalkyl and is selected from pyrrolidinyl, tetrahydrofuranyl, tetrahydrothiophenyl, imidazolidinyl, oxazolidinyl, thiazolidinyl, doxolanyl, and dithiolanyl. In certain embodiments, heterocycloalkyl is C₆ heterocycloalkyl and is selected from piperidinyl, tetrahydropyranyl, piperizinyl, oxazinyl, dithianyl, and dioxanyl. In certain embodiments a heterocycloalkyl group is C₃₋₆ heterocycloalkyl, C₃₋₅ heterocycloalkyl, C₅₋₆ heterocycloalkyl, and in certain embodiments, C₅ heterocycloalkyl or C₆ heterocycloalkyl. In certain embodiments of heterocycloalkyl, the heteroatomic group is selected from —O—, —S—, —NH—, —N(—CH₃)—, —SO—, and —SO₂—, in certain embodiments, the heteroatomic group is selected from —O— and —NH—, and in certain embodiments the heteroatomic group is —O— or —NH—.

“Heterocycloalkylalkyl” refers to a cycloalkylalkyl group in which one or more carbon atoms (and certain associated hydrogen atoms) of the cycloalkyl ring are independently replaced with the same or different heteroatom. In certain embodiments, the heterocycloalkylalkyl is C₄₋₁₂ heterocycloalkylalkyl, C₄₋₁₀ heterocycloalkylalkyl, C₄₋₈ heterocycloalkylalkyl, C₄₋₆ heterocycloalkylalkyl, or C₆₋₇ heterocycloalkylalkyl, and in certain embodiments, C₆ heterocycloalkylalkyl or C₇ heterocycloalkylalkyl. In certain embodiments of heterocycloalkylalkyl, the heteroatomic group is selected from —O—, —S—, —NH—, —N(—CH₃)—, —SO—, and —SO₂—, in certain embodiments, the heteroatomic group is selected from —O— and —NH—, and in certain embodiments, the heteroatomic group is —O— or —NH—.

“Mesyl” refers to the group —OS(O)₂Me or —OMs.

“Parent aromatic ring system” refers to an unsaturated cyclic or polycyclic ring system having a cyclic conjugated π (pi) electron system with 4n+2 electrons (Hückel rule). Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom in such a way as to maintain the continuous π-electron system characteristic of aromatic systems and a number of π-electrons corresponding to the Hückel rule (4n+2). Examples of heteroatoms to replace the carbon atoms include N, P, O, S, and Si, etc. Specifically included within the definition of“parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like.

“Patient” refers to a mammal, for example, a human. The term “patient” is used interchangeably with “subject.”

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids and one or more protonable functional groups such as primary, secondary, or tertiary amines within the parent compound. Examples of inorganic acids include hydrochloric acid, hydrobromic acid, sulfuric acid, nitric acid, phosphoric acid, and the like. In certain embodiments the salts are formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. In certain embodiments, a salt is formed when one or more acidic protons present in the parent compound are replaced by a metal ion, e.g., an alkali metal ion, an alkaline earth ion, or an aluminum ion, or combinations thereof; or coordinates with an organic base such as ethanolamine, diethanolamine, triethanolamine, N-methylglucamine, and the like. In certain embodiments, a pharmaceutically acceptable salt is the hydrochloride salt. In certain embodiments, a pharmaceutically acceptable salt is the sodium salt. In certain embodiments wherein a compound has two or more ionizable groups, a pharmaceutically acceptable salt comprises one or more counterions, such as a bi-salt, for example, a dihydrochloride salt.

The term “pharmaceutically acceptable salt” includes hydrates and other solvates, as well as salts in crystalline or non-crystalline form. Where a particular pharmaceutically acceptable salt is disclosed, it is understood that the particular salt (e.g., a hydrochloride salt) is an example of a salt, and that other salts may be formed using techniques known to one of skill in the art. Additionally, one of skill in the art would be able to convert the pharmaceutically acceptable salt to the corresponding compound, free base and/or free acid, using techniques generally known in the art. See also: Stahl and Wermuth, C. G. (Editors), Handbook of Pharmaceutical Salts, Wiley-VCH, Weinheim, Germany, 2008.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to a compound of Formula (1) or a pharmaceutically acceptable salt thereof and at least one pharmaceutically acceptable vehicle, with which the compound of Formula (1) or a pharmaceutically acceptable salt thereof is administered to a patient. Pharmaceutically acceptable vehicles are known in the art.

“Solvate” refers to a molecular complex of a compound with one or more solvent molecules in a stoichiometric or non-stoichiometric amount. Such solvent molecules are those commonly used in the pharmaceutical arts, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

“Substituted” refers to a group in which one or more hydrogen atoms are independently replaced with the same or different substituent(s). In certain embodiments, each substituent is independently selected from halogen, —OH, —CN, —CF₃, —OCF₃, ═O, —NO₂, C₁₋₆ alkoxy, C₁₋₆ alkyl, —COOR, —NR₂, and —CONR₂; wherein each R is independently selected from hydrogen and C₁₋₆ alkyl. In certain embodiments, each substituent is independently selected from halogen, —NH₂, —OH, C₁₋₃ alkoxy, and C₁₋₃ alkyl, trifluoromethoxy, and trifluoromethyl. In certain embodiments, each substituent is independently selected from —OH, methyl, ethyl, trifluoromethyl, methoxy, ethoxy, and trifluoromethoxy. In certain embodiments, each substituent is selected from C₁₋₃ alkyl, ═O, C₁₋₃ alkyl, C₁₋₃ alkoxy, and phenyl. In certain embodiments, each substituent is selected from —OH, —NH₂, C₁₋₃ alkyl, and C₁₋₃ alkoxy.

“Treating” or “treatment” of a disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter or manifestation that may or may not be discernible to the patient. In certain embodiments, “treating” or “treatment” refers to delaying the onset of the disease or at least one or more symptoms thereof in a patient who may be exposed to or predisposed to a disease or disorder even though that patient does not yet experience or display symptoms of the disease.

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation. For LAT1-transported chemotherapeutic agents of Formula (1) a therapeutically effective amount can refer to an amount in a single dose or an amount as part of a treatment regimen that is effective in treating the targeted disease. For a cell cycle inhibitor, a therapeutically effective amount can be refer to an amount in a single dose or an amount as part of a treatment regimen that is effective in protecting a population of cells such as bone marrow cells, from adverse effects of a LAT1-transported chemotherapeutic agent.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art. For a cell cycle inhibitor, a therapeutically effective dose can represent an amount is a single administration or multiple administrations during a course of a treatment regimen that is effective in protecting or ameliorating the effects of a co-administered LAT1-transported chemotherapeutic agent.

“Triflyl” refers to the group —OS(O)₂CF₃ or —OTf.

Reference is now made in detail to certain embodiments of compounds, compositions, and methods. The disclosed embodiments are not intended to be limiting of the claims. To the contrary, the claims are intended to cover all alternatives, modifications, and equivalents.

Methods provided by the present disclosure are also directed to ameliorating or reducing adverse effects of chemotherapy associated with the administration of chemotherapeutic agents such as the LAT1-transported chemotherapeutic agents provided by the present disclosure. The methods include co-administering a cell cycle inhibitor that suppresses, interrupts, and/or arrests the proliferation of normal, healthy cells and that does not suppress, interrupt, and/or arrest; or minimally suppresses, interrupts or arrests the proliferation of diseased cells such as cancer cells that are the target of the chemotherapy.

Proliferative disorders that are treated with chemotherapy include cancerous and non-cancer diseases. To improve efficacy and increase the therapeutic index of the LAT1-transported chemotherapeutic agent it is desirable that the proliferative disorder not be suppressed by the cell cycle inhibitor. Preferably, administration of a selective cell cycle inhibitor does not compromise the efficacy of the LAT1-transported chemotherapeutic agent or arrest the cancer cells being treated by the chemotherapeutic agent. It is also desirable that the cell cycle inhibitor exert its protective effects transiently or reversibly such that after a period of time the arrested normal, healthy cell or cell population returns to normal activity. While the cell cycle of the normal, healthy cells is arrested, in the quiescent period these cells are not actively metabolizing and therefore less able to incorporate LAT1-transported chemotherapeutic agents that would otherwise damage the normal, healthy cells. When the reversible cell cycle inhibitor is metabolized over time, the protective effects are diminished and dissipate such that the normal, healthy cells resume normal activity. The timing of administering the cell cycle inhibitor is done so that the growth of normal, healthy cells is interrupted during the administration of the LAT1-transported chemotherapeutic agent and when the LAT1-transported chemotherapeutic agent is exerting its therapeutic effect on the target diseased tissue such as a cancer.

An objective of methods provided by the present disclosure also includes reducing or ameliorating long-term hematological toxicity associated with chemotherapy. Long-term hematological toxicity refers to hematological toxicity affecting a patient for a period lasting more than one or more weeks, months, or years following administration of a LAT1-transported chemotherapeutic agent. Long-term hematological toxicity can result in bone marrow disorders that can cause the ineffective production of blood cells (myelodysplasia) and/or lymphocytes (lymphopenia, the reduction in the number of circulating lymphocytes, such as B- and T-cells). Hematological toxicity can manifest, for example, as anemia, reduction in platelet count (thrombocytopenia) or reduction in white blood cell count (neutropenia). In some cases, myelodysplasia can result in the development of leukemia. Long-term toxicity related to LAT1-transported chemotherapeutic agents can also damage other self-renewing cells in a subject, in addition to hematological cells.

Methods provided by the present disclosure can include the administration of at least one LAT1-transported chemotherapeutic agent and at least one cell cycle inhibitor.

Administration of a cell cycle inhibitor in conjunction with a LAT1-transported chemotherapeutic agent can result in reduced anemia, reduced lymphopenia, reduced thrombocytopenia, or reduced neutropenia associated with treatment with the LAT1-transported chemotherapeutic agent in the absence of administration of the cell cycle inhibitor. Methods provided by the present disclosure also include methods of treating a cancer in a patient, comprising administering to the patient being treated for the cancer, a therapeutically effective amount of a LAT1-transported chemotherapeutic agent and a cell cycle inhibitor effective in protecting normal/healthy cells. A LAT1-transported chemotherapeutic agent can be any suitable LAT1-transported chemotherapeutic agent appropriate for treating a certain cancer. A cell cycle inhibitor can be any suitable compound that does not obviate the efficacy of the LAT1-transported chemotherapeutic agent for treating the cancer. The cell cycle inhibitor can inhibit the cell cycle of normal/healthy cells and thereby protecting the normal/healthy cells from adverse effects caused by the LAT1-transported chemotherapeutic agent. The normal/healthy cells can include bone marrow cells, and a suitable cell cycle inhibitor can include a myelosuppressor. A cell cycle inhibitor can be transient or reversible, meaning that the cell cycle inhibitor can interrupt or arrest the cell cycle but not kill the cell. After a period of time, a cell cycle inhibitor can be metabolized and the cell can resume normal function. By interrupting or arresting the cell cycle, normal/healthy cells are not actively metabolizing and there is a lesser opportunity for LAT1-transported chemotherapeutic agents to enter and kill normal/healthy cells. The adverse effects of the LAT1-transported chemotherapeutic agent can thereby be avoided or reduced.

In can also be desirable that normal, healthy cells arrested by the cell cycle inhibitor exhibit a rapid, synchronous reentry into the cell cycle following the cessation of the LAT1-transported chemotherapeutic damaging effect. The use of such cell cycle inhibitors can allow for an accelerated cell recovery, reduced cytotoxicity risk due to replication delay, and/or a minimization of LAT1-transported chemotherapeutic agent induced cell death.

Cell cycle inhibitors include compounds effective in blocking at least one stage in cell cycle proliferation. A cell cycle inhibitor can be transient and/or reversible such that the compound experts its therapeutic protective effect for a period of time after which the normal, healthy cells being protect return to normal biological activity.

The cell cycle is a highly conserved and regulated process by which genomic integrity and replicative capacity must be maintained for proper cell maintenance and proliferation. The cell cycle includes four distinct phases: the G1 phase where cells grow and synthesize proteins in preparation for DNA synthesis; the S phase, where DNA synthesis occurs; the G2 phase where cells continue to synthesize proteins to increase mass in preparation for mitosis; and the M phase in which the DNA divides and the parent cell undergoes cytokinesis to produce two daughter cells.

Regulation of the cell cycle is maintained by proteins referred to as cyclins and catalytic binding proteins, cyclin-dependent kinases (CDKs). At the G1 to S checkpoint cells are maintained in a quiescent state until triggered to reenter into the cell cycle. Throughout G1, expression of the D-type cyclins (D1, D2, D3) increases until active complexes with CDK4/6 are formed. Active CDK4/6 complexes partially phosphorylate RB, which allows partial depression of the transcription factor E2F. This induces additional transcript production including CCNE1. Cylcin E can bind CDK2 to form active complexes that result in the hyperphosphorylation of RB driving the cells through late G1 phase into the S phase. Inhibition of CDK4/6-cyclin D by the tumor suppressor CDKN2A leads to a G1 arrest and cell-cycle progression is halted.

Other targets for inhibition of the cell cycle include inhibitors affecting the G2-phase to M-phase transition including, for example, p53 inhibitors, Mdm2 antagonists, DNA-PK inhibitors, Bcr-Abl inhibitor, Pan-PlK inhibitors, and Pan-Aurora kinase inhibitors; inhibitors affection the G1-phase to S-phase transition such as pan-GSK-3 inhibitors, Pan-CDK inhibitors, Pan-TGF-beta/Smad inhibitors, c-Myc inhibitors, Pan-Akt inhibitors, Pan-HDAC inhibitor, Dual ATM/Atr inhibitors, and pan Chk inhibitors.

A cell cycle inhibitor can be effective in arresting the cell cycle of rapidly proliferating cells such as bone marrow, T-cells, and/or renal cells. A cell cycle inhibitor can transiently or reversibly arrest growth of these cells. By arresting growth, a cell cycle inhibitor can protect otherwise rapidly proliferating normal cells by reducing uptake of a LAT1-transported chemotherapeutic agent and/or affecting a target of a LAT1-transported chemotherapeutic agent. A cell cycle inhibitor can be selective such that the cell cycle inhibitor can arrest the growth of cells such as cancer cells that are the target of chemotherapy. For example, cell cycle inhibitors can selective arrest the growth of bone marrow cells and have a lesser effect on the growth of cancer cells. A cell cycle inhibitor can arrest the growth of cells transiently or reversibly in the sense that after a period of time in which the growth cycle of a targeted cell is interrupted, normal growth can resume.

A cell cycle inhibitor can be a myelosuppressor. A myelosuppressors is c compound aht causes myelosuppression or bone marrow suppression, which is a decrease in production of cells responsible for providing immunity (leukocytes), carrying oxygen (erythrocytes), and/or those responsible for normal blood clotting (thrombocytes). Myelosuppression encompasses anemia, neutropenia, and thrombocytemia.

LAT1-transported chemotherapeutic agents can interfere with a particular stage in the cell cycle. For example, compounds effective in interfering with the synthesis of DNA precursors in the G1 phase include methotrexate, azathioprine, 6-MP, 6-TG, and 5-FU. Compounds effective in interfering with DNA synthesis in the S phase include, for example, alkylating agents, antitumor antibiotics, and platinum compounds. Compounds effective in interfering with the synthesis of intracellular components for cell division in the G2 phase include, for example, vinca alkaloids such as vinblastine, vincristine, and vinorelbine, docetaxol, and paclitaxel.

Suitable cell cycle inhibitors include, for example, CDK 4/6 inhibitors, selective inhibitors of T-cell proliferation, myelosuppressors, mitotic inhibitors, checkpoint inhibitors, and immunosuppressors.

Examples of suitable cell cycle inhibitors include Pan-CDK inhibitors such as palbociclib, roscovitine, and dinaciclib; selective CDK inhibitors such as XL 413 (CDK7) and LDC000067 (CDKs); Pan-TGF betaSmad inhibitors such as LDN-193189, LDN-212854, and K02288; selective TGF-beta?Smad inhibitors such as DMH1 (ALK2) and SB431542 (ALK5); c-Myc inhibitors such as 1005B-F4; Pan-GSK-3 inhibitors such as CHIR-99021, SB216763, CHIR-98014; selective GSK-3 inhibitors such as TWB 112 (GSK-3β) and tideglusib (GSK-3β); pan-Akt inhibitors such as MK-2206, perifosine, and GSK690693; selective Akt inhibitors such as A-674563 (Akt1) and CCT128930 (Akt2); dual ATM/ATR inhibitors such as wortmannin and CGK 733; selective ATM/ATR inhibitors such as KU-55833 (ATM) and VE-821 (ATR); Pan-Chk inhibitors such as AZD7762; selective CHk inhibitors such as LY2603618 (Chk1), MK-8776 (Chk2)m, and CHIR-124 (Chk1); pan-HDAC inhibitors such as vorinostat, entinostat, and panobinostat; selective HDAC inhibitors such as RGFP966 (HDAC3), nexturastat A (HDAC6), and PCI-34041 (HDAC8); p⁵³ activators such as JNJ-26854165 and NSC 319728; p⁵³ inhibitors such as pifithrin-α and pifithrin-μ; Mdm2 antagonists such as nutlin-3, nutlin-3a, and YH239-EE; Mdm2 activators such as NSC 207895; DNA-PK inhibitors such as NU7441, NU7026, KU-006-648, and PIK-75; Bcr-inhibitors such as imatinib, ponatinib, nilotinib, bafetinib, and dasatinib; Pan-PLK inhibitors such as BI 2536; selective PLK inhibitors such as volasertib, rigosertib, and GSK461364; pag-Aurora kinase inhibitors such as VX-680, danusertib, and ZM 447439; selective Aurora kinase inhibitors such as alisertib, barasertib, and MK-5108.

Other suitable cell cycle inhibitors include, for example, cytochalasin D, flavopiridol, CX-4945, roscovitine, RO-3306, cycloheximide, tunicamycin, KN-93, apigenin, 10058-F4, etoposide, lovastatin, ceramide C6, daidzein, genistein, colcemid, vinblastine, A77-1726, PD173074, temozolomide, scriptaid, SU-9516, CCT128930, fluorouracil, monastrol, PI-103, SL 0101-1, BMS 195614, lipase inhibitor THL, nilotinib, Met kinase inhibitor, PPlase-parvulin inhibitor, ursolic acid, isoimperatorin, noscapine pifithrin-α, L-744,832 hydrochloride, DRB, tryphostin 9, romidepsin, chidamide, methotrexate-methyl-d3, CDK4 inhibitor, dabrafenib, diosgenin, phenethyl isothiocyanate, methotrexate dehydrate, AG 494, MRN-ATM pathway inhibitor, CDC25 phosphatase inhibitor, AZD 5438, CHK2 inhibitor, LY2603618, NSC 109555 ditosylate, olomucine, indirubin-3′-monoxime, telomerase inhibitor IX, NU 6140, AZD7762, epothiolone, 7-hydroxy methotrexate, EG5 inhibitor V, tryprostatin A, TWS 119 ditrifluoroacetate, Hec1/Nek2 mitotic pathway inhibitor I, T113242, catechin, retrorsine, indole-3-carbinol, IMD-0354, dexamethasone acetate, cytochalasin A, etodolac, CDK9 inhibitor II, neoxaline, terbinafine hydrochloride, ganciclovir, 5-fluorouracil-6-d1, kazusamycin A, calpain inhibitor I, vinorelbine ditartrate, AG 555, NU2058, PD 158780, aloisine A, SU9516, EGFR inhibitor III, CDK2/9 inhibitor, reveromycin A, tangeretin, echinosporin, terpendole E, tozasertib, L-4-fluoro-phenyl-alanine, SC58125, tyrphostin 47, RK-682, epothiolone B, malvidin chloride, bohemine, DMAP, elbfluorene, mycophenolates, and leflunomide.

A cell cycle inhibitor can comprise mycophenolate, leflunomide, methotrexate, or a combination of any of the foregoing.

Examples of suitable compounds that can cause bone marrow suppression include quinapril, adriamycin, methyl-dopa, ramipril, azathioprine, alemtuzumab, carbamazepine, ciprofloxin, sulindac, penicillamine, doxorubicin, asparaginase, cyclobenzaprine, methotrexate, ofloxacin, fluorometholone, indomethacin, lotrel, trandolapril, cefoxitin, desipramine, imipenem, cilastatin, lisinopril, mefenimide acetate, trimipramine maleate, tegretol, ticlopidine, toiramate, valganciclover, vaseretic, vasotec, voriconazole, and protriptyline.

Other suitable examples of drugs that can cause bone marrow suppression include, BCNU, etoposide, fluphenazine decanoate, teniposide, 5-azacytidine, 6-mercaptopurine, 6-thioguanine, EDTA, FAMP, allopurinol, amiodarone, amiodarone, amitriptyline, amsacrine, anthracycline, azathioprine, bexarotene, busulfan, candesartan cilexetil, carbamazepine, carbimazole, carboplatin, cefoxitin, chloramphenicol, cimetidine, dacarbazine, dicloxacillin, diethylpropion, dothiepin, doxepin, doxorubicin, eslicarbazepine acetate, famotidine, fludarabine, ganciclovir, gemfibrozil, hydroxy chloroquine, hydroxy urea, idarubicin, imatinib, imipramine, indomethacin, iodide, lamivudine, lenalidomide, lercanidipine, mafenide acetate, maprotiline, maraviroc, mefenamic acid, melphalan, methazolamide, methotrexate, methldopa, metronidazole, metyrapone, mianserin, mirtazapine, mitoxantrone, mycophenolate mofetil, nafcillin, nitrous oxide, nortriptyline, ofloxacin, olmesartan, oxacillin, protryptyline, ramipril, ranitidine, sulfasalazine, sulfindac, teniposide, ticlopridine, trimethoprim-sulfamethoxazole, trimipramine, valganciclovir, valproate, vincristine, vinorelbine, voriconazole, zidovudine, and zidovudine/lamivudine.

Suitable compounds that can cause myelosuppression, i.e., myelosuppressors, include, for example, gemcitabine, 5-fluoroambucil, 5-aza-2′-deoxyctidine, 6-mercaptopurine, 6-thioguanine, BCNU, FAMP, TR-7000, actinomycin D, amsacrine, anthracycline, azathioprine, bendamustine, bleomycin hydrochloride, bosutinib, busulfan, carboplatin, cisplatin, cladribine, cochicine, cyclophosphamide, cytarabine, dacarbazine, dasatinib, dexrazoxane, docetaxel, doxorubicin, estramustine, etoposide, fludarabine, glibenclamide, hexamethylmelamine, hydroxyurea, idarubicin, ifosgamide, imatinib, ixabepilone, lenalidomide, linezolid, lomustine, melphalan, mitomycin C, mitoxantrone, nilotinib, paclitaxel, ponatinib, ruxolitinib, streptozotocin, sunitinib, tamoxifen, temozolomide, teniposide, thiotepa, topotecan, vinblastine, vincristine, vinorelbine, and vorinostate.

A cell cycle inhibitor can comprise one or more cell cycle inhibitor such as one or more of any of the foregoing cell cycle inhibitors.

In certain embodiments, a cell cycle inhibitor can be a CDK 4/6 inhibitor. Cyclin-dependent kinases (CDKs) mediate cell cycle progression, regulating transition from the G1 to S phase and G2 to M phase. There are four proliferative CDKs: CDK1 which predominately regulates the transition from the G2 to M phase, and CDK2/4/6, which regulates the transition from the G1 to S phase. Certain cells require the activity of CDK4/6 for proliferation such as hematopoietic stem and progenitor cells and pancreatic beta cells.

Bone marrow hematopoietic stem and progenitor cells (HSPCs) are highly dependent upon CDK4/6 for proliferation. Pharmacological quiescence by CDK4/6 inhibition of the G1 to S transition protects hematopoietic stem cells from chemotherapy induced proliferation exhaustion. To use G1T28 to selectively protect the HSPC while not antagonizing the intended antitumor activity of the chemotherapy, the tumor can be CDK4/6 independent.

CDK4/6-replication dependent healthy cells can be a hematopoietic stem progenitor cell. Hematopoietic stem and progenitor cells include, but are not limited to, long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs). CDK4/6-replication dependent healthy cells may be a cell in a non-hematopoietic tissue, such as, for example, the liver, kidney, pancreas, brain, lung, adrenals, intestine, gut, stomach, skin, auditory system, bone, bladder, ovaries, uterus, testicles, gallbladder, thyroid, heart, pancreatic islets, and blood vessels. CDK4/6-replication dependent healthy cells can be renal cells, and in particular a renal epithelial cells, for example, renal proximal tubule epithelial cells. CDK4/6-replication dependent healthy cells can be hematopoietic stem progenitor cells. CDK4/6-replication dependent healthy cells may be cells in a non-hematopoietic tissue, such as, for example, the liver, kidney, pancreas, brain, lung, adrenals, intestine, gut, stomach, skin, auditory system, bone, bladder, ovaries, uterus, testicles, gallbladder, thyroid, heart, pancreatic islets, blood vessels, and the like.

The transient arrest of HSPCs by a CDK4/6 inhibitor during the administration of chemotherapy to treat CDK4/6 independent cancers can protect the bone marrow and immune system form the cytotoxic effects of the chemotherapy, while not interfering with the cytotoxicity of the chemotherapy. This can result in a faster recovery of circulating blood cells, prevention of bone marrow exhaustion and the preservation of immune cell number and function, thereby allowing a more robust host immune response to the tumor. An example of a suitable CDK4/6 inhibitor is G1T28 (Bisi et al., Mol Cancer Ther, 783-793, 15(5), May 2016).

In certain embodiments, a CD4/6 inhibitor can comprise palbociclib. Palbociclib is a cyclin dependent kinase CDK4/6 inhibitor that exhibits IC₅₀ in the low nanomolar range and induces a G1 cell cycle arrest and subsequent cytostasis. Palbociclib is approved by the FDA for treatment of estrogen receptor positive, human epidermal growth factor receptor 2(ER+HER2-) advanced breast cancer and is being investigated for treatment of retinoblastoma (Rb) proficient glioblastoma (GBM). However, the brain penetration of Palbociclib has been found to be restricted by P-g and BCRP efflux transporters in the BBB. Gooijer et al., Invest New Drugs 1012-1019, 33, 2015; Parrish et al., J. Pharmacol Exp Ther, 264-271, 355, November 2015.

In certain embodiments, a cell cycle inhibitor can be an immunosuppressant. Immunosuppressants are compounds that prevent or minimize the immune response. Example of suitable immunosuppressants include alefacept, sirolimus, efalizumab, mycophenolic acid, belimumab, fingolimod, vedolizumab, natalizumab, dimethyl fumarate, leflunomide, abatacept, everolilmus, teriflunomide, lymphocyte immune globulin, beletacept, muromonab-cd3, eculizumab, and anti-thymocyte globulin.

Other suitable immunosuppressants include, for example, include azathioprine, mycophenolate mofetil, cyclosporine, methotrexate, leflunomide, cyclophosphamide, chlorambucil, and nitrogen mustard.

Other suitable immunosuppressants that can be useful in inhibiting T-cell proliferation include, for example, corticosteroids such as prednisolone and methylprednisolone; calcineruin inhibitors such as cyclosporine, tarolimus, and sirolimus; inhibitors of nucleotide synthesis (purine synthesis IMDH inhibitors) such as mycophenolate acid, mizoribine, leflunomide, and azathioprine; biological agents such a polyclonal antibodies (antithymocyte globulins), murine monoclonal anti-CD3 antibody (muromonab-CD3), humanized monoclonal anti-CD52 antibody (alemtuzumab), monoclonal anti-CD25 antibody such as basilizimab and daclizumab; and anti-CD20 antibodies such as rituximab and LEA29Y.

In certain embodiments, a cell cycle inhibitor comprises mycophenolic acid, leflunomide, or a combination thereof. Mycophenolic acid (MPA) is an immunosuppressive agent and is indicated as prophylactic agent in patients receiving allogeneic renal, cardiac or hepatic transplants. IMPDH1 and IMPDH2 are the targets of MPA and are responsible for the suppression of lymphocyte proliferation. It is a noncompetitive, selective and reversible inhibitor of inosine monophosphate dehydrogenase (IMPDH1 and IMPDH2), which is an important rate-limiting enzyme involved in purine synthesis, which converts inosine monophosphate to guanosine monophosphate, which is necessary for the growth of T-cells and B-cells. Leflunomide is an izoxazole prodrug that is converted in the cytoplasm to an active compound, N-(4-trifluoromethylphenyl-2,2-cyano-3-hydroxycrotonamide). Leflunomide causes the accumulation of T cells in the late G1 phase of the cell cycle, which results in a blockade of T-cell proliferation.

In certain embodiments, a cell cycle inhibitor can comprise a checkpoint inhibitor. Immune check points refer to a group of inhibitor pathways for maintaining self-tolerance and modulating the duration and amplitude of physiological immune responses in peripheral tissues to minimize collateral tissue damage.

Checkpoint inhibitors can affect one of four areas of immune activation: DC presentation and T cell priming, T cell activation and anti-tumor effector functions, T cell differentiation into memory T cells and tumor microenvironment antagonism.

Immune responses against tumors occur in a step-wise manner. First, dendritic cells capture tumor antigens and present them to naïve T cells under inflammatory conditions. Naïve T cells then differentiate into effector T cells, which may take up to a week before leaving the lymph node and entering the blood. At this time, some T cells further differentiate into long-lived memory T cells, which provide a pool of renewable anti-tumor T cells for an extended period after immunotherapy has ceased. Once in the periphery, tumor cells activate T cells, causing them to secrete inflammatory cytokines and/or cytotoxic granules. Throughout this process, T cells must overcome tumor-derived immunosuppression from myeloid-derived suppressor cells, regulatory T cells, and tumor cell-secreted suppressive molecules. Drugs modulating each of these areas can be delivered before and during the steps of immune maturation.

Programmed cell death protein 1 (PD-1) is an immune-inhibitory receptor that belongs to the CD28 family and is expressed on T cells, B cells, monocytes, natural killer cells and tumor-infiltrating lymphocytes. PD-1 binds to two ligands that (PD-L1 and PD-L2) and activation leads to suppression of T-cell proliferation, cytokine production, and cell adhesion.

Certain tumors upregulate expression of PD-1 ligands. Pharmacological approach in influencing this pathway, by which tumors escape immune response can be overcome resistance to tumors and help tumor-specific T cells to carry other their cytotoxic functions.

Nivolumab is a fully human immunoglobulin G4(IgG4) monoclonal antibody that selectively inhibits PD-1 activity by binding to the PD-1 receptor to block the ligands PD-L1 and PD-L2 and thereby prevent tumor binding. The negative PD-1 receptor signaling that regulates T cell activation and proliferation is therefore disrupted by nivolumab binding. Pembrolizumab is a monoclonal antibody that also binds to the PD-1 receptor and blocks its interaction with ligands, PD-L1 and PD-L2, releasing PD-1 pathway-mediated inhibition of the immune response, including the antitumor immune response. Blocking PD-1 activity is believed to prevent inhibition of T cell immune surveillance of tumors and, in some models, has resulted in decreased tumor growth. Furthermore, by arresting T-cell proliferation, these check point inhibitors can protect T cells from chemotherapeutic toxicity.

Another pharmacological target to arrest T-cell proliferation the blockade of cytotoxic T-lymphocyte antigen-r (CTLA-4), which is upregulated early during the T-cell activation and expression of the CTLA-4 antigen can suppress T-cell activation and proliferation. An example of a suitable CTLA-4 inhibitor is pilimumab.

In certain embodiments, cell cycle inhibitor such as a myelosuppressor will be a poor substrate for the LAT1 transporter and/or will have a low affinity for the LAT1-transporter, compared to a LAT1-transported chemotherapeutic agent of Formula (1). In such embodiments, the cell cycle inhibitor will be less effective in interfering with the chemotherapeutic efficacy of the chemotherapeutic agent of Formula (1). A cell cycle inhibitor can have a relative uptake and/or affinity for rapidly proliferating cell populations such as bone marrow cells compared to the target cells for the chemotherapeutic agent. In this way, the cell cycle inhibitors can have exert a greater arresting effect on the cell population to be protected than on the diseased cells targeted by the chemotherapeutic agent.

The GenBank accession number for human LAT1/4F2hc is NP_003477/NP_002385. Unless otherwise apparent from the context, reference to a transporter such as LAT1/4F2hc (as well as other transporters disclosed herein) includes the amino acid sequence described in or encoded by the GenBank reference number, and, allelic, cognate and induced variants and fragments thereof retaining essentially the same transporter activity. Usually such variants show at least 90% sequence identity to the exemplary Genbank nucleic acid or amino acid sequence. Allelic variants at the DNA level are the result of genetic variation between individuals of the same species. Some allelic variants at the DNA level that cause substitution, deletion or insertion of amino acids in proteins encoded by the DNA result in corresponding allelic variation at the protein level. Cognate forms of a gene refer to variation between structurally and functionally related genes between species. For example, the human gene showing the greatest sequence identity and closest functional relationship to a mouse gene is the human cognate form of the mouse gene.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. The sequence comparison algorithm enables calculation of the percent sequence identity for the test sequence(s) relative to the reference sequence, based on the designated program parameters. Optimal alignment of sequences for comparison may be conducted by methods known to those skilled in the art.

In certain embodiments, anti-cancer agents provided by the present disclosure are compounds of Formula (1):

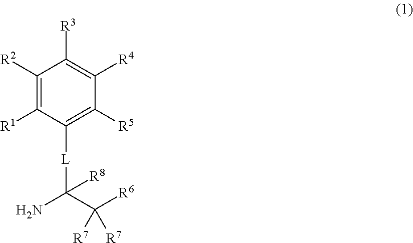

or a pharmaceutically acceptable salt thereof, wherein:

at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅₋₁₀ heteroaryl, C₆₋₁₆ heteroarylalkyl, and substituted C₆₋₁₆ heteroarylalkyl;

one of R¹, R², R³, R⁴, and R⁵ comprises a chemotherapeutic moiety;

each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;

R⁶ is selected from a carboxylic acid (—COOH), a carboxylic acid analog, and a carboxylic acid (bio)isostere;

each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;

R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;

each R¹⁰ is independently selected from hydrogen, C₁₋₄ alkyl and C₁₋₄ alkoxy, or two geminal R¹⁰ together with the nitrogen to which they are bonded form a 3- to 6-membered heterocyclic ring;

L is —(X)ₐ—, wherein,
each X is independently selected from a bond (“—”) and —C(R¹⁶)₂—, wherein each R¹⁶ is independently selected from hydrogen, deuterio, halogen, hydroxyl, C₁₋₄ alkyl, and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen, and C₁₋₄ alkyl; and
a is selected from 0, 1, 2, 3, and 4; and

each substituent is independently selected from halogen, —OH, —NH₂, —N(R¹⁰)₂, —NO₂, —CF₃, ═O (oxo), C₁₋₃ alkyl, C₁₋₃ alkoxy, and phenyl; wherein each R¹⁰ is independently selected from hydrogen and C₁₋₃ alkyl.

Anti-cancer agents of Formula (1) can also be referred to as chemotherapeutic agents of Formula (1), or LAT1-transported chemotherapeutic agents of Formula (1). Compounds of Formula (1) are β-substituted β-amino acid derivatives and β-substituted β-amino acid analogs.

In certain embodiments in compounds of Formula (1), R¹ comprises a chemotherapeutic moiety, R² comprises a chemotherapeutic moiety, R³ comprises a chemotherapeutic moiety, R⁴ comprises a chemotherapeutic moiety, and in certain embodiments, R⁵ comprises a chemotherapeutic moiety.

In certain embodiments of a compound of Formula (1), a chemotherapeutic moiety may be any suitable chemotherapeutic moiety of a chemotherapeutic drug known in the art that retains cytotoxic activity when bonded through a spacing moiety, e.g., an aryl ring and a linker L, to a β-amino acid derivative, β-amino acid analog, or β-amino acid carboxylic acid (bio)isostere as a LAT1 recognition element provided by the present disclosure. The conjugate or fusion product of the chemotherapeutic moiety with the β-amino acid derivative, β-amino acid analog, or β-amino acid carboxylic acid (bio)isostere is simultaneous a selective substrate for the LAT1/4F2hc transporter.

In certain embodiments of a compound of Formula (1), the chemotherapeutic moiety, comprises a nitrogen mustard —N(—CR₂—CR₂—X)₂, a N-monoalkyl or N,N-dialkyl triazene (—N═N—NR₂), a haloacetamide (—NR—CO—CH₂—X), an epoxide (—CROCR—R), an aziridine (—NC₂H₄), a Michael acceptor (—CR═CR-EWG-), a sulfonate or a bissulfonate ester (—OSO₂R or ROSO₂—), an N-nitrosourea (—NR—CO—N(NO)R), a bissulfonyl hydrazine (R″SO₂—NR—N(−)-SO₂R′″, —SO₂—NR—NR′—SO₂R′″, or R″SO₂—NR—NR′—SO₂—), a phosphoramidate (—O—P(═O)(N(R)—CH₂—CH₂—X)₂ or —O—P(═O)(N(—CH₂—CH₂—X)₂)₂, and a radionuclide such as, for example, 131-iodine (¹³¹[I]—) or 211-astatine (²¹¹[At]—).

In certain embodiments of a compound of Formula (1), the chemotherapeutic moiety is a moiety Formula (2):

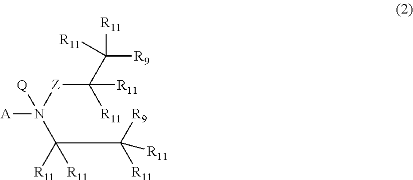

wherein,

A is selected from a bond (“—”), oxygen (—O—), sulfur (—S—), amino (—NR¹⁰—), methylene (—CH₂—), methyleneoxy (—CH₂—O—), oxycarbonyl (—O—C(═O)—), thiocarbonyl (—S—C(═O)—), aminocarbonyl (—NR¹⁰—C(═O)—), oxythiocarbonyl (—O—C(═S)—), thiothiocarbonyl (—S—C(═S)—), aminothiocarbonyl (—NR¹⁰—C(═S)—), methyleneoxycarbonyl (—CH₂—O—C(═O)—), methylenethiocarbonyl (—CH₂—S—C(═O)—), methyleneaminocarbonyl (—CH₂—NR¹⁰—C(═O)—), methyleneoxythiocarbonyl (—CH₂—O—C(═S)—), methylenethiothiocarbonyl (—CH₂—S—C(═S)—), methyleneaminothiocarbonyl (—CH₂—NR¹⁰—C(═S)—), carbonyl (—C(═O)—), methylencarbonyl (—CH₂—C(═O)—), thiocarbonyl (—C(═S)—), and methylenthiocarbonyl (—CH₂—C(═S)—);

Z is selected from a bond (“—”) and oxygen (—O—);

Q is selected from —O⁻ (a negatively charged oxygen atom) that is bound to a positively charged nitrogen atom) and a free electron pair (:), with the proviso that when Q is —O⁻ (a negatively charged oxygen atom that is bound to a positively charged nitrogen atom), A is selected from a bond (“—”) and methylene (—CH₂—), Z is a bond (“—”), and the chemotherapeutic moiety of Formula (2) is an N-oxide (-A-N⁺(—O⁻)(—C(R¹¹)₂—C(R¹¹)₂—R⁹)₂);

each R¹¹ is independently selected from hydrogen, deuterio, and C₁₋₃ alkyl; and

each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

In certain embodiments, a chemotherapeutic moiety of Formula (2) is selected from the structure -A-N(—Z—C(R¹¹)₂—C(R¹¹)₂—R⁹)(—C(R¹¹)₂—C(R¹¹)₂—R⁹ and (-A-N⁺(—O⁻)(—C(R¹¹)₂—C(R¹¹)₂—R⁹)₂, wherein,

A is selected from a bond (“—”), methylene (—CH₂—), oxygen (—O—), methyleneoxy (—CH₂—O—), oxycarbonyl (—O—C(═O)—), methyleneoxycarbonyl (—CH₂—O—C(═O)—), carbonyl (—C(═O)—), and methylenecarbonyl (—CH₂—C(═O)—);

each R¹¹ is independently selected from hydrogen and deuterio; and

each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹, wherein,

A is a bond (“—”);

Q is a free electron pair (:);

Z is a bond (“—”);

each R¹¹ is independently selected from hydrogen and deuterio; and

each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —N(—CH₂₋ₘDₘ-CH₂₋ₙDₙ-R⁹)₂, wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein,

A is methylene (—CH₂—);

Q is a free electron pair (:);

Z is a bond (“—”);

each R¹¹ is independently selected from hydrogen and deuterio; and

each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —CH₂—N(—CH₂₋ₘDₘ-CH₂₋ₙDₙ-R⁹)₂, wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is a bond (“—”), Q is a negatively charged oxygen (—O⁻), Z is a bond (“—”), each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —N⁺(—O⁻)(—CH₂₋ₘDₘ-CH₂₋ₙDₙ-R⁹)₂, wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is methylene (—CH₂—), Q is a negatively charged oxygen (—O⁻), Z is a bond (“—”), each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —CH₂—N⁺(—O⁻)(—CH₂₋ₘDₘ-CH₂₋ₙDₙ-R⁹)₂, wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is a bond (“—”), Q is a free electron pair (:), Z is oxygen, each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —N(—O—CH₂₋ₘDₘ-CH₂₋ₙDₙ-R⁹)(—CH₂₋ₘDₘ-CH₂₋ₙDₙ-R⁹), wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is methylene (—CH₂—), Q is a free electron pair (:), Z is oxygen, each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —CH₂—N(—O—CH₂₋ₘDₘ-CH₂₋ₙDₙ-R⁹)(—CH₂₋ₘDₘ-CH₂₋ₙDₙ-R⁹), wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is oxygen (—O—), Q is a free electron pair (:), Z is a bond (“—”), each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —O—N(—CH₂₋ₘDₘ-CH₂₋ₙDₙ-R⁹)₂, wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is methyleneoxy (—CH₂—O—), Q is a free electron pair (:), Z is a bond (“—”), each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from and C₁₋₄ (per)fluoroalkyl) and the chemotherapeutic moiety is —CH₂—O—N(—CH₂₋ₘDₘ-CH₂₋ₙDₙ-R⁹)₂, wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is a carbonyl (—CO—), Q is a free electron pair (:), Z is a bond (“—”), each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —CO—N(—CH₂₋ₘDₘ-CH₂₋ₙDₙ-R⁹)₂, wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is methylenecarbonyl (—CH₂—CO—), Q is a free electron pair (:), Z is a bond (“—”), each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —CH₂—CO—N(—CH₂₋ₘDₘ-CH₂₋ₙDₙ-R⁹)₂, wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is oxycarbonyl (—O—CO—), Q is a free electron pair (:), Z is a bond (“—”), each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —O—CO—N(—CH₂₋ₘDₘ-CH₂₋ₙDₙ-R⁹)₂, wherein m and n are independently selected from 0, 1, and 2.

In certain embodiments, a chemotherapeutic moiety of Formula (2) has the structure -A-NQ(-Z—C(R¹¹)₂—C(R¹¹)₂—R⁹)(—C(R¹¹)₂—C(R¹¹)₂—R⁹), wherein A is a methyleneoxycarbonyl (—CH₂—O—CO—), each R¹¹ is independently selected from hydrogen and deuterio; and each R⁹ is independently selected from chloro (—Cl), bromo (—Br), iodo (—I), alkyl sulfonate (—OSO₂R⁴⁰, wherein R⁴⁰ is selected from C₁₋₄ alkyl), and C₁₋₄ (per)fluoroalklyl sulfonate (—OSO₂R⁴⁰, wherein R⁴⁰ is selected from C₁₋₄ (per)fluoroalkyl) and the chemotherapeutic moiety is —CH₂—O—CO—N(—CH₂₋ₘDₘ-CH₂₋ₙDₙ-R⁹)₂, wherein m and n are independently selected from 0, 1, and 2.

In certain embodiments of a compound of Formula (1), the chemotherapeutic moiety comprises —N(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—N(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —N⁺(—O⁻)(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—N⁺(—O⁻)(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —N(—O—CH₂₋ₘDₘ-CH₂₋ₙDₙ-R⁹)(—CH₂₋ₘDₘ-CH₂₋ₙDₙ-R⁹), wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—N(—O—CH₂₋ₘDₘ-CH₂₋ₙDₙ-R⁹)(—CH₂₋ₘDₘ-CH₂₋ₙDₙ-R⁹), wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —O—N(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—O—N(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CO—N(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—CO—N(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —O—CO—N(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—O—CO—N(—CH₂₋ₘDₘ-CH₂₋ₙDₙ-R⁹)₂, wherein m and n are independently selected from 0, 1, and 2, and each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —N(—CH₂—CH₂—R⁹)₂, wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—N(—CH₂—CH₂—R⁹)₂, wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —N⁺(—O⁻)(—CH₂—CH₂—R⁹)₂, wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—N⁺(—O⁻)(—CH₂—CH₂—R⁹)₂, wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —N(—O—CH₂—CH₂—R⁹)(—CH₂—CH₂—R⁹), wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—N(—O—CH₂—CH₂—R⁹)(—CH₂—CH₂—R⁹), wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —O—N(—CH₂—CH₂—R⁹)₂, wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—O—N(—CH₂—CH₂—R⁹)₂, wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CO—N(—CH₂—CH₂—R⁹)₂, wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—CO—N(—CH₂—CH₂—R⁹)₂, wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —O—CO—N(—CH₂—CH₂—R⁹)₂, wherein m and n are independently selected from 0, 1, and 2, and wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), the chemotherapeutic moiety comprises —CH₂—O—CO—N(—CH₂—CH₂—R⁹)₂, wherein each R⁹ is independently selected from chloro (—Cl), bromo (—Br), iodo (—I), methylsulfonyloxy (—OSO₂CH₃), and trifluoromethylsulfonyloxy (—OSO₂CF₃).

In certain embodiments of a compound of Formula (1), R⁶ is selected from carboxylic acid (—COOH), hydroxamic acids (—CONR¹²OH), boronic acids (—B(OH)(OR¹²), phosphinic acids or derivatives thereof (—PO(OH)R¹²), and phosphonic acid or derivatives thereof (—PO(OH)(OR¹²)), sulfinic acid (—SOOH), sulfonic acid (—SO₂OH), sulfonamide (—SO₂NHR¹² or —NHSO₂R¹²), sulfonimide or acyl sulfonimide (—SO₂NHCOR¹² or —CONHSO₂R¹²), sulfonylureas (—SO₂NHCONHR¹² or —NHCONHSO₂R¹²), amide (—CONHR¹² or —NHCOR¹²), acylcyanamide (—CONHCN), 2,2,2-trifluoroethan-1-ols (—CH(CF₃)OH), 2,2,2-trifluoromethyl ketones and hydrates thereof (—COCF₃ and —C(OH)₂CF₃), acidic heterocycles and annular tautomers of any of the foregoing, and acidic oxocarbocycles or cyclic polyones and resonance forms of any of the foregoing; wherein R¹² is selected from hydrogen, C₁₋₆ alkyl, C₁₋₄ fluoroalkyl, C₃₋₆ cycloalkyl, and C₆₋₁₀ aryl.

In certain embodiments of a compound of Formula (1), the acidic heterocycle and annular tautomers is selected from 1H-tetrazole, 5-oxo-1,2,4-oxadiazole, 5-oxo-1,2,4-thiadiazole, 5-thioxo-1,2,4-oxadiazole, thiazolidinedione, oxazolidinedione, oxadiazolidinedione, 3-hydroxyisoxazole, 3-hydroxyisothiazole, 1-hydroxy-imidazole, 1-hydroxy-pyrazole, 1-hydroxy-triazole, 1H-imidazol-2-ol, tetrazole-5-thiol, 3-hydroxyquinolin-2-one, 4-hydroxyquinolin-2-ones, tetronic acid, tetramic acid, mercaptoazoles such as sulfanyl-1H-imidazole, sulfinyl-1H-imidazole, sulfonyl-1H-imidazole, sulfanyl-1H-triazole, sulfinyl-1H-triazole, sulfonyl-1H-triazole, sulfanyl-1H-1,2,4-triazole, sulfinyl-1H-1,2,4-triazole, sulfonyl-1H-1,2,4-triazole, sulfanyl-1,4-dihydro-1,2,4-triazol-5-one, sulfinyl-1,4-dihydro-1,2,4-triazol-5-one, sulfonyl-1,4-dihydro-1,2,4-triazol-5-one, sulfanyl 1H-tetrazole, sulfanyl 2H-tetrazole, sulfinyl 1H-tetrazole, sulfinyl 2H-tetrazole, sulfonyl 1H-tetrazole, sulfonyl 2H-tetrazole, and sulfonimidamide.

In certain embodiments of a compound of Formula (1), the acidic oxocarbocycle or cyclic polyone and resonance forms is selected from cyclopentane-1,3-dione, squaric acid, squareamide, mixed squaramate, and 2,6-difluorophenol.

In certain embodiments of a compound of Formula (1), R⁶ is selected from —COOH, —S(O)OH, —SO₂OH, —P(O)(OH)R¹², —P(O)(OH)(OR¹²), —SO₂NHR¹², —NHSO₂R¹², SO₂NHCOR¹², —CONHSO₂R¹², —SO₂NHCONHR¹², —CONHCN, 1H-tetrazol-yl, 5-oxo-1,2,4-oxadiazole, 5-oxo-1,2,4-thiadiazole, 5-thioxo-1,2,4-oxadiazole, thiazolidinedione, oxazolidinedione, oxadiazolidinedione, 3-hydroxyisoxazole, 3-hydroxyisothiazole, cyclopentane-1,3-dione, squaric acid, squareamide, and mixed squaramate; wherein R¹² is selected from hydrogen, C₁₋₄ alkyl, and C₃₋₅ cycloalkyl.

In certain embodiments of a compound of Formula (1), R⁶ is selected from —COOH, —S(O)OH, —P(O)(OH)H, —CONHSO₂CH₃, —CONHSO₂CF₃, —SO₂NHCOCH₃, —SO₂NHCOCF₃, —NHSO₂CH₃, —NHSO₂CF₃, 1H-tetrazol-yl, 5-oxo-1,2,4-oxadiazole-yl, 5-oxo-1,2,4-thiadiazole-yl, 5-thioxo-1,2,4-oxadiazole-yl, thiazolidinedione-yl, oxazolidinedione-yl, oxadiazolidinedione-yl, 3-hydroxyisoxazole-yl, 3-hydroxyisothiazole-yl, tetronic acid-yl, tetramic acid-yl, and cyclopentane-1,3-dione-yl.

In certain embodiments of a compound of Formula (1), R⁶ is selected from —COOH, —S(O)OH, —P(O)(OH)H, —CONHSO₂CH₃, —CONHSO₂CF₃, —SO₂NHCOCH₃, —SO₂NHCOCH₃, —SO₂NHCOCF₃, —NHSO₂CF₃, —NHSO₂CF₃, and 1H-tetrazol-5-yl.

In certain embodiments of a compound of Formula (1), R⁶ is selected from —COOH, —S(O)OH, —P(O)(OH)H, and 1H-tetrazol-yl.

In certain embodiments of a compound of Formula (1), R⁶ is —COOH.

In certain embodiments of a compound of Formula (1), each R⁷ is independently selected from hydrogen, deuterio, halogen, hydroxyl, and C₁₋₄ alkyl, or two germinal R⁷ together with the carbon atom to which they are bonded form a C₃₋₅ cycloalkyl ring.

In certain embodiments of a compound of Formula (1), each R⁷ is independently selected from hydrogen, deuterio, fluoro, hydroxyl, methyl, ethyl, propyl, isopropyl, butyl, isobutyl, and tert-butyl, or two germinal R⁷ together with the carbon atom to which they are bonded form a cyclopropyl ring or a cyclobutyl ring.

In certain embodiments of a compound of Formula (1), each R⁷ is independently selected from hydrogen, deuterio, fluoro, hydroxyl, and methyl.

In certain embodiments of a compound of Formula (1), each R⁷ is independently selected from hydrogen and deuterio.

In certain embodiments of a compound of Formula (1), each R⁷ is hydrogen.

In certain embodiments of a compound of Formula (1), R⁸ is selected from hydrogen, deuterio, halogen, hydroxyl, C₁₋₄ alkyl, C₁₋₄ alkoxy, C₁₋₄ fluoroalkyl, C₁₋₄ fluoroalkoxy, and cyclopropyl.

In certain embodiments of a compound of Formula (1), R⁸ is selected from hydrogen, deuterio, halogen, hydroxyl, methyl, ethyl, propyl, isopropyl, butyl, isobutyl, tert-butyl, trifluoromethyl, methoxy, ethoxy, isopropoxy, trifluoromethoxy, and cyclopropyl.

In certain embodiments of a compound of Formula (1), R⁸ is selected from hydrogen, methyl, ethyl, propyl, isopropyl, cyclopropyl, tert-butyl, hydroxyl, methoxy, ethoxy, isopropoxy, trifluoromethyl, and trifluoromethoxy.

In certain embodiments of a compound of Formula (1), R⁸ is methyl.

In certain embodiments of a compound of Formula (1), R⁸ is hydrogen.

In certain embodiments of a compound of Formula (1), each R¹⁰ is independently selected from hydrogen and C₁₋₄ alkyl, or two R¹⁰ together with the nitrogen atom to which they are bonded form a 3- to 5-membered heterocycle.

In certain embodiments of a compound of Formula (1), L is (—X—)ₐ wherein a is selected from 0, 1, 2, 3, and 4, and X is selected from oxygen (—O—), sulfur (—S—), sulfinyl (—SO—), sulfonyl (—SO₂—), carbonyl (—CO—), —C(R¹⁶)₂— wherein R¹⁶ is independently selected from hydrogen, deuterio, halogen, hydroxyl, and C₁₋₄ alkyl, and amino (—NR¹⁷—), wherein R¹⁷ is selected from hydrogen, methyl, and ethyl.

In certain embodiments of a compound of Formula (1), comprises is selected from a bond (“—”), methylene (—CH₂—), fluoromethylene (—CFH—), difluoromethylene (—CF₂—), hydroxymethylene (—C(OH)H—), ethane-1,1-diyl (—CHCH₃—), propane-2,2-diyl (—C(CH₃)₂—), propane-1,1-diyl (—CH(CH₂—CH₃)—), oxygen (—O—), sulfur (—S—), sulfinyl (—SO—), sulfonyl (—SO₂—), carbonyl (—CO—), and amino (—NR¹⁷—), wherein R¹⁷ is selected from hydrogen, methyl, and ethyl.

In certain embodiments of a compound of Formula (1), comprises is selected from a bond (“—”), methylene (—CH₂—), fluoromethylene (—CFH—), difluoromethylene (—CF₂—), hydroxymethylene (—C(OH)H—), ethane-1,1-diyl (—CHCH₃—), propane-2,2-diyl (—C(CH₃)₂—), oxygen (—O—), sulfonyl (—SO₂—), carbonyl (—CO—), and amino (—NR¹⁷—), wherein R¹⁷ is selected from hydrogen and methyl.

In certain embodiments of a compound of Formula (1), a is 2 and each X is methylene (—CH₂—) and L is ethane-1,2-diyl (—CH₂—CH₂—); one X is methylene (—CH₂—) and one X is ethane-1,1-diyl (—CHCH₃—) and L is propane-1,2-diyl (—CH₂—CHCH₃—); one X is ethane-1,1-diyl (—CHCH₃—) and one X is methylene (—CH₂—) and L is propane-1,2-diyl (—CHCH₃—CH₂—); one X is methylene (—CH₂—) and one X is hydroxymethylene (—CHOH—) and L is hydroxyethane-1,2-diyl (—CH₂—CHOH—); one X is hydroxymethylene (—CHOH—) and one X is methylene (—CH₂—) and L is hydroxyethane-1,2-diyl (—CHOH—CH₂—); one X is methylene (—CH₂—) and one X is fluoromethylene (—CFH—), and L is fluoroethane-1,2-diyl (—CH₂—CHF—); one X is fluoromethylene (—CFH—) and one X is methylene (—CH₂—) and L is fluoroethane-1,2-diyl (—CHF—CH₂—); one X is methylene (—CH₂—) and one X is difluoromethylene (—CF₂—), and L is difluoroethane-1,2-diyl (—CH₂—CF₂—); one X is difluoromethylene (—CF₂—) and one X is methylene (—CH₂—) and L is difluoroethane-1,2-diyl (—CF₂—CH₂—); one X is carbonyl (—CO—) and one X is amino (—NR¹⁷—) and L is carbonyl amino (—CO—NR¹⁷—); one X is amino (—NR¹⁷—) and one X is carbonyl (—CO—) and L is amino carbonyl (—NR¹⁷—CO—); one X is methylene (—CH₂—) and one X is amino (—NR¹⁷—) and L is methyleneamino (—CH₂—NR¹⁷—); one X is amino (—NR¹⁷—) and one X is methylene (—CH₂—) and L is aminomethylene (—NR¹⁷—CH₂—); one X is methylene (—CH₂—) and one X is oxygen (—O—) and L is methyleneoxy (—CH₂—O—); one X is oxygen (—O—) and one X is methylene (—CH₂—) and L is oxymethylene (—O—CH₂—); one X is methylene (—CH₂—) and one X is sulfur (—S—) and L is methylenethiyl (—CH₂—S—); one X is sulfur (—S—) and one X is methylene (—CH₂—) and L is thiylmethylene (—S—CH₂—); one X is methylene (—CH₂—) and one X is sulfinyl (—SO—) and L is methylenesulfinyl (—CH₂—SO—); one X is sulfinyl (—SO—) and one X is methylene (—CH₂—) and L is sulfinylmethylene (—SO—CH₂—); one X is methylene (—CH₂—) and one X is sulfonyl (—SO₂—) and L is methylenesulfonyl (—CH₂—SO₂—); one X is sulfonyl (—SO₂—) and one X is methylene (—CH₂—) and L is sulfonylmethylene (—SO₂—CH₂—); one X is methylene (—CH₂—) and one X is carbonyl (—CO—) and L is methylenecarbonyl (—CH₂—CO—); or one X is carbonyl (—CO—) and one X is methylene (—CH₂—) and L is carbonylmethylene (—CO—CH₂—); wherein R¹⁷ is selected from hydrogen, methyl, and ethyl.

In certain embodiments of a compound of Formula (1), a is 2 and L is selected from ethane-1,2-diyl (—CH₂—CH₂—), propane-1,2-diyl (—CH₂—CHCH₃— or —CHCH₃— CH₂—), hydroxyethane-1,2-diyl (—CH₂—CHOH— or —CHOH—CH₂—), carbonyl amino (—CO—NR¹⁷—), amino carbonyl (—NR¹⁷—CO—), methyleneamino (—CH₂—NR¹⁷—), aminomethylene (—NR¹⁷—CH₂—), methyleneoxy (—CH₂—O—), oxymethylen (—O—CH₂—), methylenethiyl (—CH₂—S—), thiylmethylene (—S—CH₂—), methylenesulfonyl (—CH₂—SO₂—), sulfonylmethylene (—SO₂—CH₂—), methylenecarbonyl (—CH₂—CO—), and carbonylmethylene (—CO—CH₂—), wherein R¹⁷ is selected from hydrogen and methyl.

In certain embodiments of a compound of Formula (1),

at least one of R¹ and R⁵ is independently selected from, halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, substituted C₁₋₄ alkyl, C₁₋₄ alkoxy, substituted C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

one of R¹, R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

In certain embodiments of a compound of Formula (1),

at least one of R¹ and R⁵ is independently selected from halogen, —N(R¹⁰)₂, —NR¹⁰(OR¹⁰), —NO₂, —NO, —OH, C₁₋₄ alkyl, C₁₋₄ alkoxy, C₁₋₄ alkylsulfanyl, C₁₋₄ alkylsulfinyl, C₁₋₄ fluoroalkyl, C₁₋₄ fluoroalkoxy, C₃₋₅ cycloalkyl, and C₃₋₅ cycloalkyloxy;

each R¹⁰ is independently selected from hydrogen and C₁₋₃ alkyl, or two R¹⁰ together with the nitrogen to which they are bonded form a 3- to 5-membered heterocyclic ring; and one of R¹, R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

In certain embodiments of a compound of Formula (1),

each of R¹ and R⁵ is independently selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

one of R², R³, and R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

In certain embodiments of a compound of Formula (1),

each of R¹ and R⁵ is independently selected from halogen, —N(R¹⁰)₂, —NR¹⁰(OR¹⁰), —NO₂, —NO, —OH, C₁₋₄ alkyl, C₁₋₄ alkoxy, C₁₋₄ alkylsulfanyl, C₁₋₄ alkylsulfinyl, C₁₋₄ fluoroalkyl, C₁₋₄ fluoroalkoxy, C₃₋₅ cycloalkyl, and C₃₋₅ cycloalkyloxy;

each R¹⁰ is independently selected from hydrogen and C₁₋₃ alkyl, or two R¹⁰ together with the nitrogen to which they are bonded form a 3- to 5-membered heterocyclic ring; and

one of R², R³, and R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

In certain embodiments of a compound of Formula (1),

one of R¹ and R⁵ is independently selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

one of R¹, R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

In certain embodiments of a compound of Formula (1),

one of R¹ and R⁵ is independently selected from halogen, —N(R¹⁰)₂, —NR¹⁰(OR¹⁰), —NO₂, —NO, —OH, C₁₋₄ alkyl, C₁₋₄ alkoxy, C₁₋₄ alkylsulfanyl, C₁₋₄ alkylsulfinyl, C₁₋₄ fluoroalkyl, C₁₋₄ fluoroalkoxy, C₃₋₅ cycloalkyl, and C₃₋₅ cycloalkyloxy;

each R¹⁰ is independently selected from hydrogen and C₁₋₃ alkyl, or two R¹⁰ together with the nitrogen to which they are bonded form a 3- to 5-membered heterocyclic ring; and

one of R¹, R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

In certain embodiments of a compound of Formula (1),

each of the other of R¹, R², R³, R⁴, and R⁵ is independently is selected from hydrogen, deuterio, halogen, —N(R¹⁰)₂, —N(R¹⁰)(OR¹⁰), —NO₂, —NO, —OH, —COOR¹⁰, —CON(R¹⁰)₂, —OH, C₁₋₄ alkylsulfanyl, C₁₋₄ alkylsulfinyl, C₁₋₄ alkylsulfonyl, C₁₋₄ fluoroalkyl, C₁₋₄ fluoroalkoxy, C₁₋₄ alkyl, C₁₋₄ alkoxy, C₁₋₄ heteroalkyl, C₁₋₄ heteroalkoxy, and C₄₋₈ cycloalkylalkyl; and

each R¹⁰ is independently selected from hydrogen and C₁₋₄ alkyl, or two R¹⁰ together with the nitrogen to which they are bonded form a 3- to 6-membered heterocyclic ring.

In certain embodiments of a compound of Formula (1),

each of the other of R¹, R², R³, R⁴, and R⁵ is independently selected from hydrogen, deuterio, halogen, —NR¹⁰₂, —N(R¹⁰)(OR¹⁰), —OH, C₁₋₄ alkyl, C₁₋₄ alkoxy, C₁₋₄ alkylsulfanyl, C₁₋₄ fluoroalkyl, and C₁₋₄ fluoroalkoxy, and

each R¹⁰ is independently selected from hydrogen and C₁₋₄ alkyl, or two R¹⁰ together with the nitrogen to which they are bonded form a 3- to 5-membered heterocyclic ring.

In certain embodiments of a compound of Formula (1), the other of R¹ and R⁵ is hydrogen.

In certain embodiments of a compound of Formula (1), each of the other of R¹, R², R³, R⁴, and R⁵ is hydrogen.

In certain embodiments of a compound of Formula (1), R², R³, and R⁵ is hydrogen.

In certain embodiments of a compound of Formula (1),

R¹ is selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅₋₁₀ heteroaryl, C₆₋₁₆ heteroarylalkyl, and substituted C₆₋₁₆ heteroarylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring; and

R⁵ is hydrogen.

In certain embodiments of a compound of Formula (1),

R¹ selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring; and

R⁵ is hydrogen.

In certain embodiments of a compound of Formula (1),

R¹ is selected from halogen, —N(R¹⁰)₂, —NR¹⁰(OR¹⁰), —NO₂, —NO, —OH, C₁₋₄ alkyl, C₁₋₄ alkoxy, C₁₋₄ alkylsulfanyl, C₁₋₄ alkylsulfinyl, C₁₋₄ fluoroalkyl, C₁₋₄ fluoroalkoxy, C₃₋₅ cycloalkyl, and C₃₋₅ cycloalkyloxy; wherein each R¹⁰ is independently selected from hydrogen and C₁₋₃ alkyl, or two R¹⁰ together with the nitrogen to which they are bonded form a 3- to 5-membered heterocyclic ring; and

R⁵ is hydrogen.

In certain embodiments of a compound of Formula (1),

each of R¹ and R⁵ is independently selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring;

one of R², R³, and R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R², R³, and R⁴ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

In certain embodiments of a compound of Formula (1),

each of R¹ and R⁵ is independently selected from halogen, —N(R¹⁰)₂, —NR¹⁰(OR¹⁰), —NO₂, —NO, —OH, C₁₋₄ alkyl, C₁₋₄ alkoxy, C₁₋₄ alkylsulfanyl, C₁₋₄ alkylsulfinyl, C₁₋₄ fluoroalkyl, C₁₋₄ fluoroalkoxy, C₃₋₅ cycloalkyl, and C₃₋₅ cycloalkyloxy; wherein each R¹⁰ is independently selected from hydrogen and C₁₋₃ alkyl, or two R¹⁰ together with the nitrogen to which they are bonded form a 3- to 5-membered heterocyclic ring;

one of R², R³, and R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

each of the other R², R³, and R⁴ is hydrogen;

R⁶ is —COOH;

each R⁷ is selected from hydrogen, methyl, hydroxyl, and fluoro;

R⁸ is selected from hydrogen, methyl, ethyl, propyl, isopropyl, cyclopropyl, tert-butyl, hydroxyl, methoxy, ethoxy, isopropoxy, trifluoromethyl, and trifluoromethoxy; and
L is selected from a bond “—”, —CH₂—, —C(OH)H—, —CHCH₃—, —C(CH₃)₂—, —CF₂—, —O—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —CH₂—CO—, and —CO—CH₂—, wherein R¹⁷ is selected from hydrogen and methyl.

In certain embodiments of a compound of Formula (1),

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring;

one of R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R², R³, R⁴, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

In certain embodiments of a compound of Formula (1),

R¹ is selected from halogen, —N(R¹⁰)₂, —NR¹⁰(OR¹⁰), —NO₂, —NO, —OH, C₁₋₄ alkyl, C₁₋₄ alkoxy, C₁₋₄ alkylsulfanyl, C₁₋₄ alkylsulfinyl, C₁₋₄ fluoroalkyl, C₁₋₄ fluoroalkoxy, C₃₋₅ cycloalkyl, and C₃₋₅ cycloalkyloxy; wherein each R¹⁰ is independently selected from hydrogen and C₁₋₃ alkyl, or two R¹⁰ together with the nitrogen to which they are bonded form a 3- to 5-membered heterocyclic ring;

one of R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

each of the other of R², R³, R⁴, and R⁵ is hydrogen;

R⁶ is —COOH;

each R⁷ is selected from hydrogen, methyl, hydroxyl, and fluoro;

R⁸ is selected from hydrogen, methyl, ethyl, propyl, isopropyl, cyclopropyl, tert-butyl, hydroxyl, methoxy, ethoxy, isopropoxy, trifluoromethyl, and trifluoromethoxy; and

L is selected from a bond “—”, —CH₂—, —C(OH)H—, —CHCH₃—, —C(CH₃)₂—, —CF₂—, —O—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —CH₂—CO—, and —CO—CH₂—, wherein R¹⁷ is selected from hydrogen and methyl.

In certain embodiments of a compound of Formula (1),

R⁵ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring;

one of R¹, R², R³, and R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R¹, R², R³, and R⁴ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy;

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

In certain embodiments of a compound of Formula (1),

R⁵ is selected from halogen, —N(R¹⁰)₂, —NR¹⁰(OR¹⁰), —NO₂, —NO, —OH, C₁₋₄ alkyl, C₁₋₄ alkoxy, C₁₋₄ alkylsulfanyl, C₁₋₄ alkylsulfinyl, C₁₋₄ fluoroalkyl, C₁₋₄ fluoroalkoxy, C₃₋₅ cycloalkyl, and C₃₋₅ cycloalkyloxy; wherein each R¹⁰ is independently selected from hydrogen and C₁₋₃ alkyl, or two R¹⁰ together with the nitrogen to which they are bonded form a 3- to 5-membered heterocyclic ring;

one of R¹, R², R³, and R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R¹, R², R³, and R⁴ is hydrogen;

R⁶ is —COOH;

each R⁷ is selected from hydrogen, methyl, hydroxyl, and fluoro;

R⁸ is selected from hydrogen, methyl, ethyl, propyl, isopropyl, cyclopropyl, tert-butyl, hydroxyl, methoxy, ethoxy, isopropoxy, trifluoromethyl, and trifluoromethoxy;

L is selected from a bond “—”, —CH₂—, —C(OH)H—, —CHCH₃—, —C(CH₃)₂—, —CF₂—, —O—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —CH₂—CO—, and —CO—CH₂—, wherein R¹⁷ is selected from hydrogen and methyl.

In certain embodiments of a compound of Formula (1),

one of R¹ and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R¹, R², R³, R⁴, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

In certain embodiments of a compound of Formula (1),

one of R¹ and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R¹, R², R³, R⁴, and R⁵ is hydrogen;

R⁶ is —COOH;

each R⁷ is selected from hydrogen, methyl, hydroxyl, and fluoro;

R⁸ is selected from hydrogen, methyl, ethyl, propyl, isopropyl, cyclopropyl, tert-butyl, hydroxyl, methoxy, ethoxy, isopropoxy, trifluoromethyl, and trifluoromethoxy; and

L is selected from a bond “—”, —CH₂—, —C(OH)H—, —CHCH₃—, —C(CH₃)₂—, —CF₂—, —O—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —CH₂—CO—, and —CO—CH₂—, wherein R¹⁷ is selected from hydrogen and methyl.

In certain embodiments of a compound of Formula (1),

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring;

R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

each of R², R³, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

In certain embodiments of a compound of Formula (1),

R¹ is selected from halogen, —N(R¹⁰)₂, —NR¹⁰(OR¹⁰), —NO₂, —NO, —OH, C₁₋₄ alkyl, C₁₋₄ alkoxy, C₁₋₄ alkylsulfanyl, C₁₋₄ alkylsulfinyl, C₁₋₄ fluoroalkyl, C₁₋₄ fluoroalkoxy, C₃₋₅ cycloalkyl, and C₃₋₅ cycloalkyloxy; wherein each R¹⁰ is independently selected from hydrogen or C₁₋₃ alkyl; or two R¹⁰ together with the nitrogen to which they are bonded form a 3- to 5-membered heterocyclic ring;

R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

each of R², R³, and R⁵ is hydrogen;

R⁶ is —COOH;

each R⁷ is selected from hydrogen, methyl, hydroxyl, and fluoro;

R⁸ is selected from hydrogen, methyl, ethyl, propyl, isopropyl, cyclopropyl, tert-butyl, hydroxyl, methoxy, ethoxy, isopropoxy, trifluoromethyl, and trifluoromethoxy; and

L is selected from a bond “—”, —CH₂—, —C(OH)H—, —CHCH₃—, —C(CH₃)₂—, —CF₂—, —O—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —CH₂—CO—, and —CO—CH₂—, wherein R¹⁷ is selected from hydrogen and methyl.

In certain embodiments, R⁸ is selected from hydrogen, deuterio, C₁₋₆ alkyl, substituted C₁₋₆ alkyl, C₁₋₆ heteroalkyl, substituted C₁₋₆ heteroalkyl, C₃₋₆ cycloalkyl, substituted C₃₋₆ cycloalkyl, —COOR¹⁰, C₁₋₄ fluoroalkyl, C₃₋₆ cycloalkyl, and phenyl;

In certain embodiments, R⁸ is selected from hydrogen, deuterio, C₁₋₄ alkyl, C₁₋₄ fluoroalkyl, and cyclopropyl.

In certain embodiments, R⁸ is selected from hydrogen, deuterio, methyl, ethyl, propyl, isopropyl, butyl, isobutyl, tert-butyl, trifluoromethyl, and cyclopropyl.

In certain embodiments, L is —(X)ₐ—, wherein, each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and a is selected from 0, 1, 2, 3, and 4.

In certain embodiments, L is selected from a bond (“—”), methylene (—CH₂—), fluoromethylene (—CFH—), difluoromethylene (—CF₂—), hydroxymethylene (—C(OH)H—), ethane-1,1-diyl (—CHCH₃—), propane-2,2-diyl (—C(CH₃)₂—), propane-1,1-diyl (—CH(CH₂—CH₃)—), sulfinyl (—SO—), sulfonyl (—SO₂—), and carbonyl (—CO—).

In certain embodiments, L is selected from a bond (“—”), methylene (—CH₂—), fluoromethylene (—CFH—), difluoromethylene (—CF₂—), hydroxymethylene (—C(OH)H—), ethane-1,1-diyl (—CHCH₃—), propane-2,2-diyl (—C(CH₃)₂—), sulfonyl (—SO₂—), and carbonyl (—CO—).

In certain embodiments, L is selected from ethane-1,2-diyl (—CH₂—CH₂—), propane-1,2-diyl (—CH₂—CHCH₃ or —CHCH₃—CH₂—), hydroxyethane-1,2-diyl (—CH₂—CHOH— or —CHOH—CH₂—), fluoroethane-1,2-diyl (—CH₂—CHF— or —CHF—CH₂—), difluoroethane-1,2-diyl (—CH₂—CF₂— or —CF₂—CH₂—), carbonyl amino (—CO—NR¹⁷—), methyleneamino (—CH₂—NR¹⁷—), methyleneoxy (—CH₂—O—), methylenethiyl (—CH₂—S—), methylenesulfinyl (—CH₂—SO—), sulfinylmethylene (—SO—CH₂—), methylenesulfonyl (—CH₂—SO₂—), sulfonylmethylene (—SO₂—CH₂—), methylenescarbonyl (—CH₂—CO—), and carbonylmethylene (—CO—CH₂—), wherein R¹⁷ is selected from hydrogen, methyl, and ethyl.

In certain embodiments of a compound of Formula (1), the absolute stereochemistry of the beta-carbon atom is (R).

In certain embodiments of a compound of Formula (1), the absolute stereochemistry of the beta-carbon atom is (S).

In certain embodiments of a compound of Formula (1), the absolute stereochemistry of the β carbon atom is of the (R) configuration, the absolute axial stereochemistry (atropisomerism) is Rₐ, and the absolute stereochemistry of a compound of Formula (1) is (R,Rₐ).

In certain embodiments of a compound of Formula (1), the absolute stereochemistry of the β-carbon atom is of the (R) configuration, the absolute axial stereochemistry (atropisomerism) is Sₐ, and the absolute stereochemistry of a compound of Formula (1) is (R,Sₐ).

In certain embodiments of a compound of Formula (1), the absolute stereochemistry of the β-carbon atom is of the (S) configuration, the absolute axial stereochemistry (atropisomerism) is Rₐ, and the absolute stereochemistry of a compound of Formula (1) is (S,Rₐ).

In certain embodiments of a compound of Formula (1), the absolute stereochemistry of the β-carbon atom is of the (S) configuration, the absolute axial stereochemistry (atropisomerism) is Sₐ, and the absolute stereochemistry of a compound of Formula (1) is (S,Sₐ).

In certain embodiments, a compound of Formula (1) is selected from:
3-Amino-3-[5-[bis(2-chloroethyl)amino]-2-methyl-phenyl]propanoic acid (1);
3-Amino-3-[4-[bis(2-chloroethyl)amino]-2-methyl-phenyl]propanoic acid (2);
3-Amino-4-[5-[bis(2-chloroethyl)amino]-2-methyl-phenyl]butanoic acid (3);
3-Amino-4-[4-[bis(2-chloroethyl)amino]-2-methyl-phenyl]butanoic acid (4);
(3S)-3-Amino-4-[5-[bis(2-chloroethyl)amino]-2-methyl-phenyl]butanoic acid (5);
(3R)-3-Amino-4-[5-[bis(2-chloroethyl)amino]-2-methyl-phenyl]butanoic acid (6);
(3S)-3-Amino-4-[5-[bis(2-chloroethyl)amino]-2-methoxy-phenyl]butanoic acid (7);
(3S)-3-Amino-4-[3-[bis(2-chloroethyl)amino]-2,6-dimethyl-phenyl]butanoic acid (8);
(3S)-3-Amino-4-[5-[bis(2-chloroethyl)amino]-2-methyl-phenyl]-3-methyl-butanoic acid (9);
[(2R)-2-Amino-3-[5-[bis(2-chloroethyl)amino]-2-methyl-phenyl]propyl]phosphinic acid (10);
(3R)-3-Amino-4-[5-(bis(2-methylsulfonyloxyethyl)amino)-2-methyl-phenyl]butanoic acid (11);
(3R)-3-Amino-4-[5-(bis(2-bromoethyl)amino)-2-methyl-phenyl]butanoic acid (12);
(3R)-3-Amino-4-[5-(2-chloroethyl(2-methylsulfonyloxyethyl)amino)-2-methyl-phenyl]butanoic acid (13);
(3R)-3-Amino-4-[5-(2-bromoethyl(2-chloroethyl)amino)-2-methyl-phenyl]butanoic acid (14);
(3R)-3-Amino-4-[5-(2-bromoethyl(2-methylsulfonyloxyethyl)amino)-2-methyl-phenyl]butanoic acid (15);
(3S)-3-Amino-4-[[5-[bis(2-chloroethyl)amino]-2-methyl-phenyl]amino]-4-oxo-butanoic acid (16);
(3S)-3-Amino-4-[2-[bis(2-chloroethyl)amino]phenoxy]butanoic acid (17);
(3R)-3-Amino-5-[5-[bis(2-chloroethyl)amino]-2-methyl-phenyl]pentanoic acid (18);
(3R)-3-Amino-4-[5-(2-chloroethyl(chloromethyl)carbamoyl)oxy-2-methyl-phenyl]butanoic acid (19);
(3R)-3-Amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21);
3-[(2R)-2-Amino-4-hydroxy-4-oxo-butyl]-N,N-bis(2-chloroethyl)-4-methyl-benzeneamine oxide (22); and
(3R)-3-Amino-4-[2-[bis(2-chloroethyl)carbamoyl]phenyl]butanoic acid (23);
or a pharmaceutically acceptable salt or salts of any of the foregoing.

In certain embodiments of any of the foregoing compounds, a pharmaceutically acceptable salt is the hydrochloride salt.

In certain embodiments of any of the foregoing compounds, a pharmaceutically acceptable salt is the dihydrochloride salt.

In certain embodiments of a compound of Formula (1), a pharmaceutically acceptable salt is the hydrochloride salt.

In certain embodiments of a compound of Formula (1), a pharmaceutically acceptable salt is the dihydrochloride salt.

In certain embodiments, compounds of Formula (1) are selective substrates for the LAT1/4F2hc transporter.

In certain embodiments, compounds provided by the present disclosure exhibit a LAT1/4F2hc-dependent Vₘₐₓ of at least 10% the Vₘₐₓ of gabapentin. In certain embodiments, compounds provided by the present disclosure exhibit a LAT1/4F2hc-dependent Vₘₐₓ of at least 20% the Vₘₐₓ of gabapentin. In certain embodiments, compounds provided by the present disclosure exhibit a LAT1/4F2hc-dependent Vₘₐₓ of at least 30% the Vₘₐₓ of gabapentin. In certain embodiments, compounds provided by the present disclosure exhibit a LAT1/4F2hc-dependent Vₘₐₓ of at least 40% the Vₘₐₓ of gabapentin. In certain embodiments, compounds provided by the present disclosure exhibit a LAT1/4F2hc-dependent Vₘₐₓ of at least 50% the Vₘₐₓ of gabapentin. In certain embodiments, compounds provided by the present disclosure exhibit a LAT1/4F2hc-dependent Vₘₐₓ of at least 60% the Vₘₐₓ of gabapentin. In certain embodiments, compounds provided by the present disclosure exhibit a LAT1/4F2hc-dependent Vₘₐₓ of at least 70% the Vₘₐₓ of gabapentin. In certain embodiments, compounds provided by the present disclosure exhibit a LAT1/4F2hc-dependent Vₘₐₓ of at least 80% the Vₘₐₓ of gabapentin. In certain embodiments, compounds provided by the present disclosure exhibit a LAT1/4F2hc-dependent Vₘₐₓ of at least 90% the Vₘₐₓ of gabapentin. In certain embodiments, compounds provided by the present disclosure exhibit a LAT1/4F2hc-dependent Vₘₐₓ of at least 100% the Vₘₐₓ of gabapentin.

In certain embodiments, compounds provided by the present disclosure exhibit a LAT1/4F2hc-dependent uptake of at least 10% that of gabapentin measured at an extracellular concentration of 1 mM (1 mmol/L) and a system A-, system N-, a system ASC-, and a LAT2/4F2hc-dependent uptake of less than 50% that of L-leucine measured at an extracellular concentration of 1 mM (1 mmol/L). In certain embodiments, compounds provided by the present disclosure exhibit a LAT1/4F2hc-dependent uptake of at least 10% that of gabapentin measured at an extracellular concentration of 1 mM (1 mmol/L); and a system A-, system N-, a system ASC-, and a LAT2/4F2hc-dependent uptake of less than 40% that of L-leucine measured at an extracellular concentration of 1 mM (1 mmol/L). In certain embodiments, compounds provided by the present disclosure exhibit a LAT1/4F2hc-dependent uptake of at least 10% that of gabapentin measured at an extracellular concentration of 1 mM (1 mmol/L); and a system A-, system N-, a system ASC-, and a LAT2/4F2hc-dependent uptake of less than 30% that of L-leucine measured at an extracellular concentration of 1 mM (mmol/L). In certain embodiments, compounds provided by the present disclosure exhibit a LAT1/4F2hc-dependent uptake of at least 10% that of gabapentin measured at an extracellular concentration of 1 mM (1 mmol/L); and a system A-, system N-, a system ASC-, and a LAT2/4F2hc-dependent uptake of less than 20% that of L-leucine measured at an extracellular concentration of 1 mM (1 mmol/L). In certain embodiments, compounds provided by the present disclosure exhibit a LAT1/4F2hc-dependent uptake of at least 10% that of gabapentin measured at an extracellular concentration of 1 mM (1 mmol/L); and a system A-, system N-, a system ASC-, and a LAT2/4F2hc-dependent uptake of less than 10% that of L-leucine measured at an extracellular concentration of 1 mM (1 mmol/L). In certain embodiments, compounds provided by the present disclosure exhibit a LAT1/4F2hc-dependent uptake of at least 10% that of gabapentin measured at an extracellular concentration of 1 mM (1 mmol/L); and a system A-, system N-, a system ASC-, and a LAT2/4F2hc-dependent uptake of less than 5% that of L-leucine measured at an extracellular concentration of 1 mM (1 mmol/L). In certain embodiments, compounds provided by the present disclosure exhibit a LAT1/4F2hc-dependent uptake of at least 10% that of gabapentin measured at an extracellular concentration of 1 mM (1 mmol/L); and a system A-, system N-, a system ASC-, and a LAT2/4F2hc-dependent uptake of less than 1% that of L-leucine measured at an extracellular concentration of 1 mM (1 mmol/L).

Compounds of Formula (1) may be adapted as prodrugs to achieve desirable pharmacokinetic properties. For example, suitable prodrugs of β-substituted β-amino acid derivatives and β-substituted β-amino acid analogs are disclosed by Gallop, et al., U.S. Pat. No. 7,109,239, U.S. Pat. No. 6,972,341, U.S. Pat. No. 6,818,787 and U.S. Pat. No. 7,227,028. Prodrugs of compounds of Formula (1) include the prodrug systems disclosed by Gallop, et al., as well as others known in the art.

Compounds disclosed herein may be obtained via the general synthetic methods illustrated in Schemes 1-10. General synthetic methods useful in the synthesis of compounds, precursors, and starting materials described herein are available in the art. Starting materials useful for preparing compounds and intermediates thereof, and/or practicing methods described herein, are commercially available or may be prepared by well-known synthetic methods (March's Advanced Organic Chemistry: Reactions, Mechanisms, M. B. Smith, 7^{th} Edition, John Wiley & Sons, Hoboken, N.J., USA, 2013; Advanced Organic Chemistry: Part B: Reaction and Synthesis, F. A. Carey and R. J. Sundberg, 5^{th} Edition, Springer, Germany, 2010; Comprehensive Organic Transformations, 2^{nd} Edition, and R. C. Larock, Wiley-VCH, Weinheim, Germany, 1999).

Additionally, as will be apparent to those skilled in the art, use of conventional protecting groups or protecting strategies may be necessary to prevent certain functional groups from undergoing undesired reactions. Suitable protecting groups for various functional groups as well as suitable conditions for protecting and deprotecting particular functional groups are well known in the art. On the other hand, many methods for selective removal of protecting groups without affecting the desired molecular architecture are also well known in the art (Wuts and Greene, Greene's Protective Groups in Organic Synthesis, ^{4th} Ed, 2007, Wiley-Interscience, John Wiley & Sons, Inc., Hoboken, N.J.).

It will be appreciated that where typical or preferred process conditions, e.g., reaction temperatures, reaction times, molar ratios of reactants, solvents, pressures, etc., are given other process conditions may also be used. Optimal reaction conditions may vary with the particular reactants, solvents, functional groups, and protecting groups used, but such conditions may be determined by one skilled in the art by routine optimization procedures.

Furthermore, certain compounds provided by the present disclosure may contain one or more stereogenic centers. Accordingly, and if desired, such compounds may be prepared or isolated as pure stereoisomers, e.g., as individual enantiomers, diastereomers, atropisomers, rotamers, or as stereoisomer enriched mixtures or racemates. All such stereoisomers are included within the scope of this disclosure. Pure stereoisomers (or enriched mixtures thereof) may be prepared using, for example, optically active starting materials, stereoselective reagents such as chiral catalysts and auxiliaries well known in the art. Alternatively, racemic mixtures of such compounds may be separated or partially enriched using, for example, chromatographic methods with chiral stationary phases, chiral resolving agents, and the like, also well known in the art and easily adaptable to the particular compound to be separated.

There has been an ever growing interest in the synthesis of β-amino acids with various substitution patterns. Depending on the location and the number of the substituents, (β-amino acids are categorized as (a) β²-(mono-α-substituted), (b) β³-(mono-β-substituted), (c) β^{2,3}-(α,β-di-substituted), (d) β^{2,2}-(α,α-di-substituted or α-geminal-disubstituted), (e) β^{3,3}-(β,β-di-substituted or β-geminal-di substituted), (f) β^{2,2,3}-(α,α,β-tri-substituted), (g) β^{2,3,3}-(α,β,β-tri-substituted), or (h) β^{2,2,3,3}-((α,α,β,β-tetra-substituted) amino acids. Many methods for the synthesis of protected and unprotected β-amino acids with a wide variety of type and number of substituents either in racemic, enantio- or diastereomerically enriched or pure form from commercial or known starting materials are well known in the art (Enantioselective Synthesis of β-Amino Acids, 2^{nd} Edition, E. Juaristi and V. Soloshonok, John Wiley & Sons, 2005, Hoboken, N.J., USA, 2005; Smith, Methods of Non-α-Amino Acid Synthesis, Marcel Dekker, Inc., New York, USA, 1995; Cole, Tetrahedron, 1994, 50 (32), 9517-9582; Juaristi, et al., Aldrich Chim. Acta, 1994, 27(1), 3-11; Lelais and Seebach, Biopolymers (Peptide Science), 2004, 76, 206-243; Sewald, Amino Acids, 1996, 11, 397-408; Seebach, et al., Synthesis, 2009, (1), 1-32; and Abele and Seebach, Eur. J. Org. Chem., 2000, (1), 1-15).

In particular, many methods of preparing protected and unprotected β³-substituted racemic or optically active β-amino acids, β-amino acids analogs, or β-amino acid carboxylic acid (bio)isosters from commercial or known starting materials are well known in the art.

In certain embodiments, such derivatives may be used as convenient starting materials for the preparation of the target compounds provided by the present disclosure. In certain embodiments, suitably functionalized protected and unprotected β³-substituted racemic or optically active β-amino acids, β-amino acids analogs, or β-amino acid carboxylic acid (bio)isosters may be used as starting materials for the preparation of the target compounds provided by the present disclosure.

In certain embodiments, starting materials may be used in their fully protected form wherein the amino group or a synthetic equivalent or a precursor thereof and the carboxylic acid, phosphinic acid, sulfinic acid, carboxylic acid (bio)isosteres or synthetic equivalents or precursors of any of the foregoing are appropriately protected.

In certain embodiments, starting materials may be used in their hemi-protected form wherein the amino group or a synthetic equivalent or a precursor thereof is protected and the carboxylic acid group, phosphinic acid, sulfinic acid, or carboxylic acid (bio)isostere functional group or synthetic equivalents or precursors of any of the foregoing are unprotected or free.

In certain embodiments, starting materials may be used in their hemi-protected form wherein the amino group is unprotected or free and the carboxylic acid, phosphinic acid, sulfinic acid, or carboxylic acid (bio)isostere or synthetic equivalents or precursors of any of the foregoing are appropriately protected.

In certain embodiments, starting materials may be used in their full unprotected form wherein the amino group and the carboxylic acid, free phosphinic acid, free sulfinic acid, or free carboxylic acid (bio)isostere or synthetic equivalents or precursors of any of the foregoing are unprotected.

In certain embodiments, protected and unprotected β³-substituted racemic or optically active β-amino acids, β-amino acids analogs, or β-amino acid carboxylic acid (bio)isosters bear a chemical functional group linking the β³-carbon atom to an aromatic ring system. In certain embodiments, the aromatic ring system is functionalized with an anchoring group in order to install a chemotherapeutic moiety.

Methods of synthetic manipulations and modifications of the underlying protected or unprotected β-amino acid scaffold are well known in the art. In certain embodiments, the underlying the underlying β-amino acid scaffold may be modified to allow for regio- and/or stereoselective incorporation of auxiliary molecular functionalities. Auxiliary molecular functionalities may, for example, be incorporated to modulate interaction with LAT1 transporter proteins, e.g., efficacy of translocation through biological membranes (binding to the LAT1-transporter protein and capacity of LAT1-mediated transport), aid the modulation of physiochemical parameter, or to modulate the activity of the physiologically active N-mustard moiety, e.g., cytotoxicity.

In certain embodiments, the underlying aryl-ring may be modified to allow for regioselective incorporation of functional groups that can be converted to chemotherapeutic moieties by using reagents, methods, and protocols well known in the art.

In certain embodiments, the underlying aryl-ring may be modified to allow for regio- and/or stereoselective incorporation of auxiliary molecular functionalities into the arene scaffold. Auxiliary molecular functionalities may, for example, be incorporated to modulate interaction with LAT1 transporter proteins, e.g., efficacy of translocation through biological membranes (binding to the LAT1-transporter protein and capacity of LAT1-mediated transport), or to modulate the activity of the physiologically active chemotherapeutic moiety, e.g., cytotoxicity.

Many other methods for the preparation of appropriately functionalized or substituted, protected and unprotected β³-substituted racemic or optically active β-amino acids, β-amino acids analogs, or β-amino acid carboxylic acid (bio)isosters, derivatives or precursors of any of the foregoing from commercial or known starting materials and employing methods and protocols are either described herein, are described in the art, or will be readily apparent to the one skilled in the art. Accordingly, the methods presented in the schemes provided by the present disclosure are illustrative rather than comprehensive.

Referring to Scheme 1, selected and representative starting materials for the preparation N-mustard functionalized β-branched β-amino acids, β-amino acid analogs, or β-amino acids carboxylic acid (bio)isosteres are compounds of Formula (A). This selection is not intended to be limiting in any way.

Referring to Scheme 1, in certain embodiments R¹ and/or R⁵, and the linker L are defined as described herein; one of R², R³, and R⁴ in compounds of Formula (A) is -E-MH, wherein E is a bond (“—”), an oxygen atom (—O—), a methylene group (—CH₂—), a methyleneoxy group (—CH₂—O—), a carbonyl group (—CO—), or a methylenecarbonyl group (—CH₂—CO—), and wherein MH is an amino group (—NH₂), a hydroxyl group (—OH), or a sulfhydryl group (—SH). Each of the other remaining R², R³, and R⁴ is hydrogen; each R⁷ and each R⁸ is hydrogen.

Referring to Scheme 1, for example, (a) -E-MH is equivalent to a primary aromatic amino group (—NH₂, aniline) when E is a bond (“—”) and MH is an amino group (—NH₂), (b) -E-MH is equivalent to a primary O-aryl hydroxylamino group (—O—NH₂) when E is an oxygen atom (—O—) and MH is an amino group (—NH₂), (c) -E-MH is equivalent to a primary aminomethyl group (—CH₂—NH₂, primary benzylic amine) when E is a methylene group (—CH₂—) and MH is an amino group (—NH₂), (d) -E-MH is equivalent to an aromatic hydroxyl group (—OH, phenol) when E is a bond (“—”) and MH is a hydroxyl group (—OH), (e) -E-MH is equivalent to a hydroxymethyl group (—CH₂—OH, benzylic alcohol) when E is a methylene group (—CH₂—) and MH is a hydroxyl group (—OH), (f) -E-MH is equivalent to a primary O-benzylic hydroxylamino group (—CH₂—O—NH₂) when E is a methyleneoxy group (—CH₂—O—) and MH is an amino group (—NH₂), (g) -E-MH is equivalent to an aromatic sulhydryl group (—SH, thiophenol derivative) when E is a bond (“—”) and MH is a hydroxyl group (—OH), (h) -E-MH is equivalent to a methylenesulhydryl group (—CH₂—SH, benzylic thiol) when E is a methylene group (—CH₂—) and MH is a sulfhydryl group (—SH), (i) -E-MH is equivalent to an aromatic carboxylic acid group (—CO—OH, benzoic acid) when E is a carbonyl group (—C(═O)—) and MH is a hydroxyl group (—OH), (j) -E-MH is equivalent to a carboxylic acid group (—CO—OH, benzoic acid) when E is a methylenecarbonyl group (—CH₂—C(═O)—) and MH is a hydroxyl group (—OH).

It will be understood by those skilled in the art that in some embodiments of the disclosure the group “-E-” in functional groups -E-MH presented in the following schemes is equivalent to the group -A- in the definition of the composition of a chemotherapeutic moiety as described herein.

Referring to Scheme 1, in certain embodiments R²⁰ in compounds of Formula (A) is a protected carboxyl group such as a lower alkyl ester of a carboxyl group, e.g., a methyl, ethyl, or tert-butyl ester, or a benzyl ester derivative, e.g., benzyl, pentamethylbenzyl, or (4-methoxy)benzyl. In certain embodiments, R²⁰ in compounds of Formula (A) is a tert-butyl ester group (CO₂tBu). In certain embodiments, R²⁰ in compounds of Formula (A) is a methyl ester group (CO₂Me).

Referring to Scheme 1, in certain embodiments, R²⁰ in compounds of Formula (A) is a protected phosphinic acid derivative, e.g., 1,1-diethyloxyethylethoxyphosphino-1-one (—P(═O)(OEt)[C(OEt)₂Me] (U.S. Pat. No. 8,344,028; Baylis, Tetrahedron Lett, 1995, 36(51), 9385-9388; and Burgos-Lepley, et al., Bioorg. Med. Chem. Lett., 2006, 16, 2333-2336). In certain embodiments, R²⁰ in compounds of Formula (A) has alternatively protected phosphonates and phosphinates as described in the art (Palacios, et al., Chem. Rev., 2005, 105,899-931; and Lejzak, et al., J. Enzyme Inhibit., 1993, 7(2), 97-103).

Referring to Scheme 1, in certain embodiments, R²⁰ in compounds of Formula (A) is a protected sulfinic acid precursor derivative, e.g., a 2-mercaptobenzothiazole (Carruthers, et al., Bioorg. Med. Chem. Lett, 1995, 5, 237-240; Carruthers, et al., Bioorg. Med. Chem. Lett, 1998, 5, 3059-3064; and Okawara, et al., Chem. Lett., 1984, 2015; C. E. Burgos-Lepley, et al., Bioorg. Med. Chem. Lett., 2006, 16, 2333-2336).

Referring to Scheme 1, in certain embodiments, R²⁰ in compounds Formula (A) is a unprotected or protected carboxylic acid (bio)isostere including a protected or unprotected 1H-tetrazole (Ballatore, et al., ChemMedChem, 2013, 8(3), 385-395; Bryans, et al., U.S. Pat. No. 6,518,289; and Burgos-Lepley, et al., Bioorg. Med. Chem. Lett., 2006, 16, 2333-2336).

Referring to Scheme 1, in certain embodiments of compounds of Formula (A) Q is N(H)-PG where PG is a suitable nitrogen protecting group, e.g., tert-butoxycarbonyl (Boc), allyloxycarbonyl (alloc), benzyloxycarbonyl (Cbz, Z), ethoxycarbonyl, methoxycarbonyl, (R/S)-1-phenyl-ethoxycarbonyl, (R)-1-phenyl-ethoxycarbonyl, (S)-1-phenyl-ethoxycarbonyl, 1-methyl-1-phenyl-ethoxycarbonyl, formyl, acetyl, trifluoroacetyl, benzoyl, triphenylmethyl (trityl), 4-methoxyphenyl-diphenylmethyl, or di-(4-methoxyphenyl)-phenylmethyl, and the like. In certain embodiments, PG in compounds of Formula (A) is tert-butoxycarbonyl (Boc) and Q is N(H)Boc (N(H)CO₂tBu). In certain embodiments of compounds of Formula (A) PG is benzyloxycarbonyl (Cbz, Z), and Q is N(H)-Cbz (N(H)COOBn). In certain embodiments of compounds of Formula (A), PG is acetyl and Q is N(H)—Ac (N(H)COMe).

Referring to Scheme 1, in certain embodiments of compounds of Formula (A) Q is N(PG)₂, where PG is a nitrogen protecting group such as an imide-type protecting group, e.g., phthalyl or tert-butoxycarbonyl (Boc). In certain embodiments of compounds of Formula (A) PG is phthalyl and Q is N(phthalyl). In certain embodiments of compounds of Formula (A) PG is tert-butoxycarbonyl and Q is N(Boc)₂.

Referring to Scheme 1, in certain embodiments of compounds of Formula (A) the protected amine functionality is an imine where Q is N is CR³⁰R³¹ and each of R³⁰ and R³¹ is independently selected from branched C₁₋₄ alkyl, non-branched C₁₋₄ alkyl, substituted aryl, non-substituted aryl, substituted heteroaryl, and non-substituted heteroaryl.

Accordingly, the structures presented in the schemes provided by the present disclosure are illustrative rather than comprehensive.

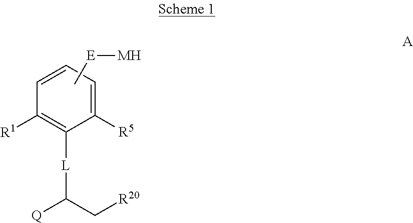

Referring to Scheme 2, in certain embodiments R¹ and/or R⁵, R²⁰, E, the linker L, and the protecting groups PG and Q are defined as described herein; one of R², R³, and R⁴ in compounds of Formula (C) is -E-NH₂, wherein E is a bond (“—”), an oxygen atom (—O—), a methylene group (—CH₂—), or methylenoxy group (—CH₂—O—), and wherein MH is an amino group (—NH₂) so that -E-NH₂ is equivalent to a) a primary aromatic amino group (—NH₂, aniline), b) a primary O-aryl hydroxylamino group (—O—NH₂), c) a primary aminomethyl group (—CH₂—NH₂), or a primary O-benzyl hydroxylamino group (—CH₂—O—NH₂). Each of the other remaining R², R³, and R⁴ is hydrogen; each R⁷ and each R⁸ is hydrogen. X is a suitable leaving group e.g., chloro (—Cl) or bromo (—Br).

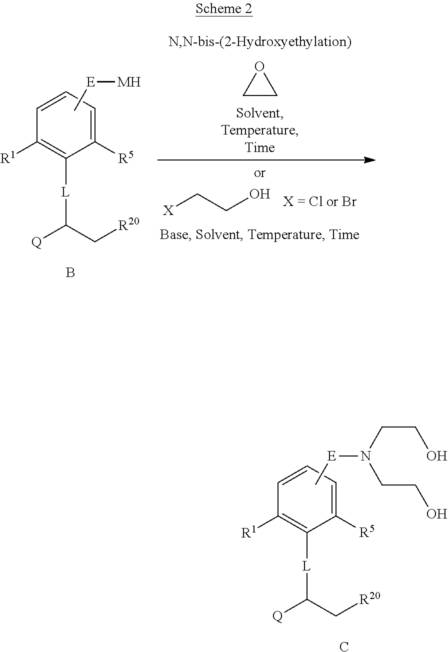

Referring to Scheme 2, conversion of the primary amino group as in compounds of Formula (B) to the N,N-bis-(2-hydroxyethyl) amino group (N,N-bis-(2-hydroxyethylation)) as in compounds of Formula (C) may be accomplished by reacting compounds of Formula (B) in suitable solvents such as about 25-75 vol.-% aqueous acetic acid (HOAc), glacial acetic acid, water, tetrahydrofuran (THF), ethanol (EtOH), 1,4-dioxane, or mixtures of any of the foregoing with an excess of ethylene oxide (oxirane) (about 4-20 equivalents) at a temperature of about −20° C. to about room temperature for about 12-48 hours. Alternatively, the reaction mixture may be heated in a sealed reaction vessel from about 80-140° C. for comparable times (Palmer, et al., J. Med. Chem. 1990, 33(1), 112-121; Jordan, et al., Bioorg. Med. Chem., 2002, 10(8), 2625-2633; Abela Medici, et al, J. Chem. Soc., Perkin Trans. 1, 1997, (20), 2258-2263; Feau, et al., Org. Biomolecular Chem., 2009, 7(24), 5259-5270; Springer, et al., J. Med. Chem., 1990, 33(2), 677-681; Taylor, et al., Chem. Biol. Drug Des., 2007, 70(3), 216-226; Buss, et al., J. Fluorine Chem., 1986, 34(1), 83-114; Larden and Cheung, Tetrahedron Lett., 1996, 37(42), 7581-7582; Spreitzer and Puschmann, Monatshefte fiir Chemie, 2007, 138(5), 517-522; Niculesscu-Duvaz, et al., J. Med. Chem., 2004, 47(10), 2651-2658; Weisz, et al., Bioorg. Med. Chem. Lett., 1995, 5(24), 2985-2988; Thorn, et al., J. Org. Chem, 1975, 40(11), 1556-1558; Baraldini, et al., J. Med., Chem., 2000, 53(14), 2675-2684; Zheng, et al., Bioorg., Med., Chem., 2010, 18(2), 880-886; Gourdi, et al., J., Med., Chem., 1990, 33(4), 1177-1186; Haines, et al., J. Med. Chem., 1987, 30, 542-547; Matharu, et al., Bioorg. Med. Chem. Lett., 2010, 20, 3688-3691; and Kupczyk-Subotkowska, et al., J. Drug Targeting, 1997, 4(6), 359-370).

Referring to Scheme 2, conversion of the primary amino group as in compounds of Formula (B) to the N,N-bis-(2-hydroxyethyl) amino group (N,N-bis-(2-hydroxyethylation)) as in compounds of Formula (C) may be accomplished by reacting compounds of Formula (B) in suitable solvents such water with an excess of about 2-5 equivalents of a suitable 2-halogeno ethanol derivative, e.g., 2-chloroethanol (ClCH₂CH₂OH) or 2-bromoethanol (BrCH₂CH₂OH), and about 2.0 equivalents of a suitable inorganic base such as sodium bicarbonate (NaHCO₃), sodium carbonate (Na₂CO₃), or calcium carbonate (CaCO₃) at about reflux temperature for about 8-24 hours. Optionally, the reaction may be carried out in the presence of a catalytic amount (about 10 mol-%) of potassium iodide (KI) (Palmer, et al., J. Med. Chem. 1990, 33(1), 112-121; Coggiola, et al., Bioorg. Med. Chem. Lett., 2005, 15(15), 3551-3554; Verny and Nicolas, J. Label. Cmpds Radiopharm, 1988, 25(9), 949-955; and Lin, Bioorg. Med. Chem. Lett., 2011, 21(3), 940-943).

Referring to Scheme 3, in certain embodiments electron-deficient aryl halides of Formula (D), activated with strongly electron withdrawing substituents for nucleophilic aromatic substitution reactions (S_{N}Ar) at the aryl ring, may be useful starting materials for incorporating N,N-bis-(2-functionalized) ethyl amino groups as in compounds of Formula (E) where the corresponding N,N-bis-(2-functionalized)ethyl amino groups are N,N-bis-(2-hydroxyethyl) amino groups. Commonly used leaving groups (—X) for S_{N}Ar-reactions include halogeno, e.g., fluoro (—F), chloro (—Cl), bromo (—Br), with accessory activating groups at the 2- or 4-position relative to the leaving group (ortho- or para-positions). Such groups decrease the electron density in the arene ring and increase the susceptibility to nucleophilic attack and displacement of the leaving group (—X). Examples of activating, strongly electron-withdrawing groups (EWG), include trifluoromethyl (—CF₃), cyano (—CN), nitro (—NO₂), amide (—CON(R¹⁰)₂), and formyl (—CHO).

Useful secondary amines for the introduction of the N,N-bis-(2-hydroxyethyl) amino functionality include diethanolamine (HN(CH₂CH₂OH)₂), protected diethanolamine derivatives, e.g., O-benzylether protected diethanolamine (HN(CH₂CH₂OBn)₂), or precursors of the putative N,N-bis-(2-hydroxyethyl)amino group, e.g., 3-pyrroline. Employing O-benzylether protected diethanolamine (HN(CH₂CH₂OBn)₂) or 3-pyrroline necessitates conversion of the corresponding intermediate substitution products to compounds of Formula (E) bearing the target N,N-bis-(2-hydroxyethyl)amino groups using methods well known in the art.

Referring to Scheme 3, in certain embodiments R¹ and/or R⁵, R¹⁰, R²⁰, the linker L, the protecting group PG, and Q, the electron withdrawing group (EWG), the leaving group (—X), and the secondary amine HNR₂ are defined as described herein; R¹ and/or R⁵ may also represent an electron withdrawing group (EWG); one or more of R², R³, and R⁴ in compounds of Formula (G) or of Formula (H) is a suitable leaving group (—X)), one or more of R², R³, and R⁴ is a electron withdrawing group (EWG) preferably in 2- or 4-position relative to the leaving group X; each of the other remaining R², R³, and R⁴ is hydrogen; each of R⁷ and R⁸ is hydrogen.

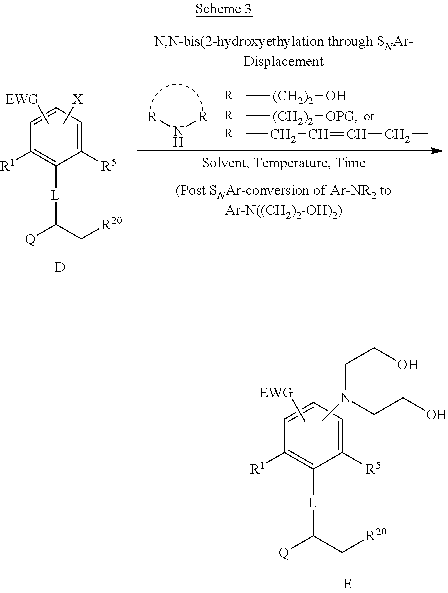

Referring to Scheme 3, N,N-bis(2-hydroxyethyl)amino derivatives as in compounds of Formula (E) may be prepared through nucleophilic aromatic substitution reactions (S_{N}Ar) of aromatic halides of Formula (D) activated by electron withdrawing groups (EWGs), by reaction with an excess of about 1.5-5 equivalents of the neat amine, e.g., HN(CH₂CH₂OH)₂, HN(CH₂CH₂OBn)₂, or 3-pyrroline, (weakly basic reaction conditions) or solutions of the secondary amine in polar aprotic anhydrous solvents, e.g., anhydrous dimethylsulfoxide (DMSO), N,N-dimethylformamide (DMF), N,N-dimethylacetamide (DMAc), acetonitrile (MeCN), 1,4-dioxane, tetrahydrofuran (THF), or mixtures of the foregoing at a temperature from about 80-200° C. (sealed tube), for about 1-12 hours to provide N,N-bis(2-hydroxyethyl)amino-functionalized compounds of Formula (E). The reaction may also be carried out in the presence of a catalyst, e.g., copper powder (about 10 mol %) (Atwell, et al., J. Med. Chem., 2007, 50(6), 1197-1212; Palmer, et al., J. Med. Chem., 1994, 37, 2175-2184; Palmer, et al., J. Med. Chem., 1992, 35(17), 3214-3222; Palmer, et al., J. Med. Chem, 1990, 33(1), 112-121; Davies, et al., J. Med. Chem. 2005, 48(16), 5321-5328; Jordan, et al., Bioorg. Med. Chem., 2002, 10(8), 2625-2633; Dheyongera, et al., Bioorg. Med. Chem., 2005, 13(3), 689-698; Lin, et al., Bioorg. Med. Chem. Lett., 2011, 21(3), 940-943; and Ferlin, et al., Bioorg. Med. Chem., 2004, 12(4), 771-777).

Referring to Scheme 3, methods to convert the N,N-bis-(2-benzyloxyethyl)amino group to a N,N-bis-(2-hydroxyethyl)amino group include, for example, catalytic hydrogenolysis of the benzyl ether groups using heterogeneous catalysts, e.g., 5-10% Pd on carbon (Pd/C) or Raney®-Nickel under standard hydrogenation reaction conditions are known in the art (Vincent and Prunet, Tetrahedron Lett, 2006, 47(24), 4075-4077).

Referring to Scheme 3, conversion the 3-pyrroline ring of the N-aryl-3-pyrroline moiety to a N,N-bis-(2-hydroxyethyl)amino group as in compounds of Formula (E) include oxidative cleavage of the C═C-double with the Lemieux-Johnson reagent (osmium tetroxide/sodium periodate, OsO₄/NaIO₄) or by ozonolysis with an O₃/O₂-gas mixture. Reductive work-up, e.g., with borane-dimethylsulfide complex (BH₃.Me₂S), triphenylphosphine (Ph₃P), thiourea (C(═S)(NH₂)₂), or zinc dust, yields intermediate N,N-bis(2-oxoethyl)amino groups which may subsequently be reduced to the desired N,N-bis-(2-hydroxyethyl)amino group as in compounds of Formula (E) with suitable reducing reagents, e.g., borane-THF complex (BH₃.THF), or sodium borohydride (NaBH₄), under standard reaction conditions (Palmer and Denny, Synth. Commun., 1987, 17(5), 601-610).

In general, the biological activity of nitrogen mustards is based upon the presence of a N,N-bis(2-chloroethyl) functionality. The chemotherapeutic and cytotoxic effects are directly associated with the alkylation of DNA due to the strong electrophilic character of the N,N-bis(2-chloroethyl) functionality. Formation of covalent linkages including interstrand crosslinks (ICLs) is highly cytotoxic and involves the disruption of fundamental cellular processes including DNA replication leading to cellular death.

Many methods and reagents for converting primary alcohols to primary alkyl chlorides including conversion of N,N-bis(2-hydroxyethyl)amino groups to N,N-bis(2-chloroethyl)amino groups are known in the art. The most common methods include the use of concentrated hydrochloric acid (HCl) and various inorganic chlorides of sulfur or phosphorus which are used either in neat form or as solutions in inert solvents such as chlorinated hydrocarbons, aromatic hydrocarbons, or polar non-protic solvents, at room temperature or at elevated temperatures. Other useful chlorination methods and reagents include, for example, combinations of triphenyl phosphine and trichloroacetonitrile (Ph₃P/Cl₃CCN), triphenylphosphine dichloride (Ph₃PCl₂) (prepared from Ph₃P and Cl₂), trimethylsilylchloride and bismuth(III) trichloride (Me₃SiCl/BiCl₃), mixtures of Ph₃P and carbon tetrachloride (CCl₄), or methanesulfonyl chloride (MeSO₂Cl) in pyridine at elevated temperatures.

Referring to Scheme 4, it will be appreciated by one skilled in the art that the presence of particular functional or protecting group in compounds of Formula (F) and Formula (G) determines the choice a particular reagent, method, or reaction condition for the chloro-de-hydroxylation reaction.

Referring to Scheme 4, in certain embodiments R¹ and/or R⁵, R²⁰, the linker L, E, the protecting groups PG and Q are defined as described herein; one of R², R³, and R⁴ in compounds of Formula (F) is a -E-N,N-bis(2-hydroxyethyl)amino group (-E-N(CH₂—CH₂—OH)₂); each of the other remaining R², R³, and R⁴ is hydrogen; and each of R⁷ and R⁸ is hydrogen.

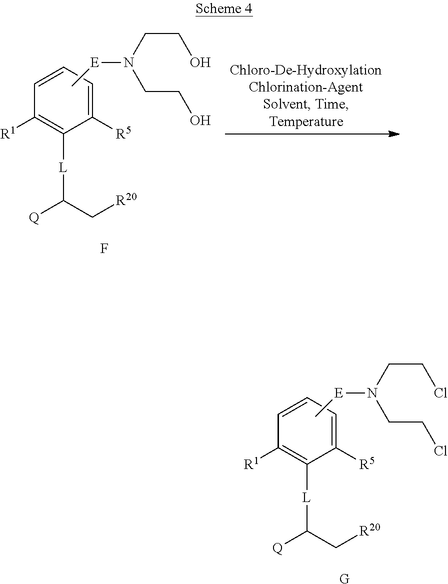

Referring to Scheme 4, in some embodiments N,N-bis(2-hydroxyethyl) compounds of Formula (F) may be reacted with an excess of about 2-15 equivalents of thionyl chloride (SOCl₂) either in neat form or as a solution in an anhydrous organic solvent, e.g., dichloromethane (DCM), chloroform (CHCl₃), 1,2-dichloroethane (DCE), benzene, or mixtures of any of the foregoing at temperatures from about 0° C. (ice bath) −40° C. or heated at reflux for about 0.5-3 hours to provide compounds of Formula (M) or of Formula (N) (Palmer, et al., J. Med. Chem. 1990, 33(1), 112-121; Jordan, et al., Bioorg. Med. Chem., 2002, 10(8), 2625-2633; Abela Medici, et al., J. Chem. Soc., Perkin Trans. 1, 1997, (20), 2258-2263; Taylor, et al., Chem. Biol. Drug Des., 2007, 70(3), 216-226; Dheyongera, Bioorg. Med. Chem. 2005, 13(3), 689-698; Zheng, Bioorg. Med. Chem. 2010, 18(2), 880-886; Gourdi, J. Med. Chem., 1990, 33(4), 1177-1186; and Lin, et al., Bioorg. Med. Chem. Lett., 2011, 21(3), 940-943). The reaction may optionally be carried out in the presence of a catalytic amount of zinc chloride (ZnCl₂) (10 mol-% to 40 mol-%) or in the presence of a catalytic amount of N,N-dimethylformamide (DMF) to facilitate the reaction (Squires, et al., J. Org. Chem., 1975, 40(1), 134-136; and Abela Medici, et al, J. Chem. Soc., Perkin Trans. 1, 1997, (20), 2258-2263).

Referring to Scheme 4, in some embodiments N,N-bis(2-hydroxyethyl) compounds of Formula (F) may also be reacted with an excess of about 2-10 equivalents of phosphorus(V)oxychloride (phosphoryl chloride, POCl₃) either in neat form or as a solution in an anhydrous organic solvent, e.g., benzene, acetonitrile, pyridine, or mixtures of any of the foregoing at a temperature from about 0° C. (ice bath) to about room temperature. The reaction mixture may also be heated from about 80° C. to about reflux temperature for about 0.5-6 hours to provide compounds of Formula (G) (Palmer, et al., J. Med. Chem. 1990, 33(1), 112-121; Feau, et al., Org. Biomolecular Chem., 2009, 7(24), 5259-5270; Valu, et al., J. Med. Chem., 1990, 33(11), 3014-3019; P. G. Baraldini, et al., J. Med., Chem., 2000, 53(14), 2675-2684; Gourdi, et al., J., Med., Chem., 1990, 33(4), 1177-1186; Haines, et al., J. Med. Chem., 1987, 30, 542-547; and Matharu, et al., Bioorg. Med. Chem. Lett., 2010, 20, 3688-3691).

Referring to Scheme 4, in some embodiments N,N-bis(2-hydroxyethyl) compounds of Formula (F) may also be reacted with an excess of carbon tetrachloride (CCl₄), optionally in an inert solvent, e.g., dichloromethane (DCM), in the presence of an excess of triphenylphosphine (Ph₃P) for about 8-24 hours at about room temperature or at reflux temperature for about 2-6 hours to provide compounds of Formula (G) (Buss, et al., J. Fluorine Chem., 1986, 34(1), 83-114; and Kupczyk-Subotkowska, et al., J. Drug Targeting, 1997, 4(6), 359-370).

Referring to Scheme 4, in some embodiments N,N-bis(2-hydroxyethyl) compounds of Formula (F) may also be reacted with methanesulfonyl chloride (MeSO₂Cl, MsCl) in anhydrous pyridine at about room temperature or at about 70-100° C. for about 1-3 hours to provide compounds of Formula (G) (Jordan, et al., Bioorg. Med. Chem., 2002, 10(8), 2625-2633; Abela Medici, et al, J. Chem. Soc., Perkin Trans. 1, 1997, (20), 2258-2263; Springer, et al., J. Med. Chem., 1990, 33(2), 677-681; and Larden and Cheung, Tetrahedron Lett., 1996, 37(42), 7581-7582).

Referring to Scheme 5, although halides are common leaving groups in nucleophilic substitution reactions for synthetic purposes, it is often more convenient to use the corresponding alcohols such as the ones found in N,N-bis(2-hydroxyethyl)amino groups of compounds of Formula (H). Since OH is usually considered a poor leaving group, unless protonated, conversion of a hydroxy group such as in N,N-bis(2-hydroxyethyl)amino groups of compounds of Formula (H) into reactive ester groups, most commonly sulfonic ester groups, converts the hydroxyl group into a functional group with a higher susceptibility to be displaced by an incoming nucleophile including halogenide ions. The N,N-bis(2-aryl- or (polyfluoro)alkylsulfonyloxy)amino groups of aryl- or (polyfluoro)alkylsulfonates of Formula (I) and similar sulfonic esters are most frequently prepared from N,N-bis(2-hydroxy)amino groups of diols of Formula (H) through reaction with an appropriate aryl- or (polyfluoro)alkyl-sulfonyl chloride or anhydride in the presence of a suitable base, e.g., pyridine (nucleophilic catalyst). Besides aromatic (R⁴⁰ is (substituted) aryl) sulfonic ester groups, aliphatic (R⁴⁰ is alkyl) sulfonic ester groups, and, in particular, (poly)fluorinated (R⁴⁰ is poly-F-alkyl) sulfonic ester groups as still more powerful leaving groups are frequently used for activation.

Referring to Scheme 5, in certain embodiments the R⁴⁰-group in compounds of Formula (I) or Formula (K) is for example phenyl and the leaving group is phenylsulfonyloxy (PhSO₂O), 4-methylphenyl (para-methylphenyl) and the leaving group is tosylate (4-methylphenylsulfonyloxy, TsO), 4-bromophenyl (para-bromophenyl) and the leaving group is brosylate (4-bromophenylsulfonyloxy, BsO), or 4-nitrophenyl (para-nitrophenyl) and the leaving group is nosylate (4-nitrophenylsulfonyloxy, NsO), methyl and the leaving group is mesylate (methanesulfonyloxy, MsO), trifluomethyl and the leaving group is triflate (trifluoromethanesulfonyloxy, TfO), nonafluoro-n-butyl and the leaving group is nonaflate (nonafluorobutanesulfonyloxy), or 2,2,2-trifluoroethyl and the leaving group is tresylate (2,2,2-trifluoroethanesulfonyloxy). In some embodiments, the R⁴⁰-group of compounds of Formula (I) and Formula (K) is methyl and the leaving group is mesylate (methansulfonyloxy, MsO). In some embodiments, the R⁴⁰-group of compounds of Formula (I) and of Formula (K) is trifluoromethyl and the leaving group is triflate (trifluoromethansulfonyloxy, TfO).

Referring to Scheme 5, N-mustard-type halides of Formula (J), Formula (K), and Formula (L) containing either (a) a N,N-bis(2-halogenoethyl)amino group (compounds of Formula (J)), (b) a N-(2-halogenoethyl)amino-, N-(2-halogeno′ethyl)amino- group (compounds of Formula (L) or mixed halogeno N-mustards), or (c) a N-(2-halogenoethyl)amino, N-(2-aryl- or (polyfluoro)alkylsulfonyloxyethyl)amino groups (compounds of Formula (K) or hybrid halogeno sulfonate N-mustards), may be prepared from the corresponding esters of sulfonic acid esters of Formula (P) through reaction with an excess or a near stoichiometric amount of an alkali metal halide (MX, MX′) in suitable protic or non-protic organic solvent at elevated temperature (halo-de-sulfonyloxy substitution)

Referring to Scheme 5, in certain embodiments M in MX or MX′ is an alkali metal cation, e.g., lithium (Li⁺) and sodium (Na⁺), X and X′ in MX or MX′ are halide anions, e.g., chloride (Cl⁻), bromide (Br⁻), and iodide (I⁻). MX or MX′ are alkali metal halides, e.g., lithium chloride (LiCl), lithium bromide (LiBr), sodium chloride (NaCl), sodium bromide (NaBr), or sodium iodide (NaI). In certain compounds of Formula (J), Formula (K), and Formula (L), X is a halogeno, e.g., chloro (—Cl), bromo (—Br), or iodo (—I) (Palmer, et al., J. Med. Chem. 1990, 33(1), 112-121; Palmer, et al., J. Med. Chem., 1994, 37, 2175-2184; Palmer, et al., J. Med. Chem., 1996, 39(13), 2518-2528; Davies, et al., J. Med. Chem. 2005, 48(16), 5321-5328; Niculesscu-Duvaz, et al., J. Med. Chem., 2004, 47(10), 2651-2658; Weisz, et al., Bioorg. Med. Chem. Lett., 1995, 5(24), 2985-2988; Thorn, J. Org. Chem, 1975, 40(11), 1556-1558; Lin, et al., Bioorg. Med. Chem. Lett., 2011, 21(3), 940-943; Gourdi, et al., J. Med. Chem. 1990, 33(4), 1177-1186; Yang, et al., Tetrahedron, 2007, 63(25), 5470-5476; Ferlin, et al., Bioorg. Med. Chem., 2004, 12(4), 771-777; and Coggiola, et al., Bioorg. Med. Chem. Lett., 2005, 15(15), 3551-3554).

Referring to Scheme 5, N-(2-halogenoethyl)amino, N-(2-aryl- or alkylsulfonyloxyethyl)amino groups of Formula (K) (hybrid halogeno sulfonate N-mustards) may also be prepared from primary alkyl halides of Formula (J) containing N,N-bis(2-halogenoethyl)amino groups through (a) a halo-de-halogenation (halide exchange reaction) or (b) a metathetical sulfonyloxy de-halogeno substitution reaction with solubilized silver sulfonates AgOSO₂R⁴⁰, wherein R⁴⁰ is defined as described herein under mild conditions in aprotic organic solvents (Emmons and Ferris, J Am. Chem. Soc., 1953, 75(9), 2257).

Referring to Scheme 5, for example in certain embodiments R¹ and/or R⁵, R²⁰, R⁴⁰, X, X′, E, the linker L, the protecting groups PG and Q are defined as herein; one of R², R³, and R⁴ in compounds of Formula (H) is -E-N(CH₂—CH₂—OH)₂ each of the other remaining R², R³, and R⁴ is hydrogen; and each of R⁷ and R⁸ is hydrogen.

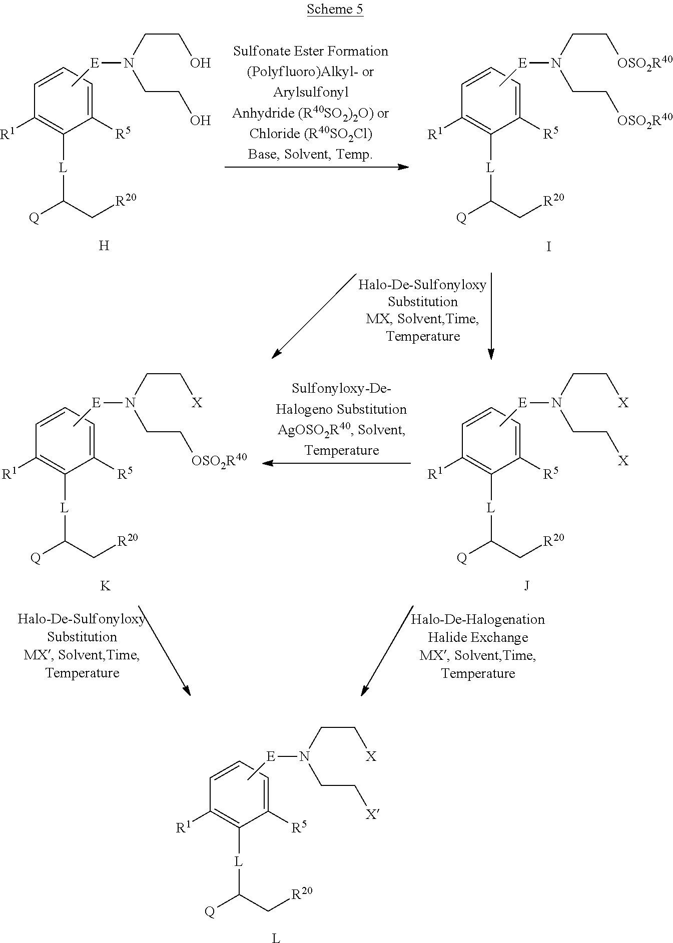

Referring to Scheme 5, in certain embodiments, the N,N-bis(2-hydroxyethyl)amino group of compounds of Formula (H) may be converted to N,N-bis(2-(polyfluoro)alkyl- or arylsulfonyloxyethyl)amino groups of compounds of Formula (I) (S-alkoxy-de-chlorination) by reacting diols of Formula (H) with an excess of a suitable (perfluoro)alkyl- or aryl-sulfonyl anhydride (R⁴⁰SO₂)₂O) (about 2.5-5 equivalents), e.g., methanesulfonyl anhydride (R⁴⁰ is methyl (Me), (MeSO₂)₂O)), in an inert solvent such anhydrous dichloromethane (DCM) or tetrahydrofuran (THF) or a mixture of any of the foregoing in the presence of an excess (about 2-10 equivalents) of a suitable base, e.g., anhydrous triethylamine (Et₃N, TEA) or anhydrous pyridine, at a temperature from about 0° C. to about room temperature for about 0.5-24 hours to afford bis-sulfonic acid esters of Formula (I). The reaction may optionally be carried out in the presence of a catalytic amount (about 20 mol-%) of 4-N,N-(dimethylamino)pyridine (DMAP).

Referring to Scheme 5, in certain embodiments, using comparable reaction conditions with respect to solvents, bases, stoichiometry of reagents, temperature, catalysts, and duration as described for the reaction of diols of Formula (H) with (ployfluoro)alkyl- or aryl-sulfonyl anhydrides, diols of Formula (H) may also be reacted with a suitable alkyl- or aryl-sulfonyl halides, e.g., methanesulfonyl chloride (mesyl chloride, MsCl) (R⁴⁰ is Me), MeSO₂Cl), to provide the desired bis-sulfonic acid esters of Formula (I).

Referring to Scheme 5, in certain embodiments N,N-bis(2-(polyfluoro)alkyl- or aryl-sulfonyloxyethyl)amino groups as in compounds of Formula (I) may be converted (halo-de-sulfonyloxy substitution) to N,N-bis(halogenoethyl)amino groups of compounds of Formula (J) by reacting bis-sulfonyl esters of Formula (I) with an excess of a suitable alkali metal halide salt MX, e.g., lithium chloride (LiCl), lithium bromide (LiBr), sodium chloride (NaCl), sodium bromide (NaBr), or sodium iodide (NaI) (4-16 equivalents) in a suitable organic solvent, e.g., N,N-dimethylformamide (DMF), N,N-dimethylacetamide (DMAc), acetone, 2-butanone (methyl ethyl ketone, MEK), 3-methyl-2-butanone (isopropyl methyl ketone, MIPK), acetonitrile (MeCN), methanol (MeOH), tetrahydrofuran (THF), ethyl acetate (EtOAc), or a mixture of any of the foregoing, at room temperature or heated to about 50-150° C. for about 0.5-6 hours to provide compounds of Formula (J).

Referring to Scheme 5, in certain embodiments using comparable reaction conditions with respect to solvents, temperature, and duration as described for the preparation of compounds of Formula (J), the reaction of bis-sulfonyl esters of Formula (I) may also be carried out in the presence of about one molar equivalent of a suitable alkali metal halide salt MX, as defined herein, to provide compounds of Formula (K) bearing N-(2-halogenoethyl)-, N-(2-methylsulfonyloxyethyl) amino groups (mixed halogeno/sulfonylato N-mustards).

Referring to Scheme 5, in some embodiments compounds of Formula (J) may be converted to mixed halogeno/sulfonylato N-mustards of Formula (K) by reacting N-mustard derivatives of Formula (J) where X is bromo (—Br) with about 1.0 equivalent or slightly less of a suitable soluble silver sulfonate salt, e.g., silver mesylate (AgOSO₂Me, AgOMs) in a polar solvent such as acetonitrile (MeCN) at about reflux temperature to provide the mixed halogeno/mesylate N-mustard of Formula (K) (methathetical reaction).

Referring to Scheme 5, in certain embodiments, using comparable reaction conditions with respect to solvents, temperature, and duration as described for the preparation of compounds of Formula (J) and of Formula (K), the reaction of bis-halogeno N-mustards of Formula (J) or of mixed halogeno/mesylate N-mustards of Formula (R) may also be carried out in the presence of about one molar equivalent of a suitable alkali metal halide salt MX′, as defined herein, to provide compounds of Formula (L) bearing N-(2-halogenoethyl)-, N-(2-halogeno′ethyl) amino groups (mixed halogeno N-mustards).

Reductive N-alkylation is a form of amination/alkylation that involves the reaction of an amino group with a carbonyl group to an amine in the presence of a suitable reducing agent via an intermediate imine or protonated imine. The carbonyl group component is most commonly an aldehyde or ketone functionality, the amino group is most commonly ammonia, a primary or secondary aliphatic amino group, or a primary or secondary aromatic amino group (aniline). For indirect reductive aminations, the intermediate imine may be isolated and reduced with a suitable reducing agent. For direct reductive aminations, the reaction may be carried out simultaneously, with the imine formation and reduction occurring concurrently, typically using reducing agents that are more reactive toward protonated imines than ketones, and that are stable under moderately acidic conditions, e.g., sodium cyanoborohydride (Na(CN)BH₃) or sodium triacetoxyborohydride (NaB(OAc)₃H.

Referring to Scheme 6, the primary amino group of compounds of Formula (M) either in a suitable salt form, e.g., a hydrochloride (HCl) salt (Ar-E-NH₂—HCl) or as a free base (Ar-E-NH₂) may be subjected to a reductive N-alkylation reaction using a suitable halocarbonyl compounds (X is F, Cl or, Br) or derivatives thereof, e.g. a dimethyl acetal, and reducing agents as they are well known in the art (Palani, et al., J. Med. Chem., 2005, 48(15), 4746-4749; van Oeveren, Bioorg. Med. Chem. Lett., 2007, 17(6), 1527-1531; Delfourne, et al., Bioorg. Med. Chem., 2004, 12(15), 3987-3994; Delfourne, et al., J. Med. Chem., 2002, 47(17), 3765-3771; and M. Jordan, et al., Bioorg. Med. Chem., 2002, 10(8), 2625-2633).

Suitable halocarbonyl compounds include, for example, 2-chloroacetic acid (ClCH₂CO₂H, X is Cl), 2-chloroacetaldehyde (ClCH₂CHO, X is Cl), or 2-bromoacetaldehyde dimethylacetal (MeO)₂CHCH₂Br, X is Br), optionally provided as solutions in suitable solvents, e.g., a 50-wt-% solution of 2-chloroacetaldehyde (ClCH₂CHO, X is Cl) in water.

Referring to Scheme 6, suitable reducing agents for reductive N-alkylations of primary amino groups such as in compounds of Formula (M) using 2-chloroacetic acid include boranes, preferably borane-tetrahydrofuran complex (H₃B.THF), and certain alkalimetal borohydrides, e.g., lithium borohydride (LiBH₄) or sodium borohydride (NaBH₄).

Referring to Scheme 6, the reaction is generally carried out in the presence of organic solvents such as protic solvents, e.g., methanol (MeOH), acetic acid, (HOAc), trifluoroacetic acid (TFA), 85 wt-% phosphoric acid (H₃PO₄), glacial acetic acid (HOAC), 98 wt-% formic acid, or water, or inert organic solvents, e.g., acetonitrile (MeCN), dichloromethane (DCM), tetrahydrofuran (THF), benzene, or equivalent mixtures of any of the foregoing at a temperature from about 0° C. to about reflux temperature and for about 0.5-18 hours. In embodiments where 2-chloroacetaldehyde is used, suitable reducing agents may include, for example, sodium cyanoborohydride (Na(CN)BH₃), sodium triacetoxyborohydride (NaB(OAc)₃H, and sodium borohydride (NaBH₄).

Reduction via hydrogenation is can also be employed. Preferred hydrogenation conditions include catalytic hydrogenation, for example, using palladium on carbon (Pd/C) as the catalyst. As the hydrogen source, gaseous hydrogen (H₂-gas) at pressures ranging from about atmospheric pressure to about 150 psi, or suitable ammonium salts, e.g., ammonium hydrogencarbonate (H₄NHCO₃), may be employed. The hydrogenation may be carried out at ambient temperature.

Referring to Scheme 6, in certain embodiments, R¹ and/or R⁵, R²⁰, E, the linker L, the halogeno group X, and the protecting group PG and Q are defined as herein; one of R², R³, and R⁴ in compounds of Formula (M) is -E-NH₂, wherein E is a bond (“—”), an oxygen atom (—O—), a methylene group (—CH₂—), or methylenoxy group (—CH₂—O—), and wherein MH is an amino group (—NH₂) so that -E-NH₂ is equivalent to a) a primary aromatic amino group (—NH₂, aniline), b) a primary O-aryl hydroxylamino group (—O—NH₂), c) a primary aminomethyl group (—CH₂—NH₂), or a primary O-benzyl hydroxylamino group (—CH₂—O—NH₂); each of the other remaining R², R³, and R⁴ is hydrogen; each of R⁷ and R⁸ is hydrogen.

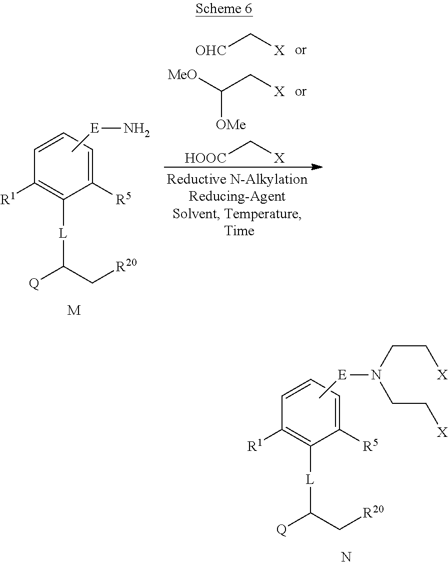

Referring to Scheme 6, in certain embodiments, the primary amino group of compounds of Formula (M) may be converted to N,N-bis(2-halogenoethyl)amino groups as in compounds of Formula (N) by reacting compounds of Formula (M) with an excess of about 4-10 equivalents of a 2-halogenocarbonyl compound, e.g., a 50 wt-% solution of 2-chloroacetaldehyde in water, and an excess of about 3-8 equivalents of a suitable reducing agent, e.g., sodium cyanoborohydride (NaB(CN)H₃). In certain embodiments, the reaction may be carried out in mixtures of methanol (MeOH) with trifluoroacetic acid (TFA), glacial acetic acid (HOAc), 98 wt-% formic acid (FA), or 85 wt-% phosphoric acid (H₃PO₄). For example, in certain embodiments, 1:1 (v/v), 2:1 (v/v), or 1:2 (v/v) mixtures MeOH/acid and reaction temperatures from about 0-40° C. and reaction times of about 0.5-18 hours are employed to provide protected N-mustards of Formula (N).

Estramustine (Emcyt®, Estracit®) is an antimicrotubule chemotherapy agent indicated in the US for the palliative treatment of metastatic and/or progressive prostate cancer. It is derivative of estrogen (specifically, estradiol) with a N-mustard-carbamate ester moiety.

Referring to Scheme 7, methods to functionalize alcohols or phenols with carbamoyl derivatives of secondary amines yielding carbamates as in, for example, compounds of Formula (Q) wherein M is oxygen (—O—) and G is oxygen (═O) include carbamoyl chlorides or p-nitrophenyl carbamates, and are well known in the art. Likewise, it is well known in the art that carbamates as in, for example, compounds of Formula (Q) wherein M is oxygen (—O—) and G is oxygen (═O) are also accessible through activation of alcohols or phenols with suitable formic ester derivatives including phosgene (COCl₂), triphosgene (bis(trichloromethyl) carbonate (BTC)), or 1,1′-carbonyldiimidazole (CDI) followed by reaction with an appropriately functionalized amine such as HN(CH₂—CH₂—R⁹)₂ wherein R⁹ is chloro (—Cl), bromo (—Br), iodo (—I), or (polyfluoro)alkyl- or aryl sulfonyloxy (—OSO₂R⁴⁰) or combinations thereof and R⁴⁰ is defined as described herein.

Likewise and referring to Scheme 7, many methods are known in the literature and are known by those skilled in the art to prepare compounds of Formula (Q) related to carbamates including a) S-thiocarbamates wherein M is sulfur (—S—) and G is oxygen (═O), b) O-thiocarbamates wherein M is oxygen (—O—) and G is sulfur (═S), c) dithiocarbamates wherein M is sulfur (—S—) and G is sulfur (═S), d) ureas wherein M is nitrogen (—NR¹⁰—), and where R¹⁰ is defined as described herein, and G is oxygen (═O), or thioureas wherein M is nitrogen (—NR¹⁰—) and G is sulfur (═S).

Referring to Scheme 7, in certain embodiments a compound of Formula (O) is, for example, a) a phenol wherein E is a bond (“—”) and MH is a hydroxyl group (—OH), b) an aniline wherein E is a bond (“—”) and MH is an amino group (—NR¹⁰H), c) a thiophenol wherein E is a bond (“—”) and MH is a sulfhydryl group (—SH), d) an O-aryl hydroxylamine wherein E is oxygen (—O—) and MH is an amino group (—NR¹⁰H), e) a benzylic alcohol wherein E is methylene (—CH₂—) and MH is a hydroxyl group (—OH), f) a benzylic amine wherein E is methylene (—CH₂—) and MH is an amino group (—NR¹⁰H), g) a benzylic thiol wherein E is methylene (—CH₂—) and MH is sulfhydryl (—SH), h) an O-benzylic hydroxylamine wherein E is methyleneoxy (—CH₂—O—) and MH is an amino group (—NR¹⁰H).

Referring to Scheme 7, in certain embodiments, R¹ and/or R⁵, R¹⁰, R²⁰, E, M, Z, the linker L, and the protecting group PG and Q are defined as described herein; one of R², R³, and R⁴ in compounds of Formula (O) is -E-MH as described herein; each of the other remaining R², R³, and R⁴ is hydrogen; each of R⁷ and R⁸ is hydrogen; LG is a suitable leaving group such as chloro (—Cl), 4-nitrophenyloxy (NO₂C₆H₄O—), or imidazole; and R⁹ is chloro (—Cl), bromo (—Br), iodo (—I), or (polyfluoro)alkyl- or aryl sulfonyloxy (—OSO₂R⁴⁰) or combinations thereof, and R⁴⁰ is defined as described herein.

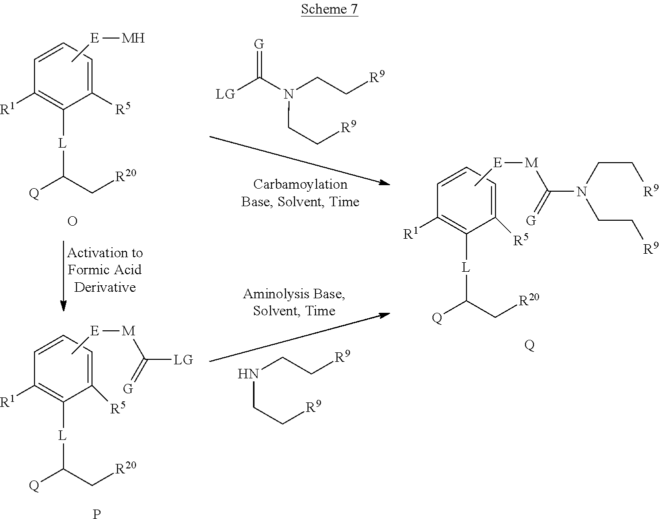

Referring to Scheme 7, in certain embodiments the alcohol, the thiol group, or the amino group of compounds of Formula (O) may be converted to the N,N-bis(2-halogeno- or 2-sulfonyloxyethyl)carbamoyl or N,N-bis(2-halogeno- or 2-sulfonyloxyethyl)thiocarbamoyl group of compounds of Formula (Q) by reacting a compound of Formula (O) with, for example, commercial N,N-bis(2-chloroethyl)carbamoyl chloride (Fex, et al., U.S. Pat. No. 3,299,104), wherein LG is chloro (—Cl), R⁹ is chloro (—Cl), and G is oxygen (═O) or known (4-nitrophenyl) N,N-bis(2-chloroethyl)carbamate where LG is 4-nitrophenol (4-NO₂-Ph-O—), R⁹ is chloro (—Cl), and G is oxygen (═O) in suitable solvents such as pyridine, or triethylamine in 1,4-dioxane/benzene mixtures and the like at temperatures of about 0-60° C. to provide carbamate, thiocarbamate, or urea derivatives of Formula (Q).

Referring to Scheme 7, in certain embodiments the MH-group of compounds of Formula (O) may be activated to their corresponding chloroformates, thiochloroformates, or carbonyl imidazoles of Formula (P) with, for example, phosgene, thiosphosgene, triphosgene, carbonyldiimidazole (CDI), thiocarbonyldiimidazole (TCDI), or the like, in the presence of a suitable base such as inorganic metal-carbonate, e.g., potassium carbonate (K₂CO₃) and bicarbonates, e.g., sodium hydrogencarbonate (NaHCO₃), in suitable inert solvents known in the art. The chloroformates or thiochloroformates of Formula (P) are subsequently converted to the corresponding carbamates of Formula (Q) through reaction with an appropriately functionalized amine such as HN(CH₂—CH₂—R⁹)₂ wherein R⁹ is chloro (—Cl), bromo (—Br), iodo (—I), or (polyfluoro)alkyl- or aryl sulfonyloxy (—OSO₂R⁴⁰) or combinations thereof, and R⁴⁰ is defined as described herein, e.g., commercial bis(2-chloroethyl)amine hydrochloride wherein R⁹ is chloro (—Cl) or 2-bromo-N-(2-bromoethyl)ethanamine wherein R⁹ is bromo (—Br), and in the presence of a base such as inorganic metal-carbonate, e.g., potassium carbonate (K₂CO₃) and bicarbonate, e.g., sodium hydrogencarbonate (NaHCO₃), ethyl acetate (EtOAc), water, or mixtures of any of the foregoing to yield carbamates of Formula (Q).

In general, the biological activity of nitrogen mustards is based upon the presence of an alkylating N,N-bis(2-chloroethyl) functionality. The chemotherapeutic and cytotoxic effects are directly associated with the alkylation of DNA due to the strong electrophilic character of the N,N-bis(2-chloroethyl) functionality. Formation of covalent linkages including interstrand crosslinks (ICLs) is highly cytotoxic and involves the disruption of fundamental cellular processes including DNA replication leading to cellular death.

Because of this property, the nitrogen mustards have been used for a number of years in laboratory investigations and in the clinical treat for malignat growth. Unfortunately, the effective dose of nitrogen mustards is in many cases close to the toxic dose and it is therefore desirable to find a nitrogen mustard or a class of nitrogen mustard type compounds possessing the high carcinolytic activity of the parent compound but having modulated toxicity.

The amide linkage masks the alkylating and toxic properties of the nitrogen mustard moiety so that the total host is not subjected to undesirable toxic effects sometime encountered with nitrogen mustard therapy: the amino acid moiety of the molecule facilitates the selective delivery of the “masked” nitrogen mustard via the amino acid transport mechanism into the tumor cells, where the higher amidase activity of the tumor cell liberates the reactivated nitrogen mustard within itself. Thus in effect it will be possible to obtain maximum effect of the nitrogen mustard on the tumor and minimum toxic effect on the host (U.S. Pat. No. 3,235,594).

Referring to Scheme 8, the amide nitrogen mustards of the present disclosure are prepared by condensing carboxylic acids of Formula (R) wherein E is a carbonyl group (—C(═O)—) or a methylenecarbonyl group (—CH₂—C(═O)—) with an appropriately functionalized amine such as HN(CH₂—CH₂—R⁹)₂ wherein X is chloro (—Cl), bromo (—Br), iodo (—I), or (polyfluoro)alkyl- or aryl sulfonyloxy (—OSO₂R⁴⁰) or combinations thereof, and R⁴⁰ is defined as described herein, to provide amides of nitrogen mustards of Formula (S).

Referring to Scheme 8, a myriad of coupling methods is known in the art to facilitate the formation of amide bonds as in compounds of Formula (S) from carboxylic acids of Formula (R) (Montalbetti and Falque, Tetrahedron, 2005, 61, 10827-10852; and Valeur and Bradley, Chem. Soc. Rev., 2009, 38, 606-631).

Referring to Scheme 8, in certain embodiments, R¹ and/or R⁵, R²⁰, E, the linker L, and the protecting group PG and Q are defined as described herein; one of R², R³, and R⁴ in compounds of Formula (R) is -E-OH as described herein; each of the other remaining R², R³, and R⁴ is hydrogen; each of R⁷ and R⁸ is hydrogen; and R⁹ is a suitable functionalization providing the alkylation properties of the nitrogen mustard.

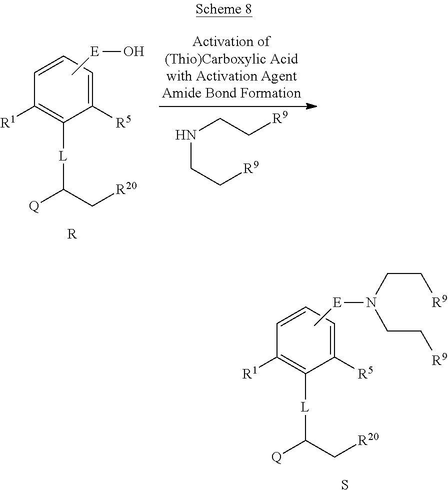

Referring to Scheme 8, in certain embodiments the (thio)carboxyl group of compounds of Formula (R) may be activated as acyl halides, acyl azides, symmetrical or unsymmetrical carboxylic, carbonic, or boronic anhydrides, acyl imidazoles, activated esters, phosphonium salts, uronium salts, or ammonium salts followed by ammonolysis of the activated intermediate either after prior isolation or in situ with an appropriately functionalized amine such as HN(CH₂—CH₂—R⁹)₂ to provide nitrogen mustard amides of Formula (S).

Referring to Scheme 9, in certain embodiments the connector group “A” of the moiety -A-N(CH₂—CH₂—R⁹)₂ is a bond (“—”), oxygen (—O—), sulfur (—S—), amino (—NR¹⁰—) methylene (—CH₂—), methyleneoxy (—CH₂—O—), oxycarbonyl (—O—C(═O)—), thiocarbonyl (—S—C(═O)—), aminocarbonyl (—NR¹⁰—C(═O)—), oxythiocarbonyl (—O—C(═S)—), thiothiocarbonyl (—S—C(═S)—), aminothiocarbonyl (—NR¹⁰—C(═S)—), methyleneoxycarbonyl (—CH₂—O—C(═O)—), methylenethiocarbonyl (—CH₂—S—C(═O)—), methyleneaminocarbonyl (—CH₂—NR¹⁰—C(═O)—), methyleneoxythiocarbonyl (—CH₂—O—C(═S)—), methylenethiothiocarbonyl (—CH₂—S—C(═S)—), methyleneaminothiocarbonyl (—CH₂—NR¹⁰—C(═S)—), carbonyl (—C(═O)—), methylencarbonyl (—CH₂—C(═O)—), thiocarbonyl (—C(═S)—), or methylenthiocarbonyl (—CH₂—C(═S)—).

Referring to Scheme 9, in certain embodiments liberation of unprotected N-mustard functionalized β-substituted β-amino acid derivatives or unprotected N-mustard functionalized β-substituted β-amino acid analogs or carboxylic acid (bio)isosteres of Formula (U) from their corresponding precursors of Formula (T) may be conducted under aqueous acidic conditions (hydrolysis) (Taylor, et al., Chem. Biol. Drug Des., 2007, 70(3), 216-226; Buss, et al., J. Fluorine Chem., 1986, 34(1), 83-114; A. J. Abela, et al, J. Chem. Soc., Perkin Trans. 1, 1997, (20), 2258-2263; Weisz, et al., Bioorg. Med. Chem. Lett., 1995, 5(24), 2985-2988; Zheng, Bioorg., Med., Chem., 2010, 18(2), 880-886; Haines, et al., J. Med. Chem., 1987, 30, 542-547; and Matharu, et al., Bioorg., Med., Chem., Lett., 2010, 20, 3688-3691).

Referring to Scheme 9, in certain embodiments liberation of unprotected N-mustard functionalized β-substituted β-amino acid derivatives or unprotected N-mustard functionalized β-substituted β-amino acid analogs or carboxylic acid (bio)isosteres of Formula (U) from their corresponding precursors of Formula (T) may also be conducted under anhydrous acidic conditions (Springer, et al., J. Med. Chem., 1990, 33(2), 677-681; Davies, et al., J. Med. Chem. 2005, 48(16), 5321-5328; Niculesscu-Duvaz, et al., J. Med. Chem., 2004, 47(10), 2651-2658; Verny and Nicolas, J. Label. Cmpds, Radiopharm., 1988, 25(9), 949-955; Thorn, et al., J. Org. Chem, 1975, 40(11), 1556-1558; Baraldini, et al., J. Med. Chem., 2000, 53(14), 2675-2684; Gourdi, et al., J. Med. Chem., 1990, 33(4), 1177-1186; and Kupczyk-Subotkowska, et al., J. Drug Targeting, 1997, 4(6), 359-370).

Referring to Scheme 9, it will be understood by those skilled in the art that protected N-mustard functionalized β-substituted β-amino acid precursors of Formula (T) or protected N-mustard β-substituted β-amino acid analog or carboxylic acid (bio)isosteres precursors of Formula (T) bearing different combinations of suitable protecting groups may also be prepared. Different combinations of protecting groups may require specific reactants and reaction conditions for effective removal of specific set of different protection groups to provide unprotected N-mustard β-substituted β-amino acid derivatives or unprotected N-mustard funtionalized β-substituted β-amino acid derivatives, analogs, or carboxylic acid (bio)isosteres of Formula (U).

Referring to Scheme 9, in certain embodiments of compounds of Formula (T) and of Formula (U) R¹ and/or R⁵, R⁹, the connector group A, the protecting groups PG and Q, and the linker L are defined as described herein; R⁶ is an unprotected carboxylic acid, a carboxylic acid analog or a carboxylic acid (bio)isostere as defined herein; R²⁰ is a protected carboxylic acid, a carboxylic acid analog or a carboxylic acid (bio)isostere as defined herein; one of R², R³, and R⁴ is a N,N-bis-(2-functionalized)ethylamino group (nitrogen mustard group) linked to a connector A (-A-N(CH₂—CH₂—R⁹)₂); each of the remaining R², R³, and R⁴ is hydrogen; each of R⁷ and R⁸ is hydrogen.

### Scheme 9

Referring to Scheme 9, hydrolytic acidic global deprotection of compounds of Formula (T) to provide N-mustard functionalized β-substituted β-amino acid derivatives or N-mustard functionalized β-substituted β-amino acid analogs or carboxylic acid (bio)isosteres of Formula (U) may be accomplished by treating protected precursors of Formula (T) at elevated temperatures from about 40-150° C. with aqueous mineral acids, e.g., 2 M to ˜12 M hydrochloric acid (HCl) for about 6-24 hours. In certain embodiments, mixtures of the mineral acid with organic solvents may be used. A useful aqueous mineral acid reaction mixture to facilitate global deprotection is, e.g., a 1:1 (v/v) mixture of concentrated hydrochloric acid (˜12 M or ˜37 wt-% HCl) with 1,4-dioxane.

Referring to Scheme 9, other aqueous mineral acids with a non-nucleophilic anion known in the art can be used to facilitate hydrolytic acidic global deprotection of compounds of Formula (T) bearing acid-labile or hydrolysis sensitive protecting groups of the protected carboxylic moiety, of the protected carboxylic acid (bio)isostere, or of the amino functionality of compounds of Formula (T) to provide N-mustard functionalized β-substituted β-amino acid derivatives or N-mustard functionalized β-substituted β-amino acid analogs or carboxylic acid (bio)isosteres of Formula (U).

Referring to Scheme 9, suitable mineral acids may for example include diluted or concentrated aqueous solutions of hydrobromic acid (HBr), hydroiodic acid (HI), sulfuric acid (H₂SO₄), perchloric acid (HClO₄), and phosphoric acid (H₃PO₄), mixtures of any of the foregoing or mixtures with suitable organic solvents, e.g., 1,4-dioxane, with any of the foregoing.

It is within the ability of one skilled in the art to select specific and suitable aqueous mineral acids and reaction conditions for hydrolytic acidic hydrolytic acidic global deprotection of compounds of Formula (T) to provide N-mustard functionalized β-substituted β-amino acid derivatives or N-mustard functionalized β-substituted β-amino acid analogs or carboxylic acid (bio)isosteres of Formula (U).

Referring to Scheme 9, simultaneous global deprotection of compounds of Formula (T) where R²⁰ is an acid labile moiety derived from a carboxylic acid, e.g., CO₂tBu, CO₂-pentamethylbenzyl, CO₂-(4-methoxy)benzyl, or CO₂-trityl, and Q is a protected amino group derived from an acid-labile N-protecting group, e.g., N(H)Boc, N(H)trityl, N(H)(4-methoxy)phenyl-diphenylmethyl, or N(H)di-((4-methoxy)phenyl)-phenylmethyl, may also be accomplished by reaction with strong organic acids under anhydrous conditions to liberate free (unprotected) N-mustard functionalized β-substituted β-amino acid derivatives or N-mustard functionalized β-substituted β-amino acid analogs or carboxylic acid (bio)isosteres of Formula (U).

In certain embodiments, strong (organic) acids useful for global deprotection under anhydrous conditions include trifluoroacetic acid (TFA), 98 wt-% formic acid (FA), methanesulfonic acid (MeSO₃H), 85 wt-% phosphoric acid (H₃PO₄), 2 M hydrogen chloride (HCl) in diethyl ether (Et₂O), 4 M hydrogen chloride (HCl) in 1,4-dioxane, or a saturated solution of HCl in ethyl acetate (EtOAc) (Li, et al., J. Org. Chem., 2006, 71, 9045-9050).

Depending of the overall sensitivity to strong (organic acids), compounds of Formula (T) may be reacted with neat either neat strong (organic) acid or with solutions of the strong organic acid in suitable inert solvents such asdichloromethane (DCM), dichloroethane (DCE), 1,4-dioxane, diethylether (Et₂O), tetrahydrofuran (THF), or toluene typically in ratios ranging from neat (organic) acid to about 10 vol-% (organic) acid in said inert solvent, and reaction temperatures ranging from about 0-50° C. for about 1-24 hours to provide unprotected N-mustard functionalized β-substituted β-amino acid derivatives or unprotected N-mustard functionalized β-substituted β-amino acid analogs or carboxylic acid (bio)isosteres of Formula (U).

Optionally, 2-5 equivalents of a suitable scavenging agent such as triethysilane (Et₃SiH) (TES), triisopropylsilane (iPr₃SiH), thioanisole, or 1,2-dithioethane (HSCH₂CH₂HS) may be added to the reaction mixture to suppress formation of unwanted side reactions and byproducts originating, for example, from alkylation of electron-rich aromatic scaffolds or sulfide groups under global deprotection conditions disclosed herein to provide unprotected N-mustard functionalized β-substituted β-amino acid derivatives or unprotected N-mustard functionalized β-substituted β-amino acid analogs or carboxylic acid (bio)isosteres of Formula (U).

Separation of unprotected N-mustard functionalized β-substituted β-amino acid derivatives or unprotected N-mustard functionalized β-substituted β-amino acid analogs or carboxylic acid (bio)isosteres of Formula (U) from unreacted starting materials, unwanted byproducts, and impurities may be accomplished using, for example, solid-phase extraction (SPE) techniques, e.g., with QMA® cartridges (Waters, USA), LiChrolut® cartridges (EMD Chemicals, USA), or Whatman SAX cartridges (Whatman, USA), preparative normal or reverse phase TLC, reverse phase (RP) semi-preparative or preparative HPLC, crystallization, precipitation, or any other suitable method known in the art.

Purified unprotected N-mustard functionalized β-substituted β amino acid derivatives or unprotected N-mustard functionalized β-substituted β-amino acid analogs or carboxylic acid (bio)isosteres of Formula (U) may be isolated using any of the methods known in the art. For example, such methods include removal of HPLC solvents (mobile phase) of the combined fractions containing the N-mustard functionalized β-substituted β-amino acid derivatives or N-mustard functionalized β-substituted β-amino acid analogs or carboxylic acid (bioisosteres) of Formula (U) under reduced pressure with a rotary evaporator, or removal of (aqueous) solvent mixtures by primary lyophilization.

Any method known in the art may be used to produce acid addition salts or salts including pharmaceutically acceptable acid addition salts or salts of compounds of Formula (U) (Handbook of Pharmaceutical Salts—Properties, Selection, and Use, Stahl and Wermuth, Wiley-VCH, Weinheim, Germany, 2008).

The lyophilization may optionally be conducted in the presence of one or more equivalents of a mineral acid, optionally with a pharmaceutically acceptable counterion, to form (pharmaceutically acceptable) acid addition salts of compounds of Formula (U). For example, one or more equivalents of hydrochloric acid (HCl) may be added prior to lyophiliation to form mono-, di-, or polyhydrochloride salts of compounds of Formula (U) or mixtures thereof.

The lyophilization may optionally be conducted in the presence of one or more equivalents of a base, optionally with a pharmaceutically acceptable counterion, to form (pharmaceutically acceptable) salts of compounds of Formula (U). For example, one or more equivalents of sodium hydrogen carbonate (NaHCO₃) may be added prior to lyophilization to form mono-, di-, or poly sodium salts of compounds of Formula (U) or mixtures thereof.

A characteristic feature of solid tumors is the presence of cells at very low oxygen concentrations (hypoxia; partial pressure of oxygen in tumorous tissue of 0.05-5.0%) often surrounding areas of necrosis. There are clear links between hypoxia and the lack of response to radiotherapy and intrinsic resistance to cytotoxic therapy. It has also been demonstrated that hypoxia in tumors tends to select for a more malignant phenotype (Wilson and Hay, Nat. Rev. Canc., 2011, 11, 393-410; and Brown and Wilson, Nat. Rev. Canc., 2004, 4, 437-447).

Reductive metabolic processes are more prevalent in the hypoxic environment of solid tumors. Reductive enzyme systems have the ability to reduce certain functional groups. For example, aromatic and aliphatic N-oxides (—N⁺(O⁻)R₂) are known to be reducible to the corresponding amines (—NR₂), and nitro groups (—NO₂) can be either reduced to the corresponding amines (—NH₂) or to hydroxylamines (—NH(OH) depending on the oxygen saturation of the tissue (Denny, et al., Br. J. Canc., 1996, 74, Suppl. XXVII, S32-S38; and Nagasawa, et al., Biol. Pharm. Bull., 2006, 29(12), 2335-2342).

One promising approach for the design of cancer-cell-selective mustards exploits selective enzymatic reduction of nitroaryl compounds in the oxygen-starved (hypoxic) cells found in solid tumors. N-Oxide derivatives of nitrogen mustards including N-oxides of melphalan (PX-478; Kirkpatrick, et al., U.S. Pat. No. 7,399,785; Koh, et al., Mol. Canc. Ther., 2008, 7(1), 90-100; www.medkoo.com) and chlorambucil (Kirkatrick, et al., Anti-Cancer Drugs, 1994, 5, 467-472; Tercel, et al., J. Med. Chem., 1995, 38, 1247-1252; and Kirckpatrick, U.S. Pat. No. 5,602,273) have been investigated as bioreductive prodrugs with reduced systemic toxicity in comparison to the parent drugs. Those drugs take advantage of a) the hypoxic nature, and b) the reductive nature, of certain tumorous cells. The N-oxide functional group deactivates the extremely reactive alkylating agent through capture of the lone electron pair of the parent nitrogen mustard moiety thus diminishing the alkylating properties and the off-target toxicities associated with that. Bioreductive activation within the hypoxic tumor environment or milieu by hypoxic cells and their reductive enzyme systems is believed to restore the cytotoxicity of the free nitrogen mustards. The overall effect is an enhanced therapeutic index of the N-oxides of nitrogen mustards relative to their parent nitrogen mustards.

Depending on the pH and the nature of the solvent, particularly aprotic organic solvents, N-oxides of nitrogen mustards are known to intramolecularly rearrange to the corresponding more stable hydroxylamines with markedly less intrinsic cytotoxic potential (Tercel, et al., J. Med. Chem., 1995, 38, 1247-1252; and Kirckpatrick, U.S. Pat. No. 5,602,273). However, it is also known that said hydroxylamines are able to convert back to the parent N-oxides in vivo where the latter can be reduced in the hypoxic and reductive environment of tumorous cells where the underlying nitrogen mustards exerts their cytoxicity.

Referring to Scheme 10, in certain embodiments of compounds of Formula (V), Formula (W), and of Formula (X) R¹ and/or R⁵, R⁶, R⁹, and the linker L are defined as described herein; one of R², R³, and R⁴ is a N,N-bis-(2-functionalized)ethylamino group (nitrogen mustard group) linked to a connector group “A” (-A-N(CH₂—CH₂—R⁹)₂) wherein the connector group “A” is a bond (“—”) or a methylene group (—CH₂—); each of the remaining R², R³, and R⁴ is hydrogen; each of R⁷ and R⁸ is hydrogen.

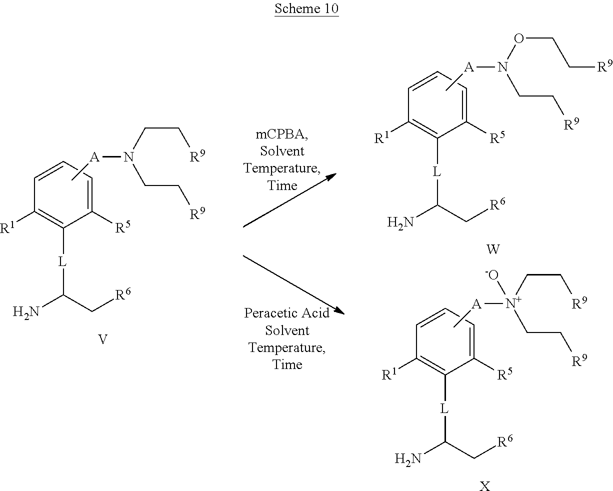

Referring to Scheme 10, N-oxidation of the N-mustard group of compounds of Formula (V) with a slight excess of 3-chloroperbezoic acid (meta-chloroperbenzoic acid, mCPBA) in a solvent such as dichloromethane (DCM) at about room temperature followed by work-up with aqueous sodium hydrogencarbonate furnishes the more stable hydroxylamine (through putative re-arrangement via a cyclic oxazetidinium species) of Formula (W).

Referring to Scheme 10, N-oxidation of the N-mustard group of compounds of Formula (V) with 3-5 equivalents of peracetic acid (MeCO(O₂H)), prepared from 35 wt-% aqueous hydrogen peroxide (H₂O₂) in glacial acetic acid (HOAc), in a solvent such as dichloromethane (DCM) at about room temperature followed by acid extraction furnishes the corresponding N-oxide of Formula (X).

To determine the extent to which compounds provided by the present disclosure enter cells via the LAT1/4F2hc transporter, amino acid uptake assays into cells that are transfected with DNA encoding the LAT1 and 4F2hc subunits may be performed using, for example, HEK (human embryonic kidney) or CHO (Chinese hamster ovary) cells. Oocytes may also be injected with cRNA LAT1 and 4F2hc to express LAT1/4F2hc transporter. Compounds may be screened either for specificity for the LAT1/4F2hc transporter or for transport into cells endogenously expressing a plurality of transporters. The results of a screening method (e.g., a competition uptake, exchange or direct uptake assay) using a cell expressing the LAT1/4F2hc transporter may be compared with the results of a control cell(s) lacking the LAT1/4F2hc transporter or in the presence of a specific inhibitor of the LAT1/4F2hc transporter.

In competition experiments, the ability of a compound to specifically bind to the LAT1/4F2hc transporter is determined. A known substrate (reference substrate) for the LAT1/4F2hc transporter and a test compound are added to cells expressing the LAT1/4F2hc transporter. For example, gabapentin may be used as a reference because it demonstrates high selectivity for LAT1/4F2hc. Gabapentin is not a substrate for the intestinal amino acid transporters B^{0,+}, ATB⁰⁺, and LAT2, whereas gabapentin may be a substrate for the organic cation transporter OCTN2 (Cundy, et al., J Pharm Exp Ther, 2004, 311(1), 315-323; and Grigat, et al., Drug Metabol Disp, 2009, 37(2), 330-337). The amount or rate of transport of the reference substrate in the presence of the test compound is compared to the amount or rate of transport of the reference substrate in the absence of the test compound. If the amount or rate of transport of the reference substrate is decreased by the presence of the test compound, the test compound binds to the LAT1/4F2hc transporter.

Compounds that bind the LAT1/4F2hc transporter can be further analyzed to determine if they are transported by the LAT1/4F2hc transporter or only compete for binding to the transporter. Transport of a compound into a cell can be determined by detecting a signal from within a cell from any of a variety of reporters. The reporter can be as simple as a label such as a fluorophore, a chromophore, a radionuclide, or a reporter can be an agent that is detected utilizing liquid chromatography-mass spectroscopy (LC/MS/MS). The same methods of detection can be used to determine if a reporter is transported from the intracellular space to the medium by administering the test compound to the outside of the cell and sampling the media for the presence of the intracellular reporter after a predetermined period of time (exchange assays).

Having determined that a compound is a substrate for LAT1/4F2hc, a further screen may be performed to determine the selectivity of the compound toward other membrane transporters. Selectivity refers to the affinities with which a compound is transported by different transporters. In order to demonstrate selectivity for LAT1/4F2hc, a compound may be tested in uptake and/or competition assays for other transporters. Transporters that could potentially transport LAT1/4F2hc substrates include SLC1A4 (ASCT1; NP_003029), SLC1A5 (ASCT2; NP_005619), SLC6A1 (GAT1; NP_003033), SLC6A5 (GlyT2; NP_004202), SLC6A6 (TauT; NP_003034), SLC6A8 (CT1; NP_005620), SLC6A9 (GlyT1; NM_008865), SLC6A11 (GAT3; NP_55044), SLC6A12 (BGT1; NP_003035), SLC6A13 (GAT2; NP_057699), SLC6A14 (ATB^{0,+}; NP_009162), SLC6A15 (B⁰AT2; NP_001139807), SLC6A17 (XT1; NP_001010898), SLC6A18 (B⁰AT3; NP_872438), SLC6A19 (B⁰AT1; NP_001003841), SLC7A6 (y⁺LAT2; NP_001070253), SLC7A7 (y⁺LAT1; NP_001119577), SLC7A8 (LAT2; NP_036376), SLC7A9 (b^{0,+}AT; NP_055085), SCL7A10 (ASC-1; NP_062823), SLC15A1(PepT1; NP_005064), SLC15A2 (PepT2; NP_066568), SLC16A1 (MCT1; NP_003042), SLC16A2 (MCT8; NP_006508), SLC16A10 (TAT1; NP_061063), SLCO1B1 (OATP1B1; NP_006437), SLCO1B3 (OATP1B3; NP_062818), SLC22A1 (OCT1; NP_003048), SLC22A2 (OCT2; NP_003049), SLC22A4 (OCTN1; NP_003050), SLC22A5 (OCTN2; NP_003051), SLC22A8 (OAT3; NP_004245), SLC36A1 (PAT1; NP_510968), SLC36A1 (PAT1; NP_510968), SLC36A2 (PAT2; NP_861441), SLC38A1 (SNAT1; NP_109599), SLC38A2 (SNAT2; NP_061849), SLC38A3 (SNAT3; NP_006832), SLC38A4 (SNAT4; NP_060488), SLC38A5 (SNAT5; NP_0277053), SLC43A1 (LAT3; NP_003618), and SLC43A2 (LAT4; NP_689559).

Human genes required for functional expression of a transporter of interest may be cloned using PCR, fully sequenced, and subcloned into plasmids that can be used for expression in mammalian cells or Xenopus laevis oocytes. Unless otherwise noted, all subunits of a transporter of interest are co-expressed in each heterologous system described in the examples. Because many mammalian cell lines exhibit high levels of amino acid transport activity, expression in Xenopus laevis oocytes can be advantageous due to the low levels of endogenous amino acid transport. To assess transport function of a specific transporter protein, it can be desirable to clone the cDNA and express the protein in cells that have low endogenous transport activity. Competition assays may be performed with labeled compounds that are optimal substrates (reference substrates) for the transporter of interest. Typically, uptake levels of a test compound are compared to uptake of a reference substrate for the transporter of interest.

Compounds of Formula (1) are substrates for LAT1/4F2hc and have a Vₘₐₓ of at least 10%, 20%, and in certain embodiments, at least 50% that of gabapentin. Concomitantly, the compounds have a low affinity toward amino acid transporters of system A, system N, system ASC, and the system L transporter LAT2/4F2hc.

Biodistribution studies with normal and tumor-bearing rats may be used to determine the disposition of actively transported compounds and the selectivity of substrate accumulation in tissue that expresses the LAT1/4F2hc transporter compared with other tissue. Imaging techniques can qualitatively and quantitatively elucidate the role of transport proteins in drug disposition, for example, whole body autoradiography (WBA). WBA allows both the visualization and the quantification of radionuclide-labeled compound levels in a thin section of the whole animal. Information obtained using WBA is analogous to data obtained from diagnostic imaging, albeit at a single point in time.

Compounds of Formula (1) or pharmaceutically acceptable salts thereof may be incorporated into pharmaceutical compositions to be administered to a patient by any appropriate route of administration including intradermal, intramuscular, intraperitoneal, intravenous, subcutaneous, intranasal, epidural, oral, peroral, sublingual, intracerebral, intravaginal, transdermal, rectal, inhalation, or topical. In certain embodiments, pharmaceutical compositions provided by the present disclosure are injectable formulations. In certain embodiments, pharmaceutical compositions provided by the present disclosure are injectable intravenous formulations. In certain embodiments, pharmaceutical compositions provided by the present disclosure are oral formulations. Oral formulations may be oral dosage forms.

Pharmaceutical compositions provided by the present disclosure may comprise a therapeutically-effective amount of a compound of Formula (1) or a pharmaceutically acceptable salt thereof together with a suitable amount of one or more pharmaceutically acceptable vehicles so as to provide a composition for proper administration to a patient. Suitable pharmaceutical vehicles and methods of preparing pharmaceutical compositions are described in the art.

In certain embodiments, a compound of Formula (1) or a pharmaceutically acceptable salt thereof may be administered by intravenous injection. Suitable forms for injection include sterile aqueous solutions or dispersions of a compound of Formula (1). In certain embodiments, a compound may be formulated in a physiological buffer solution. Prior to administration, a compound of Formula (1) or a pharmaceutically acceptable salt thereof may be sterilized by any art recognized the technique, including addition of antibacterial or antifungal agents, for example, paraben, chlorobutanol, phenol, sorbic acid, thimersol, and the like. In certain embodiments, a compound of Formula (1) or a pharmaceutically acceptable salt thereof may be sterilized by filtration before administration to a subject thereby minimizing or eliminating the need for additional sterilization agents. An injectable dosage of a compound of Formula (1) may include from about 0.01 mL to about 10 mL, from about 0.1 mL to about 10 mL, from about 0.1 mL to about 5 mL, and in certain embodiments, from about 1 mL to about 5 mL.

Pharmaceutical compositions may comprise a therapeutically effective amount of one or more compounds of Formula (1), preferably in purified form, together with a suitable amount of a pharmaceutically acceptable vehicle, so as to provide a form for proper administration to a patient. When administered to a patient, the compounds and pharmaceutically acceptable vehicles are preferably sterile. Water is a preferred vehicle when the compound is administered intravenously. Saline solutions and aqueous dextrose and glycerol solutions may also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Pharmaceutical compositions may also contain minor amounts of wetting or emulsifying agents, or pH buffering agents. In addition, auxiliary, stabilizing, thickening, lubricating and coloring agents may be used.

Pharmaceutical compositions comprising a compound may be manufactured by means of conventional mixing, dissolving, granulating, levitating, emulsifying, encapsulating, entrapping or lyophilizing processes. Pharmaceutical compositions may be formulated in a conventional manner using one or more physiologically acceptable carriers, diluents; excipients or auxiliaries, which facilitate processing of compounds into preparations which can be used pharmaceutically. Proper formulation is dependent upon the route of administration chosen.

Pharmaceutical compositions provided by the present disclosure may take the form of solutions, suspensions, emulsion, or any other form suitable for use. Examples of suitable pharmaceutical vehicles are described in the art.

For parenteral administration, compounds of Formula (1) may be incorporated into a solution or suspension. Parenteral administration refers to the administration by injection, for instance by intravenous, intracapsular, intrathecal, intrapleural, intratumoral, or intraperitoneal injection or intravesically. In certain embodiments, a compound of Formula (1) is administered intravenously.

A solution or suspension may also comprise at least one of the following adjuvants: sterile diluents such as water for injection, saline, fixed oils, polyethylene glycols, glycerol, propylene glycol or other synthetic solvents, antioxidants such as ascorbic acid or sodium bisulfite, buffers such as acetates, citrates or phosphates, and agents for adjustment of the tonicity such as sodium chloride or dextrose. A parenteral preparation may be enclosed into ampoules, disposable syringes or multiple dosage vessels made of glass or plastic.

For topical administration, a compound of Formula (1) may be formulated as a solution, gel, ointment, cream, suspension, etc. For transmucosal administration, penetrants appropriate to the barrier to be permeated may be used in the formulation. Such penetrants are generally known in the art. Systemic formulations include those designed for administration by injection, e.g., subcutaneous, intravenous, intramuscular, intrathecal or intraperitoneal injection, as well as those designed for transdermal, transmucosal, oral or pulmonary administration. Systemic formulations may be made in combination with a further active agent that improves mucociliary clearance of airway mucus or reduces mucous viscosity. These active agents include, for example, sodium channel blockers, antibiotics, N-acetyl cysteine, homocysteine, sodium 2-mercaptoethane sulfonate (MESNA), and phospholipids.

When a compound is acidic or basic it may be included in any of the above-described formulations as the free acid or free base, a pharmaceutically acceptable salt, a solvate of any of the foregoing, or a hydrate of any of the foregoing. Pharmaceutically acceptable salts substantially retain the activity of the free acid or base, may be prepared by reaction with bases or acids, and tend to be more soluble in aqueous and other protic solvents than the corresponding free acid or base form.

Assessing single patient response to therapy and qualifying a patient for optimal therapy are among the greatest challenges of modern healthcare and relate to trends in personalized medicine. The novel β-substituted β-amino acid derivatives and β-substituted β-amino acid analogs provided by the present disclosure have a high selectivity for LAT1/4F2hc. Radio-labeled compounds for positron emission tomography (PET) or Single Photon Emission Computed Tomography (SPECT) with the same selectivity toward LAT1/4F2hc may be used to predict the efficacy of the treatment based on a single-study, case-by-case patient analysis thus excluding subjects that are expected not to benefit from treatment. PET/SPECT scans using radiolabeled LAT1/4F2hc selective substrates, once correlated to the concentration β-substituted β-amino acid derivatives or β-substituted β-amino acid analogs of Formula (1) can provide a three-dimensional distribution map, which can then be used for macroscopic dose calculations.

Accordingly, it is within the capability of those of skill in the art to assay and use the compounds of Formula (1) and/or pharmaceutical compositions thereof for therapy.

A compound of Formula (1) and/or pharmaceutical composition thereof can generally be used in an amount effective to achieve the intended purpose. For use to treat a disease such as cancer, a compound of Formula (1) and/or pharmaceutical compositions thereof, may be administered or applied in a therapeutically effective amount.

The amount of a compound of Formula (1) and/or pharmaceutical composition thereof that will be effective in the treatment of a particular disorder or condition disclosed herein will depend in part on the nature of the disorder or condition, and can be determined by standard clinical techniques known in the art. In addition, in vitro or in vivo assays may optionally be employed to help identify optimal dosage ranges. The amount of a compound of Formula (1) and/or pharmaceutical composition thereof administered will depend on, among other factors, the subject being treated, the weight of the subject, the severity of the affliction, the manner of administration and the judgment of the prescribing physician.

A compound of Formula (1) may be assayed in vitro and in vivo, for the desired therapeutic activity, prior to use in humans. For example, in vitro assays may be used to determine whether administration of a specific compound or a combination of compounds is preferred. The compounds may also be demonstrated to be effective and safe using animal model systems.

In certain embodiments, a therapeutically effective dose of a compound of Formula (1) and/or pharmaceutical composition thereof will provide therapeutic benefit without causing substantial toxicity. Toxicity of compounds of Formula (1) and/or pharmaceutical compositions thereof may be determined using standard pharmaceutical procedures and may be readily ascertained by the skilled artisan. The dose ratio between toxic and therapeutic effect is the therapeutic index. In certain embodiments, a compound of Formula (1) and/or pharmaceutical composition thereof exhibits a particularly high therapeutic index in treating disease and disorders. In certain embodiments, a dose of a compound of Formula (1) and/or pharmaceutical composition thereof will be within a range of circulating concentrations that include an effective dose with minimal toxicity.

A compound of Formula (1), a pharmaceutically acceptable salt thereof, or a pharmaceutical composition of any of the foregoing may be included in a kit that may be used to administer the compound to a patient for therapeutic purposes. A kit may include a pharmaceutical composition comprising a compound of Formula (1) suitable for administration to a patient and instructions for administering the pharmaceutical composition to the patient. In certain embodiments, a kit for use in treating cancer in a patient comprises a compound of Formula (1) or a pharmaceutically acceptable salt thereof, a pharmaceutically acceptable vehicle for administering the compound, and instructions for administering the compound to a patient.

Instructions supplied with a kit may be printed and/or supplied, for example, as an electronic-readable medium, a video cassette, an audiotape, a flash memory device, or may be published on an internet web site or distributed to a patient and/or health care provider as an electronic communication.

Compounds of Formula (1) may be used for treating cancer in a patient, wherein the cancerous tissue expresses the LAT1/4F2hc. In certain embodiments, the cancerous tissue expressing the LAT1/4F2hc transporter is in the brain of the patient.

Compounds of Formula (1) may be used in the treatment of a wide variety of neoplasms where elevated LAT1/4F2hc mediated uptake occurs. Compounds of Formula (1) are particularly useful for treating brain tumors, including metastases of other solid tumors, such as lung or breast cancer, in the brain.

In certain embodiments, a compound of Formula (1) or a pharmaceutical composition comprising a compound of Formula (1) may be administered to treat a cancer known to be treated by an alkylating agent, such as, for example, melphalan.

In certain embodiments, a compound of Formula (1) or a pharmaceutical composition comprising a compound of Formula (1) may be administered to treat a cancer that is known to not be treated by an alkylating agent.

In certain embodiments, a compound of Formula (1) or a pharmaceutical composition comprising a compound of Formula (1) may be used to treat, for example, one or more of the following cancers: adult acute lymphoblastic leukemia (all), childhood acute lymphoblastic leukemia (all), childhood acute myeloid leukemia (aml), adult acute myeloid leukemia (aml), childhood adrenocortical carcinoma, a IDs-related cancers, a IDs-related lymphoma, anal cancer, appendix cancer, astrocytoma, childhood atypical teratoid/rhabdoid tumor, basal cell carcinoma (nonmelanoma), extrahepatic bile duct cancer, childhood bladder cancer, bone cancer, osteosarcoma, malignant fibrous histiocytoma, childhood craniopharyngioma, childhood brain stem glioma, adult brain tumor, childhood brain tumor, childhood brain stem glioma, childhood central nervous system embryonal tumors, childhood cerebellar astrocytoma, brain tumor, cerebral astrocytoma/malignant glioma, ductal carcinoma in situ, childhood ependymoblastoma, childhood ependymoma, childhood esthesioneuroblastoma, childhood medulloblastoma, childhood medulloepithelioma, childhood pineal parenchymal tumors of intermediate differentiation, supratentorial primitive neuroectodermal tumors and pineoblastoma, childhood visual pathway and hypothalamic glioma, childhood brain and spinal cord tumors, breast cancer, childhood breast cancer, male breast cancer, childhood bronchial tumors, hematopoetic tumors of the lymphoid lineage, hematopoetic tumors of the myeloid lineage, burkitt lymphoma, childhood carcinoid tumor, gastrointestinal carcinoid tumor, carcinoma of head and neck, childhood central nervous system embryonal tumors, primary central nervous system lymphoma, childhood cerebellar astrocytoma, cerebral astrocytoma/malignant glioma, childhood cervical cancer, childhood cancers, childhood chordoma, chronic lymphocytic leukemia (cll), chronic myeloproliferative disorders, colorectal cancer, cutaneous t-cell lymphoma, childhood central nervous system embryonal tumors, desmoplastic small round cell tumor, endometrial cancer, childhood ependymoblastoma, childhood ependymoma, esophageal cancer, childhood esophageal cancer, ewing family of tumors, childhood extracranial germ cell tumor, extragonadal germ cell tumor, extrahepatic bile duct cancer, dye cancer, Intraocular melanoma, retinoblastoma, gallbladder cancer, gastric (stomach) cancer, childhood gastric (stomach) cancer, gastrointestinal carcinoid tumor, gastrointestinal stromal tumor (gIst), childhood gastrointestinal stromal cell tumor, childhood extracranial germ cell tumor, extragonadal germ cell tumor, ovarian germ cell tumor, gestational trophoblastic tumor/disease, adult glioma, glioblastoma, childhood brain stem, childhood cerebral astrocytoma, childhood visual pathway and hypothalamic glioma, hairy cell leukemia, childhood heart cancer, head and neck cancer, childhood head and neck cancer, adult (primary) hepatocellular (liver) cancer, childhood (primary) hepatocellular (liver) cancer, adult Hodgkin lymphoma, childhood Hodgkin lymphoma, hypopharyngeal cancer, childhood hypothalamic and visual pathway glioma, intraocular melanoma, pancreatic neuroendocrine tumors (islet cell tumors), endocrine pancreas tumors (islet cell tumors), Kaposi sarcoma, kidney (renal cell) cancer, kidney cancer, laryngeal cancer, childhood laryngeal cancer, adult acute lymphoblastic leukemia, childhood acute lymphoblastic leukemia, adult acute myeloid leukemia, childhood acute myeloid leukemia, chronic myelogenous leukemia (cml), hairy cell leukemia, lip and oral cavity cancer, adult primary liver cancer, childhood primary liver cancer, non-small cell lung cancer, small cell lung cancer, a IDs-related lymphoma, Burkitt lymphoma, t-cell lymphoma, b-cell lymphoma, cutaneous t-cell lymphoma, adult Hodgkin lymphoma, childhood Hodgkin lymphoma, adult non-Hodgkin lymphoma, childhood non-Hodgkin lymphoma, primary central nervous system lymphoma, langerhans cell histiocytosis, Waldenstrom macroglobulinemia, malignant fibrous histiocytoma of bone and osteosarcoma, childhood medulloblastoma, childhood medulloepithelioma, melanoma, intraocular (dye) melanoma, Merkel cell carcinoma, adult malignant mesothelioma, childhood mesothelioma, primary metastatic squamous neck cancer with occult, mouth cancer, myelodysplastic/myeloproliferative neoplasms, midline tract carcinoma involving nUt gene, childhood multiple endocrine neoplasia syndrome, multiple myeloma/plasma cell neoplasm, mycosis fungoides, myelodysplastic syndromes myelodysplastic/myeloproliferative diseases, chronic myelogenous leukemia, adult acute myeloid leukemia, childhood acute myeloid leukemia, multiple myeloma, chronic myeloproliferative disorders, malignant germ cell tumors, nasal cavity and paranasal sinus cancer, nasopharyngeal cancer, childhood nasopharyngeal cancer, neuroblastoma, adult non-Hodgkin lymphoma, childhood non-Hodgkin lymphoma, non-small cell lung cancer, childhood oral cancer, lip and oral cavity cancer, oropharyngeal cancer, osteosarcoma and malignant fibrous histiocytoma of bone, childhood ovarian cancer, ovarian epithelial cancer, ovarian germ cell tumor, ovarian low malignant potential tumor, pancreatic cancer, childhood pancreatic cancer, islet cell tumors, childhood papillomatosis, paranasal sinus and nasal cavity cancer, parathyroid cancer, penile cancer, pharyngeal cancer, pheochromocytoma, childhood pineal parenchymal tumors of intermediate differentiation, childhood pineoblastoma and supratentorial primitive neuroectodermal tumors, pituitary tumor, paraganglioma, plasma cell neoplasm/multiple myeloma, pleuropulmonary blastoma, childhood pleuropulmonary blastoma, primary central nervous system (cns) lymphoma, pregnancy and breast cancer, primary central nervous system lymphoma, prostate cancer, rectal cancer, renal cell (kidney) cancer, childhood renal cell (kidney) cancer, renal pelvis and ureter, transitional cell cancer, respiratory tract carcinoma involving the nUt gene on chromosome 15, retinoblastoma, childhood rhabdomyosarcoma, salivary gland cancer, childhood salivary gland cancer, sarcoma (dwing family of tumors), Kaposi sarcoma, adult soft tissue sarcoma, childhood soft tissue sarcoma, uterine sarcoma, sézary syndrome, skin cancer (nonmelanoma), childhood skin cancer, melanoma, Merkel cell skin carcinoma, small cell lung cancer, small intestine cancer, adult soft tissue sarcoma, childhood soft tissue sarcoma, squamous cell carcinoma (nonmelanoma), primary and metastatic squamous neck cancer with occult, stomach (gastric) cancer, childhood stomach (gastric) cancer, childhood supratentorial primitive neuroectodermal tumors, cutaneous t-cell lymphoma, testicular cancer, throat cancer, thymoma and thymic carcinoma, childhood thymoma and thymic carcinoma, thyroid cancer, childhood thyroid cancer, gestational trophoblastic tumor, adult unknown primary site, carcinoma of, childhood cancer of unknown primary site, unusual cancers of childhood, transitional cell cancer of ureter and renal pelvis, urethral cancer, endometrial uterine cancer, uterine sarcoma, vaginal cancer, childhood vaginal cancer, childhood visual pathway and hypothalamic glioma, vulvar cancer, Waldenstrom macroglobulinemia, Wilms tumor, women's cancers, and systemic and central metastases of any of the foregoing.

In certain embodiments, a compound of Formula (1) or a pharmaceutical composition comprising a compound of Formula (1) may be used to treat, for example, astrocytoma, atypical teratoid rhabdoid tumor, chondrosarcoma, chroid plexus tumors, carniopharyngioma, ependyoma, germ cell tumor, glioblatoma, hemangioma, lipoma, primary and teastatic CNS lymphoma, medulloblastoma, meningioma, metastatic neurofibroma, neuronal and mixed neuronal glial tumors, oligoastrocytoma, oligodendroglioma, pineal tumor, pituitary tumor, PNET, and schwannoma.

In certain embodiments, a compound of Formula (1) or a pharmaceutical composition comprising a compound of Formula (1) may be used to treat, for example, one or more of the following cancers wherein the cancer is selected from any of the primary adult and childhood brain and CNS cancers including glioblastoma (GBM) and astrocystoma, skin cancers including melanoma, lung cancers including small cell lung cancers, non-small cell lung cancers (NSCLC), and large cell lung cancers, breasts cancers including triple negative breast cancer (TNBC), blood cancers including myelodysplastic syndrome (MDS), multiple myeloma (MM), and acute myeloid leukemia (AML), prostate cancer including castrate resistant prostate cancer (CRPC), liver cancers including hepatocellular carcinoma (HCC), esophageal and gastric cancers, and any systemic and central metastases of any of the foregoing.

Compounds of Formula (1) maybe used to treat a cancer in which there is differential LAT1/4F2hc transport activity relative to surrounding tissue and/or tissue in other body organs. Patients having a tumor exhibiting a greater LAT1/4F2hc transport activity than non-diseased tissue are expected to respond more favorably to treatment with a therapeutic agent that is a substrate for the LAT1/4F2hc transporter and to experience fewer adverse effects associated with the effects of the therapeutic agent on non-diseased tissue. Compounds of Formula (1) are therapeutic agents, are substrates for the LAT1/4F2hc transporter, and exhibit cytotoxicity.

The amount of a compound of Formula (1) that will be effective in the treatment of a cancer will depend, at least in part, on the nature of the disease, and may be determined by standard clinical techniques known in the art. In addition, in vitro or in vivo assays may be employed to help identify optimal dosing ranges. Dosing regimens and dosing intervals may also be determined by methods known to those skilled in the art. The amount of compound of Formula (1) administered may depend on, among other factors, the subject being treated, the weight of the subject, the severity of the disease, the route of administration, and the judgment of the prescribing physician.

For systemic administration, a therapeutically effective dose may be estimated initially from in vitro assays. Initial doses may also be estimated from in vivo data, e.g., animal models, using techniques that are known in the art. Such information may be used to more accurately determine useful doses in humans. One having ordinary skill in the art may optimize administration to humans based on animal data.

A dose of compound of Formula (1) and appropriate dosing intervals may be selected to maintain a sustained therapeutically effective concentration of the compound of Formula (1) in the blood of a patient, and in certain embodiments, without exceeding a minimum adverse concentration.

In certain embodiments, pharmaceutical compositions comprising a compound of Formula (1) may be administered once per day, twice per day, and in certain embodiments at intervals of more than once per day. Dosing may be provided alone or in combination with other drugs and may continue as long as required for effective treatment of the disease. Dosing may also be undertaken using continuous or semi-continuous administration over a period of time. Dosing includes administering a pharmaceutical composition to a mammal, such as a human, in a fed or fasted state.

A pharmaceutical composition may be administered in a single dosage form or in multiple dosage forms or as a continuous or an accumulated dose over a period of time. When multiple dosage forms are used the amount of compound of Formula (1) contained within each of the multiple dosage forms may be the same or different.

Suitable daily dosage ranges for administration may range from about 2 mg to about 50 mg of a compound of Formula (1) per kilogram body weight.

Suitable daily dosage ranges for administration may range from about 1 mg to about 100 mg of a compound of Formula (1) per square meter (m²) of body surface.

In certain embodiments, a compound of Formula (1) may be administered to treat cancer in a subject in an amount from about 50 mg to about 2,000 mg per day, from about 100 mg to about 1,500 mg per day, from about 200 mg to about 1,000 mg per day, or in any other appropriate daily dose.

In certain embodiments, pharmaceutical compositions comprising a compound of Formula (1) may be administered to treat cancer in a subject so as to provide a therapeutically effective concentration of a compound of Formula (1) in the blood or plasma of the subject. In certain embodiments, a therapeutically effective concentration of a compound of Formula (1) in the blood or plasma of a subject is from about 1 μg/mL to about 60 μg/mL, from about 2 μg/mL to about 50 μg/mL, from about 5 μg/mL to about 40 μg/mL, from about 5 μg/mL to about 20 μg/mL, and in certain embodiments, from about 5 μg/mL to about 10 μg/mL. In certain embodiments, a therapeutically effective concentration of a compound of Formula (1) in the blood or plasma of a subject is at least about 2 μg/mL, at least about 5 μg/mL, at least about 10 μg/mL, at least about 15 μg/mL, at least about 25 μg/mL, and in certain embodiments, at least about 30 μg/mL. In certain embodiments, a therapeutically effective concentration of a compound of Formula (1) in the blood or plasma of a subject is less than an amount that causes unacceptable adverse effects including adverse effects to homeostasis. In certain embodiments, a therapeutically effective concentration of a compound of Formula (1) in the blood or plasma of a subject is an amount sufficient to restore and/or maintain homeostasis in the subject.

In certain embodiments, pharmaceutical compositions comprising a compound of Formula (1) may be administered to treat cancer in a subject so as to provide a therapeutically effective concentration of a compound of Formula (1) in the blood or plasma of a subject for an extended period of time such as, for example, for at least about 4 hours, for at least about 6 hours, for at least about 8 hours, for at least about 10 hours, and in certain embodiments, for at least about 12 hours.

The amount of a compound of Formula (1) administered may vary during a treatment regimen.

Pharmaceutical compositions provided by the present disclosure may further comprise one or more pharmaceutically active compounds in addition to a compound of Formula (1). Such compounds may be provided to treat the cancer being treated with the compound of Formula (1) or to treat a disease, disorder, or condition other than the cancer being treated with the compound of Formula (1).

In certain embodiments, a compound of Formula (1) may be used in combination with at least one other therapeutic agent. In certain embodiments, a compound of Formula (1) may be administered to a patient together with another compound for treating cancer in the subject. In certain embodiments, the at least one other therapeutic agent may be a different compound of Formula (1). A compound of Formula (1) and the at least one other therapeutic agent may act additively or, and in certain embodiments, synergistically. The at least one additional therapeutic agent may be included in the same pharmaceutical composition or vehicle comprising the compound of Formula (1) or may be in a separate pharmaceutical composition or vehicle. Accordingly, methods provided by the present disclosure further include, in addition to administering a compound of Formula (1), administering one or more therapeutic agents effective for treating cancer or a different disease, disorder or condition than cancer. Methods provided by the present disclosure include administration of a compound of Formula (1) and one or more other therapeutic agents provided that the combined administration does not inhibit the therapeutic efficacy of a compound of Formula (1) and/or does not produce adverse combination effects.

In certain embodiments, pharmaceutical compositions comprising a compound of Formula (1) may be administered concurrently with the administration of another therapeutic agent, which may be part of the same pharmaceutical composition as, or in a different pharmaceutical composition than that comprising a compound of Formula (1). A compound of Formula (1) may be administered prior or subsequent to administration of another therapeutic agent. In certain embodiments of combination therapy, the combination therapy may comprise alternating between administering a compound of Formula (1) and a composition comprising another therapeutic agent, e.g., to minimize adverse drug effects associated with a particular drug. When a compound of Formula (1) is administered concurrently with another therapeutic agent that potentially may produce an adverse drug effect including, for example, toxicity, the other therapeutic agent may be administered at a dose that falls below the threshold at which the adverse drug reaction is elicited.

In certain embodiments, pharmaceutical compositions comprising a compound of Formula (1) may be administered with one or more substances to enhance, modulate and/or control release, bioavailability, therapeutic efficacy, therapeutic potency, stability, and the like of a compound of Formula (1). For example, to enhance the therapeutic efficacy of a compound of Formula (1), a compound of Formula (1) or a pharmaceutical composition comprising a compound of Formula (1) may be co-administered with one or more active agents to increase the absorption or diffusion of the compound of Formula (1) from the gastrointestinal tract to the systemic circulation, or to inhibit degradation of the compound of Formula (1) in the blood of a subject. In certain embodiments, a pharmaceutical composition comprising a compound of Formula (1) may be co-administered with an active agent having pharmacological effects that enhance the therapeutic efficacy of the compound of Formula (1).

In certain embodiments, a compound of Formula (1) or a pharmaceutical composition comprising a compound of Formula (1) may be administered in conjunction with an agent known or believed to be effective in treating cancer in a patient.

For example, in certain embodiments, a compound of Formula (1) or a pharmaceutical composition comprising a compound of Formula (1) may be administered in conjunction with another chemotherapeutic agents, such as, for example, N-acetyl cysteine (NAC), adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combination of any of the foregoing. In certain embodiments, a compound of Formula (1) and/or pharmaceutical compositions thereof can be used in combination therapy with other chemotherapeutic agents including one or more antimetabolites such as folic acid analogs; pyrimidine analogs such as fluorouracil, floxuridine, and cytosine arabinoside; purine analogs such as mercaptopurine, thiogunaine, and pentostatin; natural products such as vinblastine, vincristine, etoposide, tertiposide, dactinomycin, daunorubicin, doxurubicin, bleomycin, mithamycin, mitomycin C, L-asparaginase, and interferon alpha; platinum coordination complexes such as cis-platinum, and carboplatin; mitoxantrone; hydroxyurea; procarbazine; hormones and antagonists such as prednisone, hydroxyprogesterone caproate, medroxyprogesterone acetate, megestrol acetate, diethylstilbestrol, ethinyl estradiol, tamoxifen, testosterone propionate, fluoxymesterone, flutamide, and leuprolide, anti-angiogenesis agents or inhibitors such as angiostatin, retinoic acids, paclitaxel, estradiol derivatives, and thiazolopyrimidine derivatives; apoptosis prevention agents; and radiation therapy.

In certain embodiments, a compound of Formula (1) may be coadministered with a compound that inhibits DNA repair such as, for example, O⁶-benzylguanine (O⁶-BG).

In certain embodiments, a compound of Formula (1) may be coadministred with a compound that blocks and/or inhibits transporters other than LAT1 such as, for example, amino acids. In certain embodiments, compounds of Formula (1) may be administered to a patient together with one or more amino acids such as, for example, arginine (Arg), serine (Ser), lysine (Lys), asparagine (Asn), glutamine (Gln), threonine (Thr), or mixtures of any of the foregoing. In certain embodiments, co-administration of amino acids is intended to saturate amino acid transporters that interact with compounds of Formula (1) and thereby increase the selectivity for LAT1.

The efficacy of administering a compound of Formula (1) for treating cancer may be assessed using in vitro and animal studies and in clinical trials.

The suitability of compounds of Formula (1) and/or pharmaceutical compositions thereof in treating cancers listed above may be determined by methods described in the art. For example, screens developed to demonstrate the anti-tumor activity of oncolytic agents are known (Miller, et al., J Med Chem, 1977, 20(3), 409-413; Sweeney, et al., Cancer Res, 1978, 38(9), 2886-2891; and Weiss and Von Hoff, Semin Oncol, 1985, 12(3 Suppl 4), 69-74). Accordingly, it is well with the capability of those of skill in the art to assay and use the compounds and/or pharmaceutical compositions thereof to treat the above diseases or disorders.

Methods provided by the present disclosure have use in animals, including mammals, such as in humans.

A cell cycle inhibitor can be selected that does not mitigate or reduce the therapeutic efficacy of the LAT1-transported chemotherapeutic agent. It is therefore desirable that a suitable cell cycle inhibitor not inhibit or minimally inhibit the proliferation of the diseased cells, and have predominate effects on the inhibition of non-diseased cells such as bone marrow, T cells, and/or lymphocytes.

Cell cycle inhibitors can be administered in conjunction with a regimen for treating a brain cancer. To be effective in treating a brain cancer, a systemically administered LAT1-transported chemotherapeutic agent must pass through the blood brain barrier (BBB). The ability of a LAT1-transported chemotherapeutic agent to pass through the blood brain barrier is limited by a number of factors including limited active transport mechanisms across the brain epithelial cells and by active efflux transporters.

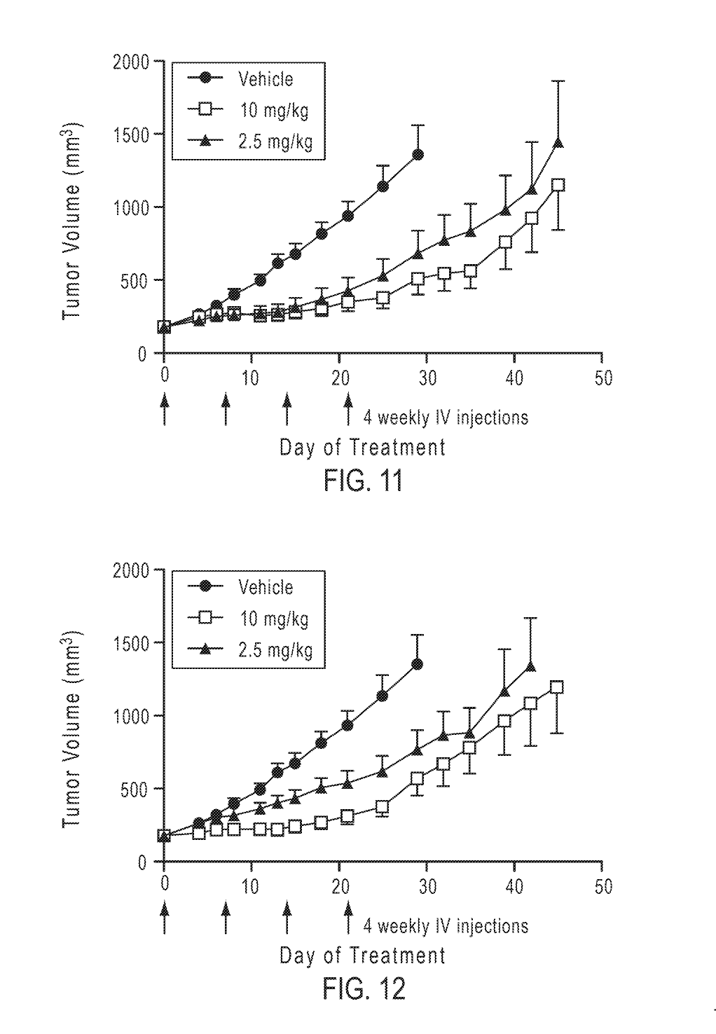

LAT1 is expressed in the brain epithelial cells and serves as a substrate for the actively transported chemotherapeutic agents provided by the present disclosure. As demonstrated by the results presented in FIGS. 11 and 12 LAT1-transported chemotherapeutic agents are effective in being transported through the BBB and reversing the growth of glioblastomas.

LAT1 is also expressed in all normal, healthy cells. The side effects of LAT1-targeted chemotherapy can be ameliorated or reduced by administering a cell cycle inhibitor effective in inhibiting the proliferation of rapidly dividing cells. Certain suitable cell cycle inhibitor may not readily pass through the blood brain barrier and thereby may not reduce the efficacy of the LAT1-transported therapeutic compound for treating brain cancers. The cell cycle inhibitor can have a protective effect on proliferating cell populations, and allow such populations to recover after or during the LAT1-transported chemotherapeutic regimen to restore normal function.

For cell cycle inhibitors that are not effectively transported across the BBB, higher doses of the LAT1-transported chemotherapeutic agent can be administered, which can lead to enhanced therapeutic efficacy. Because the cell cycle inhibitors are do not pass through the BBB and therefore cannot interfere with the therapeutic efficacy of the LAT1-transported chemotherapeutic agent, any suitable cell cycle inhibitor can be used. The BBB serves as a proxy for differentiating between cells affected by the chemotherapeutic agent and the cell cycle inhibitor, and therefore the mechanism of action or target pathway of the cell cycle inhibitor is not particularly important to the efficacy of the co-therapy.

In certain methods such as for treating a brain cancer, a cell cycle inhibitor is selected that does not effectively pass through the BBB. For example, some compounds can pass through brain epithelia but are efficiently returned to the systemic circulation by efflux transporters such that an effective amount of the compound does not enter and/or is not accumulated in the brain.

Therapeutic regimens provided by the present disclosure comprise the administration of a LAT1-transported chemotherapeutic agent and a cell cycle inhibitor.

The cell cycle inhibitor can be administered to a patient before administration of the LAT1-transported chemotherapeutic agent, during administration of the LAT1-transported chemotherapeutic agent, and/or after administration of the LAT1-transported chemotherapeutic agent. The treatment regimen can comprise a single administration of the cell cycle inhibitor, multiple administrations of cell cycle inhibitor, a single administration of a LAT1-transported chemotherapeutic agent, multiple administrations of a LAT1-transported chemotherapeutic agent, or combinations of any of the foregoing.

The dose and timing of each administration can be determined to achieve a pharmacokinetic profile of both the cell cycle inhibitor and the LAT1-transported chemotherapeutic agent that establishes a desired balance of chemotherapeutic efficacy and reducing adverse side effects.

In certain embodiments, a regiment comprises a healthy cell cycling strategy in which a subject is exposed to regular, repeated chemotherapeutic treatments, wherein the healthy cells are arrested when the healthy and diseased cells are exposed to the LAT1-transported chemotherapeutic agent and then allowed to reenter the cell-cycle before a subsequent chemotherapeutic treatment. Such cycling allows healthy cells to regenerate and in the case of bone marrow, restoring damaged blood cell lineages, between regular, repeated treatments, for example those associated with standard chemotherapeutic treatments for cancer. The shorter exposures of the cell cycle inhibitor and/or a lower concentration can reduce the risk associated with long term inhibition of healthy cells.

A dose of a cell cycle inhibitor can be selected to arrest the growth of otherwise rapidly proliferating cell populations such as bone marrow, while having less or minimal effect on other healthy cells and thereby reduce the toxicity of the cell cycle inhibitor. A lower dose of the cell cycle inhibitor can also minimize the potential for the cell cycle inhibitor to arrest the growth of the target diseased cell population such as a cancer.

The amount of a LAT1-transported chemotherapeutic agent that will be effective in the treatment of a cancer and/or a dose of a cell cycle inhibitor effective for protecting normal, healthy cells can depend, at least in part, on the nature of the disease, and may be determined by standard clinical techniques known in the art. In addition, in vitro or in vivo assays may be employed to help identify optimal dosing ranges. Dosing regimens and dosing intervals may also be determined by methods known to those skilled in the art. The amount of a LAT1-transported chemotherapeutic agent administered may depend on, among other factors, the subject being treated, the weight of the subject, the severity of the disease, the route of administration, and the judgment of the prescribing physician.

Doses and dosing regimens of the LAT1-transported chemotherapeutic agent and the cell cycle inhibitor can be selected to balance therapeutic efficacy of the chemotherapy. This can involve balancing the chemotherapeutic efficacy with the risk or severity of adverse side effects. For example, using a cell cycle inhibitor to transiently suppress the growth of bone marrow cells, can allow the use of higher concentrations of the LAT1-transported chemotherapeutic agent, thereby increasing the therapeutic efficacy of the LAT1-transported chemotherapeutic agent, and avoid or ameliorate the adverse consequences of the chemotherapy from myelosuppression. The dose and regimen of the LAT1-transported chemotherapeutic agent and the cell cycle inhibitor may also be selected to balance the efficacy of the LAT1-transported chemotherapeutic agent on the target cell population such as a cancer, while minimizing the effects of the cell cycle inhibitor on the target cell population, such as on arresting the growth of the target cell population. An objective of the combined administration can be to select the dose and/or regimen of the LAT1-transported chemotherapeutic agent to maximize therapeutic efficacy on the target cell population, select the dose and/or regimen of the cell cycle inhibitor to protect certain desired cell populations such as bone marrow cells, and without unduly interfering with the therapeutic efficacy of the LAT1-transported chemotherapeutic agent on the target cell population. The selection of the particular LAT1-transported chemotherapeutic agent and the cell cycle inhibitor can also affect the selection of the suitable dose and or/regiment of both the LAT1-transported chemotherapeutic agent and the cell cycle inhibitor.

For systemic administration, a therapeutically effective dose may be estimated initially from in vitro assays. Initial doses may also be estimated from in vivo data, e.g., animal models, using techniques that are known in the art. Such information may be used to more accurately determine useful doses in humans. One having ordinary skill in the art may optimize administration to humans based on animal data.

A dose of a LAT1-transported chemotherapeutic agent and a cell cycle inhibitor and appropriate dosing intervals may be selected to maintain a sustained therapeutically effective concentration of the LAT1-transported chemotherapeutic agent and the cell cycle inhibitor in the blood of a patient, and in certain embodiments, without exceeding a minimum adverse concentration.

In certain embodiments, a LAT1-transported chemotherapeutic agent and/or a cell cycle inhibitor may be administered once per day, twice per day, and in certain embodiments at intervals of more than once per day. Dosing may be provided alone or in combination with other drugs and may continue as long as required for effective treatment of the disease. Dosing may also be undertaken using continuous or semi-continuous administration over a period of time. Dosing includes administering a pharmaceutical composition to a mammal, such as a human, in a fed or fasted state.

A LAT1-transported chemotherapeutic agent and/or a cell cycle inhibitor may be administered in a single dosage form or in multiple dosage forms or as a continuous or an accumulated dose over a period of time. When multiple dosage forms are used the amount of a LAT1-transported chemotherapeutic agent and/or a cell cycle inhibitor contained within each of the multiple dosage forms may be the same or different.

Suitable daily dosage ranges for administration may range from about 2 μg to about 20 mg of a LAT1-transported chemotherapeutic agent and/or a cell cycle inhibitor per kilogram body weight.

Suitable daily dosage ranges for administration may range from about 1 μg to about 50 mg of a LAT1-transported chemotherapeutic agent and/or a cell cycle inhibitor per square meter (m²) of body surface.

In certain embodiments, a LAT1-transported chemotherapeutic agent and/or a cell cycle inhibitor may be administered to treat cancer in a subject in an amount from about 1 mg to about 2,000 mg per day, from about 100 μg to about 1,500 mg per day, from about 20 μg to about 1,000 mg per day, or in any other appropriate daily dose.

In certain embodiments, pharmaceutical compositions comprising a LAT1-transported chemotherapeutic agent and/or a cell cycle inhibitor may be administered to treat cancer in a subject so as to provide a therapeutically effective concentration of a chemotherapeutic agent and/or a cell cycle inhibitor in the blood or plasma of the subject. In certain embodiments, a therapeutically effective concentration of a LAT1-transported chemotherapeutic agent and/or a cell cycle inhibitor in the blood or plasma of a subject is from about 1 μg/mL to about 60 μg/mL, from about 2 μg/mL to about 50 μg/mL, from about 5 μg/mL to about 40 μg/mL, from about 5 μg/mL to about 20 μg/mL, and in certain embodiments, from about 5 μg/mL to about 10 μg/mL. In certain embodiments, a therapeutically effective concentration of a LAT1-transported chemotherapeutic agent and/or a cell cycle inhibitor in the blood or plasma of a subject is at least about 2 μg/mL, at least about 5 μg/mL, at least about 10 μg/mL, at least about 15 μg/mL, at least about 25 μg/mL, and in certain embodiments, at least about 30 μg/mL. In certain embodiments, a therapeutically effective concentration of a LAT1-transported chemotherapeutic agent and/or a cell cycle inhibitor in the blood or plasma of a subject is less than an amount that causes unacceptable adverse effects including adverse effects to homeostasis. In certain embodiments, a therapeutically effective concentration of a LAT1-transported chemotherapeutic agent and/or a cell cycle inhibitor in the blood or plasma of a subject is an amount sufficient to restore and/or maintain homeostasis in the subject.

In certain embodiments, pharmaceutical compositions comprising a LAT1-transported chemotherapeutic agent and/or a cell cycle inhibitor may be administered to treat cancer in a subject so as to provide a therapeutically effective concentration of a LAT1-transported chemotherapeutic agent and/or a cell cycle inhibitor in the blood or plasma of a subject for an extended period of time such as, for example, for at least about 4 hours, for at least about 6 hours, for at least about 8 hours, for at least about 10 hours, and in certain embodiments, for at least about 12 hours.

The amount of a LAT1-transported chemotherapeutic agent and/or a cell cycle inhibitor administered may vary during a treatment regimen.

In certain embodiments, a regimen can comprise the administration of a blood proliferation compound. Following administration of a cell cycle inhibitor to arrest or suppress proliferation of non-target normal, healthy cells and tissue, and following administration of a LAT1-transported chemotherapeutic agent, a blood growth factor can be administered to stimulate the proliferation of previously arrested or suppressed cells. Examples of suitable hematopoietic growth factors include granulocyte colony stimulating factor (G-CSF, commercially available as Neupogen® (filgrastin), Neulasta® (peg-filgrastin), or lenograstin), granulocyte-macrophage colony stimulating factor such as molgramostim and sargramostim, M-CSF (macrophage colony stimulating factor), thrombopoietin (megakaryocyte growth development factor (MGDF), commercially available as Romiplostim® and Eltrombopag®) interleukin (IL)-12, interleukin-3, interleukin-11 (adipogenesis inhibiting factor or oprelvekin), SCF (stem cell factor, steel factor, kit-ligand, or KL) and erythropoietin (EPO), and their derivatives (commercially available as epoetin-α as Darbopoetin®, Epocept®, Nanokine®, Epofit®, Epogin®, Eprex® and Procrit®; epoetinβ commercially available as NeoRecormon®, Recormon® and Micera®), epoetin-δ (Dynepo®), epoetin-ω (Epomax®), epoetin zeta (Silapo ω and Reacrit ω).

A potential advantage of using certain cell cycle inhibitors to temporarily arrest cell growth is that following dissipation of the effects of the cell cycle inhibitors, the arrested cell population can reenter the cell growth cycle in a synchronous manner. This synchronous reentry can, in the case of bone marrow cells, enhance the effects of administered growth factors such as hematopoietic growth factors to reconstitute hematopoietic cell lines to maximize the growth factor effect. As such, the use of cell cycle inhibitors and LAT1-transported chemotherapeutic agents can be combined with the use of hematopoietic growth factors such as granulocyte colony stimulating factor (G-CSF), granulocyte-macrophage colony stimulating factor (GM-CSF), thrombopoietin, interleukin (IL)-12, steel factor, and erythropoietin (EPO), or their derivatives. A cell cycle inhibitor can be administered prior to administration of a hematopoietic growth factor and the administration of the hematopoietic growth factor can be timed so that the arrest of the cell population has dissipated.

In aspect of the present invention, methods of reducing the effects of chemotherapy on normal/healthy cells in a patient being treated for cancer or abnormal cell proliferation, comprise administering to the patient a therapeutically effective amount of a cell cycle inhibitor; and administering to the patient a therapeutically effective amount of a chemotherapeutic compound selected from:
3-amino-3-[5-[bis(2-chloroethyl)amino]-2-methyl-phenyl]propanoic acid (1);
3-amino-3-[4-[bis(2-chloroethyl)amino]-2-methyl-phenyl]propanoic acid (2);
3-amino-4-[5-[bis(2-chloroethyl)amino]-2-methyl-phenyl]butanoic acid (3);
3-amino-4-[4-[bis(2-chloroethyl)amino]-2-methyl-phenyl]butanoic acid (4);
(3S)-3-amino-4-[5-[bis(2-chloroethyl)amino]-2-methyl-phenyl]butanoic acid (5);
(3R)-3-amino-4-[5-[bis(2-chloroethyl)amino]-2-methyl-phenyl]butanoic acid (6);
(3S)-3-amino-4-[5-[bis(2-chloroethyl)amino]-2-methoxy-phenyl]butanoic acid (7);
(3S)-3-amino-4-[3-[bis(2-chloroethyl)amino]-2,6-dimethyl-phenyl]butanoic acid (8);
(3S)-3-amino-4-[5-[bis(2-chloroethyl)amino]-2-methyl-phenyl]-3-methyl-butanoic acid (9);
[(2R)-2-amino-3-[5-[bis(2-chloroethyl)amino]-2-methyl-phenyl]propyl]phosphinic acid (10);
(3R)-3-amino-4-[5-(bis(2-methyl sulfonyloxyethyl)amino)-2-methyl-phenyl]butanoic acid (11);
(3R)-3-amino-4-[5-(bis(2-bromoethyl)amino)-2-methyl-phenyl]butanoic acid (12);
(3R)-3-amino-4-[5-(2-chloroethyl(2-methylsulfonyloxyethyl)amino)-2-methyl-phenyl]butanoic acid (13);
(3R)-3-amino-4-[5-(2-bromoethyl(2-chloroethyl)amino)-2-methyl-phenyl]butanoic acid (14);
(3R)-3-amino-4-[5-(2-bromoethyl(2-methylsulfonyloxyethyl)amino)-2-methyl-phenyl]butanoic acid (15);
(3S)-3-amino-4-[[5-[bis(2-chloroethyl)amino]-2-methyl-phenyl]amino]-4-oxo-butanoic acid (16);
(3S)-3-amino-4-[2-[bis(2-chloroethyl)amino]phenoxy]butanoic acid (17);
(3R)-3-amino-5-[5-[bis(2-chloroethyl)amino]-2-methyl-phenyl]pentanoic acid (18);
(3R)-3-amino-4-[5-(2-chloroethyl(chloromethyl)carbamoyl)oxy-2-methyl-phenyl]butanoic acid (19);
(3R)-3-amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21);
3-[(2R)-2-amino-4-hydroxy-4-oxo-butyl]-N,N-bis(2-chloroethyl)-4-methyl-benzeneamine oxide (22); and
(3R)-3-amino-4-[2-[bis(2-chloroethyl)carbamoyl]phenyl]butanoic acid (23);
or a pharmaceutically acceptable salt of any of the foregoing.

In any of the preceding aspects, the cell cycle inhibitor comprises a myelosuppressor, an immunosuppressor, or a combination thereof.

In any of the preceding aspects, the therapeutically effective amount of the cell cycle inhibitor is effective in reducing the level of myelosuppression associated with the administration of the chemotherapeutic agent, compared to the level of myelosuppression associated with the administration of the chemotherapeutic agent without the administration of the cell cycle inhibitor.

In any of the preceding aspects, the method results in a higher therapeutic index for the chemotherapeutic agent compared to a therapeutic index for the chemotherapeutic agent without administering the cell cycle inhibitor.

In any of the preceding aspects, the cell cycle inhibitor comprises a myelosuppressor.

In any of the preceding aspects, the cancer comprises brain cancer.

In any of the preceding aspects, the cell cycle inhibitor is effective in arresting the growth of hematopoietic stem cells, hematopoietic progenitor cells, T-cells, multipotent progenitors, common myeloid progenitors, common lymphoid progenitors, granulocyte-monocyte progenitors, and megakaryocyte-erythroid progenitors, renal epithelial cells, T-cells, and a combination of any of the foregoing.

In any of the preceding aspects, the cell cycle inhibitor is reversible.

In any of the preceding aspects, the method reduces myelosuppression induced by the chemotherapeutic agent.

In an aspect of the present invention, methods of promoting recovery from the effects of a chemotherapeutic regimen for treating cancer in a patient comprise administering to the patient a therapeutically effective amount of a cell cycle inhibitor to inhibit the proliferation of normal, healthy cells; and a therapeutically effective about of a chemotherapeutic agent selected from:
3-amino-3-[5-[bis(2-chloroethyl)amino]-2-methyl-phenyl]propanoic acid (1);
3-amino-3-[4-[bis(2-chloroethyl)amino]-2-methyl-phenyl]propanoic acid (2);
3-amino-4-[5-[bis(2-chloroethyl)amino]-2-methyl-phenyl]butanoic acid (3);
3-amino-4-[4-[bis(2-chloroethyl)amino]-2-methyl-phenyl]butanoic acid (4);
(3S)-3-amino-4-[5-[bis(2-chloroethyl)amino]-2-methyl-phenyl]butanoic acid (5);
(3R)-3-amino-4-[5-[bis(2-chloroethyl)amino]-2-methyl-phenyl]butanoic acid (6);
(3S)-3-amino-4-[5-[bis(2-chloroethyl)amino]-2-methoxy-phenyl]butanoic acid (7);
(3S)-3-amino-4-[3-[bis(2-chloroethyl)amino]-2,6-dimethyl-phenyl]butanoic acid (8);
(3S)-3-amino-4-[5-[bis(2-chloroethyl)amino]-2-methyl-phenyl]-3-methyl-butanoic acid (9);
[(2R)-2-amino-3-[5-[bis(2-chloroethyl)amino]-2-methyl-phenyl]propyl]phosphinic acid (10);
(3R)-3-amino-4-[5-(bis(2-methyl sulfonyloxyethyl)amino)-2-methyl-phenyl]butanoic acid (11);
(3R)-3-amino-4-[5-(bis(2-bromoethyl)amino)-2-methyl-phenyl]butanoic acid (12);
(3R)-3-amino-4-[5-(2-chloroethyl(2-methylsulfonyloxyethyl)amino)-2-methyl-phenyl]butanoic acid (13);
(3R)-3-amino-4-[5-(2-bromoethyl(2-chloroethyl)amino)-2-methyl-phenyl]butanoic acid (14);
(3R)-3-amino-4-[5-(2-bromoethyl(2-methylsulfonyloxyethyl)amino)-2-methyl-phenyl]butanoic acid (15);
(3S)-3-amino-4-[[5-[bis(2-chloroethyl)amino]-2-methyl-phenyl]amino]-4-oxo-butanoic acid (16);
(3S)-3-amino-4-[2-[bis(2-chloroethyl)amino]phenoxy]butanoic acid (17);
(3R)-3-amino-5-[5-[bis(2-chloroethyl)amino]-2-methyl-phenyl]pentanoic acid (18);
(3R)-3-amino-4-[5-(2-chloroethyl(chloromethyl)carbamoyl)oxy-2-methyl-phenyl]butanoic acid (19);
(3R)-3-amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21);
3-[(2R)-2-amino-4-hydroxy-4-oxo-butyl]-N,N-bis(2-chloroethyl)-4-methyl-benzeneamine oxide (22); and
(3R)-3-amino-4-[2-[bis(2-chloroethyl)carbamoyl]phenyl]butanoic acid (23);
or a pharmaceutically acceptable salt of any of the foregoing.

In any of the preceding aspects, the method comprises administering a therapeutically effective amount of a compound effective in stimulating recovery of inhibited normal, healthy cells.

In any of the preceding aspects, the compound is effective in stimulating the recovery of the hematopoietic cell population.

In any of the preceding aspects, the cancer comprises brain cancer.

In an aspect of the present invention, methods of treating cancer in a patient comprise administering to the patient being treated for the cancer, a therapeutically effective amount of a cell cycle inhibitor; and a therapeutically effective amount of a chemotherapeutic agent selected from:
3-amino-3-[5-[bis(2-chloroethyl)amino]-2-methyl-phenyl]propanoic acid (1);
3-amino-3-[4-[bis(2-chloroethyl)amino]-2-methyl-phenyl]propanoic acid (2);
3-amino-4-[5-[bis(2-chloroethyl)amino]-2-methyl-phenyl]butanoic acid (3);
3-amino-4-[4-[bis(2-chloroethyl)amino]-2-methyl-phenyl]butanoic acid (4);
(3S)-3-amino-4-[5-[bis(2-chloroethyl)amino]-2-methyl-phenyl]butanoic acid (5);
(3R)-3-amino-4-[5-[bis(2-chloroethyl)amino]-2-methyl-phenyl]butanoic acid (6);
(3S)-3-amino-4-[5-[bis(2-chloroethyl)amino]-2-methoxy-phenyl]butanoic acid (7);
(3S)-3-amino-4-[3-[bis(2-chloroethyl)amino]-2,6-dimethyl-phenyl]butanoic acid (8);
(3S)-3-amino-4-[5-[bis(2-chloroethyl)amino]-2-methyl-phenyl]-3-methyl-butanoic acid (9);
[(2R)-2-amino-3-[5-[bis(2-chloroethyl)amino]-2-methyl-phenyl]propyl]phosphinic acid (10);
(3R)-3-amino-4-[5-(bis(2-methyl sulfonyloxyethyl)amino)-2-methyl-phenyl]butanoic acid (11);
(3R)-3-amino-4-[5-(bis(2-bromoethyl)amino)-2-methyl-phenyl]butanoic acid (12);
(3R)-3-amino-4-[5-(2-chloroethyl(2-methylsulfonyloxyethyl)amino)-2-methyl-phenyl]butanoic acid (13);
(3R)-3-amino-4-[5-(2-bromoethyl(2-chloroethyl)amino)-2-methyl-phenyl]butanoic acid (14);
(3R)-3-amino-4-[5-(2-bromoethyl(2-methylsulfonyloxyethyl)amino)-2-methyl-phenyl]butanoic acid (15);
(3S)-3-amino-4-[[5-[bis(2-chloroethyl)amino]-2-methyl-phenyl]amino]-4-oxo-butanoic acid (16);
(3S)-3-amino-4-[2-[bis(2-chloroethyl)amino]phenoxy]butanoic acid (17);
(3R)-3-amino-5-[5-[bis(2-chloroethyl)amino]-2-methyl-phenyl]pentanoic acid (18);
(3R)-3-amino-4-[5-(2-chloroethyl(chloromethyl)carbamoyl)oxy-2-methyl-phenyl]butanoic acid (19);
(3R)-3-amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21);
3-[(2R)-2-amino-4-hydroxy-4-oxo-butyl]-N,N-bis(2-chloroethyl)-4-methyl-benzeneamine oxide (22); and
(3R)-3-amino-4-[2-[bis(2-chloroethyl)carbamoyl]phenyl]butanoic acid (23);
or a pharmaceutically acceptable salt of any of the foregoing.

In any of the preceding aspects, the cell cycle inhibitor is effective in ameliorating myelosuppression induced by the chemotherapeutic agent.

In any of the preceding aspects, the cell cycle inhibitor is effective in arresting the growth of hematopoietic cells.

In any of the preceding aspects, the cancer comprises brain cancer.

### EXAMPLES

The following examples describe in detail the synthesis of compounds of Formula (1), characterization of compounds of Formula (1), and uses of compounds of Formula (1). It will be apparent to those skilled in the art that many modifications, both to materials and methods, may be practiced without departing from the scope of the disclosure.

### General Experimental Protocols

All reagents and solvents were purchased from commercial suppliers and used without further purification or manipulation.

Proton NMR spectra were recorded on a Varian Mercury Plus300 MHz Spectrometer equipped with an Oxford magnet, a Sun Sunblade 150 host computer, a Solaris operating system, VNMR data processing software, and a HP LaserJet printer. Where specifically noted, a Varian VNMRS 400 Spectrometer was used (400 MHz). CDCl₃ (99.8% D), MeOH-d⁴ (CD₃OD, 99.8+% D), deuteroxide (D₂O) (99.8+% D) were used as recording solvents unless otherwise noted. The CHCl₃, MeOH-d³, HDO solvent signals or tetramethylsilane (TMS) were used for calibration of the individual spectra.

Analytical thin layer chromatography (TLC) was performed using EMD Millipore aluminum-backed TLC sheets (EMD5554-7) pre-coated with silica gel 60 F254 (200 μm thickness, 60 Å pore size) where F254 is a fluorescent indicator with a 254 nm excitation wavelength. An ENF-240C Spectroline® UV-lamp (Spectronics Corporation, USA) was used for TLC detection and visualization. Dyeing or staining reagents for TLC detection and visualization, e.g., an ethanolic ninhydrin solution or a 0.2 wt-% aqueous potassium permanganate (KMnO₄) solution, were prepared according methods known in the art.

Analytical LC/MS was performed on a Shimadzu LC/MS-2020 Prominence Series system equipped with CBM-20A communication bus module (Shimadzu 228-45012-32), a SPD-20AV UV/VIS detector (Shimadzu 228-45004-32), a SIL-20AC autosampler (Shimadzu 228-45136-32), DGU-20A5 degasser (Shimadzu 228-45019-32), two LC-20AD XP HPLC pumps (Shimadzu 228-45137-32), an Agilent Zorbax 5 μm XDB-C18 2.1×50 mm column (Agilent 960 967-902), and a commercial desktop computer and printer for data computation. Gradients of water (solvent A) (Arrowhead, Nestle North America, Inc.) and acetonitrile (MeCN; solvent B) (EMD AX0145-1 or Aldrich CHROMASOLV® 439134) containing 0.075 vol-% of formic acid (EMD FX0440-7) were used in analytical LC/MS analyses.

Analytical LC/UV was performed on an Agilent 1100 Series system equipped with an Agilent 1100 Series degasser (Agilent G1379A), an Agilent 1100 Series quad pump (Agilent G1311A), an Agilent 1100 Series autosampler (ALS) (Agilent G1329A), an Agilent 1100 Series COLCOM (Agilent G1316A), a Phenomenex Gemini C18 5 μm 110 Å pore size 150×4.6 mm HPLC column (Phenomenex 00F-4435-E0), a Compaq Presario personal computer, and a HP LaserJet P2015 printer for data computation. Gradients of water (solvent A) (Arrowhead, Nestle North America, Inc.) and acetonitrile (MeCN; solvent B) (EMD AX0145-1 or Aldrich CHROMASOLV® 439134) containing 0.075 vol-% of formic acid (EMD FX0440-7) were used in analytical LC/UV analyses.

Preparative HPLC was conducted with a Varian ProStar Series system equipped with a Model 340 UV-C UV-VIS detector, a Model 210 solvent delivery module, a Hamilton PRP-112-20 μm 100 Å 21.2×250 mm preparative HPLC column (Hamilton 79428), and a commercial desktop personal computer for data computation. Gradients of water (solvent A) (Arrowhead, Nestle North America, Inc.) and acetonitrile (MeCN; solvent B) (EMD AX0145-1 or Aldrich CHROMASOLV® 439134) containing 0.1 vol-% of formic acid (EMD FX0440-7) were used for preparative HPLC purifications.

Compound isolation from aqueous solvent mixtures, e.g., acetonitrile/water/0.1 vol-% formic acid, was accomplished by primary lyophilization of pooled and frozen (after freeze drying) fractions under reduced pressure at room temperature using manifold freeze dryers such as Heto Drywinner DW 6-85-1, Heto FD4, or VIRTIS Freezemobile 25 ES equipped with a high vacuum pump. Optionally, and if the isolated compound had ionizable functional groups such as an amino group or a carboxylic acid, the lyophilization process was conducted in the presence of an excess (about 1.1 to 5.0 equivalents) of 1.0 M hydrochloric acid (HCl) to yield the purified compound(s) as the corresponding hydrochloride salt (HCl-salt), dihydrochloride salts, and/or the corresponding protonated free carboxylic acid. Melting points were determined in duplicate with a SRS OptiMelt MPA-100 automated melting point system with digital imaging processing technology and are uncorrected (Stanford Research Systems, USA).

Filtrations were conducted using commercial Celite® 545 (EMD CX0574-1) which was compressed in to glass Büchner-funnels to create a plug of 2-5 cm thickness. Reaction mixtures containing precipitated reaction side products or heterogenous catalyst residues were filtered off using standard techniques. Care must be taken filtering off activated catalysts or finely dispersed metals (ignition!).

Unless otherwise noted, aqueous work-up typically constitutes dilution of a crude reaction product, with or without residual reaction solvent, with 1.0 M hydrochloric acid (HCl) or a saturated aqueous solution of ammonium chloride (NH₄Cl), multiple extraction with an organic solvent, e.g., ethyl acetate (EtOAc), diethyl ether (Et₂O), or dichloromethane (DCM), washing with water, a saturated aqueous solution of sodium hydrogencarbonate (NaHCO₃), and brine (saturated aqueous solution of sodium chloride (NaCl)), drying of the organic phase (combined organic extracts) over anhydrous magnesium sulfate (MgSO₄) (EMD MX0075-1) or sodium sulfate (Na₂SO₄) (EMD SX0760E-3), filtration, washing of the filter residue, and evaporation of the combined filtrates under reduced pressure using a rotary evaporator at room or elevated temperature followed by compound purification e.g., silica gel column chromatography, crystallization or titruation.

Silica gel column chromatography was conducted with silica gel (about 100-200 mL silica gel per gram of compound) 600.04-0.063 mm (40-63 μm, 230-400 mesh) (EMD Millipore EM1.09385.9026/EM1.09385.1033/EM1.09385.2503) using single solvents or mixtures of suitable solvents, e.g., ethyl acetate (EtOAc) and hexane or dichloromethane (DCM) and methanol (MeOH), as determined by TLC. Samples/fractions containing desired product detected by analytical TLC and/or analytical LC/MS, or LC/UV were pooled and the solvents were removed under reduced pressure using a Heidolph Laborota 4001 Efficient rotary evaporator (Heidolph, Germany) (Heidolph 519-10000-01-5) equipped with a HB digit heating bath (Heidolph 517-01002-01-4), and a Rotavac valve control vacuum pump (Heidolph 591-00130-01-0).

Chemical names were generated using the ChemDraw Ultra 12.0 (CambridgeSoft, Cambridge, Mass., USA) nomenclature program.

### Description 1

### General Procedure for the Reduction of Benzoic Acids to Benzylic Alcohols

Adapting literature known protocols (Hay, et al., J. Chem. Soc., Perkin Trans. 1, 1999, 2759-2770; Fujikawa, et al., J. Am. Chem. Soc., 2008, 130, 14533-14543; Allen, et al., International Publication No. WO 2010/122089; and Gerspacher, et al., International Publication No. WO2008/031594), commercial borane dimethylsulfide (BH₃.DMS, BH₃.SMe₂) (2.0 M in THF) (50 mL, 100 mmol) or borane tetrahydrofurane complex (BH₃.THF) (1.0 M in THF) (100 mL, 100 mmol) is added dropwise at room temperature to a stirred solution of the nitrobenzoic acid (50 mmol) in anhydrous THF (250 mL). Optionally, the reaction is performed in the presence of trimethyl borate (B(OMe)₃) (200 mmol). The solution is heated at reflux for 4-6 hours (˜75° C. oil bath temperature). The reaction is monitored by TLC and/or LCMS to completion. After cooling to about 5° C. (ice bath), the reaction is carefully quenched with a 1:1 (v/v) mixture of methanol (MeOH)/water (25 mL) followed by 5 N hydrochloric acid (HCl) (50 mL). The mixture is heated at about 50° C. for about 30-60 min and the majority of the volatile solvents are removed under reduced pressure. Water is added and the aqueous phase is extracted with ethyl acetate (3×). The combined organic extracts are successively washed with a saturated aqueous sodium hydrogencarbonate (NaHCO₃) solution (1×) and with brine (1×), dried over anhydrous magnesium sulfate (MgSO₄), filtered, and the solvents are evaporated to dryness under reduced pressure. If needed, the crude material is purified by silica gel column chromatography or is re-crystallized.

### Description 2

### General Procedure for the Oxidation of Benzylic Alcohols to Aromatic Aldehydes

Variant A: Adapting literature known protocols (Parikh, et al., J. Am. Chem. Soc.1967, 89, 5505-5507; and Jandeleit, et al., U.S. Pat. No. 8,168,617), to a solution of the alcohol (50 mmol), dimethylsulfoxide (DMSO) (28.5 mL, 400 mmol), triethylamine (Et₃N, TEA) (34.8 mL, 250 mmol) in anhydrous dichloromethane (DCM) (300 mL) is added at 0° C. (ice bath) in small portions commercial sulfur trioxide-pyridine complex (Pyr.SO₃) (23.9 g, 150 mmol). The reaction mixture is stirred with gradual warming to room temperature for about 4-12 hours. The reaction is monitored by TLC and/or LCMS to completion. The majority of volatile is evaporated under reduced pressure and the residue is diluted with 2 M hydrochloric acid until acidic. The aqueous phase is extracted with ethyl acetate (EtOAc) (3×). The combined organic extracts are successively washed with a saturated aqueous sodium hydrogencarbonate (NaHCO₃) solution (1×) and with brine (1×), dried over anhydrous magnesium sulfate (MgSO₄), filtered, and the solvents are evaporated to dryness under reduced pressure. If needed, the crude material is purified by silica gel column chromatography or is re-crystallized.

Variant B: Adapting literature known protocol (Aoyama, et al., Synlett, 1998, 35-36), commercial activated manganese(IV) oxide (MnO₂) (250-275 mmol) is added at room temperature to a solution of the benzylic alcohol (25 mmol) in dichloromethane (DCM) (100 mL). The reaction mixture is stirred for 12-24 h. The reaction is monitored by TLC and/or LCMS to completion. The reaction mixture is filtered over a short path of Celite® 545 and the filtrate is concentrated under reduced pressure. The material is often of sufficient purity to be used directly in the next step without further isolation and purification. If needed, the crude material is purified by silica gel column chromatography or is re-crystallized.

Variant C: Adapting a literature known protocol (Corey and Suggs, Tetrahedron Lett., 1975, 16(31), 2647-2650; and Fujikawa, et al., J. Am. Chem. Soc., 2008, 130, 14533-14543), to a solution of the benzylic alcohol (20 mmol) in dichloromethane (DCM) (100 mL) is added commercial pyridinium chlorochromate (Pyr⁺CrO₃Cl⁻, PCC) (28-40 mmol). The reaction mixture is heated to reflux (55° C. oil bath temperature) for 1-4 hours. The reaction is monitored by TLC and/or LCMS to completion. The reaction is cooled to room temperature. Work-up and product isolation and purification are conducted as described for Variant B.

### Description 3

### General Procedure for 3-Amino-3-Arylpropionic Acids Via Rodionov Reaction

Adapting literature known protocols (Tran and Weaver, Tetrahedron, 2002, 58, 7449-7461; and Lebedev, et al., Russian J. Gen. Chem, 2005, 75(7), 1113-1124), 3-amino-3-arylpropionic acids are prepared in one-pot according to Rodionov by heating a mixture of the aromatic aldehyde (30 mmol, malonic acid (30 mmol), and ammonium acetate (NH₄OAc) (4.7 g, 60.7 mmol) in ethanol (about 50-100 mL) at reflux for about 12-48 hours (oil bath). The reaction is followed by LC/MS to completion. The reaction mixture is cooled to room temperature upon the target compound precipitates generally out. The precipitate is filtered off using a Bichner-funnel and the filter residue is washed with additional EtOH (2×). The collected product is dried under reduced pressure to afford of the target compound generally as a colorless solids which are often of sufficient purity to be used directly in the next step without further purification and isolation procedures.

### Description 4

### General Procedure for the Preparation of Amino Acid Methyl Esters

Adapting literature protocols (Fuchs, et al., U.S. Publication No. 2010/144681; and Allison, et al., U.S. Publication No. 2006/069286), the free (unprotected) or N-(tert-butoxycarbonyl)-protected amino acids (10 mmol) is suspended in anhydrous methanol (MeOH) (about 30-80 mL) and cooled to about 0° C. (ice bath). Neat thionyl chloride (SOCl₂) (40-50 mmol) is added carefully, and the reaction mixture is heated at reflux for about 1-6 h before cooling down to room temperature. The reaction was followed by LC/MS to completion. The solvents are evaporated under reduced pressure using a rotary evaporator. The residue is co-evaporated with additional MeOH (2×50) to remove residual volatiles and solvent. Residual solvents are removed under reduced pressure to afford the amino acid methyl esters generally as colorless solids, which are generally of sufficient purity to be used directly in the next step without further purification and isolation procedures.

### Description 5

### General Procedure for the Amino Acid N-Protection with Alkyl Chloroformates

Adapting literature protocols well known in the art, the unprotected amino acid derivative or a salt thereof, e.g. a hydrochloride salt, (10 mmol) is suspended in anhydrous dichloromethane (DCM) (about 30-50 mL) and the mixture is cooled to about 0° C. (ice bath). Neat diisopropylethylamine (DIPEA, Hünigs-base) (20-50 mmol) is added followed by the appropriate alkyl chloroformate (15 mmol), e.g., benzylchloroformate (ZCl or CbzCl) or ethylchloroformate, is added dropwise and the reaction mixture is stirred with gradual warming to room temperature for overnight. The reaction is monitored by TLC and/or LC/MS to completion. The solvents are removed under reduced pressure using a rotary evaporator. The residue is diluted with 1.0 molar hydrochloric acid (HCl) and the aqueous phase is extracted with ethyl acetate (EtOAc) (3×). The combined organic extracts are dried over anhydrous sodium sulfate (Na₂SO₄) or anhydrous magnesium sulfate (MgSO₄), and filtered using a Büchner funnel. The filter residue is washed with additional EtOAc, and the combined organic filtrates are evaporated under reduced pressure using a rotary evaporator. The crude material is purified by silica gel column chromatography or is re-crystallized to afford the target compounds.

### Description 6

### General Procedure for the Reduction of Nitro-Aromates to Anilines

Variant A: Adapting a literature known protocol (Chandrappa, et al., Synlett, 2010, (20), 3019-3022), to a suspension of the nitro aromatic derivative (10 mmol) in a mixture of ethanol (EtOH) or methanol (MeOH) with water (10-20 mL alcohol:0.5-3 mL water), iron powder (Fe) (30-100 mmol), and calcium chloride dihydrate (CaCl₂.2H₂O) (5-10 mmol) are added. The resulting reaction mixture is heated from about 50° C. to about reflux (oil bath) for about 0.5-3 h. The reaction is followed by TLC (nihydrin stain) and/or analytical LC/MS to completion. The reaction mixture is cooled to room temperature and filtered through a short path of Celite® 545 to remove iron residues. The filter aid is washed with additional alcohol/water mixture or ethyl acetate (EtOAc) (3×). The combined organic filtrates are dried over anhydrous sodium sulfate (Na₂SO₄) or anhydrous magnesium sulfate (MgSO₄), the drying agent is filtered off, the filter residue is washed with additional MeOH or EtOAc, filtered over a Bichner funnel, and the combined filtrates are evaporated under reduced pressure using a rotary evaporator. The crude material may be purified by silica gel column chromatography preferentially using dichloromethane (DCM) and methanol mixtures optionally containing 1-5 vol-% of triethylamine or is re-crystallized.

Variant B: Adapting literature protocols well known in the art, the nitro aromatic derivative (10 mmol) is dissolved in methanol (MeOH), ethanol (EtOH), ethyl acetate (EtOAc), or mixtures of any of the foregoing (25-50 mL). The heterogeneous catalyst (5 or 10 wt-% palladium on charcoal containing ˜50 wt-% water) (about 25-50 wt-% with respect to the nitro aromatic derivative) is added. Optionally, a small amount of acidic additives, e.g. few drops of HOAc or 1.0 M hydrochloric acid (HCl) are added to activate the catalyst. The atmosphere is exchanged to hydrogen (3× evacuation/refill technique) and the reaction mixture is stirred at room temperature under about 15 psi (H₂-balloon) for 1-12 h. Optionally, the reaction is carried out in a stainless steel reactor or a Parr-hydrogenation apparatus if higher pressures of H₂ are required. The reaction is monitored by TLC and/or LCMS to completion. The reaction mixture is filtered over a short plug of Celite® 545, the filtration aid is washed with MeOH, and the combined filtrates are evaporated under reduced pressure. The crude material is purified as described under Variant A.

### Description 7

### General Procedure for the Reductive N-Alkylation

Adapting literature known protocols (Palani, et al., J. Med. Chem., 2005, 48(15), 4746-4749; van Oeveren, Bioorg. Med. Chem. Lett., 2007, 17(6), 1527-1531; Delfourne, et al., Bioorg. Med. Chem., 2004, 12(15), 3987-3994; Delfourne, et al., J. Med. Chem., 2002, 47(17), 3765-3771; and Jordan, et al., Bioorg. Med. Chem., 2002, 10(8), 2625-2633), to a solution of the aniline (or a suspension of an aniline addition salt, e.g., a hydrochloride salt) (10 mmol) in methanol (MeOH) (30 mL) at about 5-15° C. (water bath with some ice) is added trifluoroacetic acid (TFA) (15 mL) (Variant A), acetic acid (15-20 mL) (HOAc) (Variant B), or 85 wt-% phosphoric acid (H₃PO₄) (Variant C). To the cooled solution, is added commercial 2-chloroacetaldehyde (ClCH₂CHO) (˜50 wt-% in water, ˜7.87 M) (˜6.5 mL, ˜50 mmol). The reaction mixture is stirred for about 15-30 min at this temperature when sodium cyanoborohydride (NaBH₃CN) (2.51 g, 40 mmol) was added in small portions (exothermic hydrogen evolution!). The reaction mixture is stirred for 15-120 min with gradual warming to room temperature. In some case copious amounts of a precipitate are generated during the reaction. The reaction is monitored by TLC and/or LC/MS to completion. The majority of the volatiles (Variants A and B) are evaporated under reduced pressure (rotary evaporator; ambient to 35° C. bath temperature). The residue is dissolved in ethyl acetate (EtOAc) and the organic phase is successively washed with a saturated aqueous solution of sodium hydrogencarbonate (NaHCO₃) (2×) and brine (1×). The organic solution is dried over anhydrous magnesium sulfate (MgSO₄), filtered, and the organic solvents were evaporated to dryness under reduced pressure. If non non-volatile acids are used (Variant C), the reaction mixture is diluted with water and neutralized (pH 5-7) with solid sodium hydrogencarbonate (NaHCO₃). The aqueous phase is extracted with ethyl acetate (EtOAc) (3×) and the combined organic extracts are treated as described for Variants A and B. The crude material is purified by silica gel column chromatography or is re-crystallized.

### Description 8

### General Procedure for Deprotection by Acid Hydrolysis with Strong Aqueous Acids

Adapting literature known protocols (Taylor, et al., Chem. Biol. Drug Des., 2007, 70(3), 216-226; Buss, et al., J. Fluorine Chem., 1986, 34(1), 83-114; Abela, et al, J. Chem. Soc., Perkin Trans. 1, 1997, (20), 2258-2263; Weisz, et al., Bioorg. Med. Chem. Lett., 1995, 5(24), 2985-2988; Zheng, Bioorg., Med., Chem., 2010, 18(2), 880-886; Haines, et al., J. Med. Chem., 1987, 30, 542-547; and Matharu, et al., Bioorg., Med., Chem., Lett., 2010, 20, 3688-3691), hydrolytic removal of protecting groups is conducted through heating a suspension or solution of the corresponding protected N-mustard (1 mmol) in 2-12 M of an aqueous hydrohalogenic acid (5-10 mL/mmol) or a 20-80 vol-% mixture of a 2-12 M of an aqueous hydrohalogenic acid with 1,4-dioxane (5-10 mL/mmol) at an elevated temperature from about 30° C. to about 150° C. (sealed tube) for 1-24 h. The reaction e is be followed by TLC and/or LC/MS to completion. Organic side products, e.g., phthalic acid or benzoic acid, may be extracted with an organic solvent, e.g., ethyl acetate (EtOAc) or chloroform (CHCl₃). The aqueous solution or organic volatile solvents are evaporated using a rotary evaporator (40° C. to 60° C. water bath temperature) to yield the crude target product which may be dissolved in a ˜50 vol-% aqueous acetonitrile (MeCN) followed by lyophilization. Where applicable, the crude target compound is further purified by RP-HPLC purification using acetonitrile/water mixtures containing 0.05-0.1 vol-% formic acid (FA) or trifluoroacetic acid (TFA) followed by primary lyophilization, optionally in the presence of 1.0 or an excess of an acid capable of forming pharmaceutically acceptable salt addition products. Where applicable, the crude material is purified by re-crystallization, titruation, or repeated precipitation.

### Description 9

### Global Deprotection of Under Anhydrous Conditions with Strong Acids

Variant A: Adapting literature known protocols (Springer, et al., J. Med. Chem., 1990, 33(2), 677-681; Davies, et al., J. Med. Chem. 2005, 48(16), 5321-5328; Niculesscu-Duvaz, et al., J. Med. Chem., 2004, 47(10), 2651-2658; Verny and Nicolas, J. Label. Cmpds, Radiopharm., 1988, 25(9), 949-955; Thorn, et al., J. Org. Chem, 1975, 40(11), 1556-1558; Baraldini, et al., J. Med. Chem., 2000, 53(14), 2675-2684; Gourdi, et al., J. Med. Chem., 1990, 33(4), 1177-1186; and Kupczyk-Subotkowska, et al., J. Drug Targeting, 1997, 4(6), 359-370), a solution of the corresponding protected N,N-bis(2-chloroethyl)aryl-substituted β-substituted β-amino acid precursor (1.0 mmol) in neat trifluoroacetic acid (TFA), a mixture of TFA and dichloromethane (DCM) or 1,2-dichloroethane (DCE) (90 vol.-% TFA to 90 vol.-% organic solvent), or 98% formic acid (HCO₂H) (10-25 mL/mmol) is stirred at about room temperature for about 1-24 h. Optionally, scavengers (2-5 mmol) such as triethysilane (Et₃SiH), triisopropylsilane (iPr₃SiH), thioanisole (PhSMe), or 1,2-dithioethane (HSCH₂CH₂HS) are added to the reaction mixture to suppress unwanted side reactions (Metha, Tetrahedron Lett., 1992, 33(37), 5411-5444). The reaction is be followed by TLC and/or analytical LC/MS to completion. The solvent is removed under reduced pressure using a rotary evaporator (water bath temperature at about 30° C.). Optionally, residual acid traces are azeotropically removed through repeated co-evaporation (5-10×) under reduced pressure using a suitable co-solvent, e.g., ethyl acetate (EtOAc), toluene, or DCM to yield the crude target compound, which may be used directly in in vitro or in vivo experiments. Further purification is conducted as described for Description 8.

Variant B: Adapting literature known protocols, a solution of the corresponding protected N,N-bis(2-chloroethyl)aryl-substituted β-substituted γ-amino acid precursor (1.0 mmol) in 2 M hydrogen chloride in diethyl ether (2.0 M HCl in Et₂O) or 4 M hydrogen chloride in 1,4-dioxane (4.0 M HCl in 1,4-dioxane) is stirred at about room temperature for about 1-36 h. Optionally scavengers are the same as in Variant A. The reaction is be followed by TLC and/or analytical LC/MS to completion. The reaction mixture is centrifuged for about 10 min at 3000 rpm, the supernatant decanted or pipetted off, and the precipitate is suspended in anhydrous Et₂O repeating the centrifugation/washing sequence (2-3×). The crude target compound may be used directly in in vitro or in vivo experiments. Further purification is conducted as described for Description 8.

### Description 10

### General Procedure for the Bromination of Benzylic Alcohols to Benzylic Bromides

Adapting literature known protocols (Harrison and Diehl, Org. Synth., 1955, Coll. Vol. 3, 370), the benzylic alcohol (50 mmol) is dissolved in anhydrous dichloromethane (DCM) (about 100-150 mL) and the solution is cooled to about 00 (ice bath). To the solution is dropwise added a commercial 1.0 M solution of phosphorus tribromide (PBr₃) (50 mmol) and the resulting mixture is stirred for about 1-2 h at this temperature. The reaction is followed by TLC to completion. The reaction mixture is poured onto a mixture of crushed ice and a saturated sodium hydrogencarbonate solution. After phase separation, the aqueous phase is extracted with DCM or ethyl acetate (EtOAc) and the combined organic extracts are washed with a saturated aqueous solution of sodium hydrogencarbonate (NaHCO₃) (1×) and brine (1×), dried over anhydrous magnesium sulfate (MgSO₄), filtered, the filter residue is washed with DCM, and the combined organic filters are evaporated under reduced pressure. If needed, the crude material is purified by silica gel column chromatography or is re-crystallized.

### Description 11

### General Procedure for the Arndt-Eistert Homologation of Amino Acids

Part A: Adapting literature protocols (Aldrich Technical Bulletin: Diazald® and Diazomethane Generators; Black, Aldrichchimica Acta, 1983, 16(1), 3-10; and Lombardy, Chem. Ind., 1990, 708), a solution of diazomethane (CH₂N₂) in diethyl ether (Et₂O) is freshly prepared prior to use in an Aldrich Diazald® apparatus through addition of a solution of commercial N-methyl-N-nitrosotoluene-4-sulphonamide (Diazald®) (15 g, 70.0 mmol) in Et₂O (150 mL) to a reaction mixture containing potassium hydroxide (KOH) (15 g, 267 mmol) in Et₂O (25 mL), water (30 mL), and 2-(2-ethoxyethoxy)ethanol (50 mL) at about 65° C. (oil bath). The reaction is completed when the yellow color subsided. The CH₂N₂ is trapped in Et₂O.

Part B: Adapting literature protocols (Podlech and Seebach, Liebigs Ann., 1995, 1217-1228; Limbach, et al., Liebigs Ann., 2006, 89(7), 1427-1441; Podlech and Seebach, Angew. Chem. Int. Ed. Engl., 1995, 34(4), 471-472; Müller, et al., Synthesis, 1998, (6), 837-841); and Bartosz-Bechowski and Konopinska, J. Prakt. Chem., 1989, 331(3), 532-536), an N-protected amino acid derivative (10 mmol) is dissolved under a nitrogen atmosphere in anhydrous tetrahydrofuran (THF) and the solution is cooled to about −20° C. (dry ice/acetone bath). To the solution is added N-methylmorpholine (NMM) (13 mmol), followed by neat isobutyl chloroformate (12 mmol). The reaction mixture is stirred at about −20° C. for about 2 h, when an excess of (5-10 equivalents) of the freshly prepared ethereal solution of diazomethane is added. Optionally, the precipitated NMM hydrochloride (NMM.HCl) is filtered off under a nitrogen atmosphere prior to diazotation. The reaction mixture is gradually warmed to room temperature and stirred for an additional 2 h. Excess diazomethane is quenched with a few drops of acetic acid (HOAc). The solvents are removed under reduced pressure using a rotary evaporator. The residue is dissolved in a mixture of Et₂O and ethyl acetate (EtOAc). Basic aqueous work-up with a saturated aqueous solution of sodium hydrogencarbonate (NaHCO₃) and silica gel column chromatography furnish the diazoketones typically as light yellow solids.

Part C: Adapting literature protocols (see Part B), an N-protected diazoketone (10 mmol) is dissolved under a nitrogen atmosphere in anhydrous methanol (MeOH) (about 2-4 mL) and anhydrous tetrahydrofuran (THF) (about 20-25 mL) and the solution is degassed and placed under a nitrogen atmosphere (3 times evacuation/refill cycling) and under exclusion from (sun)light. A mixture of silver benzoate (AgBz) (5.0 mmol) in THF (about 5-10 mL) and triethylamine (TEA) (20 mmol) is added slowly at room temperature. Gas evolution! The reaction mixture is stirred for about 1-4 hours at room temperature and concentrated under reduced pressure using a rotary evaporator. The residue is purified by silica gel column chromatography using (EtOAc) and hexane mixtures.

### Description 12

### General Procedures for the Preparation of Succinimidyl Esters

Adapting a literature protocol (Dexter and Jackson, J. Org. Chem., 1999, 64, 7579-7585), to a stirred solution of the N-protected aspartic acid β-alkyl ester (25 mmol) in ethyl acetate (EtOAc) or acetonitrile (MeCN) (about 25-75 mL) is added solid N-hydroxysuccinimide (NHS, HOSu) (26-28 mmol) at about 00 (ice bath). A solution of dicyclohexylcarbodimide (DCC) (25-26 mmol) in EtOAc or MeCN (about 25 mL) is added slowly. Optionally, solid DCC is added in small portions. Optionally, any of the common carboxylic acid activation agents can be used for this reaction (Montalbetti and Falque, Tetrahedron, 2005, 61, 10827-10852; and Valeur and M Bradley, Chem. Soc. Rev., 2009, 38, 606-631). The reaction is stirred with gradual warming to room temperature for about 6-24 hours. The reaction is monitored by TLC to completion. The precipitated dicyclohexylurea (DCU) is filtered off using a Bichner-funnel, and the filtrate is washed with a saturated aqueous solution of sodium hydrogencarbonate (NaHCO₃) (3×), brine (1×), dried over anhydrous magnesium sulfate (MgSO₄), filtered, and evaporated under reduced pressure using a rotary evaporator. The OSu-esters are usually obtained in quantitative yield and may be of sufficient purity to be used directly in the next steps without further isolation and purification.

### Description 13

### General Procedures for the Reduction of Succinimidyl Esters to Alcohols

Adapting a literature protocol (Dexter and Jackson, J. Org. Chem., 1999, 64, 7579-7585), sodium borohydride (NaBH₄) (15-20 mmol) is dissolved in water (about 3-6 mL) and tetrahydrofuran (about 25-50 mL) at about 0° C. (ice bath). A solution of the succimidyl-ester (10.0 mmol) in THF (about 5-10 mL) is added dropwise over about 1 minute. The reaction is monitored by TLC to completion (<30 min). The reaction is quenched through addition of 1.0 M hydrochloric acid (pH ˜1-2) or a saturated aqueous solution of ammonium chloride (NH₄Cl). Volatiles (THF) is partially removed under reduced pressure using a rotary evaporator. The aqueous phase is extracted with ethyl acetate (EtOAc) (3×). The combined organic extracts are washed with a saturated aqueous solution of sodium hydrogencarbonate (NaHCO₃) (1×), brine (1×), dried over anhydrous magnesium sulfate (MgSO₄), filtered, and evaporated under reduced pressure using a rotary evaporator. The residue is purified by silica gel column chromatography using EtOAc and hexane mixtures.

### Description 14

### General Procedures for the Preparation of Iodides from Alcohols

Adapting a literature protocol (Dexter and Jackson, J. Org. Chem., 1999, 64, 7579-7585), triphenylphosphine (40 mmol), imidazole (40 mmol), and iodine (40 mmol) are added to anhydrous dichloromethane (DCM) (about 100-120 mL). A solution of the alcohol (40 mmol) in DCM (about 40 mL) is added at room temperature. The reaction is monitored by TLC to completion (about 1-2 h). The reaction mixture is filtered (Büchner-funnel) to remove precipitated triphenylphosphine oxide (Ph₃PO) and the filtrate is washed with a 1.0 M aqueous solution of sodium thiosulfate (Na₂S₂O₃) (2×), brine (1×), dried over anhydrous magnesium sulfate (MgSO4), filtered, and evaporated under reduced pressure using a rotary evaporator. The residue is first slurried in diethyl ether (removal of additional Ph₃PO), filtered through over a short bed of silica gel or purified by silica gel column chromatography.

### Description 15

### General Procedure for the Negishi-Coupling with Aromatic Halides

Part A: Adapting literature protocols (Dexter and Jackson, J. Org. Chem., 1999, 64, 7579-7585; Dexter, et al., J. Org. Chem., 2000, 65, 7417-7421; Jackson and M. Perez-Gonzales, Org. Synth., 2005, 81, 77-88; Ross, J. Org. Chem., 2010, 75, 245-248; Anzalone, et al., U.S. Pat. No. 8,710,256; Hoepping, et al., International Publication No. WO 2014/095739; and Jackson and Perez-Gonzales, Org. Synth., 2005, 81, 77-88), zinc dust (Zn) (30 mmol, 3-6 equivalents) is suspended under an atmosphere of inert gas (nitrogen or argon) in anhydrous degassed N,N-dimethylformamide (DMF), N,N-dimethyl acetamide (DMAc or DMA), tetrahydrofuran (THF), or 2-methyl-tetrahydrofuran (2-Me-THF) (about 5-10 mL). The zinc metal is activated by addition of elemental iodine (I₂) (about 1.5-3.0 mmol, 15-30 mol-%) and trimethyl silylchloride (MeSiCl, TMSCl) (about 1.5-3.0 mmol, 15-30 mol-%). After subsiding of the exotherm, the appropriate iodo-compound (5-10 mmol) is added, optionally as a solution in a small amount of the same anhydrous an degassed solvent, followed by addition of the same amounts of I₂ and TMSCl. Optionally, a combination of 1,2-dibromoethane (3 mmol, 30 mol-%) and TMSCl (6 mol %) may be used to activate the zinc dust. After subsiding of the exotherm to room temperature and settling of the zinc dust, the supernatant containing the appropriate zinc organic compound is ready to use in the subsequent Negishi cross-coupling reaction.

Part B: Adapting literature protocols (see Part A), the supernatant containing the appropriate zinc organic compound is transferred to a solution of the aryl halide (6.5-13 mmol, 1.3 equivalents), tris(benzylideneacetone) dipalladium (Pd₂(dba)₃) (0.125-0.25 mmol, 2.5 mol-%) and tris(o-tolyl)phosphine (P(o-tol)₃) (0.5-1 mmol, 10 mol-%) or SPhos (2-dicyclohexylphosphino-2′,6′-dimethoxybiphenyl) (0.25-0.5 mmol, 5 mol-%) in anhydrous dry degassed N,N-dimethylformamide (DMF), N,N-dimethyl acetamide (DMAc or DMA), tetrahydrofuran (THF), or 2-methyl-tetrahydrofuran (2-Me-THF) (about 5-10 mL). The reaction mixture is stirred at room temperature for 1-12 hours or heated under an inert gas atmosphere to about 40-60° C. for about 1-12 hours. Heating is required to cross-couple aryl bromides. He reaction is followed by TLC and/or LCMS to completion. Dilution with water is followed by extraction of the aqueous phase with ethyl acetate (EtOAc) (3×). The combined organic extracts are washed with a saturated aqueous solution of sodium hydrogencarbonate (NaHCO₃) (1×), brine (1×), dried over anhydrous magnesium sulfate (MgSO₄), filtered, and evaporated under reduced pressure using a rotary evaporator. The residue is purified by silica gel column chromatography using EtOAc and hexane mixtures.

### Description 16

### General Procedure for the N,N-Bis-(2-Hydroxyethylation) of Anilines with Ethylene Oxide

Adapting literature known protocols (Palmer, et al., J. Med. Chem. 1990, 33(1), 112-121; Jordan, et al., Bioorg. Med. Chem., 2002, 10(8), 2625-2633; Abela Medici, et al, J. Chem. Soc., Perkin Trans. 1, 1997, (20), 2258-2263; Feau, et al., Org. Biomolecular Chem., 2009, 7(24), 5259-5270; Springer, et al., J. Med. Chem., 1990, 33(2), 677-681; Taylor, et al., Chem. Biol. Drug Des., 2007, 70(3), 216-226; Buss, et al., J. Fluorine Chem., 1986, 34(1), 83-114; Larden and Cheung, Tetrahedron Lett., 1996, 37(42), 7581-7582; Spreitzer and Puschmann, Monatshefte fiir Chemie, 2007, 138(5), 517-522; Niculesscu-Duvaz, et al., J. Med. Chem., 2004, 47(10), 2651-2658; Weisz, et al., Bioorg. Med. Chem. Lett., 1995, 5(24), 2985-2988; Thorn, et al., J. Org. Chem, 1975, 40(11), 1556-1558; Baraldini, et al., J. Med., Chem., 2000, 53(14), 2675-2684; Zheng, et al., Bioorg., Med., Chem., 2010, 18(2), 880-886; Gourdi, et al., J., Med., Chem., 1990, 33(4), 1177-1186; Haines, et al., J. Med. Chem., 1987, 30, 542-547; Matharu, et al., Bioorg. Med. Chem. Lett., 2010, 20, 3688-3691; and Kupczyk-Subotkowska, et al., J. Drug Targeting, 1997, 4(6), 359-370), a mixture of the corresponding aniline (25.0 mmol) in aqueous acetic acid (HOAc) (25-75 vol-%) (25-100 mL) is cooled to about −20° C. (ice/sodium chloride bath) to about 0° C. (ice bath). Optionally, the solvent may also glacial acetic acid (HOAc), water, tetrahydrofuran (THF), ethanol (EtOH), 1,4-dioxane (for higher temperature reactions), or mixtures of any of the foregoing. An excess of ethylene oxide (oxirane) (100-400 mmol) is added to the reaction mixture either neat in pre-cooled form or dissolved in any of the foregoing solvents or mixtures thereof. The reaction mixture is stirred at about room temperature for about 12-48 h. Alternatively, the reaction mixture may be heated in a sealed reaction vessel at 80-140° C. for a similar time. The reaction is followed by TLC and/or LC/MS and is usually complete when the reaction mixture turns clear. The solvents are removed under reduced pressure using a rotary evaporator (40-60° C. water bath temperature). The residue is diluted with ethyl acetate (EtOAc), washed with brine, dried over anhydrous magnesium sulfate (MgSO₄) or sodium sulfate (Na₂SO₄), filtered, and the solvents removed under reduced pressure using a rotary evaporator to yield the target compound, which may be used directly in the next step. The crude material may be further purified by silica gel column chromatography using EtOAc, methanol (MeOH), dichloromethane and hexanes, or mixtures of any of the foregoing to furnish the purified target compound. Alternatively, the crude target compound may be further purified by re-crystallization.

### Description 17

### General Procedures for Chlorination of N,N-Bis(2-Hydroxyethyl)-Groups

Variant A: Chlorination with Thionyl Chloride (SOCl₂)

Adapting literature known protocols (Palmer, et al., J. Med. Chem. 1990, 33(1), 112-121; Jordan, et al., Bioorg. Med. Chem., 2002, 10(8), 2625-2633; Abela Medici, et al., J. Chem. Soc., Perkin Trans. 1, 1997, (20), 2258-2263; Taylor, et al., Chem. Biol. Drug Des., 2007, 70(3), 216-226; Dheyongera, Bioorg. Med. Chem. 2005, 13(3), 689-698; Zheng, Bioorg. Med. Chem. 2010, 18(2), 880-886; Gourdi, J. Med. Chem., 1990, 33(4), 1177-1186; and Lin, et al., Bioorg. Med. Chem. Lett., 2011, 21(3), 940-943), to a solution of thionyl chloride (SOCl₂) (10-75 mmol) in an anhydrous organic solvent, e.g., dichloromethane (DCM), chloroform (CHCl₃), 1,2-dichloroethane (DCE), benzene, or mixtures of any of the foregoing (25-100 mL) is added at a temperature from about 0° C. (ice bath) to about room temperature the corresponding N,N-bis(2-hydroxyethyl) derivative (5.0 mmol), either in neat form (portions) or as a solution in a small volume in any of the foregoing solvents. The reaction mixture is stirred at about room temperature to about 40° C. or heated to reflux for about 10 minutes to about 3 h. Optionally, the reaction is carried out using neat SOCl₂ directly as the solvent. Optionally, the reaction is carried out in the presence of a catalytic amount of zinc chloride (ZnCl₂) (10 mol-% to 40 mol-%) or N,N-dimethylformamide (about 1 to 3 drops) to facilitate the reaction (Squires, et al., J. Org. Chem., 1975, 40(1), 134-136; and Abela Medici, et al, J. Chem. Soc., Perkin Trans. 1, 1997, (20), 2258-2263). The reaction is followed by TLC and/or LC/MS to completion. Volatiles (solvents and excess of SOCl₂) are removed under reduced pressure using a rotary evaporator. Optionally, a small amount of co-solvent, e.g., of benzene, is added to assist in azeotropic co-evaporation and removal of residual excess chlorination agent. The residue is diluted with 1.0 M hydrochloric acid (HCl). The aqueous phase is extracted with ethyl acetate (EtOAc) (3×), and the combined organic extracts are washed with a saturated aqueous solution of sodium hydrogen carbonate (NaHCO₃) (2×) and brine (1×). The organic layer is dried over anhydrous magnesium sulfate (MgSO₄) or sodium sulfate (Na₂SO₄), filtered, and the solvents removed under reduced pressure using a rotary evaporator. The residue is purified by silica gel column chromatography using EtOAc and hexanes mixtures.

Variant B: Chlorination with Phosphoryl Chloride (POCl₃)

Adapting literature known protocols (Palmer, et al., J. Med. Chem. 1990, 33(1), 112-121; Feau, et al., Org. Biomolecular Chem., 2009, 7(24), 5259-5270; Valu, et al., J. Med. Chem., 1990, 33(11), 3014-3019; Baraldini, et al., J. Med., Chem., 2000, 53(14), 2675-2684; Gourdi, et al., J., Med., Chem., 1990, 33(4), 1177-1186; Haines, et al., J. Med. Chem., 1987, 30, 542-547; and Matharu, et al., Bioorg. Med. Chem. Lett., 2010, 20, 3688-3691), to a solution of phosphorus(V) oxychloride (phosphoryl chloride, POCl₃) (10-50 mmol) in an anhydrous organic solvent, e.g., benzene, acetonitrile, pyridine, or mixtures of any of the foregoing (25-100 mL) is added at a temperature from about 0° C. (ice bath) to about room temperature the corresponding N,N-bis(2-hydroxyethyl) derivative (5.0 mmol) either in neat form (portions) or as a solution in a small volume in any of the foregoing solvents. The remainder of the reaction, work-up, and product isolation are essentially conducted as described in Variant A.

Variant C: Chlorination with Methanesulfonyl Chloride/Pyridine

Adapting literature known protocols (Jordan, et al., Bioorg. Med. Chem., 2002, 10(8), 2625-2633; Abela Medici, et al, J. Chem. Soc., Perkin Trans. 1, 1997, (20), 2258-2263; Springer, et al., J. Med. Chem., 1990, 33(2), 677-681; Larden and Cheung, Tetrahedron Lett., 1996, 37(42), 7581-7582), a solution of methanesulfonyl chloride (MsCl) (20.0 mmol) in anhydrous pyridine (about 10 mL) is drop-wise added with stirring and at a temperature of about 0° C. (ice bath) to a solution of the corresponding N,N-bis(2-hydroxyethyl) derivative (5 mmol) in anhydrous pyridine (about 10 mL). After about 30 minutes, the reaction mixture is heated at 50-100° C. for about 1-3 h. After cooling to room temperature, potential precipitates, if any, e.g., pyridinium methansulfonate, are filtered off before the solvents are partially removed under reduced pressure using a rotary evaporator. The remainder of the reaction, work-up, and product isolation are essentially conducted as described in Variant A.

Variant D: Chlorination with Triphenylphosphine/Tetrachlorocarbon (PPh₃/CCl₄)

Adapting literature known protocols (Buss, et al., J. Fluorine Chem., 1986, 34(1), 83-114; and Kupczyk-Subotkowska, et al., J. Drug Targeting, 1997, 4(6), 359-370), a solution of the corresponding N,N-bis(2-hydroxyethyl) derivative (5 mmol) in anhydrous dichloromethane (DCM) (about 25 mL) containing carbon tetrachloride (CCl₄) (15-25 mmol) is cooled to about 0° C. (ice bath). Alternatively, neat carbon tetrachloride (CCl₄) (25 mL) is used as a reaction solvent. The reaction mixture is stirred, and triphenylphosphine (Ph₃P) (10-15 mmol) is added in portions. The reaction mixture is stirred for about 8-14 h with gradual warming to room temperature. Alternatively, the reaction mixture is heated at reflux for about 2-6 h. The reaction is followed by TLC and/or LC/MS to completion. The reaction mixture is cooled to room temperature and the solvents are removed under reduced pressure using a rotary evaporator. The residue is triturated with diethyl ether (Et₂O) (3×) to remove some of the triphenylphosphine oxide (Ph₃PO). The organic phase is evaporated under reduced pressure using a rotary evaporator. The remainder of the reaction, work-up, and product isolation are essentially conducted as described in Variant A.

### Description 18

### General Procedure for the Mesylation of N,N-Bis(2-Hydroxyethyl)-Groups

Variant A: Adapting literature protocols (Davies, et al., J. Med. Chem. 2005, 48(16), 5321-5328; Springer, et al., J. Med. Chem., 1990, 33(2), 677-681; Niculesscu-Duvaz, et al., J. Med. Chem., 2004, 47(10), 2651-2658; and Yang, et al., Tetrahedron, 2007, 63(25), 5470-5476), to a cooled solution (about 0° C. (ice bath)) of the corresponding N,N-bis(2-hydroxyethyl) derivative (5.0 mmol) in anhydrous dichloromethane (DCM) (25-50 mL) are added triethylamine (Et₃N, TEA) (25.0 mmol) or anhydrous pyridine (25.0 mmol), and a catalytic amount of 4-N,N-(dimethylamino)pyridine (DMAP) (1.0 mmol, 20 mol-%). Methanesulfonyl anhydride (Ms₂O) (20.0 mmol) is added portion-wise or as a solution in DCM (5-10 mL). The reaction mixture is stirred with gradual warming to room temperature for about 8-24 h. The reaction is be followed by TLC and/or LC/MS. Solvents are removed under reduced pressure using a rotary evaporator. The residue is diluted with 1.0 M hydrochloric acid (HCl), and the aqueous phase is extracted with ethyl acetate (EtOAc) (3×). The combined organic extracts are washed with a saturated aqueous solution of sodium hydrogen carbonate (NaHCO₃), and brine, dried over anhydrous magnesium sulfate (MgSO₄) or sodium sulfate (Na₂SO₄), filtered, and the solvents are removed under reduced pressure using a rotary evaporator to yield the target compound, which may be used directly in the next step. Alternatively, the crude residue may be further purified by silica gel column chromatography using EtOAc, methanol (MeOH), dichloromethane (DCM), and hexanes, or mixtures of any of the foregoing to furnish the purified target compound. Alternatively, the crude target compound may be further purified by re-crystallization.

Variant B: Adapting literature known protocols (Palmer, et al., J. Med. Chem. 1990, 33(1), 112-121; B. D. Palmer, et al., J. Med. Chem., 1994, 37, 2175-2184; Palmer, et al., J. Med. Chem, 1996, 39(13), 2518-2528; Spreitzer and Puschmann, Monatshefte fiir Chemie, 2007, 138(5), 517-522; Lin, et al., Bioorg. Med. Chem. Lett., 2011, 21(3), 940-943; Gourdi, et al., J. Med. Chem., 1990, 33(4), 1177-1186; Ferlin, et al., Bioorg. Med. Chem., 2004, 12(4), 771-777; Thorn, et al., J. Org. Chem, 1975, 40(11), 1556-1558; Coggiola, et al., Bioorg. Med. Chem. Lett., 2005, 15(15), 3551-3554), to a cooled solution (about 0° C. (ice bath)) of the corresponding N,N-bis(2-hydroxyethyl) derivative (5.0 mmol) in anhydrous dichloromethane (DCM), tetrahydrofuran (THF), ethyl acetate (EtOAc), or a mixture thereof (20-40 mL) are added triethylamine (Et₃N, TEA) (15.0 mmol) or anhydrous pyridine (25.0 mmol). Methanesulfonyl chloride (MsCl) (12.5 mmol) is added drop-wise to the reaction mixture. The reaction mixture is stirred for about 1-2 h at this temperature. The reaction may be followed by TLC and/or LC/MS. Aqueous work-up and purification by silica gel chromatography are performed as described for Variant A.

### Description 19

### General Procedure for the Finkelstein Conversion to N,N-Bis(2-Halogenoethyl)-Groups

Adapting literature known protocols (Palmer, et al., J. Med. Chem. 1990, 33(1), 112-121; Palmer, et al., J. Med. Chem., 1994, 37, 2175-2184; Palmer, et al., J. Med. Chem., 1996, 39(13), 2518-2528; Davies, et al., J. Med. Chem. 2005, 48(16), 5321-5328; Niculesscu-Duvaz, et al., J. Med. Chem., 2004, 47(10), 2651-2658; Weisz, et al., Bioorg. Med. Chem. Lett., 1995, 5(24), 2985-2988; Thorn, J. Org. Chem, 1975, 40(11), 1556-1558; Lin, et al., Bioorg. Med. Chem. Lett., 2011, 21(3), 940-943; Gourdi, et al., J. Med. Chem. 1990, 33(4), 1177-1186; Yang, et al., Tetrahedron, 2007, 63(25), 5470-5476; Ferlin, et al., Bioorg. Med. Chem., 2004, 12(4), 771-777; and Coggiola, et al., Bioorg. Med. Chem. Lett., 2005, 15(15), 3551-3554), a slurry of the corresponding N,N-bis(2-methylsulfonyloxyethyl) derivative (5.0 mmol) and an alkali metal halide, e.g., lithium chloride (LiCl), lithium bromide (LiBr), sodium chloride (NaCl), sodium bromide (NaBr), or sodium iodide (NaI) (20-80 mmol) in an anhydrous organic solvent, e.g., N,N-dimethylformamide (DMF), N,N-dimethylacetamide (DMAc), acetone, 2-butanone (methyl ethyl ketone, MEK), 3-methyl-2-butanone (isopropyl methyl ketone, MIPK), acetonitrile (MeCN), methanol (MeOH), tetrahydrofuran (THF), ethyl acetate (EtOAc) or a mixture of any of the foregoing (10-30 mL), is stirred at room temperature or heated at 50-150° C. for about 1-12 h. The reaction is followed by TLC and/or LC/MS to completion. Solvents are partially or completely removed under reduced pressure using a rotary evaporator. The residue is diluted with 1.0 M hydrochloric acid (HCl), and the aqueous phase is extracted with ethyl acetate (EtOAc) (3×). The combined organic extracts are washed with a saturated aqueous solution of sodium hydrogen carbonate (NaHCO₃), and brine, dried over anhydrous magnesium sulfate (MgSO₄) or sodium sulfate (Na₂SO₄), filtered, and the solvents are removed under reduced pressure using a rotary evaporator to yield the target compound, which may be used directly in the next step. Alternatively, the crude residue may be further purified by silica gel column chromatography using EtOAc, methanol (MeOH), dichloromethane (DCM), and hexanes, or mixtures of any of the foregoing to furnish the purified target compound. Alternatively, the crude target compound may be further purified by re-crystallization.

### Example 1

### 3-Amino-3-[5-[bis(2-chloroethyl)amino]-2-methyl-phenyl]propanoic acid (1)

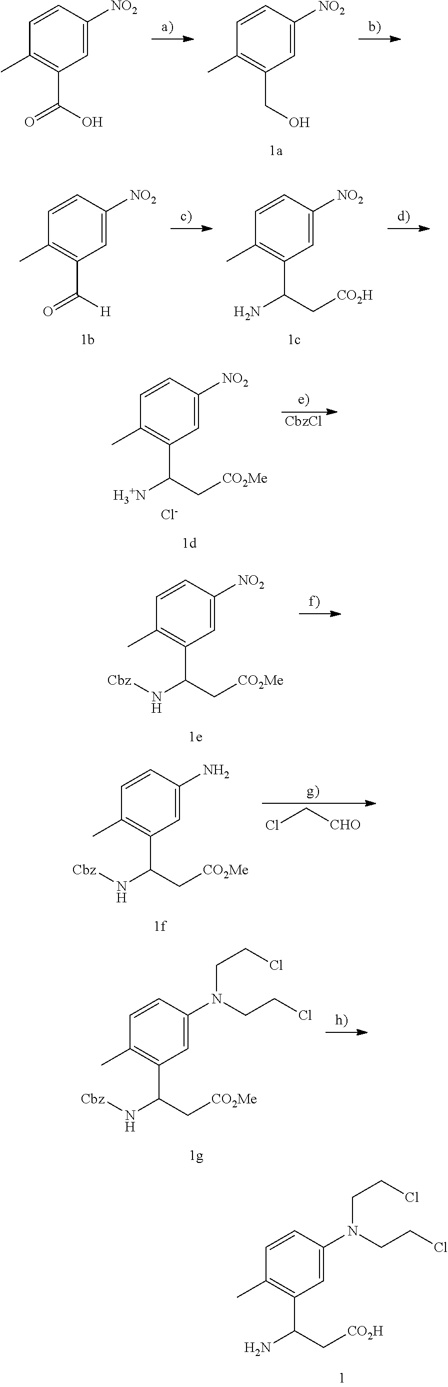

### Step A: (2-Methyl-5-nitro-phenyl)methanol (1a)

Following the General Procedure of Description 1, 2-methyl-5-nitro-phenyl)methanol (1a) was prepared from commercial 2-methyl-5-nitro benzoic acid (50.0 g, 276 mmol) with borane dimethylsulfide complex (2.0 M BH₃.SMe₂ in THF) (166 mL, 332 mmol) in anhydrous tetrahydrofuran (400 mL) to yield 44.0 g (˜quantitative yield) of the target compound (1a) as a pale yellow solid which was of sufficient purity to be used directly in the next step without further isolation and purification. R_{f}: ˜0.50 (EtOAc/Hxn=1:1 v/v). ¹H NMR (300 MHz, CDCl₃): δ 8.30 (d, J=2.4 Hz, 1H), 8.05 (dd, J=8.4, 2.4 Hz, 1H), 7.31 (d, J=8.1 Hz, 1H), 4.78 (d, J=5.1 Hz, 2H), 2.41 (s, 3H), 1.87 (br. t, J=5.1 Hz, 1H) ppm. The compound is also commercially available.

### Step B: 2-Methyl-5-nitro-benzaldehyde (1b)

Following the General Procedure of Description 2 (Variant A), 2-methyl-5-nitro-benzaldehyde (1b) (Beech, J. Chem. Soc. (C), 1967, 2374-2375) was prepared from 2-methyl-5-nitro-phenyl)methanol (1a) (16.3 g, 97.3 mmol) in the presence of dimethylsulfoxide (DMSO) (56.8 mL, 62.6 g, 0.80 mol), triethylamine (TEA, Et₃N) (69.5 mL, 50.6 g, 0.50 mmol), and sulfur trioxide pyridine complex (SO₃.pyridine) (47.8 g, 0.30 mol) in dichloromethane (600 mL). Purification by silica gel column chromatography using a mixture of ethyl acetate (EtOAc) and hexane (EtOAc/hexane=1:4 v/v) afforded 12.6 g (78% yield) of the target compound (1b) as a yellow-beige solid.

Following the General Procedure of Description 2 (Variant B), 2-methyl-5-nitro-benzaldehyde (1b) (Beech, J. Chem. Soc. (C), 1967, 2374-2375) was prepared from 2-methyl-5-nitro-phenyl)methanol (1b) (4.03 g, 24.1 mmol) in the presence of manganese dioxide (MnO₂) (22 g, 254 mmol) in dichloromethane (DCM) (100 mL). Work-up afforded 3.56 g (89% yield) of the target compound (1b) as a pale yellow to beige solid. The material was of sufficient purity to be used directly in the next step without further isolation and purification.

Following the General Procedure of Description 2 (Variant C), 2-methyl-5-nitro-benzaldehyde (1b) (Beech, J. Chem. Soc. (C), 1967, 2374-2375) was prepared from 2-methyl-5-nitro-phenyl)methanol (1a) (5.00 g, 29.9 mmol) in the presence of pyridinium chlorochromate (PCC) (9.02 g, 41.9 mmol) in dichloromethane (DCM) (150 mL). Purification by silica gel column chromatography using mixtures of ethyl acetate (EtOAc) and hexane (EtOAc/hexane=1:4 v/v→EtOAc/hexane=1:4 v/v) afforded 4.67 g (94% yield) of the target compound (1b) as a yellow-beige solid. R_{f}: ˜0.76 (EtOAc/Hxn=1:2 v/v). ¹H NMR (300 MHz, CDCl₃): δ 10.32 (s, 1H), 8.65 (dd, J=2.7 Hz, 1H), 8.31 (dd, J=8.4, 2.4 Hz, 1H), 7.47 (d, J=8.4 Hz, 1H), 2.79 (s, 3H) ppm. The compound is also commercially available.

### Step C: 3-Amino-3-(2-methyl-5-nitro-phenyl)propanoic acid (1c)

Following the General Procedure of Description 3, 3-amino-3-(2-methyl-5-nitro-phenyl)propanoic acid (1c) was prepared from 2-methyl-5-nitro-benzaldehyde (1b) (5.0 g, 30.3 mmol), malonic acid (3.2 g, 30.3 mmol), and ammonium acetate (NH₄OAc) (4.7 g, 60.7 mmol) in ethanol (EtOH) (70 mL) at reflux for 48 hours (oil bath). The reaction was followed by LC/MS to completion. Filtrative work-up afforded 2.2 g (32% yield) of the target compound (1c) as a colorless solid which was of sufficient purity to be used directly in the next step without further purification and isolation procedures. ¹H NMR (300 MHz, D₂O): δ 8.20 (d, J=2.4 Hz, 1H), 8.01 (dd, J=8.1, 2.1 Hz, 1H), 7.38 (d, J=8.7 Hz, 1H), 4.84 (t, J=6.9 Hz, 1H), 2.80-2.60 (m, 2H), 2.37 (s, 3H) ppm. LC/MS: Rₜ=0.480 min; ESI (pos.) m/z=225.1 (M+H⁺)⁺, ESI (neg.) m/z=223.0 (M−H⁺)⁻, 447.1 (2M−H⁺)⁻.

### Step D: Methyl 3-amino-3-(2-methyl-4-nitro-phenyl)propanoate Hydrochloride (1d)

Following the General Procedure of Description 4, methyl 3-amino-3-(2-methyl-4-nitro-phenyl)propanoate hydrochloride (1d) was prepared in a suspension in anhydrous methanol (MeOH) (40 mL) from 3-amino-3-(2-methyl-5-nitro-phenyl)propanoic acid (1c) (2.2 g, 9.81 mmol) with neat thionyl chloride (SOCl₂) (3.54 mL, 5.8 g, 49.1 mmol). Evaporative work-up afforded 2.73 g (about quantitative yield) of the target compound (1d) as a colorless solid, which was of sufficient purity to be used directly in the next step without further purification and isolation procedures. ¹H NMR (300 MHz, DMSO-d⁶): δ 8.86 (br. s, 3H), 8.60 (d, J=2.1 Hz, 1H), 8.11 (dd, J=8.4, 2.1 Hz, 1H), 7.53 (d, J=8.4 Hz, 1H), 4.86 (br. m, 1H), 3.53 (s, 3H), 3.29 (dd, J=16.8, 6.0 Hz, 1H), 3.13 (dd, J=16.8, 8.7 Hz, 1H) ppm. LC/MS: Rₜ=0.492 min; ESI (pos.) m/z=239.1 (M+H⁺)⁺.

### Step E: Methyl 3-benzyloxycarbonylamino-3-(2-methyl-5-nitro-phenyl)propanoate (1e)

Following the General Procedure of Description 5, methyl 3-benzyloxycarbonylamino-3-(2-methyl-5-nitro-phenyl)propanoate (1e) was prepared from crude methyl 3-amino-3-(2-methyl-4-nitro-phenyl)propanoate hydrochloride (1d) (2.7 g, 9.81 mmol), benzyl chloroformate (ZCl, CbzCl) (2.20 mL, 2.63 g of 95% purity=2.5 g, 14.7 mmol), and diisopropylethylamine (DIPEA, Hünigs-base) (6.87 mL, 5.1 g, 39.2 mmol) in anhydrous dichloromethane (DCM) (50 mL). Acidic aqueous work-up and purification by silica gel column chromatography afforded 3.4 g (92% yield) of the target compound (1e) as a colorless solid. R_{f}=0.44 (EtOAc/Hxn=1:2 v/v). ¹H NMR (300 MHz, CDCl₃): δ 8.16 (d, J=2.7 Hz, 1H), 8.24 (dd, J=8.4, 2.4 Hz, 1H), 7.38-7.26 (m, 6H), 5.86 (br. d, 1H), 5.42-5.36 (br. m, 1H), 5.09 (d, J=12.0 Hz, 1H), 5.04 (d, J=12.0 Hz, 1H), 3.64 (s, 3H), 2.84-2.78 (br. m, 2H) ppm. LC/MS: Rₜ=1.790 min; ESI (pos.) m/z=373.2 (M+H⁺)⁺, 767.6 (2M+Na⁺)⁺, ESI (neg.) m/z=743.2 (2M−H⁺)⁻.

### Step F: Methyl 3-(5-amino-2-methyl-phenyl)-3-benzyloxycarbonylamino-propanoate (1f)

Following the General Procedure for of Description 6 (Variant A), methyl 3-(5-amino-2-methyl-phenyl)-3-benzyloxycarbonylamino-propanoate (1f) was prepared from methyl 3-benzyloxycarbonylamino-3-(2-methyl-5-nitro-phenyl)propanoate (1e) (3.35 g, 8.99 mmol), iron powder (Fe) (4.5 g, 81.1 mmol), and calcium chloride dihydrate (CaCl₂ 2H₂O) (0.6 g, 4.05 mmol) in a mixture of methanol (MeOH)/water (68 mL: 12 mL v/v). The reaction mixture was heated at reflux for 2 hours (oil bath). Removal of the iron residues by filtration and compound isolation procedures yielded 3.1 g (about quantitative yield) of the target compound (1f) as a light yellow solid which was of sufficient purity to be used directly in the nest step without further isolation and purification. ¹H NMR (300 MHz, DMSO-d⁶): δ 7.85 (d, J=8.1 Hz, 1H), 7.36-7.24 (m, 5H), 6.74 (d, J=7.8 Hz, 1H), 6.51 (d, J=2.1 Hz, 1H), 6.33 (dd, J=8.4, 2.4 Hz, 1H), 5.10-5.00 (m, 1H), 4.98 (d, J=12.3 Hz, 1H), 4.92 (d, J=12.9 Hz, 1H), 4.79 (br. s, 2H), 3.54 (s, 3H) ppm. LC/MS: Rₜ=1.072 min; ESI (pos.) m/z=365.1 (M+Na⁺)⁺, 685.2 (2M+Na⁺)⁺, 702.2 (2M+Na⁺)⁺.

### Step G: Methyl 3-benzyloxycarbonylamino-3-[5-[bis(2-chloroethyl)amino]-2-methyl-phenyl]propanoate (1g)

Following the General Procedure for of Description 7 (Variant A), methyl 3-benzyloxycarbonylamino-3-[5-[bis(2-chloroethyl)amino]-2-methyl-phenyl]propanoate (1g) was prepared from methyl 3-(5-amino-2-methyl-phenyl)-3-benzyloxycarbonylamino-propanoate (1f) (3.1 g, 9.0 mmol), 2-chloroacetaldehyde (˜50 wt-% in water, ˜7.87 M) (5.8 mL, 45.6 mmol), and sodium cyanoborohydride (NaBH₃CN) (2.4 g of 95% purity=2.3 g, 36.6 mmol) in a mixture of methanol (MeOH) (60 mL) and trifluoroacetic acid (TFA) (30 mL). Aqueous work-up and purification by silica gel column chromatography with an ethyl acetate (EtOAc) hexane mixture (EtOAc/hexane=1:2, v/v) afforded 2.90 g (69% yield) of the title compound (1g) as a colorless solid. R_{f}=0.55 (EtOAc/hexane=1:2, v/v, ninhydrin negative). ¹H NMR (300 MHz, CDCl₃): δ 7.40-7.32, (br. m, 5H), 7.03 (d, J=8.4 Hz, 1H), 6.58 (d, J=2.4 Hz, 1H), 6.52 (dd, J=8.4, 2.7 Hz, 1H), 5.78-5.62 (br. m, 1H), 5.34-5.26 (m, 1H), 5.09 (d, J=12.6 Hz, 1H), 5.07 (d, J=12.6 Hz, 1H), 3.78-3.54 (m, 11H), 2.84-2.78 (m, 2H) ppm. LC/MS: Rₜ=2.271 min; ESI (pos.) m/z=467.1 (M+H⁺)⁺, 489.1 (M+Na⁺)⁺. LC/UV: Rₜ=12.939 min, 100.0% AUC at λ=254 nm.

### Step H: 3-Amino-3-[5-[bis(2-chloroethyl)amino]-2-methyl-phenyl]propanoic acid (1)

Following the General Procedure of Description 8, 3-amino-3-[5-[bis(2-chloroethyl)amino]-2-methyl-phenyl]propanoic acid (1) was prepared through hydrolytic deprotection of methyl 3-benzyloxycarbonylamino-3-[5-[bis(2-chloroethyl)amino]-2-methyl-phenyl]propanoate (1g) (2.9 g, 6.2 mmol) in a mixture of concentrated hydrochloric acid (HCl) (20 mL) and 1,4-dioxane (20 mL) at about 100° C. (oil bath) in 48 hours. The residue was purified by preparative HPLC, immediately frozen after collection, followed by primary lyophilization to afford 728 mg (33% yield) of the target compound (1) as a colorless solid. ¹H NMR (300 MHz, DMSO-d⁶): δ 6.98 (d, J=8.4 Hz, 1H), 6.85 (d, J=2.4 Hz, 1H), 6.56 (dd, J=8.4, 2.4 Hz, 1H), 4.36 (dd, J=9.9, 4.5 Hz, 1H), 3.56-3.53 (br. m, 8H), 2.48-2.44 (m, 2H) ppm. LC/MS: Rₜ=1.226 min; ESI (pos.) m/z=319.2 (M+H⁺)⁺, ESI (neg.) m/z=316.9 (M−H⁺)⁻, 635.1 (2M−H⁺)⁻. LC/UV: Rₜ=6.723 min, 99.3% AUC at λ=254 nm. Various batches of mono- or dihydrochloride salts of (1) were prepared by primary lyophilization of solutions of (5) in aqueous acetonitrile (MeCN) containing either 1.0 eq. of 1.0 N hydrochloric acid (HCl) or an excess of 1.0 N or higher concentrated hydrochloric acid (HCl).

### Example 2

### 3-Amino-3-[4-[bis(2-chloroethyl)amino]-2-methyl-phenyl]propanoic acid (2)

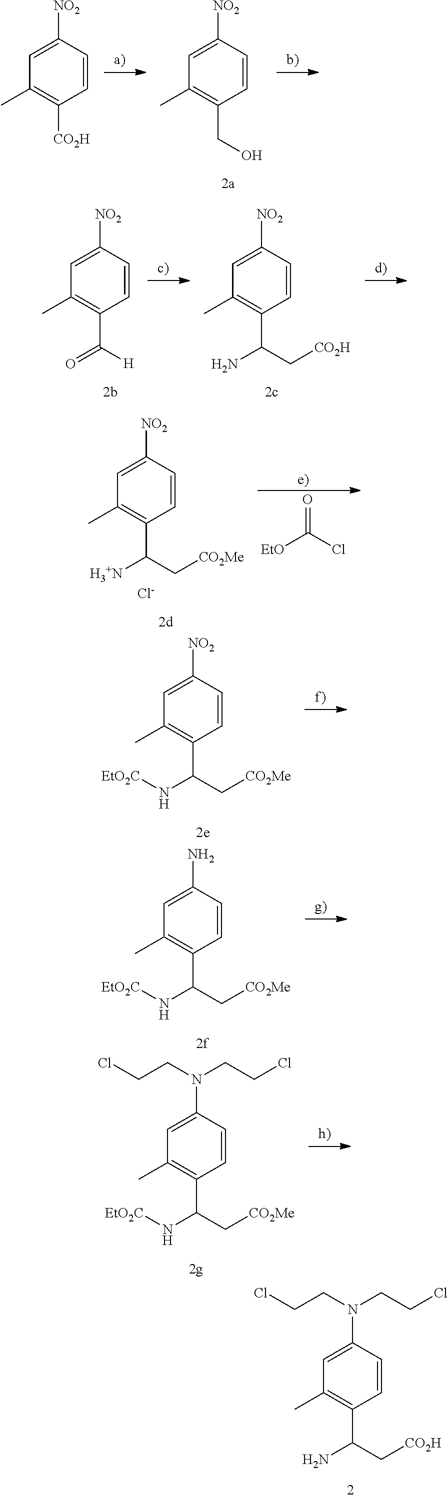

### Step A: (2-Methyl-4-nitro-phenyl)methanol (2a)

Following the General Procedure of 1, 2-methyl-4-nitro-phenyl)methanol (2a) was prepared from commercial 2-methyl-4-nitro benzoic acid (5.0 g, 27.6 mmol) with borane dimethylsulfide complex (2.0 M BH₃.SMe₂ in THF) (27.6 mL, 55.2 mmol) in anhydrous tetrahydrofuran (100 mL) to yield 4.62 g (quantitative yield) of the target compound (7a) as a pale yellow solid which was of sufficient purity to be used directly in the next step without further isolation and purification. R_{f}: ˜0.50 (EtOAc/Hxn=1:1 v/v). ¹H NMR (300 MHz, CDCl₃): δ 8.07 (dd, J=8.4, 2.1 Hz, 1H), 8.02 (d, J=2.1 Hz, 1H), 7.62 (d, J=8.1 Hz, 1H), 4.79 (s, 2H), 2.38 (s, 3H), 1.87 (br. s, 1H) ppm. The spectroscopic data correspond to the data provided in the literature. The compound is also commercially available.

### Step B: 2-Methyl-4-nitro-benzaldehyde (2b)

Following the General Procedure of Description 2 (Variant B), 2-methyl-4-nitro-benzaldehyde (2b) was prepared from 2-methyl-4-nitro-phenyl)methanol (1a) (8.4 g, 50.3 mmol) in the presence of manganese dioxide (MnO₂) (48.1 g, 553 mmol). Work-up afforded 7.5 g (90% yield) of the target compound (7b) as a yellow solid. The material was of sufficient purity to be used directly in the next step without further isolation and purification. R_{f}: ˜0.58 (EtOAc/Hxn=1:2 v/v). ¹H NMR (300 MHz, CDCl₃): δ 10.39 (s, 1H), 8.20 (dd, J=8.4, 2.1 Hz, 1H), 8.14 (br. s, 1H), 7.98 (d, J=8.1 Hz, 1H), 2.79 (s, 3H) ppm. The spectroscopic data correspond to the data provided in the literature. The compound is also commercially available.

### Step C: 3-Amino-3-(2-methyl-4-nitro-phenyl)propanoic acid (2c)

Following the General Procedure of Description 3, 3-amino-3-(2-methyl-4-nitro-phenyl)propanoic acid (2c) was prepared from 2-methyl-4-nitro-benzaldehyde (2b) (800 mg, 5.0 mmol), malonic acid (520 mg, 5.0 mmol), and ammonium acetate (NH₄OAc) (578 mg, 7.5 mmol) in ethanol (EtOH) (10 mL) at reflux for 48 h (oil bath). The reaction was followed by LC/MS to completion. Filtrative work-up afforded 510 mg (45% yield) of the target compound (1c) as a near colorless solid which was of sufficient purity to be used directly in the next step without further purification and isolation. ¹H NMR (300 MHz, D₂O): δ 8.01-7.97 (m, 2H), 7.46 (d, J=8.4 Hz, 1H), 4.83 (t, J=7.2 Hz, 1H), 2.70-2.65 (m, 2H), 2.33 (s, 3H) ppm. LC/MS: Rₜ=1.274 min; ESI (pos.) m/z=225.1 (M+H⁺)⁺.

### Step D: Methyl 3-amino-3-(2-methyl-4-nitro-phenyl)propanoate Hydrochloride (2d)

Following the General Procedure of Description 4, methyl 3-amino-3-(2-methyl-4-nitro-phenyl)propanoate hydrochloride (2d) was prepared in a suspension in anhydrous methanol (MeOH) (10 mL) from 3-amino-3-(2-methyl-4-nitro-phenyl)propanoic acid (2c) (510 mg, 2.27 mmol) with neat thionyl chloride (SOCl₂) (2.0 mL, 3.28 g, 27.5 mmol). Evaporative work-up afforded 2.73 g (about quantitative yield) of the target compound (1d) as a colorless solid, which was of sufficient purity to be used directly in the next step without further purification and isolation. LC/MS: Rₜ=0.508 min; ESI (pos.) m/z=239.1 (M+H⁺)⁺.

### Step E: Methyl 3-(ethoxycarbonylamino)-3-(2-methyl-4-nitro-phenyl)propanoate (2e)

Following the General Procedure of Description 5, methyl 3-(ethoxycarbonylamino)-3-(2-methyl-4-nitro-phenyl)propanoate (2e) was prepared from crude methyl 3-amino-3-(2-methyl-4-nitro-phenyl)propanoate hydrochloride (2e) (624 mg, 2.27 mmol), ethyl chloroformate (EtOCOCL) (327 μL, 371 mg 3.42 mmol), and diisopropylethylamine (DIPEA, Hünigs-base) (1.12 mL, 885 mg, 6.84 mmol) in anhydrous dichloromethane (DCM) (10 mL). Silica gel column chromatography afforded 701 mg (about quantitative yield) of the target compound (2e) as a colorless solid. R_{f}=0.42 (EtOAc/Hxn=1:1 v/v).

### Step F: Methyl 3-(4-amino-2-methyl-phenyl)-3-(ethoxycarbonylamino)propanoate (2f)

Following the General Procedure of Description 6 (Variant B), methyl 3-(4-amino-2-methyl-phenyl)-3-(ethoxycarbonylamino)propanoate (2f) is prepared from methyl 3-(ethoxycarbonylamino)-3-(2-methyl-4-nitro-phenyl)propanoate (2e) (701 mg, 2.26 mmol) through hydrogenation (about 15 psi; H₂-filled balloon) in the presence 10 wt-% Pd/C containing 50-wt-% water (˜70 mg) and at room temperature for about 12 hours to afford 632 mg (about quantitative yield) of the target compound (2f) as a brownish oil, which was of sufficient purity to be used in the next step without additional purification and isolation. LC/MS: Rₜ=0.533 min; ESI (pos.) m/z=303.1 (M+H⁺)⁺.

### Step G: Methyl 3-[4-[bis(2-chloroethyl)amino]-2-methyl-phenyl]-3-(ethoxycarbonylamino)-propanoate (2g)

Following the General Procedure for of Description 7 (Variant A), methyl 3-[4-[bis(2-chloroethyl)amino]-2-methyl-phenyl]-3-(ethoxycarbonylamino)-propanoate (2g) was prepared from methyl 3-(4-amino-2-methyl-phenyl)-3-(ethoxycarbonylamino)propanoate (2f) (632 mg, 2.26 mmol), 2-chloroacetaldehyde (˜50 wt-% in water, ˜7.87 M) (1.44 mL, 11.3 mmol), and sodium cyanoborohydride (NaBH₃CN) (598 mg of 95% purity=568 g, 9.04 mmol) in a mixture of methanol (MeOH) (20 mL) and trifluoroacetic acid (TFA) (10 mL). Purification by silica gel column chromatography with an ethyl acetate (EtOAc)/hexane mixture (EtOAc/hexane=1:1, v/v) afforded 714 mg (78% yield) of the title compound (2g) as a colorless solid. R_{f}=0.54 (EtOAc/Hxn=1:2 v/v, ninhydrin negative). ¹H NMR (300 MHz, CDCl₃): δ 7.11 (d, J=8.4 Hz, 1H), 6.49 (dd, J=8.7, 2.7 Hz, 1H), 6.44 (d, J=2.4 Hz, 1H), 5.36-5.22 (m, 2H), 4.08 (q, J=7.2 Hz, 2H), 3.76-3.54 (m, 11H), 2.90-2.70 (m, 2H), 2.39 (s, 3H), 1.21 (t, J=7.2 Hz, 3H) ppm. LC/MS: Rₜ=2.174 min; ESI (pos.) m/z=405.1 (M+H⁺)⁺.

### Step H: 3-Amino-3-[4-[bis(2-chloroethyl)amino]-2-methyl-phenyl]propanoic acid (2)

Following the General Procedure of Description 8, 3-amino-3-[4-[bis(2-chloroethyl)amino]-2-methyl-phenyl]propanoic acid (2) was prepared through hydrolytic deprotection of methyl 3-[4-[bis(2-chloroethyl)amino]-2-methyl-phenyl]-3-(ethoxycarbonylamino)-propanoate (2g) (150 mg, 0.37 mmol) in concentrated hydrochloric acid (HCl) (5 mL) at about 100° C. (oil bath) in 48 h. The residue was partially purified by preparative HPLC, immediately frozen after collection, followed by primary lyophilization to afford 40 mg of the target compound (1) as a colorless solid. ¹H NMR (300 MHz, DMSO-d⁶): δ 7.30 (d, J=6.3 Hz, 1H), 6.63 (dd, J=6.6, 2.1 Hz, 1H), 6.56 (d, J=1.8 Hz, 1H), 4.55 (t, J=5.7 Hz, 1H), 3.76-3.62 (br. m, 8H), 2.84 (dd, J=12.3, 5.1 Hz, 1H), 2.71 (dd, J=12.0, 5.7 Hz, 1H), 2.29 (s, 3H) ppm. LC/MS: Rₜ=1.094 min; ESI (neg.) m/z=317.0 (M−H⁺)⁻. LC/UV: Rₜ=7.393 min, 98.6% AUC at λ=254 nm.

### Example 3

### 3-Amino-4-[5-[bis(2-chloroethyl)amino]-2-methyl-phenyl]butanoic acid (3)

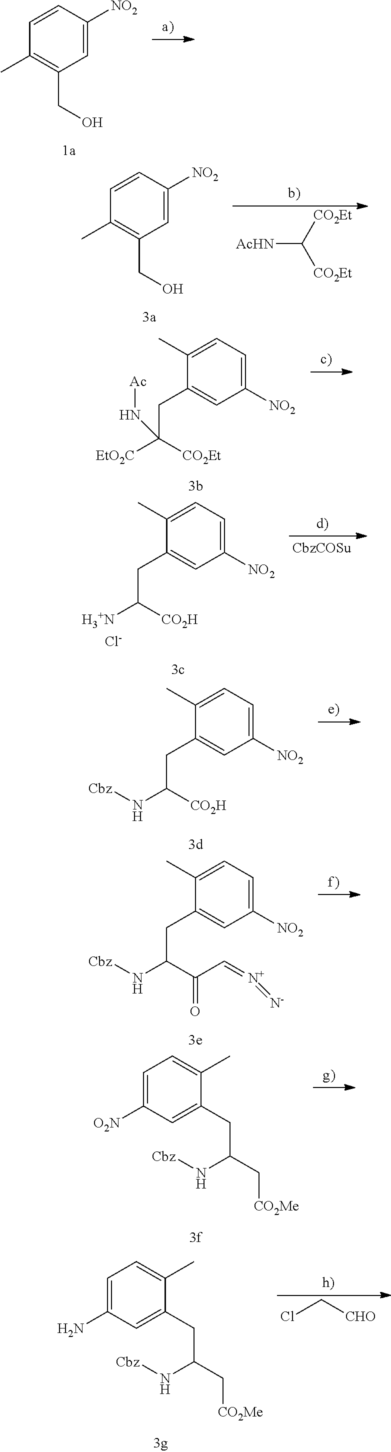

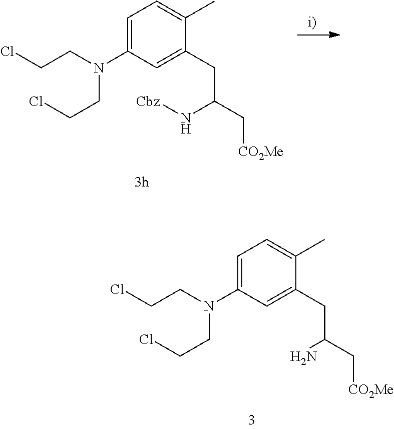 [SMILES: COC(=O)CC(Cc1cc(N(CCCl)CCCl)ccc1C)NC(=O)OCc1ccccc1 ; COC(=O)CC(N)Cc1cc(N(CCCl)CCCl)ccc1C]

### Step A: 2-(Bromomethyl)-1-methyl-4-nitro-benzene (3a)

Following the General Procedure of Description 10, 2-(bromomethyl)-1-methyl-4-nitro-benzene (3a) was prepared through bromination of (2-methyl-5-nitro-phenyl)methanol (1a) (11.0 g, 65.8 mmol) (prepared as described in Example 1) dissolved in dichloromethane (DCM) (110 mL) with a solution of phosphorus tribromide (PBr₃) in (1.0 M PBr₃ in DCM) (65.8 mL). Aqueous work-up yielded 11.3 g (75% yield) of a light yellow solid which was of sufficient purity to be used directly and without further isolation and purification in the next step. R_{f}=0.56 (EtOAc/Hxn=1:5 v/v). ¹H NMR (300 MHz, CDCl₃): δ 8.19 (d, J=2.4 Hz, 1H), 8.07 (dd, J=8.4, 2.7 Hz, 1H), 7.36 (d, J=8.7 Hz, 1H), 4.53 (s, 2H), 2.52 (s, 2H) ppm. The spectroscopic data correspond to the data provided in the literature. The compound is also commercially available.

### Step B: Diethyl 2-acetamido-2-[(2-methyl-5-nitro-phenyl)methyl]propanedioate (3b)

Adapting a literature protocol (Haudegond, et al., J. Org. Chem., 1979, 44(17), 3063-3065), an ethanolic solution of sodium ethanolate (NaOEt) (35.6 mmol) was freshly prepared from elemental sodium (Na) (819 mg, 35.6 mmol) in anhydrous ethanol (EtOH) (80 mL) under an atmosphere of nitrogen at room temperature. When the H₂-evolution was ceased, commercial diethyl 2-acetamidopropanedioate (7.9 g, 36.4 mmol) was added in small portions. The reaction mixture was heated at about 75° C. (oil bath) for about 30 min before 2-(bromomethyl)-1-methyl-4-nitro-benzene (3a) (8.2 g, 35.6 mmol) was added, and the reaction mixture was heated at reflux (oil bath) for about 10 h. The reaction was followed by LC/MS to completion. The solid was collected by filtration using a Büchner-funnel and the residue was washed successively with EtOH (2×) and ethyl acetate (EtOAc) (1×), and dried under reduced pressure to afford 8.4 g (64% yield) of the target compound diethyl 2-acetamido-2-[(2-methyl-5-nitro-phenyl)methyl]propanedioate (3b) as a colorless solid. ¹H NMR (300 MHz, DMSO-d⁶): δ 8.29 (s, 1H), 8.00 (dd, J=8.1, 2.4 Hz, 1H), 7.72 (d, J=2.4 Hz, 1H), 7.45 (d, J=8.7 Hz, 1H), 4.15 (q, J=7.2 Hz, 4H), 3.58 (s, 2H), 2.26 (s, 3H), 1.90 (s, 3H), 1.17 (t, J=7.2 Hz, 6H) ppm. LC/MS: Rₜ=1.818 min; ESI (pos.) m/z=367.1 (M+H⁺)⁺, 755.3 (2M+Na⁺)⁺.

### Step C: 2-Amino-3-(2-methyl-5-nitro-phenyl)propanoic acid hydrochloride (3c)

Following the General Procedure of Description 8, 2-amino-3-(2-methyl-5-nitro-phenyl)propanoic acid hydrochloride (3c) was prepared by acid hydrolysis of diethyl 2-acetamido-2-[(2-methyl-5-nitro-phenyl)methyl]propanedioate (3b) (8.4 g, 22.9 mmol) with concentrated (˜37 wt-%) hydrochloric acid (HCl) (150 mL). The suspension was heated at reflux (oil bath) for about 6 h. The reaction was followed by LC/MS to completion. The cooled clear solution was evaporated under reduced pressure using a rotary evaporator to yield 6.7 g (about quantitative yield) of the target compound (3c) as a colorless solid. ¹H NMR (300 MHz, DMSO-d⁶): δ 8.58 (br. s, 3H), 8.12 (d, J=2.1 Hz, 1H), 8.03 (dd, J=8.4, 2.4 Hz, 1H), 7.47 (d, J=8.7 Hz, 1H), 4.20-4.10 (m, 1H), 3.25 (d, J=7.2 Hz, 2H), 2.42 (s, 3H) ppm. LC/MS: Rₜ=0.705 min; ESI (pos.) m/z=225.1 (M+H⁺)⁺, 449.1 (2M+H⁺)⁺; ESI (neg.) m/z=223.0 (M−H⁺)⁻, 447.1 (2M−H⁺)⁻.

### Step D: 2-Benzyloxycarbonylamino-3-(2-methyl-5-nitro-phenyl)propanoic acid (3d)

Adapting a literature protocol, 2-benzyloxycarbonylamino-3-(2-methyl-5-nitro-phenyl)propanoic acid (3d) was prepared from 2-amino-3-(2-methyl-5-nitro-phenyl)propanoic acid hydrochloride (3c) (6.7 g, 25.7 mmol) in 1,4-dioxane (50 mL) and a 10 wt-% aq. solution of sodium hydroxide (NaOH) (˜3.75 M, 13.7 mL, 51.4 mmol) at about 0° C. (ice bath). Water (32 mL) was added followed by solid sodium hydrogencarbonate (NaHCO₃) (2.15 g, 25.7 mmol), and commercial benzyl (2,5-dioxopyrrolidin-1-yl) carbonate (CbzOSu) (6.4 g, 25.7 mmol). The reaction mixture was stirred overnight at room temperature. The volatiles were removed under reduced pressure using a rotary evaporator. Acid work up at a pH of about 3 and tritruation of the crude product with ethyl acetate (EtOAc) and hexane (Hxn) (EtOAc/Hxn=3:7) at about 50° C. (oil bath), the solid was collected by filtration (Büchner-funnel) to afford 6.1 g (65% yield) of the target compound (3d) as a colorless solid. ¹H NMR (300 MHz, CDCl₃): δ 8.02-7.98 (m, 2H), 7.40-7.21 (m, 6H), 5.33 (d, J=8.4 Hz, 1H), 5.06 (d, J=12.0 Hz, 1H), 5.03 (d, J=12.0 Hz, 1H), 4.74-4.70 (m, 1H), 3.57 (dd, J=14.7, 5.4 Hz, 1H), 3.08 (dd, J=14.4, 7.8 Hz, 1H), 2.45 (s, 3H) ppm. LC/MS: Rₜ=1.812 min; ESI (neg.) m/z=357.1 (M−H⁺)⁻, 715.1 (2M−H⁺)⁻.

### Step E: Benzyl N-[3-diazo-1-[(2-methyl-5-nitro-phenyl)methyl]-2-oxo-propyl]carbamate (3e)

Following the general procedure of Description 11 (Part A), a solution of diazomethane (CH₂N₂) in diethyl ether (Et₂O) was freshly prepared prior to use in an Aldrich Diazald® apparatus from commercial N-methyl-N-nitrosotoluene-4-sulphonamide (Diazald®) (15 g, 70.0 mmol), potassium hydroxide (KOH) (15 g, 267 mmol) in a mixture of Et₂O (25 mL), water (30 mL), and 2-(2-ethoxyethoxy)ethanol (50 mL) at about 65° C. (oil bath). The etheral distillate was trapped in Et2O (150 mL) in Et₂O (150 mL).

Following the general procedure of Description 11 (Part B), the mixed anhydride of (3d) is prepared from 2-benzyloxycarbonylamino-3-(2-methyl-5-nitro-phenyl)propanoic acid (3d) (3.0 g, 8.38 mmol), N-methylmorpholine (NMM) (1.20 mL, 1.1 g, 10.9 mmol), neat isobutyl chloroformate (1.34 mL, 1.4 g, 10.1 mmol) at about −20° C. (dry ice/acetone bath) under a nitrogen atmosphere. After the 2 hours −20° C., an excess of (˜6 equivalents) of the freshly prepared ethereal solution of diazomethane was added (˜100 mL). Aqueous work and purification by silica gel column chromatography (EtOAc/Hxn=2:3 v/v) afforded 2.5 g (85% yield of the target compound benzyl N-[3-diazo-1-[(2-methyl-5-nitro-phenyl)methyl]-2-oxo-propyl]carbamate (3e) as a light yellow solid. R_{f}=0.25 (EtOAc/Hxn=2:3 v/v). ¹H NMR (300 MHz, CDCl₃): δ 8.02-7.98 (m, 2H), 7.40-7.24 (m, 6H), 5.46 (d, J=8.4 Hz, 1H), 5.29 (s, 1H), 5.05 (d, J=12.0 Hz, 1H), 5.02 (d, J=12.6 Hz, 1H), 4.52-4.46 (m, 1H), 3.23 (dd, J=14.1, 6.6 Hz, 1H), 2.97 (dd, J=13.8, 7.8 Hz, 1H), 2.44 (s, 3H) ppm.

### Step F: Methyl 3-benzyloxycarbonylamino-4-(2-methyl-5-nitro-phenyl)butanoate (3f)

Following the general procedure of Description 11 (Part C), methyl 3-benzyloxycarbonylamino-4-(2-methyl-5-nitro-phenyl)butanoate (3f) is prepared from benzyl N-[3-diazo-1-[(2-methyl-5-nitro-phenyl)methyl]-2-oxo-propyl]carbamate (3e) (2.5 g, 6.55 mmol) and a mixture of silver benzoate (AgBz) (0.75 g, 3.3 mmol) in THF (5 mL) and triethylamine (TEA) (1.93 mL, 1.4 g, 13.1 mmol) in a mixture of degassed anhydrous methanol (MeOH) (2.1 mL) and degassed anhydrous tetrahydrofuran (THF) (15 mL) at room temperature and under a nitrogen atmosphere. Evaporative work-up followed by silica gel column chromatography purification (EtOAc/Hxn=2:3, v/v) afforded 2.1 g (82% yield) of the target compound (3f) as a colorless solid. R_{f}=0.33 (EtOAc/Hxn=2:3 v/v). ¹H NMR (300 MHz, CDCl₃): δ 8.00-87.95 (m, 2H), 7.38-7.24 (m, 6H), 5.48 (d, J=9.3 Hz, 1H), 5.02 (s, 2H), 4.30-4.21 (m, 1H), 3.72 (s, 3H), 3.06-3.01 (m, 1H), 2.97-2.54 (m, 1H), 2.64-2.50 (m, 2H), 2.48 (s, 3H) ppm.

### Step G: Methyl 4-(5-amino-2-methyl-phenyl)-3-benzyloxycarbonylamino-butanoate (3g)

Following the General Procedure for of Description 6 (Variant A), methyl 4-(5-amino-2-methyl-phenyl)-3-benzyloxycarbonylamino-butanoate (3g) was prepared from methyl 3-benzyloxycarbonylamino-4-(2-methyl-5-nitro-phenyl)butanoate (3f) (2.1 g, 5.4 mmol), iron powder (Fe) (2.7 g, 48.9 mmol), and calcium chloride dihydrate (CaCl₂.2H₂O) (0.35 g, 2.4 mmol) in a mixture of methanol (MeOH)/water (41 mL:7.5 mL, v/v). The reaction mixture was heated at reflux for about 2 hours (oil bath). Removal of the iron residues by filtration and compound isolation procedures yielded 1.9 g (˜quantitative yield) of the target compound (3g) as a light yellow solid which was of sufficient purity to be used directly in the nest step without further isolation and purification. ¹H NMR (300 MHz, DMSO-d⁶): δ 7.38-7.24 (m, 5H), 6.75 (d, J=7.5 Hz, 1H), 6.36-6.30 (m, 2H), 4.97 (s, 2H), 4.72 (br. s, 2H), 4.15-3.85 (m, 1H), 3.50 (s, 3H), 3.18-3.14 (m, 2H), 2.68-2.64 (m, 1H), 2.50-2.35 (m, 1H, superimposed with solvent), 2.09 (s, 3H) ppm. LC/MS: Rₜ=1.158 min; ESI (pos.) m/z=379.1 (M+H⁺)⁺, 713.4 (2M+H⁺)⁺.

### Step H: Methyl 3-benzyloxycarbonylamino-4-[5-[bis(2-chloroethyl)amino]-2-methyl-phenyl]butanoate (3h)

Following the General Procedure for of Description 7 (Variant A), methyl 3-benzyloxycarbonylamino-4-[5-[bis(2-chloroethyl)amino]-2-methyl-phenyl]butanoate (3i) was prepared from methyl 4-(5-amino-2-methyl-phenyl)-3-benzyloxycarbonylamino-butanoate (3h) (1.9 g, 5.3 mmol), 2-chloroacetaldehyde (˜50 wt-% in water, ˜7.87 M) (3.4 mL, 26.5 mmol), and sodium cyanoborohydride (NaBH₃CN) (1.41 g of 95% purity=1.34 g, 21.3 mmol) in a mixture of methanol (MeOH) (34 mL) and trifluoroacetic acid (TFA) (17 mL). Purification by silica gel column chromatography with an ethyl acetate (EtOAc)/hexane mixture (EtOAc/hexane=1:2, v/v) afforded 2.16 g (85% yield) of the title compound (3h) as a colorless solid. R_{f}=0.37 (EtOAc/hexane=1:2, v/v, ninhydrin negative). ¹H NMR (300 MHz, CDCl₃): δ 7.36-7.24 (m, 5H), 7.03 (d, J=8.4 Hz, 1H), 6.50 (dd, J=8.4, 2.7 Hz, 1H), 6.44-6.41 (br. m, 1H), 5.50 (d, J=8.7 Hz, 1H), 5.08 (s, 2H), 4.26-418 (br. m, 1H), 3.70 (s, 3H), 3.70-3.54 (m, 8H), 2.96 (dd, J=13.8, 6.3 Hz, 1H), 2.76 (dd, J=13.8, 8.4 Hz, 1H), 2.55 (br. d, J=4.8 Hz, 2H), 2.26 (s, 3H) ppm. LC/MS: Rₜ=2.526 min; ESI (pos.) m/z=503.1 (M+H⁺)⁺. LC/UV: Rₜ=6.552 min, 100.0% AUC at λ=254 nm.

### Step I: 3-Amino-4-[5-[bis(2-chloroethyl)amino]-2-methyl-phenyl]butanoic acid (3)

Following the General Procedure for of Description 8, 3-amino-4-[5-[bis(2-chloroethyl)amino]-2-methyl-phenyl]butanoic acid (3) was prepared through acidic hydrolysis of methyl 3-benzyloxycarbonylamino-4-[5-[bis(2-chloroethyl)amino]-2-methyl-phenyl]butanoate (3i) (2.16 g, 4.15 mmol) in a mixture of concentrated hydrochloric acid (HCl) (30 mL) and 1,4-dioxane (30 mL). The residue was purified by preparative HPLC, immediately frozen after collection, followed by primary lyophilization to afford 722 mg of the target compound (3) as a colorless powder. ¹H NMR (300 MHz, DMSO-d⁶): δ 7.30 (d, J=9.0 Hz, 1H), 6.56-6.50 (m, 2H), 3.76-3.60 (br. m, 10H), 3.65-3.36 (br. m, 1H), 2.75 (dd, J=13.5, 6.6 Hz, 1H), 2.65 (dd, J=13.2, 7.8 Hz, 1H), 2.13 (s, 3H), 2.06 (d, J=3.9 Hz, 1H), 2.00 (dd, J=16.2, 9.3 Hz, 1H) ppm. LC/MS: Rₜ=1.094 min; ESI (pos.) m/z=333.1 (M+H⁺)⁺; ESI (neg.) m/z=330.9.0 (M−H⁺)⁻. LC/UV: Rₜ=7.134 min, 95.5% AUC at λ=254 nm. The analytical data correspond to the analytical data of the (S)-isomer (5) and the (R)-isomer (6). Various batches of mono- or dihydrochloride salts of (3) can be prepared by primary lyophilization of solutions of (3) in aqueous acetonitrile (MeCN) containing either 1.0 eq. of 1.0 N hydrochloric acid (HCl) or an excess of 1.0 N or higher concentrated hydrochloric acid (HCl).

### Example 4

### 3-Amino-4-[4-[bis(2-chloroethyl)amino]-2-methyl-phenyl]butanoic acid (4)

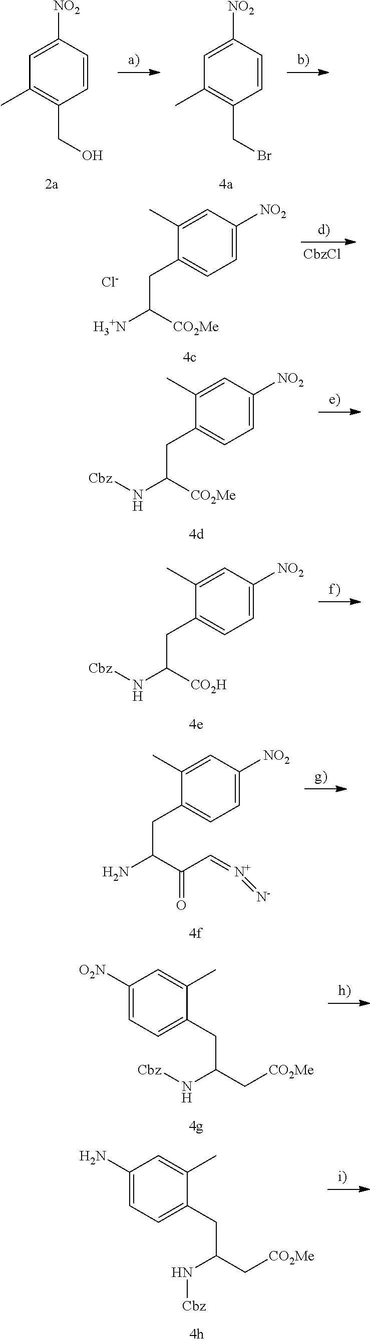

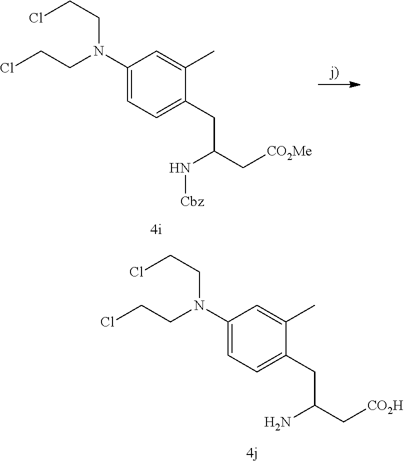 [SMILES: COC(=O)CC(Cc1ccc(N(CCCl)CCCl)cc1C)NC(=O)OCc1ccccc1 ; Cc1cc(N(CCCl)CCCl)ccc1CC(N)CC(=O)O]

### Step A: 1-(Bromomethyl)-4-nitro-benzene (4a)

Following the General Procedure of Description 10, 1-(bromomethyl)-4-nitro-benzene (4a) was prepared through bromination of (2-methyl-4-nitro-phenyl)methanol (2a) (18.0 g, 108 mmol) (prepared as described in Example 2) dissolved in dichloromethane (DCM) (200 mL) with a solution of phosphorus tribromide (PBr₃) in (1.0 M PBr₃ in DCM) (108 mL). Aqueous work-up yielded 16.0 g (64% yield) of a light yellow solid which was of sufficient purity to be used directly and without further isolation and purification in the next step. R_{f}=0.51 (EtOAc/Hxn=1:5 v/v). The spectroscopic data correspond to the data provided in the literature.

### Step C: Methyl 2-amino-3-(2-methyl-4-nitro-phenyl)propanoate Hydrochloride (4c)

Adapting literature protocols (, methyl 2-amino-3-(2-methyl-4-nitro-phenyl)propanoate Hydrochloride (4c) was prepared through alkylation commercial methyl [(phenylmethylidene)amino]acetate (1.84 g, 10.4 mmol), with 1-(bromomethyl)-4-nitro-benzene (4b) (2.86 g, 12.5 mmol), potassium carbonate (K₂CO₃) (4.31 g, 31.2 mmol), benzyltriethylammonium chloride (BTEAC) (237 mg, 1.04 mmol) in acetonitrile (MeCN) (30 mL). The reaction mixture was stirred for about 6 hours at room temperature, filtered, and concentrated under reduced pressure using a rotary evaporator. The residue was diluted with diethyl ether (Et₂O) and the organic layer was washed with brine. The phases were separated and the organic layer was concentrated to a total volume of about 20 mL. 1.0 M Hydrochloric acid (HCl) (50 mL) was added, and the reaction mixture was kept overnight at room temperature. The reaction mixture was further diluted with diethyl ether (Et₂O) and the phases were separated. The aqueous phase was concentrated under reduced pressure using a rotary evaporator.

Following the General Synthesis of Description 4, the crude material was diluted with anhydrous methanol (MeOH) (20 mL) and treated with excess thionyl chloride (SOCl₂) at about 0° C. (ice bath). The reaction mixture was subsequently heated to about 80° C. (oil bath) for about 1 h before solvents and volatiles were removed under reduced pressure using a rotary evaporator to afford 2.18 g (76% yield) of the target compound (4c) as a colorless solid. LC/MS: Rₜ=0.687 min; ESI (pos.) m/z=239.1 (M+H⁺)⁺.

### Step D: Methyl 2-benzyloxycarbonylamino-3-(2-methyl-4-nitro-phenyl)propanoate (4d)

Following the General Procedure of Description 5, methyl 2-benzyloxycarbonylamino-3-(2-methyl-4-nitro-phenyl)propanoate (4d) was prepared from methyl 2-amino-3-(2-methyl-4-nitro-phenyl)propanoate hydrochloride (4c) (2.18 g, 7.94 mmol), benzyl chloroformate (CbzCl, ZCl) (1.65 mL, 1.97 g, 11.9 mmol), and diisopropylethylamine (DIPEA, Hünigs-base) (3.92 mL, 3.07 g, 23.7 mmol) in dichloromethane (DCM) (50.0 mL). Aqueous work-up and purification by silica gel colunchromatography (EtOAc/Hxn=1:2 v/v) afforded 1.94 g (40% yield) of the target compound (4d) as a colorless solid. R_{f}=0.44 (EtOAc/Hxn=1:2 v/v). ¹H NMR (400 MHz, CDCl₃): δ 8.06-8.00 (m, 1H), 7.94-7.86 (m, 1H), 7.40-7.20 (m, 6H), 5.36 (d, 1H), 5.06 (d, 1H), 5.00 (d, 1H), 4.70-4.60 (m, 1H), 3.68 (s, 3H), 3.26 (dd, 1H), 3.04 (dd, 1H), 2.40 (s, 3H) ppm. LC/MS: Rₜ=2.085 min; ESI (pos.) m/z=373.3 (M+H⁺)⁺; ESI (neg.) m/z=371.1 (M−H⁺)⁻.

### Step E: 2-Benzyloxycarbonylamino-3-(2-methyl-4-nitro-phenyl)propanoic acid (4e)

Adapting a literature protocol (Dayal, et al., Steroids, 1990, 55(5), 233-237), a reaction mixture of methyl 2-benzyloxycarbonylamino-3-(2-methyl-4-nitro-phenyl)propanoate (4d) (1.94 g, 5.20 mmol) and commercial lithium hydroxide monohydrate (LiOH.H₂O) (436 mg, 10.4 mmol) in a mixture of tetrahydrofuran (THF)/methanol (MeOH)/water (20:10:10 mL v/v/v) was stirred at room temperature. The reaction was followed by TLC and LC/MS to completion. Acidic aqueous work-up at about pH 4 and subsequent crystallization from ethyl acetate (EtOAc) furnished 900 mg (48% yield) of the target compound (4e) as a colorless solid. ¹H NMR (400 MHz, CDCl₃): δ 7.96-7.92 (m, 1H), 7.90-7.80 (m, 1H), 7.36-7.18 (m, 6H), 5.62 (d, 1H), 5.00 (d, 1H), 4.93 (d, 1H), 4.60-4.50 (m, 1H), 3.26 (dd, 1H), 2.98 (dd, 1H), 2.38 (s, 3H) ppm. LC/MS: Rₜ=1.818 min; ESI (pos.) m/z=359.1 (M+H⁺)⁺; ESI (neg.) m/z=357.0 (M−H⁺)⁻.

### Step F: Benzyl N-[3-diazo-1-[(2-methyl-4-nitro-cyclohexa-2,4-dien-1-yl)methyl]-2-oxo-propyl]carbamate (4f)

Following the General Procedure of Description 12 (Parts A-B), benzyl N-[3-diazo-1-[(2-methyl-4-nitro-cyclohexa-2,4-dien-1-yl)methyl]-2-oxo-propyl]carbamate (4f) was prepared from 2-benzyloxycarbonylamino-3-(2-methyl-4-nitro-phenyl)propanoic acid (4e) (700 mg, 1.97 mmol), N-methylmorpholine (NMM) (433 μL, 398 mg, 3.94 mmol), isobutyl chloroformate (515 μL, 538 mg, 3.94 mmol) in anhydrous tetrahydrofuran (THF) (10 mL) and about 16 mmol of freshly prepared diazomethane in Et₂O. Silica gel column chromatography (EtOAc/Hxn=1:2 v/v) afforded 350 mg (46% yield) of the target compound (4f) as a colorless solid. R_{f}=0.24 (EtOAc/Hxn=1:2, v/v). ¹H NMR (400 MHz, CDCl₃): δ 8.02-7.98 (m, 1H), 7.96-7.88 (m, 1H), 7.38-7.20 (m, 6H), 5.40 (d, 1H), 5.20 (s, 1H), 5.08 (d, 1H), 5.02 (d, 1H), 4.50-4.40 (m, 1H), 3.18 (dd, 1H), 2.96 (dd, 1H), 2.42 (s, 3H) ppm. LC/MS: Rₜ=1.991 min; ESI (pos.) m/z=405.0 (M+Na⁺)⁺.

### Step G: Methyl 3-benzyloxycarbonylamino-4-(2-methyl-4-nitro-phenyl)butanoate (4g)

Following the General Procedure of Description 12 (Part C), methyl 3-benzyloxycarbonylamino-4-(2-methyl-4-nitro-phenyl)butanoate (4g) was prepared from benzyl N-[3-diazo-1-[(2-methyl-4-nitro-cyclohexa-2,4-dien-1-yl)methyl]-2-oxo-propyl]carbamate (4f) (350 mg, 0.916 mmol) in Methanol (MeOH) (10 mL) and silver benzoate (AgBz) (0.75 g, 3.3 mmol) dissolved in triethylamine (TEA) (3.0 mL, 2.29 g, 4.32 mmol). Silica gel column chromatography (EtOAc/Hxn=2:3 v/v) afforded 220 mg (62% yield) of the target compound (4g) as pale yellow solid. ¹H NMR (400 MHz, CDCl₃): δ 8.02-7.98 (m, 1H), 7.92-7.86 (m, 1H), 7.40-7.18 (m, 6H), 5.46 (d, 1H), 5.04-4.96 (m, 2H), 4.28-4.18 (m, 1H), 3.69 (s, 3H), 3.08 (dd, 1H), 2.90 (dd, 1H), 2.60 (dd, 1H), 2.54 (dd, 1H), 2.44 (s, 3H) ppm. LC/MS: Rₜ=2.082 min; ESI (pos.) m/z=387.2 (M+H⁺)⁺; ESI (neg.) m/z=384.9 (M−H⁺)⁻.

### Step H: Methyl 4-(4-amino-2-methyl-phenyl)-3-benzyloxycarbonylamino-butanoate (4h)

Following the General Procedure for of Description 6 (Variant A), methyl 4-(4-amino-2-methyl-phenyl)-3-benzyloxycarbonylamino-butanoate (4h) was prepared from methyl 3-benzyloxycarbonylamino-4-(2-methyl-4-nitro-phenyl)butanoate (4g) (220 mg, 0.570 mmol), iron powder (Fe) (286 mg, 5.13 mmol), and anhydrous calcium chloride (CaCl₂) (28 mg, 0.257 mmol) in 85 vol-% aqueous methanol (MeOH) (20 mL). The reaction mixture was heated at reflux for about 2 hours (oil bath). Removal of the iron residues by filtration and compound isolation procedures yielded 200 mg (about quantitative yield) of the target compound (4h) as a light yellow oil which was of sufficient purity to be used directly in the nest step without further isolation and purification. LC/MS: Rₜ=1.034 min; ESI (pos.) m/z=357.1 (M+H⁺)⁺, 379.1 (M+Na⁺)⁺.

### Step I: Methyl 3-benzyloxycarbonylamino-4-[4-[bis(2-chloroethyl)amino]-2-methyl-phenyl]butanoate (4i)

Following the General Procedure for of Description 7 (Variant A), methyl 3-benzyloxycarbonylamino-4-[4-[bis(2-chloroethyl)amino]-2-methyl-phenyl]butanoate (4i) was prepared from methyl 4-(4-amino-2-methyl-phenyl)-3-benzyloxycarbonylamino-butanoate (4h) (200 mg, 0.561 mmol), 2-chloroacetaldehyde (˜50 wt-% in water, ˜7.87 M) (357 μL, 2.87 mmol), and sodium cyanoborohydride (NaBH₃CN) (148 mg of 95% purity=141 mg, 2.24 mmol) in a mixture of methanol (MeOH) (20 mL) and trifluoroacetic acid (TFA) (10 mL). Aqueous work-up and purification by silica gel column chromatography with an ethyl acetate (EtOAc)/hexane mixture (EtOAc/hexane=2:3, v/v) afforded 260 mg (96% yield) of the title compound (4i) as a colorless oil. R_{f}=0.41 (EtOAc/Hxn=1:2, v/v). ¹H NMR (400 MHz, CDCl₃): δ 7.40-7.28 (m, 5H), 6.92-6.88 (d, 1H), 6.46-6.38 (m, 2H), 5.38 (d, 1H), 5.10-5.00 (m, 2H), 4.10-4.00 (m, 1H), 3.70-3.56 (m, 11H), 2.84 (dd, 1H), 2.70 (dd, 1H), 2.58-2.42 (m, 2H), 2.30 (s, 3H) ppm. LC/MS: Rₜ=2.470 min; ESI (pos.) m/z=481.2 (M+H⁺)⁺.

### Step J: 3-Amino-4-[4-[bis(2-chloroethyl)amino]-2-methyl-phenyl]butanoic acid (4)

Following the General Procedure for of Description 8, 3-amino-4-[4-[bis(2-chloroethyl)amino]-2-methyl-phenyl]butanoic acid (4) was prepared through hydrolysis of methyl 3-benzyloxycarbonylamino-4-[4-[bis(2-chloroethyl)amino]-2-methyl-phenyl]butanoate (4i) (260 mg, 0.54 mmol) in a mixture of concentrated hydrochloric acid (HCl) (1 mL) and 1,4-dioxane (1 mL). Purification by preparative HPLC afforded 82 mg (46% recovery) of the target compound (4) after primary lyophilization as a colorless solid. ¹H NMR (400 MHz, DMSO-d⁶): δ 6.96-6.90 (d, 1H), 6.56-6.46 (m, 2H), 3.70-3.56 (br. m, 9H), 3.30 (br. s, superimposed with water signal, 3H), 2.70 (dd, 1H), 2.56 (dd, 1H), 2.18 (s, 3H), 2.10-1.98 (m, 2H) ppm. LC/MS: Rₜ=1.195 min; ESI (pos.) m/z=333.1 (M+H⁺)⁺; ESI (neg.) m/z=331.0 (M−H⁺)⁻. LC/UV: Rₜ=7.896 min, 96.5% AUC at λ=254 nm. Various batches of mono- or dihydrochloride salts of (4) can be prepared by primary lyophilization of solutions of (4) in aqueous acetonitrile (MeCN) containing either 1.0 eq. of 1.0 N hydrochloric acid (HCl) or an excess of 1.0 N or higher concentrated hydrochloric acid (HCl).

### Example 5

### (3S)-3-Amino-4-[5-[bis(2-chloroethyl)amino]-2-methyl-phenyl]butanoic acid (5)

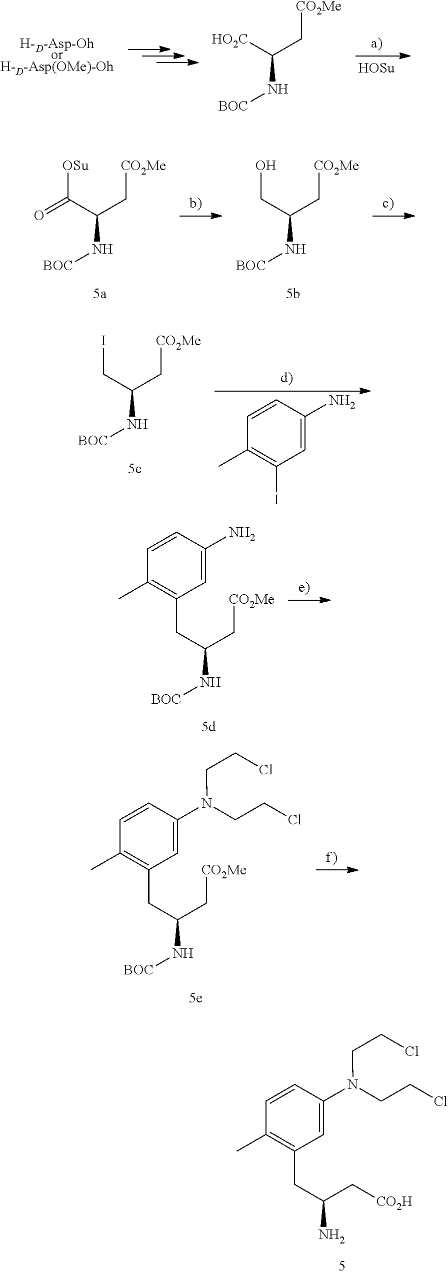

### Step A: O¹-(2,5-Dioxopyrrolidin-1-yl) O⁴-methyl (2R)-2-(tert-butoxycarbonylamino)-butanedioate (5a)

Following the General Procedure of Description 12, O¹-(2,5-Dioxopyrrolidin-1-yl) O⁴-methyl (2R)-2-(tert-butoxycarbonylamino)-butanedioate (5a) was prepared from (2R)-2-(tert-butoxycarbonylamino)-4-methoxy-4-oxo-butanoic acid (9.46 g, 38.3 mmol) (commercially available or prepared from commercial H-D-Asp(OMe)-OH—HCl (10.5 g, 57.3 mmol) (preparable following the General Procedure of Description 4) and Boc₂O (12.5 g, 57.3 mmol) in a mixture of 1,4-dioxane (100 mL) and a freshly prepared 1.0 N aqueous sodium hydroxide (NaOH) solution (126 mL, 126 mmol) (9.46 g (67% yield) (Keller, et al., Org. Synth., 1985, 63, 160)), N-hydroxysuccinimide (1-hydroxypyrrolidine-2,5-dione, HOSu, NHS) (4.69 g, 40.8 mmol), and dicyclohexylcarbodiimide (DCC) (8.02 g, 38.9 mmol in ethyl acetate (EtoAc) (120 mL) at room temperature. Filtration and aqueous work-up furnished 13.2 g (quantitative yield) of the title compound (5a) as a colorless solid which was of sufficient purity to be used directly and without further isolation and purification in the next step. R_{f} ˜0.45 (EtOAc/hexane=1:1, v/v). ¹H NMR (300 MHz, CDCl₃): δ 5.64 (br. d, J=9.3 Hz, 1H), 5.03-4.96 (m, 1H), 3.75 (s, 3H), 3.12 (dd, J=17.4, 4.5 Hz, 1H), 3.12 (dd, J=17.7, 4.5 Hz, 1H), 2.83 (br. s, 4H), 1.45 (s, 9H) ppm. LC/MS: Rₜ=1.463 min; ESI (pos.) m/z=367.15 (M+Na⁺)⁺.

### Step B: Methyl (3R)-3-(tert-butoxycarbonylamino)-4-hydroxy-butanoate (5b)

Following the General Procedure of Description 13, methyl (3R)-3-(tert-butoxycarbonylamino)-4-hydroxy-butanoate (5b) was prepared through reduction of O¹-(2,5-dioxopyrrolidin-1-yl) O⁴-methyl (2R)-2-(tert-butoxycarbonylamino)-butanedioate (5a) (13.2 g, 38.3 mmol) with sodium borohydride (NaBH₄) (2.41 g, 63.7 mmol) in tetrahydrofuran (THF)/water (133 mL/17 mL). Aqueous work-up and purification by silica gel column chromatography with an ethyl acetate (EtOAc)/hexane mixture (EtOAc/hexane=4:3, v/v) furnished 5.73 g (43% yield over 3 steps) of the title compound (5b) as a colorless oil. R_{f} ˜0.34 (EtOAc/hexane=1:1, v/v). ¹H NMR (400 MHz, CDCl₃): δ 5.30 (br. d, 1H), 4.06-3.92 (m, 1H), 3.70-3.68 (m, superimposed, 5H), 2.63 (d, J=5.7 Hz, 2H), 1.43 (s, 9H) ppm. LC/MS: Rₜ=1.027 min; ESI (pos.) m/z=489.25 (2M+Na⁺)⁺. The analytical data correspond with the analytical data for the (S)-enantiomer in the literature (Dexter and Jackson, J. Org. Chem., 1999, 64, 7579-7585).

### Step C: Methyl (3R)-3-(tert-butoxycarbonylamino)-4-iodo-butanoate (5c)

Following the General Procedure of Description 14, methyl (3R)-3-(tert-butoxycarbonylamino)-4-iodo-butanoate (5c) was prepared from methyl (3R)-3-(tert-butoxycarbonylamino)-4-hydroxy-butanoate (5b) (5.73 g, 24.6 mmol), iodine (I₂) (6.23 g, 24.6 mmol), triphenylphosphine (PPh₃) (6.45 g, 24.6 mmol), and imidazole (1.67 g, 24.6 mmol) in anhydrous dichloromethane (DCM) (100 mL). Aqueous reductive work-up and purification by silica gel column chromatography with an ethyl acetate (EtOAc)/hexane mixture (EtOAc/hexane=7:3, v/v) furnished 4.30 g (51% yield) of the title compound (5c) as a colorless to beige solid. R_{f} ˜0.79 (EtOAc/hexane=7:3, v/v). ¹H NMR (400 MHz, CDCl₃): δ 5.10 (br. d, J=7.2 Hz, 1H), 4.00-3.80 (m, 1H), 3.69 (s, 3H), 3.50-3.36 (m, 2H), 2.76 (dd, J=16.5, 5.4 Hz, 1H), 2.62 (dd, J=16.5, 6.3 Hz, 1H), 1.43 (s, 9H) ppm. The analytical data correspond with the analytical data for the (S)-enantiomer in the literature (Dexter and Jackson, J. Org. Chem., 1999, 64, 7579-7585).

### Step D: Methyl (3S)-4-(5-amino-2-methyl-phenyl)-3-(tert-butoxycarbonylamino)butanoate (5d)

Following the General Procedure of Description 15 (Part A), zinc dust (Zn) (1.96 g, 30.0 mmol) was activated with elemental iodine (I₂) (190 mg, 0.75 mmol, 15 mol-%) and trimethyl silylchloride (MeSiCl, TMSCl) (95 μL, 81 mg, 0.75 mmol, 15 mol-%) in degassed anhydrous N,N-dimethylformamide (DMF) (6 mL). The zinc insertion product was prepared from methyl (3R)-3-(tert-butoxycarbonylamino)-4-iodo-butanoate (5c) (1.72 g, 5.0 mmol) in the presence of additional I₂ (190 mg, 0.75 mmol, 15 mol-%) and TMSCl (95 μL, 81 mg, 0.75 mmol, 15 mol-%).

Following the General Procedure of Description 15 (Part B), the zinc insertion product of (5c) was used in situ to cross couple with commercial 3-iodo-4-methyl-aniline (583 mg, 2.5 mmol) in the presence of tris(benzylideneacetone) dipalladium (Pd₂(dba)₃) (57 mg, 0.03 mmol, 2.5 mol-%) and tris(o-tolyl)phosphine (P(o-tol)₃) (76 mg, 0.25 mmol, 10 mol-%) in anhydrous degassed DMF (6 mL). Filtration, aqueous work-up, and purification by silica gel column chromatography with ethyl acetate (EtOAc)/hexane mixtures (EtOAc/hexane=7:3→1:1, v/v) furnished 1.04 g (65% yield) of the title compound (5d) as a yellow viscous oil. R_{f} ˜0.28 (EtOAc/hexane=1:1, v/v). ¹H NMR (400 MHz, CDCl₃): δ 6.89 (d, J=8.4 Hz, 1H), 6.48-6.44 (m, 2H), 5.10-5.02 (br. m, 1H), 4.18-4.08 (m, 1H), 3.65 (s, 3H), 3.30 (br. s, 2H), 2.82-2.78 (br. dd, 1H), 2.70 (dd, J=10.2, 6.0 Hz, 1H), 2.51 (dd, J=16.0, 5.2 Hz, 1H), 2.45 (dd, J=16.0, 5.6 Hz, 1H), 2.19 (s, 3H), 1.38 (s, 9H) ppm. LC/MS: Rₜ=1.320 min. LC/MS: m/z=323.20 (M+H⁺)⁺, 345.15 (M+Na⁺)⁺.

### Step E: Methyl (3S)-4-[5-[bis(2-chloroethyl)amino]-2-methyl-phenyl]-3-(tert-butoxycarbonylamino)butanoate (5e)

Following the General Procedure of Description 7 (Variant C), methyl (3S)-4-[5-[bis(2-chloroethyl)amino]-2-methyl-phenyl]-3-(tert-butoxycarbonylamino)butanoate (5e) was prepared from methyl (3S)-4-(5-amino-2-methyl-phenyl)-3-(tert-butoxycarbonylamino)-butanoate (5d) (967 mg, 3.0 mmol), 2-chloroacetaldehyde (˜50 wt-% in water, ˜7.87 M) (3.05 mL, 24.0 mmol), and sodium cyanoborohydride (NaBH₃CN) (624 mg of 95% purity=593 mg, 9.43 mmol) in a mixture of methanol (MeOH) (18 mL) and 85 wt-% phosphoric acid (H₃PO₄) (8.1 mL). Aqueous work-up and purification by silica gel column chromatography with an ethyl acetate (EtOAc)/hexane mixture (EtOAc/hexane=1:4, v/v) afforded 1.4 g (97% yield) of the title compound (5e) as a colorless oil. R_{f} ˜0.32 (EtOAc/Hxn=4:1, v/v). ¹H NMR (400 MHz, CDCl₃): δ 7.00 (d, J=8.5 Hz, 1H), 6.49 (d, J=2.4 Hz, 1H), 6.42 (s, 1H), 5.10-5.04 (br. m, 1H), 3.69 (s, 3H), 3.67-3.59 (m, 8H), 2.90-2.80 (m, 1H), 2.78-2.70 (m, 1H), 2.60-2.40 (m, 2H), 2.23 (s, 3H), 1.37 (s, 9H) ppm. LC/MS: Rₜ=2.533 min; ESI (pos.) m/z=447.15 (M+H⁺)⁺, 469.15 (M+Na⁺)⁺.

### Step F: (3S)-3-Amino-4-[5-[bis(2-chloroethyl)amino]-2-methyl-phenyl]butanoic acid (5)

Following the General Procedure of Description 8, (3S)-3-amino-4-[5-[bis(2-chloroethyl)amino]-2-methyl-phenyl]butanoic acid (5) was prepared through hydrolytic deprotection of methyl (3S)-4-[5-[bis(2-chloroethyl)amino]-2-methyl-phenyl]-3-(tert-butoxycarbonylamino)butanoate (5e) (˜1.4 g, 3.13 mmol) in a mixture of concentrated hydrochloric acid (HCl) (7.5 mL) and 1,4-dioxane (7.5 mL). Part of the crude material obtained after work-up was purified by preparative HPLC to afford ˜20 mg of the target compound (5) as a colorless solid after primary lyophilization. ¹H NMR (400 MHz, MeOH-d⁴): δ 7.04 (d, J=8.4 Hz, 1H), 6.59 (d, J=8.4 Hz, 1H), 6.54 (s, 1H), 3.74-3.68 (br. m, 4H), 3.67-3.62 (br. m, 4H), 3.58-3.50 (m, 1H), 2.92-2.86 (m, 2H), 2.44 (dd, J=16.8, 4.0 Hz, 1H), 2.31 (dd, J=16.8, 8.4 Hz, 1H), 2.22 (s, 3H) ppm. The analytical data correspond to the analytical data obtained for racemic compound (3). Various batches of mono- or dihydrochloride salts of (6) can be prepared by primary lyophilization of solutions of (5) in aqueous acetonitrile (MeCN) containing either 1.0 eq. of 1.0 N hydrochloric acid (HCl) or an excess of 1.0 N or higher concentrated hydrochloric acid (HCl).

### Example 6

### (3R)-3-Amino-4-[5-[bis(2-chloroethyl)amino]-2-methyl-phenyl]butanoic acid (6)

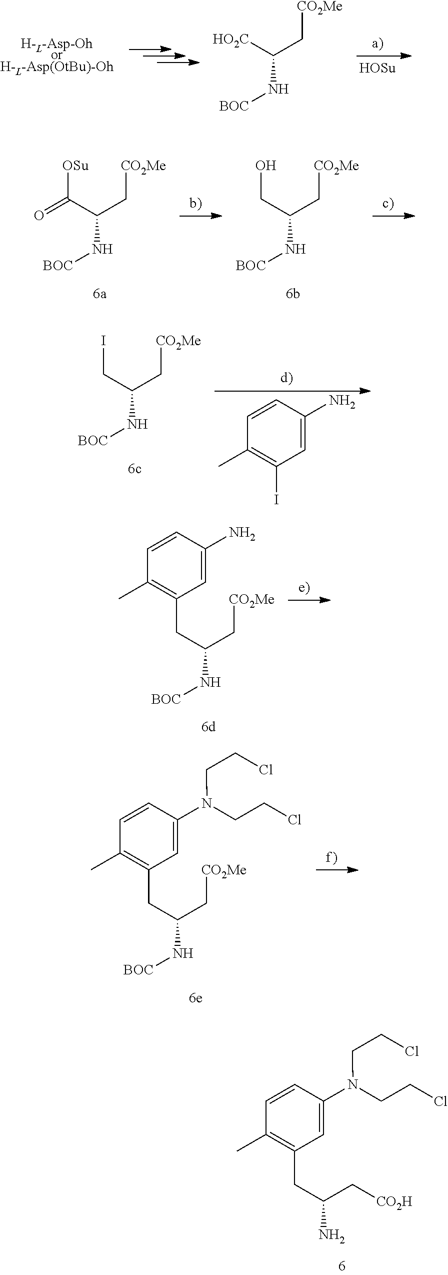

### Step A: O¹-(2,5-Dioxopyrrolidin-1-yl) O⁴-tert-butyl (2S)-2-(tert-butoxycarbonylamino)-butanedioate (6a)

Following the General Procedure of Description 12, O¹-(2,5-Dioxopyrrolidin-1-yl) O⁴-tert-butyl (2S)-2-(tert-butoxycarbonylamino)-butanedioate (6a) was prepared from (2S)-2-(tert-butoxycarbonylamino)-4-tert-butoxy-4-oxo-butanoic acid (8.32 g, 28.8 mmol) (commercially available or prepared following the General Procedure of Description 4 from commercial H-L-Asp(OtBu)-OH (5.68 g, 30.0 mmol) and Boc₂O (6.55 g, 30.0 mmol) in a mixture of 1,4-dioxane (25 mL) and a freshly prepared 1.0 N aqueous sodium hydroxide (NaOH) solution (33 mL, 33 mmol) (8.33 g, 96% yield) (Keller, et al., Org. Synth., 1985, 63, 160)), N-hydroxysuccinimide (1-hydroxypyrrolidine-2,5-dione, HOSu, NHS) (3.53 g, 30.7 mmol), and dicyclohexylcarbodiimide (DCC) (6.03 g, 29.2 mmol in ethyl acetate (EtoAc) (100 mL) at room temperature. Filtration and aqueous work-up furnished 11.8 g (quantitative yield) of the title compound (6a) as a colorless solid which was of sufficient purity to be used directly and without further isolation and purification in the next step. R_{f} ˜0.56 (EtOAc/hexane=1:1, v/v); R_{f} ˜0.34 (EtOAc/hexane=1:2, v/v). ¹H NMR (300 MHz, CDCl₃): δ 5.63 (d, J=9.3 Hz, 1H), 5.00-4.92 (m, 1H), 3.01 (dd, J=17.4, 5.1 Hz, 1H), 2.84 (dd, superimposed, J=17.4, 4.8 Hz, 1H), 2.84 (s, superimposed, 4H), 1.47 (s, 9H), 1.45 (s, 9H) ppm. LC/MS: Rₜ=2.567 min; ESI (pos.) m/z=409.15 (M+Na⁺)⁺, 795.35 (2M+Na⁺)⁺; ESI (neg.) m/z=384.90.

### Step B: tert-Butyl (3S)-3-(tert-butoxycarbonylamino)-4-hydroxy-butanoate (6b)

Following the General Procedure of Description 13, tert-butyl (3S)-3-(tert-butoxycarbonylamino)-4-hydroxy-butanoate (6b) was prepared through reduction of O¹-(2,5-dioxopyrrolidin-1-yl) O⁴-tert-butyl (2S)-2-(tert-butoxycarbonylamino)-butanedioate (6a) (11.8 g, 30.5 mmol) with sodium borohydride (NaBH₄) (2.31 g, 61.0 mmol) in tetrahydrofuran (THF)/water (110 mL/16 mL). Aqueous work-up and purification by silica gel column chromatography with an ethyl acetate (EtOAc)/hexane mixture (EtOAc/hexane=11:9, v/v) furnished 7.30 g (87% yield) of the title compound (6b) as a colorless viscous oil. R_{f} ˜0.52 (EtOAc/hexane=1:1, v/v). ¹H NMR (400 MHz, CDCl₃): δ 5.23 (br. d, J=5.1 Hz, 1H), 4.02-3.90 (m, 1H), 3.67 (d, J=4.8 Hz, 2H), 2.55 (dd, superimposed, J=15.3, 6.0 Hz, 1H), 2.48 (dd, superimposed, J=15.3, 6.3 Hz, 1H), 1.44 (s, 9H), 1.43 (s, 9H) ppm. LC/MS: Rₜ=1.887 min; ESI (pos.) m/z=298.10 (M+Na⁺)⁺; m/z=573.35 (2M+Na⁺)⁺.

### Step C: tert-Butyl (3S)-3-(tert-butoxycarbonylamino)-4-iodo-butanoate (6c)

Following the General Procedure of Description 14, tert-butyl (3S)-3-(tert-butoxycarbonylamino)-4-iodo-butanoate (6c) was prepared from tert-butyl (3S)-3-(tert-butoxycarbonylamino)-4-hydroxy-butanoate (6b) (4.46 g, 16.2 mmol), iodine (I₂) (4.10 g, 16.2 mmol), triphenylphosphine (PPh₃) (4.25 g, 16.2 mmol), and imidazole (1.10 g, 16.2 mmol) in anhydrous dichloromethane (DCM) (70 mL). Aqueous reductive work-up and purification by silica gel column chromatography with ethyl acetate (EtOAc)/hexane mixtures (EtOAc/hexane=7:3→1:1, v/v) furnished 4.20 g (67% yield) of the title compound (6c) as a colorless to beige solid. R_{f} ˜0.79 (EtOAc/hexane=7:3, v/v). ¹H NMR (400 MHz, CDCl₃): δ 5.09 (br. d, J=8.4 Hz, 1H), 3.90-3.80 (m, 1H), 3.44-3.30 (m, 2H), 2.60 (dd, J=15.9, 6.0 Hz, 1H), 2.51 (dd, J=15.9, 6.0 Hz, 1H), 1.45 (s, 9H), 1.43 (s, 9H) ppm. LC/MS: Rₜ=2.332 min; ESI (neg.) m/z=384.80 (M−H⁺)⁻.

### Step D: tert-Butyl (3R)-4-(5-amino-2-methyl-phenyl)-3-(tert-butoxycarbonylamino)-butanoate (6d)

Following the General Procedure of Description 15 (Part A), zinc dust (Zn) (4.07 g, 62.3 mmol) is activated with elemental iodine (I₂) (396 mg, 1.56 mmol, 15 mol-%) and trimethyl silylchloride (MeSiCl, TMSCl) (197 μL, 169 mg, 0.75 mmol, 15 mol-%) in degassed anhydrous N,N-dimethylformamide (DMF) (6 mL). The zinc insertion product was prepared from tert-butyl (3S)-3-(tert-butoxycarbonylamino)-4-iodo-butanoate (6c) (4.01 g, 10.4 mmol) in the presence of additional elemental I₂ (396 mg, 1.56 mmol, 15 mol-%) and TMSCl (197 μL, 169 mg, 0.75 mmol, 15 mol-%).

Following the General Procedure of Description 15 (Part B), The zinc insertion product of (6c) was used in situ to cross couple with commercial 3-iodo-4-methyl-aniline (1.21 g, 5.2 mmol) in the presence of tris(benzylideneacetone) dipalladium (Pd₂(dba)₃) (119 mg, 0.13 mmol, 2.5 mol-%) and tris(o-tolyl)phosphine (P(o-tol)₃) (158 mg, 0.52 mmol, 10 mol-%) in anhydrous degassed DMF (6 mL). Filtration, aqueous work-up, and purification by silica gel column chromatography with an etlyl acetate (EtOAc)/hexane mixture (EtOAc/hexane=7:3, v/v) furnished 1.15 g (61% yield) of the title compound (6d) as a yellow viscous oil. R_{f} ˜0.28 (EtOAc/hexane=1:1, v/v). ¹H NMR (300 MHz, CDCl₃): δ 6.91 (d, J=8.1 Hz, 1H), 6.50-6.46 (m, 2H), 5.20-5.10 (br. m, 1H), 4.18-4.00 (m, 1H), 3.24 (br. s, 2H), 2.88-2.78 (br. dd, 1H), 2.70 (dd, 1H), 2.44 (dd, J=15.4 Hz, 5.4 Hz, 1H), 2.36 (dd, J=15.4 Hz, 5.4 Hz, 1H), 2.22 (s, 3H), 1.45 (s, 9H), 1.40 (s, 9H) ppm. LC/MS: Rₜ=1.433 min; ESI (pos.) m/z=365.20 (M+H⁺)⁺.

### Step E: tert-Butyl (3R)-4-[5-[bis(2-chloroethyl)amino]-2-methyl-phenyl]-3-(tert-butoxycarbonylamino)butanoate (6e)

Following the General Procedure of Description 7 (Variant C), tert-butyl (3R)-4-[5-[bis(2-chloroethyl)amino]-2-methyl-phenyl]-3-(tert-butoxycarbonylamino)butanoate (6e) was prepared from tert-butyl (3R)-4-(5-amino-2-methyl-phenyl)-3-(tert-butoxycarbonylamino)-butanoate (6d) (1.07 g, 2.92 mmol), 2-chloroacetaldehyde (˜50 wt-% in water, ˜7.87 M) (3.0 mL, 23.6 mmol), and sodium cyanoborohydride (NaBH₃CN) (1.25 g of 95% purity=1.19 g, 18.9 mmol) in a mixture of methanol (MeOH) (18 mL) and 85 wt-% phosphoric acid (H₃PO₄) (9 mL). Aqueous work-up and purification by silica gel column chromatography with an ethyl acetate (EtOAc)/hexane mixture (EtOAc/hexane=1:6, v/v) afforded 1.06 g (74% yield) of the title compound (6e) as a colorless oil. R_{f} ˜0.55 (EtOAc/hexane=1:4, v/v). ¹H NMR (400 MHz, CDCl₃): δ 6.98 (d, J=8.4 Hz, 1H), 6.45 (d, J=8.4 Hz, 1H), 6.42 (s, 1H), 5.00 (br. d, 1H), 4.18-4.00 (m, 1H), 3.70-3.50 (m, 8H), 2.80-2.60 (m, 2H), 2.41 (dd, J=16.0, 5.6 Hz, 1H), 2.32 (dd, J=16.0, 6.0 Hz, 1H), 2.21 (s, 3H), 1.42 (s, 9H), 1.32 (s, 9H) ppm. LC/MS: Rₜ=2.944 min; ESI (pos.) m/z=489.20 (M+H⁺)⁺.

### Step F: (3R)-3-Amino-4-[5-[bis(2-chloroethyl)amino]-2-methyl-phenyl]butanoic acid (6)

Following the General Procedure of Description 8, (3R)-3-amino-4-[5-[bis(2-chloroethyl)amino]-2-methyl-phenyl]butanoic acid (6) was prepared through hydrolytic deprotection of tert-butyl (3R)-4-[5-[bis(2-chloroethyl)amino]-2-methyl-phenyl]-3-(tert-butoxycarbonylamino)butanoate (6e) (160 mg, 0.33 mmol) in a mixture of concentrated hydrochloric acid (HCl) (1 mL) and 1,4-dioxane (1 mL). The crude material obtained after work-up was purified by preparative HPLC to afford ˜86 mg (79% recovery) of the target compound (6) as a colorless solid after primary lyophilization. ¹H NMR (400 MHz, MeOH-d⁴): δ 7.04 (d, J=8.4 Hz, 1H), 6.59 (d, J=8.4 Hz, 1H), 6.54 (s, 1H), 3.74-3.68 (br. m, 4H), 3.67-3.62 (br. m, 4H), 3.60-3.52 (m, 1H), 2.92-2.86 (m, 2H), 2.46 (dd, J=16.8, 4.0 Hz, 1H), 2.34 (dd, J=16.8, 8.4 Hz, 1H), 2.22 (s, 3H) ppm. LC/MS: Rₜ=1.317 min; 100% AUC at λ=254 nm; ESI (pos.) m/z=333.05 (M+H⁺)⁺. LC/UV: Rₜ=8.489 min, 99.1% AUC at λ=254 nm. The analytical data correspond to the analytical data obtained for racemic compound (3).

Various batches of mono- or dihydrochloride salts of (6) can be prepared by primary lyophilization of solutions of (6) in aqueous acetonitrile (MeCN) containing either 1.0 eq. of 1.0 N hydrochloric acid (HCl) or an excess of 1.0 N or higher concentrated hydrochloric acid (HCl). Following the General Procedure of Description 9 (Variant B), dihydrochloride salts of (6) can also be prepared through deprotection with 2 N HCl in diethyl ether (2 N HCl in Et₂O) to yield the target compound (6) as a solid dihydrochloride salt after evaporation of the solvents and lyophilization from an aqueous solution. The material is generally of sufficient purity to be used directly and without further isolation and purification in in vitro and/or in vivo evaluation.

### Example 7

### (3S)-3-Amino-4-[5-[bis(2-chloroethyl)amino]-2-methoxy-phenyl]butanoic acid (7)

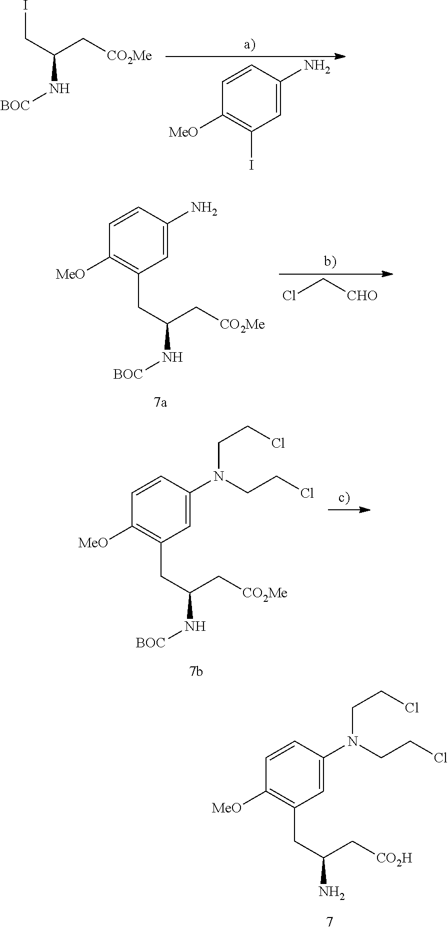

### Step A: Methyl (3S)-4-(5-amino-2-methoxy-phenyl)-3-(tert-butoxycarbonylamino)-butanoate (7a)

Following the General Procedure of Description 15 (Part A), zinc dust (Zn) (392 mg, 6.0 mmol) was activated with elemental iodine (I₂) (38 mg, 0.15 mmol, 15 mol-%) and trimethyl silylchloride (MeSiCl, TMSCl) (19 μL, 16 mg, 0.15 mmol, 15 mol-%) in degassed anhydrous N,N-dimethylformamide (DMF) (2 mL). The zinc insertion product was prepared from methyl (3R)-3-(tert-butoxycarbonylamino)-4-iodo-butanoate (5c) (343 mg, 1.0 mmol) in the presence of additional I₂ (38 mg, 0.15 mmol, 15 mol-%) and TMSCl (19 μL, 16 mg, 0.15 mmol, 15 mol-%).

Following the General Procedure of Description 15 (Part A), the zinc insertion product of (5c) was used in situ to cross couple with commercial 3-iodo-4-methoxy-aniline (249 mg, 1.0 mmol) in the presence of tris(benzylideneacetone) dipalladium (Pd₂(dba)₃) (23 mg, 0.025 mmol, 2.5 mol-%) and tris(o-tolyl)phosphine (P(o-tol)₃) (30 mg, 0.10 mmol, 10 mol-%) in anhydrous degassed DMF (3 mL). Filtration, aqueous work-up, and purification by silica gel column chromatography with ethyl acetate (EtOAc)/hexane and dichloromethane(DCM)/EtOAc mixtures (EtOAc/hexane=1:1, v/v→DCM/EtOAc=1:1, v/v) furnished ˜280 mg (66% yield; ˜80% purity by AUC) of the title compound (7a) as a yellow viscous oil. R_{f} ˜0.23 (EtOAc/hexane=1:1, v/v). ¹H NMR (300 MHz, CDCl₃): δ 6.90 (br s, 1H), 6.78 (br. d, J=8.1 Hz, 1H), 6.70 (d, J=8.7 Hz, 1H), 5.28 (br. d, J=8.1 Hz, 1H), 4.40-4.10 (m, 1H), 3.37 (s, 3H), 2.90-2.80 (br. m, 1H), 2.75 (dd, J=12.6, 6.3 Hz, 1H), 2.50 (d, J=5.1 Hz, 2H), 1.35 (s, 9H) ppm. LC/MS: Rₜ=0.908 min; ESI (pos.) m/z=339.15 (M+H⁺)⁺, 677.40 (2M+H⁺)⁺, 699.35 (2M+Na⁺)⁺.

### Step B: Methyl (3S)-4-[5-[bis(2-chloroethyl)amino]-2-methoxy-phenyl]-3-(tert-butoxycarbonylamino)butanoate (7b)

Following the General Procedure of Description 7 (Variant C), methyl (3S)-4-[5-[bis(2-chloroethyl)amino]-2-methoxy-phenyl]-3-(tert-butoxycarbonylamino)butanoate (7b) was prepared from methyl (3S)-4-(5-amino-2-methoxy-phenyl)-3-(tert-butoxycarbonylamino)-butanoate (7a) (280 mg, 0.83 mmol, ˜80% purity), 2-chloroacetaldehyde (˜50 wt-% in water, ˜7.87 M) (842 μL, 6.63 mmol), and sodium cyanoborohydride (NaBH₃CN) (105 mg of 95% purity=100 mg, 1.59 mmol) in a mixture of methanol (MeOH) (5 mL) and 85 wt-% phosphoric acid (H₃PO₄) (2.5 mL). Aqueous work-up and purification by silica gel column chromatography with an ethyl acetate (EtOAc)/hexane mixture (EtOAc/hexane=1:4, v/v) afforded 104 mg (27% yield) of the title compound (7b) as a colorless oil. R_{f} ˜0.30 (EtOAc/hexane=1:4); LC/MS: Rₜ=2.493 min; ESI (pos.) m/z=463.20 (M+H⁺)⁺.

### Step C: (3S)-3-Amino-4-[5-[bis(2-chloroethyl)amino]-2-methoxy-phenyl]butanoic acid (7)

Following the General Procedure of Description 8, (3S)-3-amino-4-[5-[bis(2-chloroethyl)amino]-2-methoxy-phenyl]butanoic acid (7) was prepared from methyl (3S)-4-[5-[bis(2-chloroethyl)amino]-2-methoxy-phenyl]-3-(tert-butoxycarbonylamino)butanoate (7b) (104 mg, 0.224 mmol) by hydrolysis in a mixture of concentrated hydrochloric acid (HCl) (3 mL) and 1,4-dioxane (3 mL) at about 60° C. (oil bath) for about 6 hours to afford ˜90 mg (˜95% yield) the title compound (7) as a dihydrochloride salt after evaporation of the solvents under reduced pressure. LC/MS: Rₜ=1.207 min; ˜100% purity by AUC at λ=254 nm. ESI (pos.) m/z=349.05 (M+H⁺)⁺.

### Example 8

### (3S)-3-Amino-4-[3-[bis(2-chloroethyl)amino]-2,6-dimethyl-phenyl]butanoic acid (8)

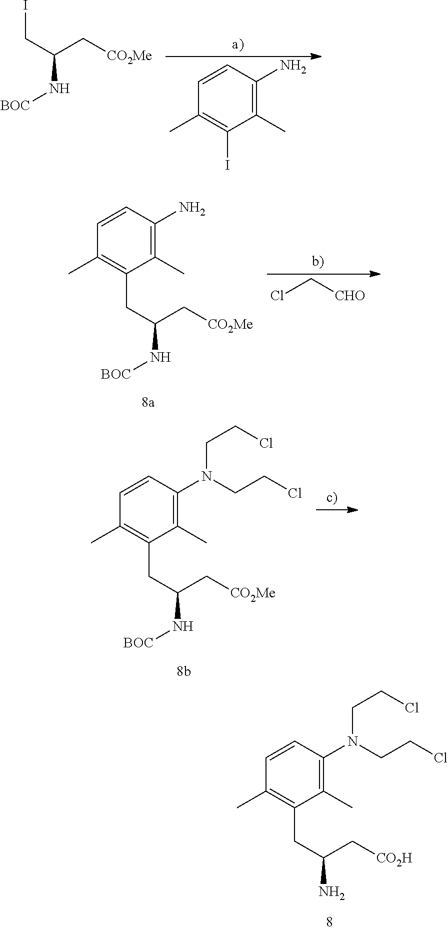

### Step A: Methyl (3S)-4-(3-amino-2,6-dimethyl-phenyl)-3-(tert-butoxycarbonylamino)-butanoate (8a)

Following the General Procedure of Description 15 (Part A), zinc dust (Zn) (392 mg, 6.0 mmol) is activated with elemental iodine (I₂) (38 mg, 0.15 mmol, 15 mol-%) and trimethyl silylchloride (MeSiCl, TMSCl) (19 μL, 16 mg, 0.15 mmol, 15 mol-%) in degassed anhydrous N,N-dimethylformamide (DMF) (3 mL). The zinc insertion product is prepared from methyl (3R)-3-(tert-butoxycarbonylamino)-4-iodo-butanoate (5c) (343 mg, 1.0 mmol) in the presence of additional I₂ (38 mg, 0.15 mmol, 15 mol-%) and TMSCl (19 μL, 16 mg, 0.15 mmol, 15 mol-%).

Following the General Procedure of Description 15 (Part B), the zinc insertion product of (5c) is used in situ to cross couple with 3-iodo-2,4-dimethyl-aniline (247 mg, 1.0 mmol; preparable following Description 6 from commercial 2-iodo-1,3-dimethyl-4-nitro-benzene (2.78 g, 10.0 mmol), 5.6 g iron powder (Fe), and calcium chloride dehydrate (CaCl₂.2H₂O) (1.47 g, 10.0 mmol) in a mixture of ethanol (EtOH) (20 mL) and water (1 mL)) in the presence of tris(benzylideneacetone) dipalladium (Pd₂(dba)₃) (23 mg, 0.025 mmol, 2.5 mol-%) and tris(o-tolyl)phosphine (P(o-tol)₃) (30 mg, 0.10 mmol, 10 mol-%) in anhydrous degassed DMF (3 mL). Filtration, aqueous work-up, and purification by silica gel column chromatography furnish the title compound (8a).

### Step B: Methyl (3S)-4-[3-[bis(2-chloroethyl)amino]-2,6-dimethyl-phenyl]-3-(tert-butoxycarbonylamino)butanoate (8b)

Following the General Procedure of Description 7 (Variant C), methyl (3S)-4-[3-[bis(2-chloroethyl)amino]-2,6-dimethyl-phenyl]-3-(tert-butoxycarbonylamino)butanoate (8b) is prepared from methyl (3S)-4-(3-amino-2,6-dimethyl-phenyl)-3-(tert-butoxycarbonylamino)-butanoate (8a) (336 mg, 1.0 mmol), 2-chloroacetaldehyde (˜50 wt-% in water, ˜7.87 M) (700 μL, 5.51 mmol), and sodium cyanoborohydride (NaBH₃CN) (264 mg of 95% purity=251 mg, 4.0 mmol) in a mixture of methanol (MeOH) (6 mL) and 85 wt-% phosphoric acid (H₃PO₄) (3 mL). Aqueous work-up and purification by silica gel column chromatography furnish the title compound (8b).

### Step C: (3S)-3-Amino-4-[3-[bis(2-chloroethyl)amino]-2,6-dimethyl-phenyl]butanoic acid (8)

Following the General Procedure of Description 8, (3S)-3-amino-4-[3-[bis(2-chloroethyl)amino]-2,6-dimethyl-phenyl]butanoic acid (8) is prepared from methyl (3S)-4-[3-[bis(2-chloroethyl)amino]-2,6-dimethyl-phenyl]-3-(tert-butoxycarbonylamino)butanoate (8b) (461 mg, 1.0 mmol) by hydrolysis in a mixture of concentrated hydrochloric acid (HCl) (about 5 mL) and 1,4-dioxane (about 5 mL) at about 60° C. for about 15 hours to afford the title compound (8) as a solid dihydrochloride salt after isolation using evaporation and lyophilization. The material thus obtained is purified by preparative RP-HPLC using a water/acetonitrile/0.1 vol-% formic acid gradient to afford the title compound (8) as a dihydrochloride salt after final lyophilization of the solvents in the presence of an excess of 1.0 M hydrochloric acid (HCl).

### Example 9

### (3S)-3-Amino-4-[5-[bis(2-chloroethyl)amino]-2-methyl-phenyl]-3-methyl-butanoic acid (9)

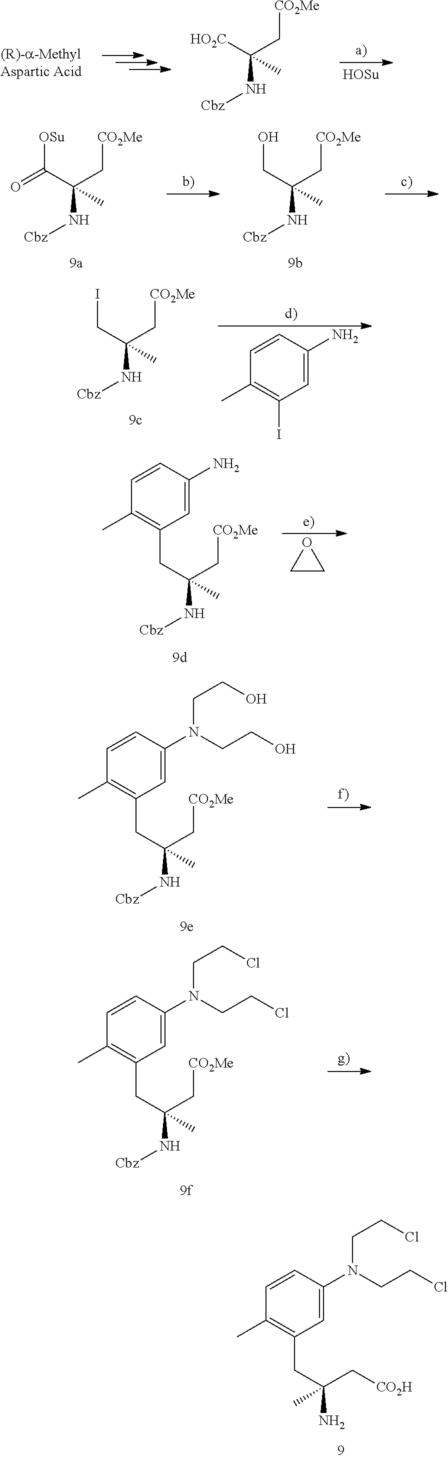

### Step A: O¹-(2,5-Dioxopyrrolidin-1-yl) O⁴-methyl (2R)-2-benzyloxycarbonylamino-2-methyl-butanedioate (9a)

Following the General Procedure of Description 12, O¹-(2,5-Dioxopyrrolidin-1-yl) O⁴-methyl (2R)-2-benzyloxycarbonylamino-2-methyl-butanedioate (9a) is prepared from (2R)-2-benzyloxycarbonylamino-4-methoxy-2-methyl-4-oxo-butanoic acid (2.95 g, 10.0 mmol) (preparable in two steps from commercial (R)-α-methyl aspartic acid: i) SOCl₂, MeOH, 0° C.→room temperature, 3 h; ii) Cbz-OSu (N-(benzyloxycarbonyloxy)succinimide), aq. K₃PO₄/toluene, 0° C.→room temperature, 14 h) following a literature known protocol (Gauvreau, et al., International Publication No. WO 2008/088690)), N-hydroxysuccinimide (1-hydroxypyrrolidine-2,5-dione, HOSu, NHS) (1.21 g, 10.5 mmol), and dicyclohexylcarbodiimide (DCC) (2.06 g, 10.0 mmol in ethyl acetate (EtoAc) (40 mL) at room temperature. Filtration and aqueous work-up furnish the title compound (9a) which may be of sufficient purity to be used directly in the next step without further isolation and purification.

### Step B: Methyl (3R)-3-benzyloxycarbonylamino-4-hydroxy-3-methyl-butanoate (9b)

Following the General Procedure of Description 13, methyl (3R)-3-benzyloxycarbonylamino-4-hydroxy-3-methyl-butanoate (9b) is prepared through reduction of O¹-(2,5-Dioxopyrrolidin-1-yl) O⁴-methyl (2R)-2-benzyloxycarbonylamino-2-methyl-butanedioate (9a) (3.92 g, 10.0 mmol) with sodium borohydride (NaBH₄) (757 mg, 20.0 mmol) in tetrahydrofuran (THF)/water (40 mL/5 mL). Aqueous work-up and purification by silica gel column chromatography furnish the title compound (9b).

### Step C: Methyl (3R)-3-benzyloxycarbonylamino-4-iodo-3-methyl-butanoate (9c)

Following the General Procedure of Description 14, methyl (3R)-3-benzyloxycarbonylamino-4-iodo-3-methyl-butanoate (9c) is prepared from methyl (3R)-3-benzyloxycarbonylamino-4-hydroxy-3-methyl-butanoate (9b) (2.81 g, 10.0 mmol), iodine (I₂) (2.54 g, 10.0 mmol), triphenylphosphine (PPh₃) (2.62 g, 10.0 mmol), and imidazole (681 mg, 10.0 mmol) in anhydrous dichloromethane (DCM) (50 mL). Aqueous reductive work-up and purification by silica gel column chromatography furnish the title compound (9c).

### Step D: Methyl (3S)-4-(5-amino-2-methyl-phenyl)-3-benzyloxycarbonylamino-3-methyl-butanoate (9d)

Following the General Procedure of Description 15 (Part A), zinc dust (Zn) (784 mg, 12.0 mmol) is activated with elemental iodine (I₂) (76 mg, 0.30 mmol, 15 mol-%) and trimethyl silylchloride (MeSiCl, TMSCl) (38 μL, 32 mg, 0.30 mmol, 15 mol-%) in degassed anhydrous N,N-dimethylformamide (DMF) (6 mL). The zinc insertion product is prepared from methyl (3R)-3-benzyloxycarbonylamino-4-iodo-3-methyl-butanoate (9c) (782 mg, 2.0 mmol) in the presence of additional I₂ (76 mg, 0.30 mmol, 15 mol-%) and TMSCl (38 μL, 32 mg, 0.30 mmol, 15 mol-%).

Following the General Procedure of Description 15 (Part B), the zinc insertion product of (9c) is used in situ to cross couple with commercial 3-iodo-4-methyl-aniline (466 mg, 2.0 mmol) in the presence of tris(benzylideneacetone) dipalladium (Pd₂(dba)₃) (46 mg, 0.05 mmol, 2.5 mol-%) and tris(o-tolyl)phosphine (P(o-tol)₃) (60 mg, 0.20 mmol, 10 mol-%) in anhydrous degassed DMF (6 mL). Filtration, aqueous work-up, and purification by silica gel column chromatography furnish the title compound (9d).

### Step E: Methyl (3S)-3-benzyloxycarbonylamino-4-[5-(bis(2-hydroxyethyl)amino)-2-methyl-phenyl]-3-methyl-butanoate (9e)

Following General Procedure of Description 16, methyl (3S)-3-benzyloxycarbonylamino-4-[5-(bis(2-hydroxyethyl)amino)-2-methyl-phenyl]-3-methyl-butanoate (9e) is prepared from methyl (3S)-4-(5-amino-2-methyl-phenyl)-3-benzyloxycarbonylamino-3-methyl-butanoate (9d) (3.70 g, 10.0 mmol) through reaction with ethylene oxide (12.5 mL, 11.0 g, 100.0 mmol) in 15 mL of 50 vol.-% aqueous acetic acid (HOAc) for 24 hours at room temperature to yield the title compound (9e) after aqueous work-up and purification by silica gel chromatography.

### Step F: Methyl (3S)-3-benzyloxycarbonylamino-4-[5-[bis(2-chloroethyl)amino]-2-methyl-phenyl]-3-methyl-butanoate (9f)

Following the General Procedure of Description 17, methyl (3S)-3-benzyloxycarbonylamino-4-[5-[bis(2-chloroethyl)amino]-2-methyl-phenyl]-3-methyl-butanoate (9f) is prepared from methyl (3S)-4-(5-amino-2-methyl-phenyl)-3-benzyloxycarbonylamino-3-methyl-butanoate (9d) (1.85 g, 5.0 mmol) through reaction with i) thionyl chloride (SOCl₂) (3.63 mL, 5.93 g, 50 mmol) in 25 mL of anhydrous chloroform (CHCl₃) for 2 hours at reflux temperature (Variant A), ii) phosphoryl chloride (POCl₃) (2.34 mL, 3.83 g, 25.0 mmol) in anhydrous benzene (20 mL) for about 5 h at a temperature of about 80° C. (Variant B), iii) methanesulfonyl chloride (MsCl) (1.94 mL, 2.86 g, 25.0 mmol) in anhydrous pyridine (20 mL) for 2 hours at 90° C. (Variant C), or iv) triphenylphosphine (Ph₃P) (2.62 g, 10.0 mmol) and carbon tetrachloride (CCl₄) (1.45 mL, 2.31 g, 15.0 mmol) in anhydrous dichloromethane (DCM) (20 mL) at room temperature for 8 hours (Variant D) to yield the target compound (9f) after work-up and purification by silica gel column chromatography.

### Step G: (3S)-3-Amino-4-[5-[bis(2-chloroethyl)amino]-2-methyl-phenyl]-3-methyl-butanoic acid (9)

Following the General Procedure of Description 8, (3S)-3-amino-4-[5-[bis(2-chloroethyl)amino]-2-methyl-phenyl]-3-methyl-butanoic acid (9) is prepared through hydrolytic deprotection of methyl (3S)-3-benzyloxycarbonylamino-4-[5-[bis(2-chloroethyl)amino]-2-methyl-phenyl]-3-methyl-butanoate (9f) (495 mg, 1.0 mmol) in a mixture of concentrated hydrochloric acid (HCl) (5 mL) and 1,4-dioxane (5 mL) and obtained as a solid dihydrochloride salt after isolation using evaporation and lyophilization. The material thus obtained is purified by preparative RP-HPLC using a water/acetonitrile/0.1 vol-% formic acid gradient to afford the title compound (9) as a dihydrochloride salt after final lyophilization of the solvents in the presence of an excess of 1.0 M hydrochloric acid (HCl).

### Example 10

### [(2R)-2-amino-3-[5-[bis(2-chloroethyl)amino]-2-methyl-phenyl]propyl]phosphinic acid (10)

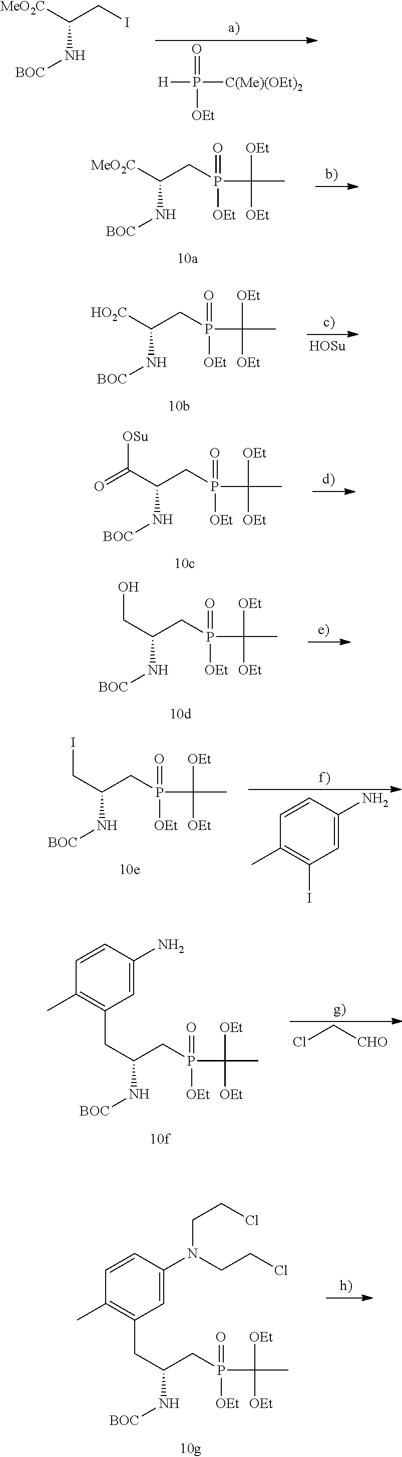

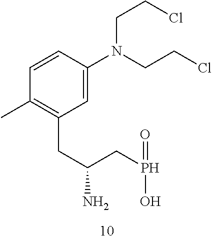

### Step A: Methyl (2R)-2-(tert-butoxycarbonylamino)-3-(1,1-diethoxyethyl(ethoxy)-phosphoryl)propanoate (10a)

Adapting literature protocols, methyl (2R)-2-(tert-butoxycarbonylamino)-3-(1,1-diethoxyethyl(ethoxy)-phosphoryl)propanoate (10a) is prepared form commercial methyl (2R)-2-(tert-butoxycarbonylamino)-3-iodo-propanoate (Jackson and Perez-Gonzalez, Org. Synth., 2005, 81, 77-88) (3.29 g, 10.0 mmol) and 1-(1-ethoxy-1-ethoxyphosphonoyl-ethoxy)ethane (2.10 g, 10.0 mmol) (preparable from 80-90 wt-% aqueous hypophosphorous acid (H₃PO₂), triethylorthoacetate and BF₃-etherate (BF₃.OEt₂) catalyst; (Baylis, Tetrahedron Lett., 1995, 36(51), 9385-9388) in the presence of sodium hydride (NaH) (60 wt-% suspension in mineral oil) (400 mg, 10.0 mmol) in anhydrous toluene (50 mL). The reaction if followed by TLC and/or LC/MS to completion. Aqueous work-up and purification by silica gel column chromatography furnish the title compound (10a).

### Step B: (2R)-2-(tert-Butoxycarbonylamino)-3-(1,1-diethoxyethyl(ethoxy)-phosphoryl)propanoic acid (10b)

Adapting a literature known protocol (Dayal, et al., Steroids, 1990, 55(5), 233-237), a reaction mixture of methyl (2R)-2-(tert-butoxycarbonylamino)-3-(1,1-diethoxyethyl(ethoxy)-phosphoryl)propanoate (10a) (4.11 g, 10.0 mmol) and commercial lithium hydroxide monohydrate (LiOH.H₂O) (839 mg, 20.0 mmol) in a mixture of water (20 mL) and methanol (MeOH) (5 mL) is stirred at room temperature. The reaction is monitored by TLC and/or LC/MS to completion. Acidic aqueous work-up and purification by silica gel column chromatography furnish the title compound (2R)-2-(tert-butoxycarbonylamino)-3-(1,1-diethoxyethyl(ethoxy)phosphoryl)propanoic acid (10b) which may be used directly in the next step without further isolation and purification.

### Step C: (2,5-Dioxopyrrolidin-1-yl) (2R)-2-(tert-butoxycarbonylamino)-3-(1,1-diethoxyethyl(ethoxy)phosphoryl)propanoate (10c)

Following the General Procedure of Description 12, (2,5-dioxopyrrolidin-1-yl) (2R)-2-(tert-butoxycarbonylamino)-3-(1,1-diethoxyethyl(ethoxy)phosphoryl)propanoate (10c) is prepared from (2R)-2-(tert-butoxycarbonylamino)-3-(1,1-diethoxyethyl(ethoxy)-phosphoryl)propanoic acid (10b) (3.97 g, 10.0 mmol), N-hydroxysuccinimide (1-hydroxypyrrolidine-2,5-dione, HOSu, NHS) (1.21 g, 10.5 mmol), and dicyclohexylcarbodiimide (DCC) (2.06 g, 10.0 mmol in ethyl acetate (EtoAc) (40 mL) at room temperature. Filtration and aqueous work-up furnish the title compound (10c) which may be of sufficient purity to be used directly in the next step without further isolation and purification.

### Step D: tert-Butyl N-[(1R)-1-[(1,1-diethoxyethyl(ethoxy)phosphoryl)methyl]-2-hydroxy-ethyl]carbamate (10d)

Following the General Procedure of Description 13, tert-butyl N-[(1R)-1-[(1,1-diethoxyethyl(ethoxy)phosphoryl)methyl]-2-hydroxy-ethyl]carbamate (10d) is prepared through reduction of (2,5-dioxopyrrolidin-1-yl) (2R)-2-(tert-butoxycarbonylamino)-3-(1,1-diethoxyethyl(ethoxy)phosphoryl)propanoate (10c) (4.95 g, 10.0 mmol) with sodium borohydride (NaBH₄) (757 mg, 20.0 mmol) in tetrahydrofuran (THF)/water (40 mL/5 mL). Aqueous work-up and purification by silica gel column chromatography furnish the title compound (10d).

### Step E: tert-Butyl N-[(1S)-1-[(1,1-diethoxyethyl(ethoxy)phosphoryl)methyl]-2-iodo-ethyl]carbamate (10e)

Following the General Procedure of Description 14, tert-butyl N-[(1S)-1-[(1,1-diethoxyethyl(ethoxy)phosphoryl)methyl]-2-iodo-ethyl]carbamate (10e) is prepared from tert-butyl N-[(1R)-1-[(1,1-diethoxyethyl(ethoxy)phosphoryl)methyl]-2-hydroxy-ethyl]carbamate (10d) (3.83 g, 10.0 mmol), iodine (I₂) (2.54 g, 10.0 mmol), triphenylphosphine (PPh₃) (2.62 g, 10.0 mmol), and imidazole (681 mg, 10.0 mmol) in anhydrous dichloromethane (DCM) (50 mL). Aqueous reductive work-up and purification by silica gel column chromatography furnish the title compound (10e).

### Step F: tert-Butyl N-[(1R)-1-[(5-amino-2-methyl-phenyl)methyl]-2-(1,1-diethoxyethyl(ethoxy)phosphoryl)ethyl]carbamate (10f)

Following the General Procedure of Description 15 (Part A), zinc dust (Zn) (784 mg, 12.0 mmol) is activated with elemental iodine (I₂) (76 mg, 0.30 mmol, 15 mol-%) and trimethyl silylchloride (MeSiCl, TMSCl) (38 μL, 32 mg, 0.30 mmol, 15 mol-%) in degassed anhydrous N,N-dimethylformamide (DMF) (6 mL). The zinc insertion product is prepared from tert-butyl N-[(1S)-1-[(1,1-diethoxyethyl(ethoxy)phosphoryl)methyl]-2-iodo-ethyl]carbamate (10e) (987 mg, 2.0 mmol) in the presence of additional I₂ (76 mg, 0.30 mmol, 15 mol-%) and TMSCl (38 μL, 32 mg, 0.30 mmol, 15 mol-%).

Following the General Procedure of Description 15 (Part B), which is used in situ to cross couple with commercial 3-iodo-4-methyl-aniline (466 mg, 2.0 mmol) in the presence of tris(benzylideneacetone) dipalladium (Pd₂(dba)₃) (46 mg, 0.05 mmol, 2.5 mol-%) and tris(o-tolyl)phosphine (P(o-tol)₃) (60 mg, 0.20 mmol, 10 mol-%) in anhydrous degassed DMF (6 mL). Filtration, aqueous work-up, and purification by silica gel column chromatography furnish the title compound (10f).

### Step G: tert-Butyl N-[(1R)-1-[[5-[bis(2-chloroethyl)amino]-2-methyl-phenyl]methyl]-2-(1,1-diethoxyethyl(ethoxy)phosphoryl)ethyl]carbamate (10g)

Following the General Procedure of Description 7 (Variant C), tert-butyl N-[(1R)-1-[[5-[bis(2-chloroethyl)amino]-2-methyl-phenyl]methyl]-2-(1,1-diethoxyethyl(ethoxy)phosphoryl)ethyl]carbamate (10g) is prepared from tert-butyl N-[(1R)-1-[(5-amino-2-methyl-phenyl)methyl]-2-(1,1-diethoxyethyl(ethoxy)phosphoryl)ethyl]carbamate (10f) (472 mg, 1.0 mmol), 2-chloroacetaldehyde (˜50 wt-% in water, ˜7.87 M) (700 μL, 5.51 mmol), and sodium cyanoborohydride (NaBH₃CN) (264 mg of 95% purity=251 mg, 4.0 mmol) in a mixture of methanol (MeOH) (6 mL) and 85 wt-% phosphoric acid (H₃PO₄) (3 mL). Aqueous work-up and purification by silica gel column chromatography furnish the title compound (10g).

### Step H: [(2R)-2-Amino-3-[5-[bis(2-chloroethyl)amino]-2-methyl-phenyl]propyl]phosphinic acid (10)

Following the General Procedure of Description 8, [(2R)-2-amino-3-[5-[bis(2-chloroethyl)amino]-2-methyl-phenyl]propyl]phosphinic acid (10) is prepared through hydrolytic deprotection of tert-butyl N-[(1R)-1-[[5-[bis(2-chloroethyl)amino]-2-methyl-phenyl]methyl]-2-(1,1-diethoxyethyl(ethoxy)phosphoryl)ethyl]carbamate (10g) (598 mg, 1.0 mmol) in a mixture of concentrated hydrochloric acid (HCl) (5 mL) and 1,4-dioxane (5 mL) and obtained as a solid dihydrochloride salt after isolation using evaporation and lyophilization. The material thus obtained is purified by preparative RP-HPLC using a water/acetonitrile/0.1 vol-% formic acid gradient to afford the title compound (9) as a dihydrochloride salt after final lyophilization of the solvents in the presence of an excess of 1.0 M hydrochloric acid (HCl).

### Example 11

### (3R)-3-Amino-4-[5-(2-methylsulfonyloxyethyl(propyl)amino)-2-methyl-phenyl]butanoic acid (11)

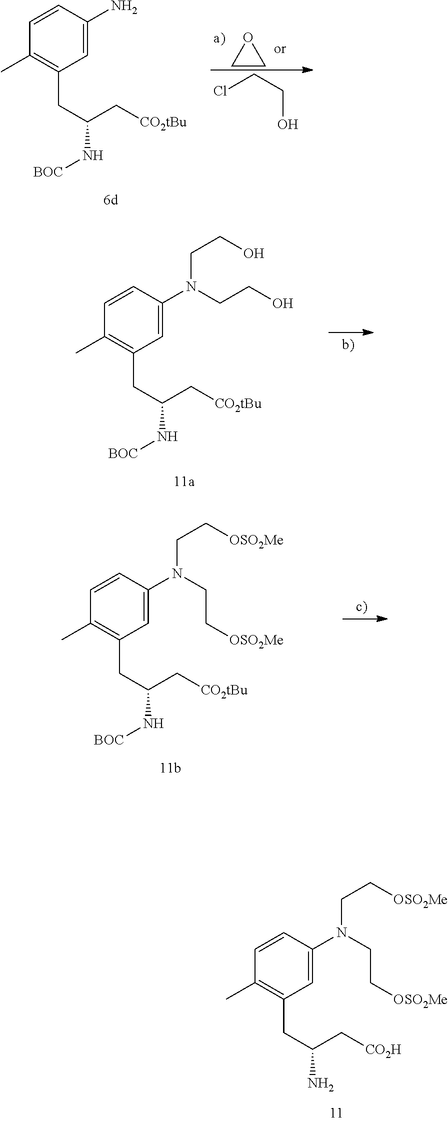

### Step A: tert-Butyl (3R)-4-[5-(bis(2-hydroxyethyl)amino)-2-methyl-phenyl]-3-(tert-butoxycarbonylamino)butanoate (11a)

Variant A: Following General Procedure of Description 16, tert-butyl (3R)-4-[5-(bis(2-hydroxyethyl)amino)-2-methyl-phenyl]-3-(tert-butoxycarbonylamino)butanoate (11a) is prepared from tert-butyl (3R)-4-(5-amino-2-methyl-phenyl)-3-(tert-butoxycarbonylamino)-butanoate (6d) (3.64 g, 10.0 mmol) through reaction with ethylene oxide (12.5 mL, 11.0 g, 100.0 mmol) in 15 mL of 50 vol.-% aqueous acetic acid (HOAc) for 24 hours at room temperature to yield the title compound (11) after aqueous work-up and purification by silica gel chromatography.

Variant B: Adapting literature known protocols (Palmer, et al., J. Med. Chem. 1990, 33(1), 112-121; Coggiola, et al., Bioorg. Med. Chem. Lett., 2005, 15(15), 3551-3554; Verny and Nicolas, J. Label. Cmpds Radiopharm, 1988, 25(9), 949-955; and Lin, Bioorg. Med. Chem. Lett., 2011, 21(3), 940-943), a reaction mixture of tert-butyl (3R)-4-(5-amino-2-methyl-phenyl)-3-(tert-butoxycarbonylamino)-butanoate (6d) (3.64 g, 10.0 mmol) and commercial 2-chloroethanol (2.68 mL, 3.22 g, 40.0 mmol), calcium carbonate (CaCO₃) (2.0 g, 20.0 mmol, 2.0 equivalents) in water (about 35 mL), and a catalytic amount of potassium iodide (KI) (166 mg, 1.0 mmol, 10 mol-%) is heated at reflux for about 12-24 hours. The reaction is followed by TLC and/or LC/MS to completion. The pH of the reaction mixture is adjusted to ˜7 with a 2.5 M (10 wt-%) aqueous solution of sodium hydroxide (NaOH). Aqueous work-up and purification by silica gel column chromatography furnish the target compound (11a).

### Step B: tert-Butyl (3R)-4-[5-(bis(2-methylsulfonyloxyethyl)amino)-2-methyl-phenyl]-3-(tert-butoxycarbonylamino)butanoate (11b)

Following the General Procedures of Description 18, tert-butyl (3R)-4-[5-(bis(2-methylsulfonyloxyethyl)amino)-2-methyl-phenyl]-3-(tert-butoxycarbonylamino)butanoate (11b) is prepared from tert-butyl (3R)-4-[5-(bis(2-hydroxyethyl)amino)-2-methyl-phenyl]-3-(tert-butoxycarbonylamino)butanoate (11a) (2.26 g, 5.0 mmol) and either methanesulfonyl anhydride (Ms₂O) (3.48 g, 20.0 mmol) in the presence of triethylamine (TEA, Et₃N) (3.48 mL, 2.54 g, 25.0 mmol) and 4-N,N-(dimethylamino)pyridine (DMAP) (122 mg, 1.0 mmol, 20 mol-%) in anhydrous dichloromethane (DCM) (30 mL) (Variant A) or methanesulfonyl chloride (MsCl) (0.96 mL, 1.44 g, 12.5 mmol) in the presence of triethylamine (TEA, Et₃N) (2.10 mL, 1.52 g, 15.0 mmol) or pyridine (4.0 mL, 3.96 g, 50.0 mmol) in anhydrous dichloromethane (DCM) (30 mL) (Variant B) to yield the target compound (11b) after aqueous work-up and purification by silica gel column chromatography.

### Step C: (3R)-3-Amino-4-[5-(bis(2-methylsulfonyloxyethyl)amino)-2-methyl-phenyl]butanoic acid (11)

Following the General Procedure of Description 9 (Variant B), (3R)-3-amino-4-[5-(bis(2-methylsulfonyloxyethyl)amino)-2-methyl-phenyl]butanoic acid (11) is prepared from tert-butyl (3R)-4-[5-(bis(2-methylsulfonyloxyethyl)amino)-2-methyl-phenyl]-3-(tert-butoxycarbonylamino)butanoate (11b) (609 mg, 1.0 mmol) in 2 N HCl in diethyl ether (2 N HCl in Et₂O) (10 mL, 20 mmol) to yield the target compound (11) as an solid dihydrochloride salt after evaporation of the solvents and lyophilization from an aqueous solution. The material may be further purified by preparative HPLC followed by lyophilization. Optionally, the lyophilization is conducted in the presence of 1 equivalent or an excess of 1.0 M hydrochloric acid (HCl).

### Example 12

### (3R)-3-Amino-4-[5-(bis(2-bromoethyl)amino)-2-methyl-phenyl]butanoic acid (12)

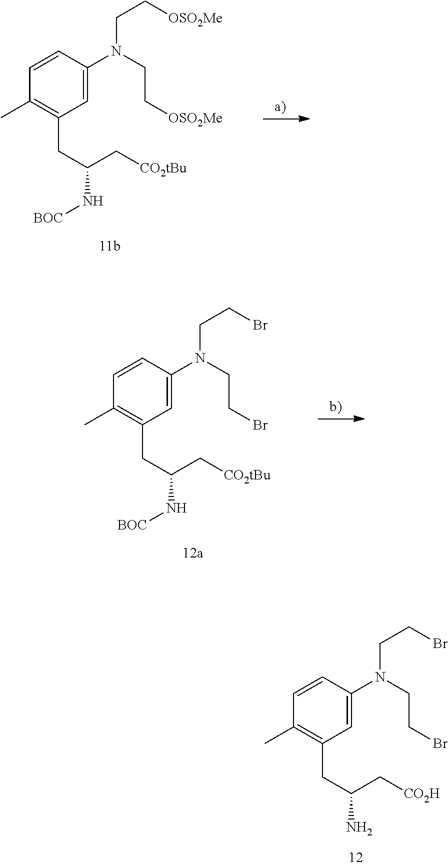

### Step A: tert-Butyl (3R)-4-[5-(bis(2-bromoethyl)amino)-2-methyl-phenyl]-3-(tert-butoxycarbonylamino)butanoate (12a)

Following the General Procedure of Description 19, tert-butyl (3R)-4-[5-(bis(2-bromoethyl)amino)-2-methyl-phenyl]-3-(tert-butoxycarbonylamino)butanoate (12a) is prepared from tert-butyl (3R)-4-[5-(bis(2-methyl sulfonyloxyethyl)amino)-2-methyl-phenyl]-3-(tert-butoxycarbonylamino)butanoate (11b) (1.22 g, 2.0 mmol) through reaction with lithium bromide (LiBr) (1.74 g, 20.0 mmol) in tetrahydrofuran (THF) (10 mL) at reflux temperature for about 6 h to yield the title compound (12a) after aqueous work-up and purification by silica gel column chromatography with ethyl acetate (EtOAc) and hexane mixtures.

### Step B: (3R)-3-Amino-4-[5-(bis(2-bromoethyl)amino)-2-methyl-phenyl]butanoic acid (12)

Following the General Procedure of Description 9 (Variant A), (3R)-3-amino-4-[5-(bis(2-bromoethyl)amino)-2-methyl-phenyl]butanoic acid (12) is prepared from tert-butyl (3R)-4-[5-(bis(2-bromoethyl)amino)-2-methyl-phenyl]-3-(tert-butoxycarbonylamino)butanoate (12a) (578 mg, 1.0 mmol) through deprotection in a trifluoroacetic acid (TFA)/dichloromethane (DCM) mixture (TFA/DCM=1:1 v/v, 10 mL) at room temperature for about 6 h to yield the target compound (12) as a ditrifluoroacetate salt after evaporation and lyophilization from an aqueous acetonitrile solution. The material may be further purified by preparative RP-HPLC using a water/acetonitrile/0.1 vol-% formic acid gradient followed by lyophilization in the presence of 1.0 equivalent or an excess of 1.0 M hydrobromic acid (HBr).

### Example 13

### (3R)-3-Amino-4-[5-(2-chloroethyl(2-methylsulfonyloxyethyl)amino)-2-methyl-phenyl]butanoic acid (13)

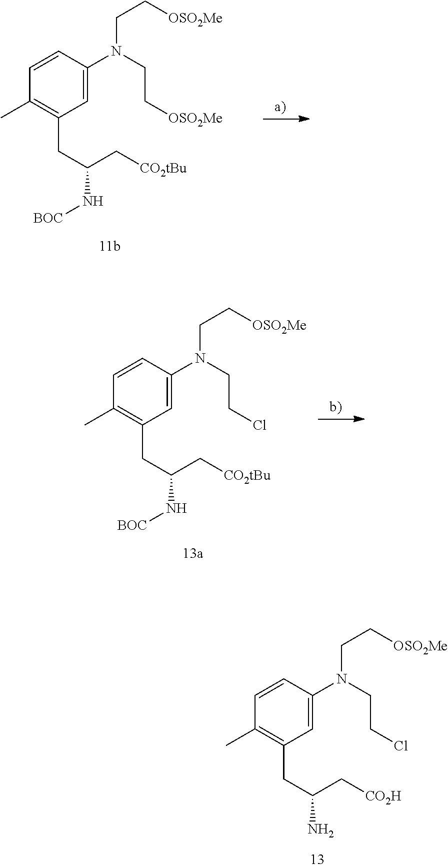

### Step A: tert-Butyl (3R)-3-(tert-butoxycarbonylamino)-4-[5-(2-chloroethyl(2-methylsulfonyloxyethyl)amino)-2-methyl-phenyl]butanoate (13a)

Following the General Procedure of Description 19, tert-butyl (3R)-3-(tert-butoxycarbonylamino)-4-[5-(2-chloroethyl(2-methylsulfonyloxyethyl)amino)-2-methyl-phenyl]butanoate (13a) is prepared from tert-butyl (3R)-4-[5-(bis(2-methylsulfonyloxyethyl)amino)-2-methyl-phenyl]-3-(tert-butoxycarbonylamino)butanoate (11b) (2.44 g, 4.0 mmol) through reaction with lithium chloride (LiCl) (186 mg, 2.2 mmol) in anhydrous acetonitrile (MeCN) (20 mL) at reflux temperature for 1.5 h to yield the title compound (13a) after aqueous work-up and purification by silica gel column chromatography.

### Step B: (3R)-3-Amino-4-[5-(2-chloroethyl(2-methylsulfonyloxyethyl)amino)-2-methyl-phenyl]butanoic acid (13)

Following the General Procedure of Description 9 (Variant A), (3R)-3-amino-4-[5-(2-chloroethyl(2-methylsulfonyloxyethyl)amino)-2-methyl-phenyl]butanoic acid (13) is prepared from tert-butyl (3R)-3-(tert-butoxycarbonylamino)-4-[5-(2-chloroethyl(2-methylsulfonyloxyethyl)amino)-2-methyl-phenyl]butanoate (13a) (549 mg, 1.0 mmol) through deprotection in a trifluoroacetic acid (TFA)/dichloromethane (DCM) mixture (TFA/DCM=1:1 v/v, 10 mL) at room temperature for about 6 h to yield the target compound (13) as a ditrifluoroacetate salt after evaporation and lyophilization from an aqueous acetonitrile solution.

### Example 14

### (3R)-3-Amino-4-[5-(2-bromoethyl(2-chloroethyl)amino)-2-methyl-phenyl]butanoic acid (14)

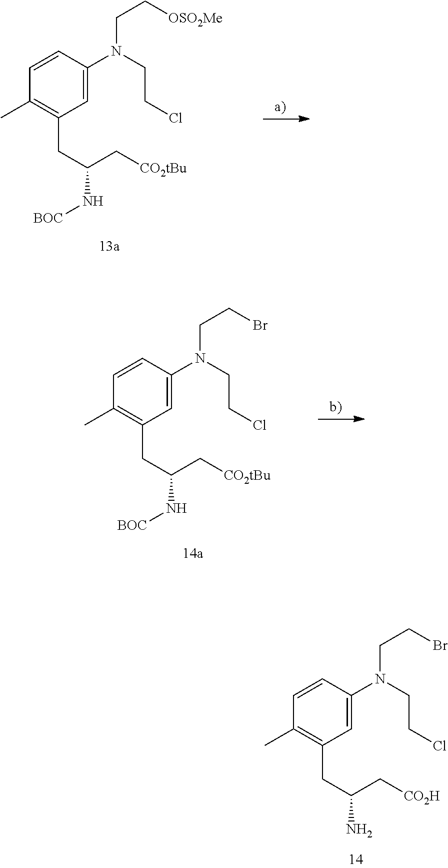

### Step A: tert-Butyl (3R)-4-[5-(2-bromoethyl(2-chloroethyl)amino)-2-methyl-phenyl]-3-(tert-butoxycarbonylamino)butanoate (14a)

Following the General Procedure of Description 19, tert-butyl (3R)-4-[5-(2-bromoethyl(2-chloroethyl)amino)-2-methyl-phenyl]-3-(tert-butoxycarbonylamino)butanoate (14a) is prepared from tert-Butyl (3R)-3-(tert-butoxycarbonylamino)-4-[5-(2-chloroethyl(2-methylsulfonyloxyethyl)amino)-2-methyl-phenyl]butanoate (13a) (1.10 g, 2.0 mmol) through reaction with lithium chloride (LiBr) (191 mg, 2.2 mmol) in anhydrous acetonitrile (MeCN) (10 mL) at reflux temperature for about 2 h to yield the title compound (14a) after aqueous work-up and purification by silica gel column chromatography.

### Step B: (3R)-3-Amino-4-[5-(2-bromoethyl(2-chloroethyl)amino)-2-methyl-phenyl]butanoic acid (14)

Following the General Procedure of Description 9 (Variant A), (3R)-3-amino-4-[5-(2-bromoethyl(2-chloroethyl)amino)-2-methyl-phenyl]butanoic acid (14) is prepared from tert-butyl (3R)-4-[5-(2-bromoethyl(2-chloroethyl)amino)-2-methyl-phenyl]-3-(tert-butoxycarbonylamino)butanoate (14a) (533 mg, 1.0 mmol) through deprotection in a trifluoroacetic acid (TFA)/dichloromethane (DCM) mixture (TFA/DCM=1:1 v/v, 10 mL) at room temperature for about 6 h to yield the target compound (14) as a ditrifluoroacetate salt after evaporation and lyophilization from an aqueous acetonitrile solution. The material may be further purified by preparative RP-HPLC followed using a water/acetonitrile/0.1 vol-% formic acid gradient followed by lyophilization.

### Example 15

### (3R)-3-Amino-4-[5-(2-bromoethyl(2-methylsulfonyloxyethyl)amino)-2-methyl-phenyl]butanoic acid (15)

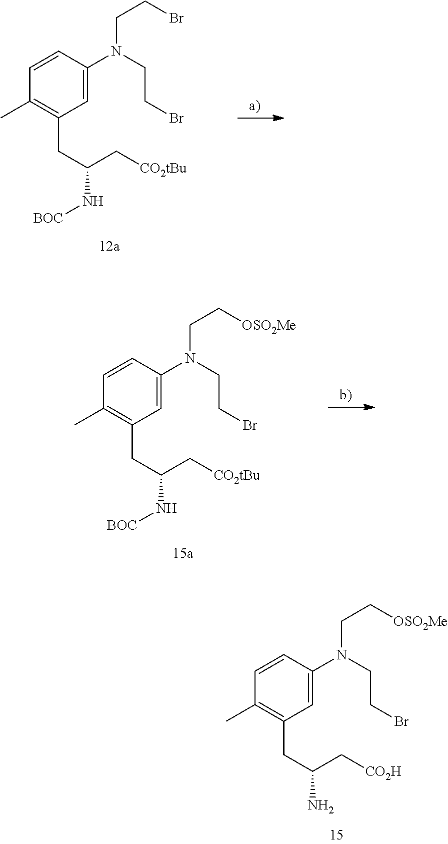

### Step A: tert-Butyl (3R)-4-[5-(2-bromoethyl(2-methylsulfonyloxyethyl)amino)-2-methyl-phenyl]-3-(tert-butoxycarbonylamino)butanoate (15a)

Adapting literature known protocols (Emmons and A. F. Ferris, J. Am Chem. Soc. 1953, 75(9), 2257-2257), tert-butyl (3R)-4-[5-(2-bromoethyl(2-methylsulfonyloxyethyl)amino)-2-methyl-phenyl]-3-(tert-butoxycarbonylamino)butanoate (15a) is prepared from tert-butyl (3R)-4-[5-(bis(2-bromoethyl)amino)-2-methyl-phenyl]-3-(tert-butoxycarbonylamino)butanoate (12a) (1.16 g, 2.0 mmol) with silver methanesulfonate (silver mesylate, AgOMs) (365 mg, 1.8 mmol) in anhydrous acetonitrile (MeCN) (8 mL) at reflux temperature for about 1 h under exclusion of light. Aqueous work-up and purification by silica gel column chromatography afford the title compound (15a).

### Step B: (3R)-3-Amino-4-[5-(2-bromoethyl(2-methylsulfonyloxyethyl)amino)-2-methyl-phenyl]butanoic acid (15)

Following the General Procedure of Description 9 (Variant A), (3R)-3-amino-4-[5-(2-bromoethyl(2-methylsulfonyloxyethyl)amino)-2-methyl-phenyl]butanoic acid (15) is prepared from tert-butyl (3R)-4-[5-(2-bromoethyl(2-methylsulfonyloxyethyl)amino)-2-methyl-phenyl]-3-(tert-butoxycarbonylamino)butanoate (15a) (594 mg, 1.0 mmol) through deprotection in a trifluoroacetic acid (TFA)/dichloromethane (DCM) mixture (TFA/DCM=1:1 v/v, 10 mL) at room temperature for about 6 h to yield the target compound (15) as a ditrifluoroacetate salt after evaporation and lyophilization from an aqueous acetonitrile solution. The material may be further purified by preparative RP-HPLC followed using a water/acetonitrile/0.1 vol-% formic acid gradient followed by lyophilization.

### Example 16

### (3S)-3-Amino-4-[[5-[bis(2-chloroethyl)amino]-2-methyl-phenyl]amino]-4-oxo-butanoic acid (16)

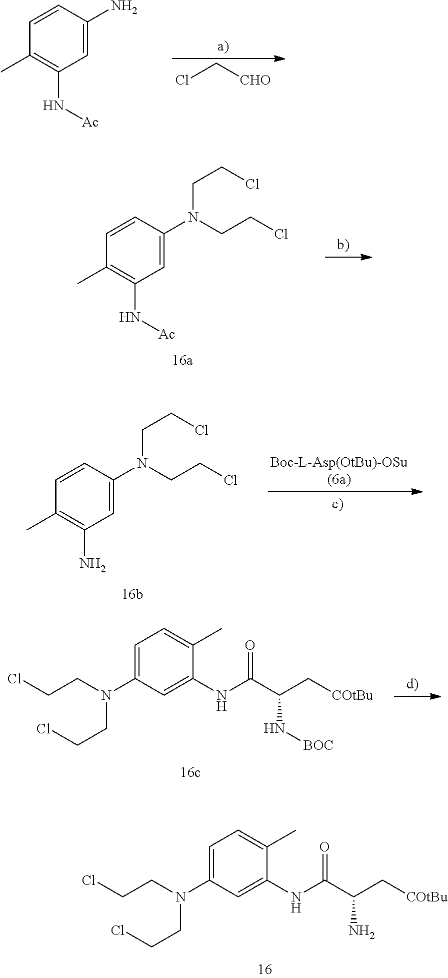

### Step A: N-[5-[Bis(2-chloroethyl)amino]-2-methyl-phenyl]acetamide (16a)

Following the General Procedure of Description 7 (Variant A), N-[5-[bis(2-chloroethyl)amino]-2-methyl-phenyl]acetamide (16a) is prepared from commercial N-(5-amino-2-methylphenyl)acetamide (161 mg, 1.0 mmol), 2-chloroacetaldehyde (˜50 wt-% in water, ˜7.87 M) (700 μL, 5.51 mmol), and sodium cyanoborohydride (NaBH₃CN) (264 mg of 95% purity=251 mg, 4.0 mmol) in a mixture of methanol (MeOH) (6 mL) and trifluoroacaetic acid (3 mL). Aqueous work-up and purification by silica gel column chromatography furnish the title compound (16a).

### Step B: N¹,N¹-Bis(2-chloroethyl)-4-methyl-benzene-1,3-diamine (16b)

Following the General Procedure of Description 8, N¹,N¹-bis(2-chloroethyl)-4-methyl-benzene-1,3-diamine (16b) is prepared from methyl N-[5-[bis(2-chloroethyl)amino]-2-methyl-phenyl]acetamide (16a) (289 mg, 1.0 mmol) by hydrolysis in concentrated hydrochloric acid (HCl) (about 5 mL) at reflux for about 2 hours to afford the title compound (16b) as a solid dihydrochloride salt after isolation using evaporation and lyophilization. The material thus obtained can be used directly in the nest step without further isolation and purification in the next step.

### Step C: tert-Butyl (3S)-4-[[5-[bis(2-chloroethyl)amino]-2-methyl-phenyl]amino]-3-(tert-butoxycarbonylamino)-4-oxo-butanoate (16c)

Adapting a literature known protocol (Levi and Weed, U.S. Pat. No. 3,235,594 (1966)), to a solution of O¹-(2,5-Dioxopyrrolidin-1-yl) O⁴-tert-butyl (2S)-2-(tert-butoxycarbonylamino)-butanedioate (Boc-L-Asp(Osu)-OtBu) (6a) (386 mg, 1.0 mmol) in anhydrous acetonitrile (MeCN) (10 mL) is added N¹,N¹-bis(2-chloroethyl)-4-methyl-benzene-1,3-diamine (16b) as a bis hydrochloride salt (320 mg, 1.0 mmol) followed by neat triethylamine (Et₃N, TEA) (321 μL, 233 mg, 2.3 mmol). The reaction mixture is stirred for about 12 h at room temperature. The reaction is followed by TLC and/or LC/MS to completion. The volatile solvents are removed under reduced pressure using a rotary evaporator. Aqueous work-up and purification by silica gel column chromatography furnish the target compound (16c).

### Step D: (3S)-3-Amino-4-[[5-[bis(2-chloroethyl)amino]-2-methyl-phenyl]amino]-4-oxo-butanoic acid (16)

Following the General Procedure of Description 9 (Variant B), (3S)-3-amino-4-[[5-[bis(2-chloroethyl)amino]-2-methyl-phenyl]amino]-4-oxo-butanoic acid (16) is prepared from tert-butyl (3S)-4-[[5-[bis(2-chloroethyl)amino]-2-methyl-phenyl]amino]-3-(tert-butoxycarbonylamino)-4-oxo-butanoate (16c) (518 mg, 1.0 mmol) in 2.0 N HCl in diethyl ether (2.0 N HCl in Et₂O) (10 mL, 20 mmol) to yield the target compound (15) as an solid dihydrochloride salt after evaporation of the solvents and lyophilization from an aqueous solution. The material may be further purified by preparative HPLC followed by lyophilization. Optionally, the lyophilization is conducted in the presence of 1 equivalent of 1.0 M hydrochloric acid (HCl).

### Example 17

### (3S)-3-Amino-4-[2-[bis(2-chloroethyl)amino]phenoxy]butanoic acid (17)

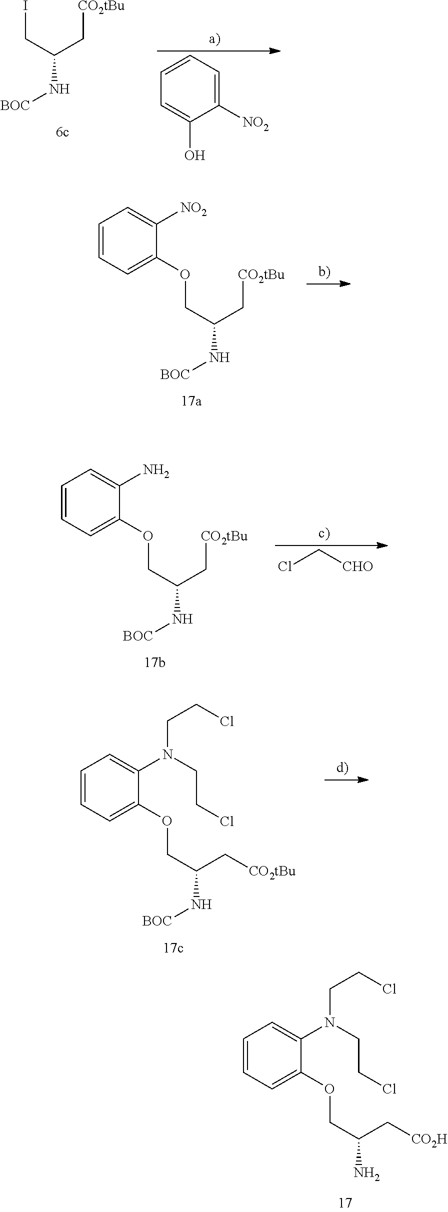

### Step A: tert-Butyl (3S)-3-(tert-butoxycarbonylamino)-4-(2-nitrophenoxy)butanoate (17a)

Adapting literature procedures (Bookster, et al., International Publication No. WO 2010/047982), tert-butyl (3S)-3-(tert-butoxycarbonylamino)-4-(2-nitrophenoxy)butanoate (17a) is prepared from tert-butyl (3S)-3-(tert-butoxycarbonylamino)-4-iodo-butanoate (6c) (1.16 g, 3.0 mmol) and commercial 2-nitrophenol (558 mg, 4.0 mmol) in the presence of cesium carbonate (Cs₂CO₃) (1.63 g, 5.0 mmol) in anhydrous N,N-dimethylformamide (DMF) (10 mL) at 50° C. (oil bath). Aqueous work-up and purification by silica gel chromatography furnish the title compound (17a).

### Step B: tert-Butyl (3S)-4-(2-aminophenoxy)-3-(tert-butoxycarbonylamino)butanoate (17b)

Following the General Procedure of Description 6 (Variant B), tert-butyl (3S)-4-(2-aminophenoxy)-3-(tert-butoxycarbonylamino)butanoate (17b) is prepared by catalytic reduction of tert-butyl (3S)-3-(tert-butoxycarbonylamino)-4-(2-nitrophenoxy)butanoate (17a) (793 mg, 2.0 mmol) in the presence of 10 wt-% palladium on charcoal (Pd/C) containing ˜50 wt-% water (˜350 mg) in methanol (MeOH) (20 mL) and under an atmosphere of hydrogen (˜15 psi, H₂-balloon). The crude material, after filtration over Celite® 545, may be used directly and without further isolation in the next step or may be purified by silica gel chromatography to afford the title compound (17b).

### Step C: tert-Butyl (3S)-4-[2-[bis(2-chloroethyl)amino]phenoxy]-3-(tert-butoxycarbonyl-amino)butanoate (17c)

Following the General Procedure of Description 7 (Variant C), tert-butyl (3S)-4-[2-[bis(2-chloroethyl)amino]phenoxy]-3-(tert-butoxycarbonyl-amino)butanoate (17c) is prepared from tert-butyl (3S)-4-(2-aminophenoxy)-3-(tert-butoxycarbonylamino)butanoate (17b) (733 mg, 2.0 mmol), 2-chloroacetaldehyde (˜50 wt-% in water, ˜7.87 M) (1.27 mL, 10.0 mmol), and sodium cyanoborohydride (NaBH₃CN) (529 mg of 95% purity=503 mg, 8.0 mmol) in a mixture of methanol (MeOH) (10 mL) and 85 wt-% phosphoric acid (H₃PO₄) (3 mL). Aqueous work-up and purification by silica gel column chromatography furnish the title compound (17c).

### Step D: (3S)-3-Amino-4-[2-[bis(2-chloroethyl)amino]phenoxy]butanoic acid (17)

Following the General Procedure of Description 9 (Variant B), (3S)-3-amino-4-[2-[bis(2-chloroethyl)amino]phenoxy]butanoic acid (17) is prepared from tert-butyl (3S)-4-[2-[bis(2-chloroethyl)amino]phenoxy]-3-(tert-butoxycarbonyl-amino)butanoate (17c) (491 mg, 1.0 mmol) in 2.0 N HCl in diethyl ether (2.0 N HCl in Et₂O) (10 mL, 20 mmol) to yield the target compound (17) as an solid dihydrochloride salt after evaporation of the solvents and lyophilization from an aqueous solution. The material may be further purified by preparative HPLC followed by lyophilization. Optionally, the lyophilization is conducted in the presence of 1 equivalent of 1.0 M hydrochloric acid (HCl).

### Example 18

### (3R)-3-Amino-5-[5-[bis(2-chloroethyl)amino]-2-methyl-phenyl]pentanoic acid (18)

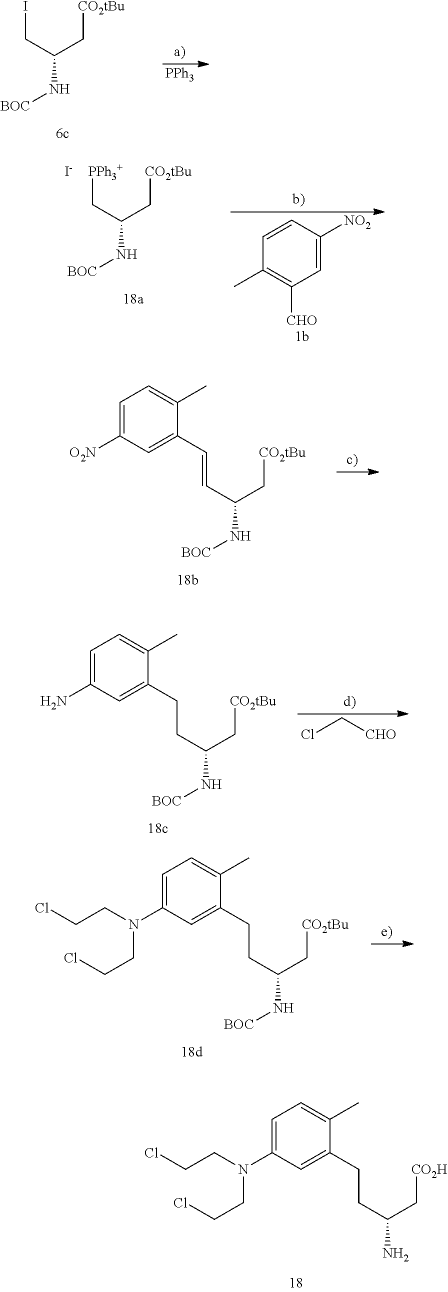

### Step A: [(2S)-4-tert-Butoxy-2-(tert-butoxycarbonylamino)-4-oxo-butyl]-triphenyl-phosphonium iodide (18a)

Adapting a literature procedure (Rémond, et al, J. Org. Chem., J. Org. Chem. 2012, 77, 7579-7587), [(2S)-4-tert-butoxy-2-(tert-butoxycarbonylamino)-4-oxo-butyl]-triphenyl-phosphonium iodide (18a) is prepared from tert-butyl (3S)-3-(tert-butoxycarbonylamino)-4-iodo-butanoate (6c) (1.16 g, 3.0 mmol) and triphenylphosphine (Ph₃P) (1.81 g, 6.9 mmol) at 80° C. under a nitrogen atmosphere. After cooling to room temperature and tritruation with toluene and diethyl ether (Et₂O), the residue is purified by silica gel column chromatography with an acetone and hexane mixture.

### Step B: tert-Butyl (3S)-3-(tert-butoxycarbonylamino)-5-(2-methyl-5-nitro-phenyl)pent-4-enoate (18b)

Adapting a literature procedure (Jandeleit et al., U.S. Pat. No. 8,168,617 (2012)), tert-butyl (3S)-3-(tert-butoxycarbonylamino)-5-(2-methyl-5-nitro-phenyl)pent-4-enoate (18b) is prepared from [(2S)-4-tert-butoxy-2-(tert-butoxycarbonylamino)-4-oxo-butyl]-triphenyl-phosphonium iodide (18a) (1.30 g, 2.0 mmol), 2-methyl-5-nitro-benzaldehyde (1b) (495 mg, 3.0 mmol) (commercial or prepared in two steps from commercial 2-methyl-5-nitro benzoic acid (i) BH₃.SMe₂, THF, reflux, ii) MnO₂, DCM, room temperature) as described in Example 1) with a commercial ˜1.0 M solution of potassium tert-butoxide (KOtBu) (3.0 mL, 3.0 mmol) in anhydrous tetrahydrofuran (THF) (10 mL) at with gradual warming from 00 (ice bath) to room temperature for 24 hours. Aqueous work and purification by silica gel column chromatography furnish the title compound (18b) as a mixture of geometric isomers ((E)/(Z)-isomers).

### Step C: tert-Butyl (3R)-5-(5-amino-2-methyl-phenyl)-3-(tert-butoxycarbonylamino)-pentanoate (18c)

Following the General Procedure of Description 6 (Variant B), tert-butyl (3R)-5-(5-amino-2-methyl-phenyl)-3-(tert-butoxycarbonylamino)-pentanoate (18c) is prepared by catalytic reduction of tert-butyl (3S)-3-(tert-butoxycarbonylamino)-5-(2-methyl-5-nitro-phenyl)pent-4-enoate (18b) (813 mg, 2.0 mmol) in the presence of 10 wt-% palladium on charcoal (Pd/C) containing ˜50 wt-% water (˜40 mg) in methanol (MeOH) (20 mL) and under an atmosphere of hydrogen (˜15 psi, H₂-balloon). The crude material, after filtration over Celite® 545, may be used directly and without further isolation in the next step or may be purified by silica gel chromatography to afford the title compound (18c).

### Step D: tert-Butyl (3R)-5-[5-[bis(2-chloroethyl)amino]-2-methyl-phenyl]-3-(tert-butoxycarbonylamino)pentanoate (18d)

Following the General Procedure of Description 7 (Variant C), tert-butyl (3R)-5-[5-[bis(2-chloroethyl)amino]-2-methyl-phenyl]-3-(tert-butoxycarbonylamino)pentanoate (18d) is prepared from tert-butyl (3R)-5-(5-amino-2-methyl-phenyl)-3-(tert-butoxycarbonylamino)-pentanoate (18c) (757 mg, 2.0 mmol), 2-chloroacetaldehyde (˜50 wt-% in water, ˜7.87 M) (1.27 mL, 10.0 mmol), and sodium cyanoborohydride (NaBH₃CN) (529 mg of 95% purity=503 mg, 8.0 mmol) in a mixture of methanol (MeOH) (10 mL) and 85 wt-% phosphoric acid (H₃PO₄) (3 mL). Aqueous work-up and purification by silica gel column chromatography furnish the title compound (18d).

### Step D: (3R)-3-Amino-5-[5-[bis(2-chloroethyl)amino]-2-methyl-phenyl]pentanoic acid (18)

Following the General Procedure of Description 9 (Variant B), (3R)-3-amino-5-[5-[bis(2-chloroethyl)amino]-2-methyl-phenyl]pentanoic acid (18) is prepared from tert-butyl (3R)-5-[5-[bis(2-chloroethyl)amino]-2-methyl-phenyl]-3-(tert-butoxycarbonylamino)pentanoate (18d) (504 mg, 1.0 mmol) in 2.0 N HCl in diethyl ether (2.0 N HCl in Et₂O) (10 mL, 20 mmol) to yield the target compound (18) as an solid dihydrochloride salt after evaporation of the solvents and lyophilization from an aqueous solution. The material may be further purified by preparative HPLC followed by lyophilization. Optionally, the lyophilization is conducted in the presence of 1 equivalent of 1.0 M hydrochloric acid (HCl).

### Example 19

### (3R)-3-Amino-4-[5-(2-chloroethyl(chloromethyl)carbamoyl)oxy-2-methyl-phenyl]butanoic acid (19)

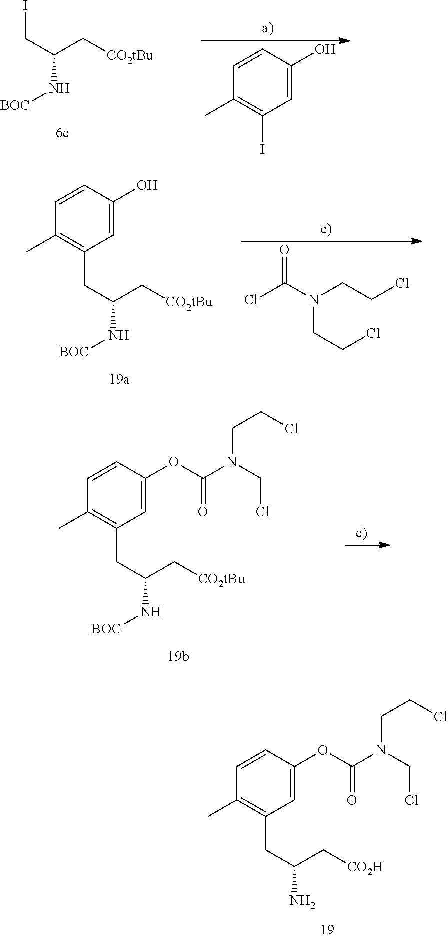

### Step A: tert-Butyl (3R)-3-(tert-butoxycarbonylamino)-4-(5-hydroxy-2-methyl-phenyl)-butanoate (19a)

Following the General Procedure of Description 15 (Part A), zinc dust (Zn) (392 mg, 6.0 mmol) is activated with elemental iodine (I₂) (38 mg, 0.15 mmol, 15 mol-%) and trimethyl silylchloride (MeSiCl, TMSCl) (19 μL, 16 mg, 0.15 mmol, 15 mol-%) in degassed anhydrous N,N-dimethylformamide (DMF) (3 mL). The zinc insertion product is prepared from tert-butyl (3S)-3-(tert-butoxycarbonylamino)-4-iodo-butanoate (6c) (385 mg, 1.0 mmol) in the presence of additional I₂ (38 mg, 0.15 mmol, 15 mol-%) and TMSCl (19 μL, 16 mg, 0.15 mmol, 15 mol-%). Following the General Procedure of Description 15 (Part B), the zinc insertion product of (6c) is used in situ to cross couple with commercial 3-iodo-4-methyl-phenol (234 mg, 1.0 mmol) in the presence of tris(benzylideneacetone) dipalladium (Pd₂(dba)₃) (23 mg, 0.025 mmol, 2.5 mol-%) and tris(o-tolyl)phosphine (P(o-tol)₃) (30 mg, 0.10 mmol, 10 mol-%) in anhydrous degassed DMF (3 mL). Filtration, aqueous work-up, and purification by silica gel column chromatography furnish the title compound (19a).

### Step B: tert-Butyl (3R)-3-(tert-butoxycarbonylamino)-4-[5-(2-chloroethyl(chloromethyl)-carbamoyl)oxy-2-methyl-phenyl]butanoate (19b)

Adapting a literature known protocol (Fex, et al., U.S. Pat. No. 3,299,104), tert-butyl (3R)-3-(tert-butoxycarbonylamino)-4-[5-(2-chloroethyl(chloromethyl)-carbamoyl)oxy-2-methyl-phenyl]butanoate (19b) is prepared through carbamoylation of tert-butyl (3S)-3-(tert-butoxycarbonylamino)-4-(5-hydroxy-2-methyl-phenyl)-butanoate (19a) (731 mg, 2.0 mmol) with commercial N,N-bis(2-chloroethyl)carbamoyl chloride (439 μL, 614 mg, 3.0 mmol) in anhydrous pyridine (15 mL) at about 0° C. The reaction mixture is stirred with gradual warming to room temperature. The reaction is monitored by TLC and/or LC/MS to completion. Excess of the carbamoyl chloride is destroyed with crushed ice. Aqueous work-up followed by purification through silica gel column chromatography afford the title compound (19b).

### Step C: (3R)-3-Amino-4-[5-(2-chloroethyl(chloromethyl)carbamoyl)oxy-2-methyl-phenyl]butanoic acid (19)

Following the General Procedure of Description 9 (Variant B), (3R)-3-amino-4-[5-(2-chloroethyl(chloromethyl)carbamoyl)oxy-2-methyl-phenyl]butanoic acid (19) is prepared from tert-butyl (3R)-3-(tert-butoxycarbonylamino)-4-[5-(2-chloroethyl(chloromethyl)-carbamoyl)oxy-2-methyl-phenyl]butanoate (19b) (519 mg, 1.0 mmol) in 2.0 N HCl in diethyl ether (2.0 N HCl in Et₂O) (10 mL, 20 mmol) to yield the target compound (19) as an solid dihydrochloride salt after evaporation of the solvents and lyophilization from an aqueous solution. The material may be further purified by preparative HPLC followed by lyophilization. Optionally, the lyophilization is conducted in the presence of 1 equivalent of 1.0 M hydrochloric acid (HCl).

### Example 20

### (3R)-3-Amino-4-[5-[bis(2-chloroethyl)carbamoyloxymethyl]-2-nitro-phenyl]butanoic acid (20)

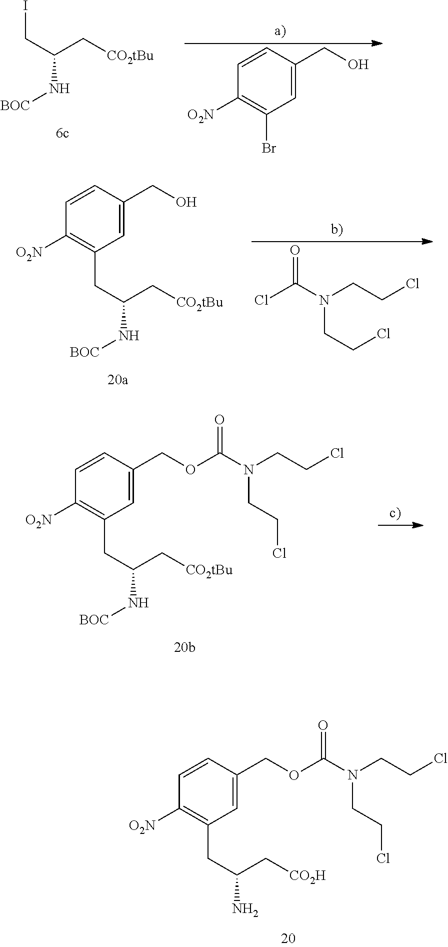 [SMILES: CC(C)(C)OC(=O)C[C@@H](CI)NC(=O)OC(C)(C)C ; O=[N+]([O-])c1ccc(CO)cc1Br ; CC(C)(C)OC(=O)C[C@@H](Cc1cc(CO)ccc1[N+](=O)[O-])NC(=O)OC(C)(C)C ; O=C(Cl)N(CCCl)CCCl ; CC(C)(C)OC(=O)C[C@@H](Cc1cc(COC(=O)N(CCCl)CCCl)ccc1[N+](=O)[O-])NC(=O)OC(C)(C)C ; N[C@@H](CC(=O)O)Cc1cc(COC(=O)N(CCCl)CCCl)ccc1[N+](=O)[O-]]

### Step A: tert-Butyl (3R)-3-(tert-butoxycarbonylamino)-4-[5-(hydroxymethyl)-2-nitro-phenyl]butanoate (20a)

Following the General Procedure of Description 15 (Part A), zinc dust (Zn) (392 mg, 6.0 mmol) is activated with elemental iodine (I₂) (38 mg, 0.15 mmol, 15 mol-%) and trimethyl silylchloride (MeSiCl, TMSCl) (19 μL, 16 mg, 0.15 mmol, 15 mol-%) in degassed anhydrous N,N-dimethylformamide (DMF) (3 mL). The zinc insertion product is prepared from tert-butyl (3S)-3-(tert-butoxycarbonylamino)-4-iodo-butanoate (6c) (385 mg, 1.0 mmol) in the presence of additional I₂ (38 mg, 0.15 mmol, 15 mol-%) and TMSCl (19 μL, 16 mg, 0.15 mmol, 15 mol-%).

Following the General Procedure of Description 15 (Part A), the zinc insertion product of (6c) is used in situ to cross couple with commercial (3-bromo-4-nitro-phenyl)methanol (232 mg, 1.0 mmol) in the presence of tris(benzylideneacetone) dipalladium (Pd₂(dba)₃) (23 mg, 0.025 mmol, 2.5 mol-%) and tris(o-tolyl)phosphine (P(o-tol)₃) (30 mg, 0.10 mmol, 10 mol-%) in anhydrous degassed DMF (3 mL). Filtration, aqueous work-up, and purification by silica gel column chromatography furnish the title compound (20a).

### Step B: tert-Butyl (3R)-4-[5-[bis(2-chloroethyl)carbamoyloxymethyl]-2-nitro-phenyl]-3-(tert-butoxycarbonylamino)butanoate (20b)

Adapting a literature known protocol (Fex, et al., U.S. Pat. No. 3,299,104), tert-butyl (3R)-4-[5-[bis(2-chloroethyl)carbamoyloxymethyl]-2-nitro-phenyl]-3-(tert-butoxycarbonylamino)butanoate (20b) is prepared through carbamoylation of tert-butyl (3R)-3-(tert-butoxycarbonylamino)-4-[5-(hydroxymethyl)-2-nitro-phenyl]butanoate (20a) (731 mg, 2.0 mmol) with commercial N,N-bis(2-chloroethyl)carbamoyl chloride (439 μL, 614 mg, 3.0 mmol) in anhydrous pyridine (15 mL) at about 0° C. The reaction mixture is stirred with gradual warming to room temperature. The reaction is monitored by TLC and/or LC/MS to completion. Excess of the carbamoyl chloride is destroyed with crushed ice. Aqueous work-up followed by purification through silica gel column chromatography afford the title compound (20b).

### Step C: (3R)-3-Amino-4-[5-[bis(2-chloroethyl)carbamoyloxymethyl]-2-nitro-phenyl]butanoic acid (20)

Following the General Procedure of Description 9 (Variant B), (3R)-3-amino-4-[5-[bis(2-chloroethyl)carbamoyloxymethyl]-2-nitro-phenyl]butanoic acid (20) is prepared from tert-Butyl (3R)-4-[5-[bis(2-chloroethyl)carbamoyloxymethyl]-2-nitro-phenyl]-3-(tert-butoxycarbonylamino)butanoate (20b) (578 mg, 1.0 mmol) in 2.0 N HCl in diethyl ether (2.0 N HCl in Et₂O) (10 mL, 20 mmol) to yield the target compound (20) as an solid dihydrochloride salt after evaporation of the solvents and lyophilization from an aqueous solution. The material may be further purified by preparative HPLC followed by lyophilization. Optionally, the lyophilization is conducted in the presence of 1 equivalent of 1.0 M hydrochloric acid (HCl).

### Example 21

### (3R)-3-amino-4-[5-(2-chloroethoxy(2-chloroethyl)amino)-2-methyl-phenyl]butanoic acid (21)

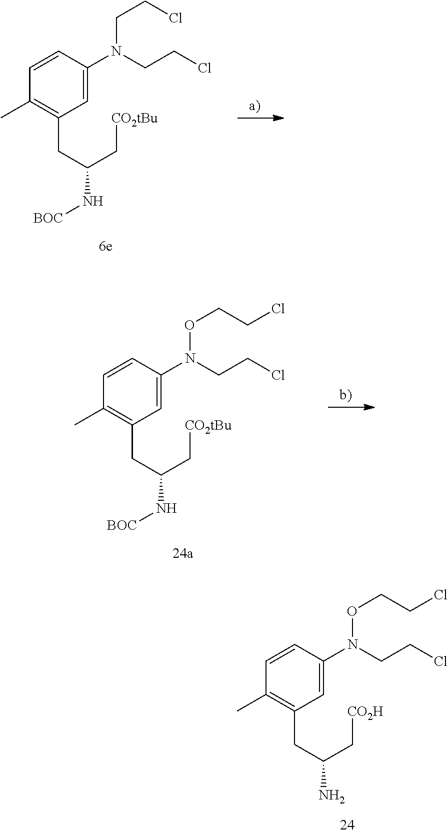 [SMILES: Cc1ccc(N(CCCl)CCCl)cc1C[C@H](CC(=O)OC(C)(C)C)NC(=O)OC(C)(C)C ; Cc1ccc(N(CCCl)OCCCl)cc1C[C@H](CC(=O)OC(C)(C)C)NC(=O)OC(C)(C)C ; Cc1ccc(N(CCCl)OCCCl)cc1C[C@@H](N)CC(=O)O]

### Step A: tert-Butyl (3R)-3-(tert-butoxycarbonylamino)-4-[5-(2-chloroethoxy(2-chloroethyl)amino)-2-methyl-phenyl]butanoate (21a)

Adapting literature known protocols (Tercel, et al., J. Med. Chem. 1995, 38, 1247-1252; Kirkpatrick, U.S. Pat. No. 5,602,278; Kirkpatrick, et al., Anti-Cancer Drugs, 1994, 5, 467-472; and Kirkpatrick, et al., U.S. Pat. No. 7,399,785), tert-butyl (3R)-3-(tert-butoxycarbonylamino)-4-[5-(2-chloroethoxy(2-chloroethyl)amino)-2-methyl-phenyl]butanoate (21a) is prepared by adding 3-chloroperoxybenzoic acid (1.42 g, 80 wt-%, 6.6 mmol) to a solution of tert-butyl (3R)-4-[5-[bis(2-chloroethyl)amino]-2-methyl-phenyl]-3-(tert-butoxycarbonylamino)butanoate (6e) (2.43 g, 5.0 mmol) in dichloromethane (DCM) (30 mL) at about room temperature for about 2 h. The reaction is followed by TLC and/or LC/MS to completion. After quenching with a saturated aqueous solution of sodium hydrogencarbonate (NaHCO₃), the reaction mixture is extracted with DCM (3×). Further aqueous work-up and purification by silica gel column chromatography yield the title compound (21a).

### Step B: (3R)-3-amino-4-[5-(2-chloroethoxy(2-chloroethyl)amino)-2-methyl-phenyl]butanoic acid (21)

Following the General Procedure of Description 9 (Variant B), (3R)-3-amino-4-[5-(2-chloroethoxy(2-chloroethyl)amino)-2-methyl-phenyl]butanoic acid (21) is prepared from tert-butyl (3R)-3-(tert-butoxycarbonylamino)-4-[5-(2-chloroethoxy(2-chloroethyl)amino)-2-methyl-phenyl]butanoate (21a) (506 mg, 1.0 mmol) in 2 N HCl in diethyl ether (2.0 N HCl in Et₂O) (10 mL, 20 mmol) to yield the target compound (21) as an solid dihydrochloride salt after evaporation of the solvents and lyophilization from an aqueous solution. The material may be further purified by preparative HPLC followed by lyophilization. Optionally, the lyophilization is conducted in the presence of 1 equivalent or an excess of 1.0 M hydrochloric acid (HCl).

### Example 22

### 4-[1-(Aminomethyl)-3-hydroxy-1-methyl-3-oxo-propyl]-N,N-bis(2-chloroethyl)-3-methyl-benzeneamine oxide (22)

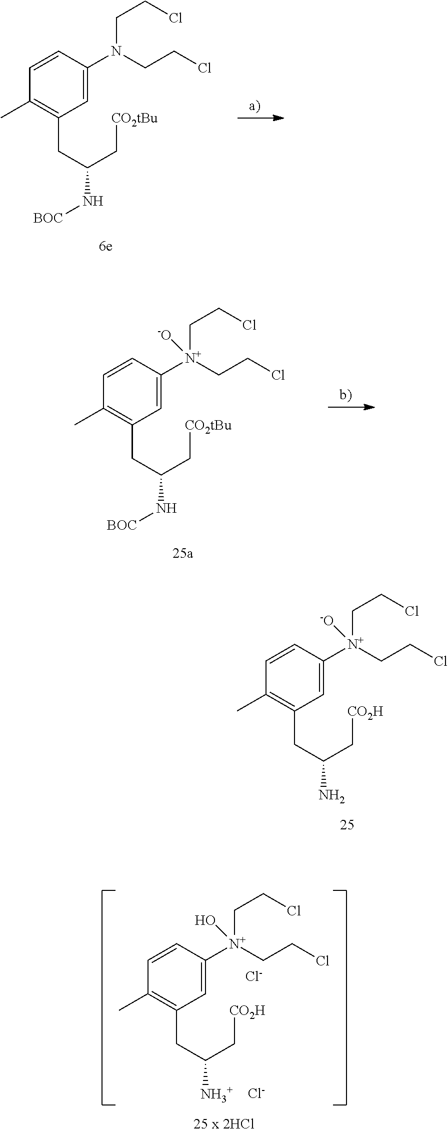

### Step A: 3-[(2R)-4-tert-Butoxy-2-(tert-butoxycarbonylamino)-4-oxo-butyl]-N,N-bis(2-chloroethyl)-4-methyl-benzeneamine oxide (22a)

Adapting literature known protocols (Tercel, et al., J. Med. Chem. 1995, 38, 1247-1252; and Kirkpatrick, et al., U.S. Pat. No. 7,399,785), peracetic acid (H₃CCO₃H) is freshly prepared by adding hydrogen peroxide (H₂O₂) (1.5 mL of a 35 wt-% aqueous solution, 14.0 mmol) dropwise to acetic anhydride (Ac₂O) (1.52 mL, 1.65 g, 16.0 mmol). When the reaction mixture is homogeneous, a solution of tert-butyl (3R)-4-[5-[bis(2-chloroethyl)amino]-2-methyl-phenyl]-3-(tert-butoxycarbonylamino)butanoate (6e) (1.61 g, 3.29 mmol) in dichloromethane (DCM) (20 mL) is added with vigorous stirring at about room temperature for about 2 h. The reaction is followed by TLC and/or LC/MS to completion. The reaction is quenched with 2.0 N hydrochloric acid (HCl), and the aqueous layer separated and repeatedly washed with DCM to the organic extracts are colorless. The aqueous phase is evaporated to dryness under reduced pressure, dried over anhydrous sodium sulfate (Na₂SO₄), and partially reduced in volume. Diethyl ether (Et₂O) is added to separate the title compound 3-[(2R)-4-tert-butoxy-2-(tert-butoxycarbonylamino)-4-oxo-butyl]-N,N-bis(2-chloroethyl)-4-methyl-benzeneamine oxide (22a). The material may be purified by silica gel column chromatography.

### Step B: 3-[(2R)-2-Amino-4-hydroxy-4-oxo-butyl]-N,N-bis(2-chloroethyl)-4-methyl-benzeneamine oxide (22)

Following the General Procedure of Description 9 (Variant B), 3-[(2R)-2-amino-4-hydroxy-4-oxo-butyl]-N,N-bis(2-chloroethyl)-4-methyl-benzeneamine oxide (22) is prepared from 3-[(2R)-4-tert-Butoxy-2-(tert-butoxycarbonylamino)-4-oxo-butyl]-N,N-bis(2-chloroethyl)-4-methyl-benzeneamine oxide (22a) (506 mg, 1.0 mmol) in 2 N HCl in diethyl ether (2 N HCl in Et₂O) (10 mL, 20 mmol) to yield the target compound (22) as an solid dihydrochloride salt (22.2HCl) after evaporation of the solvents and lyophilization from an aqueous solution. The material may be further purified by preparative HPLC followed by lyophilization. Optionally, the lyophilization is conducted in the presence of 1 equivalent or an excess of 1.0 M hydrochloric acid (HCl).

### Example 23

### (3R)-3-Amino-4-[2-[bis(2-chloroethyl)carbamoyl]phenyl]butanoic acid (23)

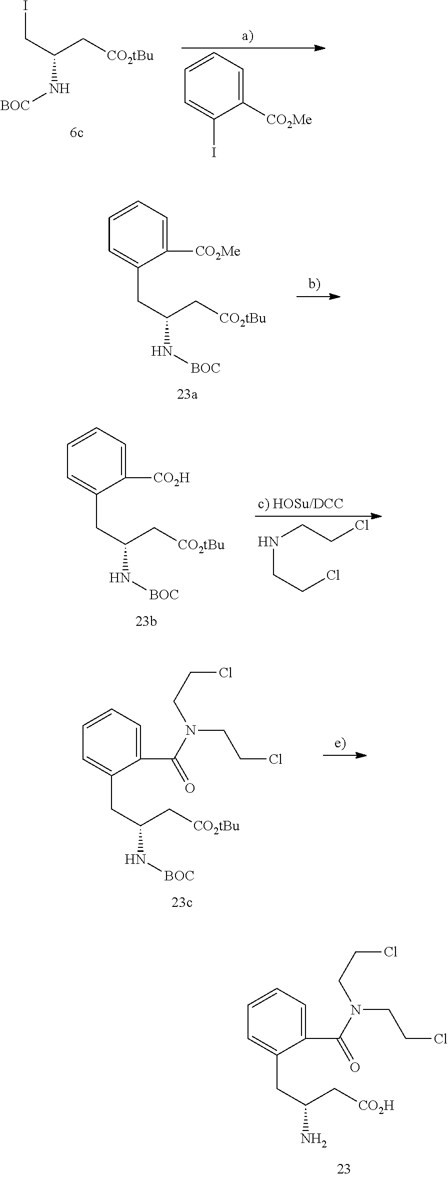

### Step A: Methyl 2-[(2R)-4-tert-butoxy-2-(tert-butoxycarbonylamino)-4-oxo-butyl]benzoate (23a)

Following the General Procedure of Description 15 (Part A), zinc dust (Zn) (392 mg, 6.0 mmol) is activated with elemental iodine (I₂) (38 mg, 0.15 mmol, 15 mol-%) and trimethyl silylchloride (MeSiCl, TMSCl) (19 μL, 16 mg, 0.15 mmol, 15 mol-%) in degassed anhydrous N,N-dimethylformamide (DMF) (3 mL). The zinc insertion product is prepared from tert-butyl (3S)-3-(tert-butoxycarbonylamino)-4-iodo-butanoate (6c) (385 mg, 1.0 mmol) in the presence of additional I₂ (38 mg, 0.15 mmol, 15 mol-%) and TMSCl (19 μL, 16 mg, 0.15 mmol, 15 mol-%).

Following the General Procedure of Description 15 (Part B), the zinc insertion product is used in situ to cross couple with commercial methyl 2-iodobenzoate (262 mg, 1.0 mmol) in the presence of tris(benzylideneacetone) dipalladium (Pd₂(dba)₃) (23 mg, 0.025 mmol, 2.5 mol-%) and tris(o-tolyl)phosphine (P(o-tol)₃) (30 mg, 0.10 mmol, 10 mol-%) in anhydrous degassed DMF (3 mL). Filtration, aqueous work-up, and purification by silica gel column chromatography furnish the title compound (23a).

### Step B: 2-[(2R)-4-tert-Butoxy-2-(tert-butoxycarbonylamino)-4-oxo-butyl]benzoic acid (23b)

Adapting a literature known protocol (Dayal, et al., Steroids, 1990, 55(5), 233-237), a reaction mixture of methyl 2-[(2R)-4-tert-butoxy-2-(tert-butoxycarbonylamino)-4-oxo-butyl]benzoate (23a) (1.97 g, 5.0 mmol) and commercial lithium hydroxide monohydrate (LiOH.H₂O) (420 mg, 10.0 mmol) in a mixture of water (10 mL) and methanol (MeOH) (3 mL) is stirred at room temperature. The reaction is monitored by TLC and/or LC/MS to completion. The solvent is partially removed under reduced pressure using a rotary evaporator. Acidic aqueous work-up and purification by silica gel column chromatography furnish the title compound 2-[(2R)-4-tert-butoxy-2-(tert-butoxycarbonylamino)-4-oxo-butyl]benzoic acid (23b) which may be used directly in the next step without further isolation and purification.

### Step C: tert-Butyl (3R)-4-[2-[bis(2-chloroethyl)carbamoyl]phenyl]-3-(tert-butoxycarbonylamino)butanoate (23c)

Adapting a literature known protocol (Levi and Weed, U.S. Pat. No. 3,235,594), to a reaction mixture of 2-[(2R)-4-tert-butoxy-2-(tert-butoxycarbonylamino)-4-oxo-butyl]benzoic acid (23b) (759 mg, 2.0 mmol), N-hydroxysuccinimide (NHS, HOSu) (235 mg, 2.2 mmol) in anhydrous acetonitrile (MeCN) (10 mL) is added solid dicyclohexylcarbodiimide (DCC) (433 mg, 2.1 mmol) in small portions at about room temperature. The reaction mixture is stirred for about 12 hours and the precipitated dicyclohexylurea side product is filtered off using a Bichner funnel. The filtrate is treated with commercial di-(2-chloroethyl)amine hydrochloride (2-chloro-N-(2-chloroethyl)ethanamine hydrochloride; HN(CH₂—CH₂—Cl)₂.HCl) (393 mg, 2.2 mmol) followed by neat triethylamine (Et₃N, TEA) (321 μL, 233 mg, 2.3 mmol). The reaction mixture is stirred for about 12 hours at room temperature. The reaction is followed by TLC and/or LC/MS to completion. Acidic aqueous work-up and purification by silica gel column chromatography furnish the title compound tert-butyl (3R)-4-[2-[bis(2-chloroethyl)carbamoyl]phenyl]-3-(tert-butoxycarbonylamino)butanoate (23c).

### Step E: (3R)-3-Amino-4-[2-[bis(2-chloroethyl)carbamoyl]phenyl]butanoic acid (23)

Following the General Procedure of Description 9 (Variant B), (3R)-3-amino-4-[2-[bis(2-chloroethyl)carbamoyl]phenyl]butanoic acid (23) is prepared from tert-butyl (3R)-4-[2-[bis(2-chloroethyl)carbamoyl]phenyl]-3-(tert-butoxycarbonylamino)butanoate (23c) (504 mg, 1.0 mmol) in 2 N HCl in diethyl ether (2 N HCl in Et₂O) (10 mL, 20 mmol) to yield the target compound (23) as an solid dihydrochloride salt after evaporation of the solvents and lyophilization from an aqueous solution. The material may be further purified by preparative HPLC followed by lyophilization. Optionally, the lyophilization is conducted in the presence of 1 equivalent or an excess of 1.0 M hydrochloric acid (HCl).

### Example 24

### LAT1 Uptake Inhibition Assays

The ability of compounds to interact with LAT1 was measured using a radiolabeled competition uptake assay with [³H]-Gabapentin (GP) in 96-well plates with LLCPK cells conditionally expressing hLAT1. Five (5)×10⁴ cells/well were plated in white, clear bottom plates in the presence or absence of tetracycline or doxycycline to induce hLAT1 expression. The next day, cells were treated with sodium butyrate to stimulate additional hLAT1 expression. On the third day, the cells were washed and then incubated with 50,000 cpm of [³H]-GP in PBS in the presence or absence of 1 mM of test compound in at least triplicate for 15 min. At end of the assay time, the incubation solution was removed, and the plates were washed three times with 100 μL of ice-cold phosphate buffered saline (PBS) buffer. 150 μL of scintillation fluid was added to each well, and the radioactivity retained within the cells was measured on a 96-well scintillation counter. The data are expressed as a percent of specific [³H]-GP uptake. Unlabeled GP and other large amino acids (phenylalanine and leucine) were used as controls.

The ability of various compounds to interact with LAT1 was assessed by measuring the inhibition of [³H]-GP uptake into LAT1-expressing cells in the presence of 1 mM test compound. Unlabeled GP and phenylalanine (Phe) and leucine (Leu) were used as controls. After incubation for 15 min, cells were washed, scintillation fluid added, and cell-bound radioactivity determined in a scintillation counter. Data are expressed as a percent of specific GP uptake.

The specific uptake of radiolabeled gabapentin into LAT1-expressing cells was inhibited by 1 mM of unlabeled gabapentin, phenylalanine, leucine, and the compounds of Examples 1-4. Treatment with gabapentin, phenylalanine, leucine, and the compound of Example 3 resulted in specific uptake of less than 10%. The compounds of Examples 1, 2, and 4 resulted in specific uptake of greater than 20% but less than 50% at this concentration. The specific uptake of radiolabeled gabapentin in the absence of any compound was 100%.

### Example 25

### LAT1-Specific In Vitro Cytotoxicity Assays

The LAT1-specific in vitro cytotoxicity of compounds was assessed by using a modified clonigenic assay in 96-well plates with LLCPK cells conditionally expressing hLAT1. 1000 cells/well were plated in clear bottom plates in the presence or absence of tetracycline or doxycycline to induce hLAT1 expression. The next day, cells were treated with sodium butyrate to stimulate additional hLAT1 expression. On the third day, cells were washed and incubated with various concentrations of test compounds in PBS buffer in at least quadruplicate for 30 minutes. At the end of the treatment, test compounds were removed and growth media was added to the cells. Clonal populations were allowed to grow until the control wells (mock treatment) were near confluency (7 to 10 days). Cell growth was quantified by fixing and staining the cells post-wash with crystal violent in aqueous glutaraldehyde, washing away unadhered dye, solubilizing the stained cells in acetic acid and monitoring absorbance at 530 nm. Data from each test concentration were expressed as the percent of live, mock-treated controls (% surviving cells). LAT1 specificity was determined by the differential toxicity in cells induced (LAT1+) vs. non-induced (no LAT1) to express hLAT1. Melphalan, a N-mustard compound, was used as a control.

The LAT1-specific cytotoxicity of various compounds was assessed by treating cells expressing or not expressing LAT1 with 3 μM of test compound for 30 min. Melphalan was used as a control compound. Following treatment, cells were washed and growth media was added. Surviving cells were allowed to proliferate for 7-10 days, and then stained and quantified. Results were expressed as the percent of untreated cells (% surviving cells).

The percent surviving cells for melphalan and the compound of Example 2 was about the same in cells expressing LAT1 and in cells not expressing LAT1. The percent surviving cells for the compounds of Examples 1, 3, and 4 was significantly reduced by at least 25% in cells expressing LAT1 compared to cells not expressing LAT1.

The in vitro cytotoxicity of the two enantiomers of QBS10072 was assessed by treating LAT1-expressing cells with various concentrations of the S (solid circles) or the R (open circles) isomer for 30 min. Following treatment, cells were washed and growth media was added. Surviving cells were allowed to proliferate for 7-10 days, and then stained and quantified. Results were expressed as the percent of untreated cells (% surviving cells) and graphed vs. test concentration.

The in vitro cytotoxicity of the two single enantiomers of Example 3 was assessed by treating LAT1-expressing cells with various concentrations of the S (solid circles) or the R (open circles) isomer for 30 min. Following treatment, cells were washed and growth media was added. Surviving cells were allowed to proliferate for 7-10 days, and then stained and quantified. Results were expressed as the percent of untreated cells (% surviving cells) and graphed vs. test concentration.

The in vitro cytotoxicity of the two single enantiomers of compound (3) was assessed by treating LAT1-expressing cells with various concentrations of the S (compound (5)) or the R (compound (6)) isomer for 30 min. Following treatment, cells were washed and growth media was added. Surviving cells were allowed to proliferate for 7-10 days, and then stained and quantified. The S isomer of Example 5 exhibited an IC50 that was significantly less than the IC50 of the R isomer of compound (6). Compounds (1), (3), (5), (7), (9), and (22) exhibited an LC50, the concentration of test compound at which the percent surviving cells was 50%, less 1 μM.

The selectivity of the test compounds for LAT-mediated cytotoxicity was evaluated by comparing the LC50 (μM) for cells high LAT1-expressing cells and low LAT1-expressing cells. Compounds (1), (3), (5), (7), and (9) exhibited an LC50 selectivity ratio (low LAT1/high LAT1) of greater than 5.

### Example 26

### In Vivo Tumor Growth Suppression Assays

The ability to suppress the growth of tumors in vivo was measured using a B16 efficacy model (Kato, et al., Cancer Res., 1994, 54, 5143-5147). Briefly, the hind flank of C57BL/6 mice were injected with 5×10⁵ B16 melanoma cells subcutaneously. Once the tumors reached 40 mm³, animals were separated into various treatment arms (n=5) and dosed IP daily with vehicle or test compound (5 and 10 mg/kg) for 12 days. Tumor sizes were monitored every third day for up to three weeks. Melphalan was used as a control compound (2.5 mg/kg). The results are presented in Table 1.

TABLE 1
Tumor Suppression by QBS Compounds in vivo

Tumor Growth (% Control)

| | End of | |
|---|---|---|
| Treatment | dosing | 5 days post-dosing |
| Vehicle | 100 | 100 |
| QBS10072 | 11 | 11 |
| Melphalan | 33 | 56 |

### Example 27

### In Vitro Bone Marrow Toxicity

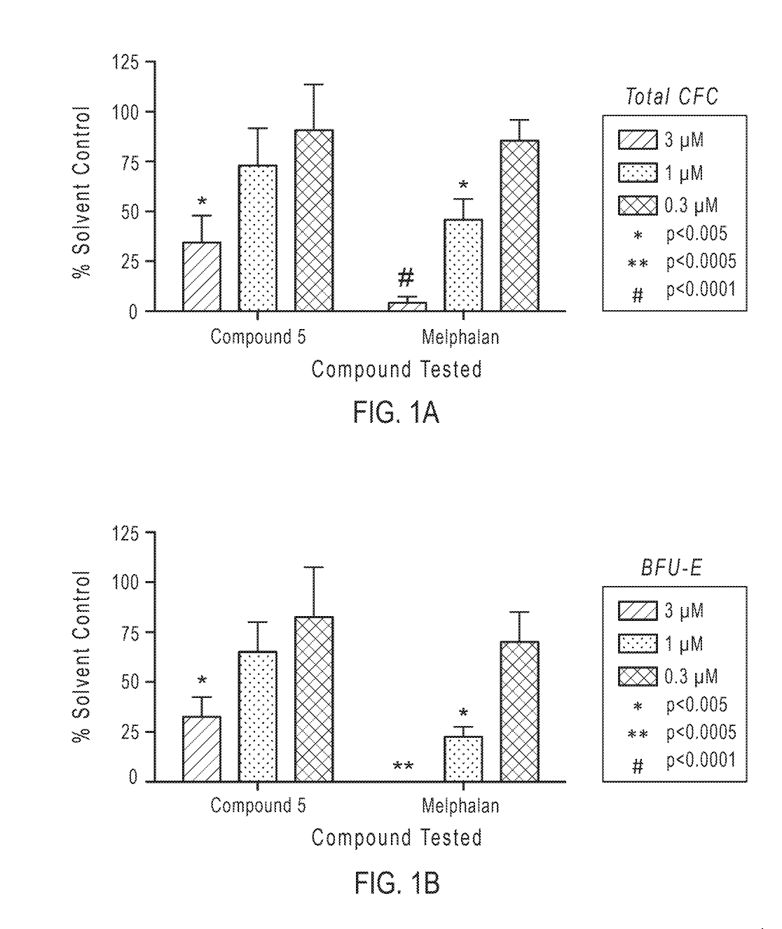

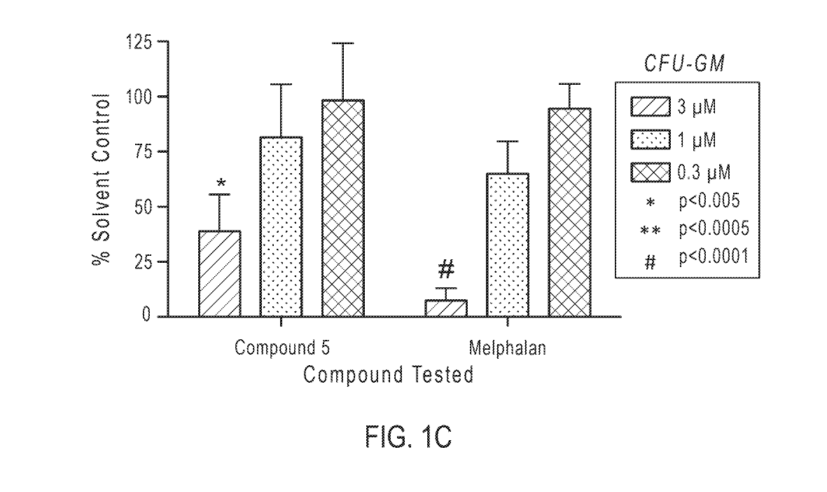

N-mustards and other cytotoxic agents are known to cause bone marrow toxicity, which may be due to non-specific or non-LAT1-mediated transport. The cytotoxicity of compounds on erythroid and myeloid progenitors using human bone marrow cells in in vitro colony forming cell assays was evaluated. Human bone marrow cells were incubated with multiple concentrations of test compounds in the presence of hematopoietic growth factors in a methylcellulose media. Following 14 days in culture, both erythroid and myeloid hematopoietic colonies were assessed and scored. The effects of melphalan (control) and compound (5) were assessed. The results are presented in FIGS. 1A-1C, which demonstrate that melphalan is more toxic to bone marrow progenitor cells than compound (5). In FIGS. 1A-1C, BFU-E refers to blast-forming unit-erythroid; CFU-GM refers to colony-forming unit-granulocytes, macrophages; and Total CFC refers to the sum Total CFC=BFU-E+CFU-GM+CFU-GEMM.

### Example 28

### Mouse Melanoma Efficacy

The efficacy of compounds provided by the present disclosure on mouse melanoma cells was assessed.

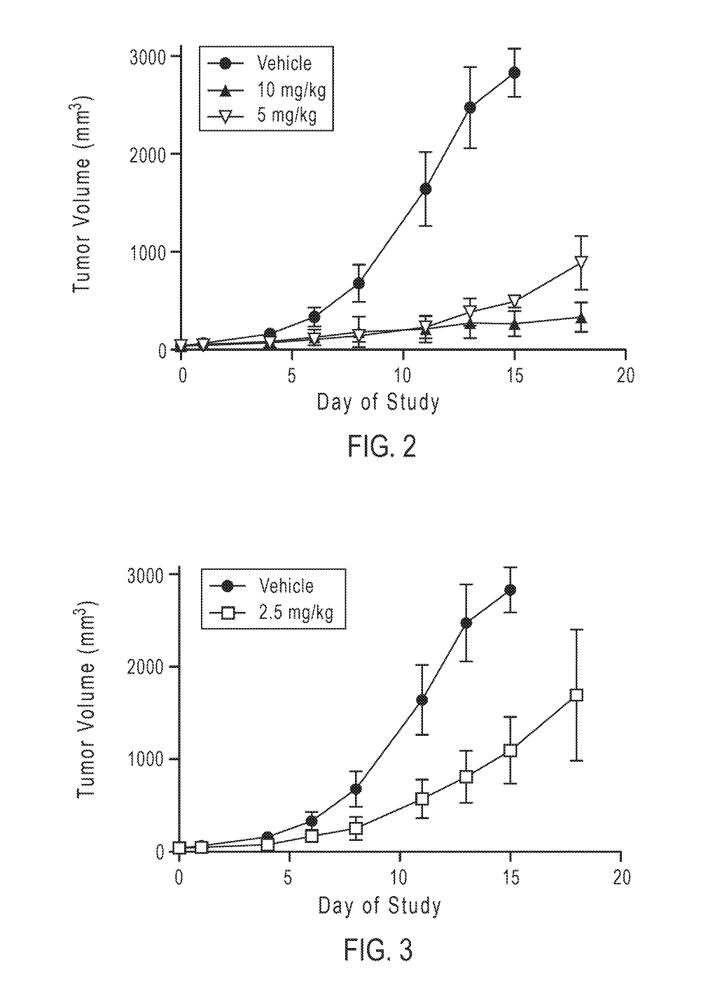

Mouse melanoma cells were implanted into syngeneic mice. Treatment began when the tumors reached a volume of 40 mm³ at which time the animals were dosed daily for 12 days with either melphalan (control) or compound (5). The size of the tumor and the white blood count were measured. The results are presented in FIG. 2 and FIG. 3. The results show that compound 5 suppressed tumor growth. Tumors began to grow after dosing of compound 5 ended at 12 days. The white blood cell count remained within the normal range during treatment with compound (5).

### Example 29

### Acute Toxicity Study

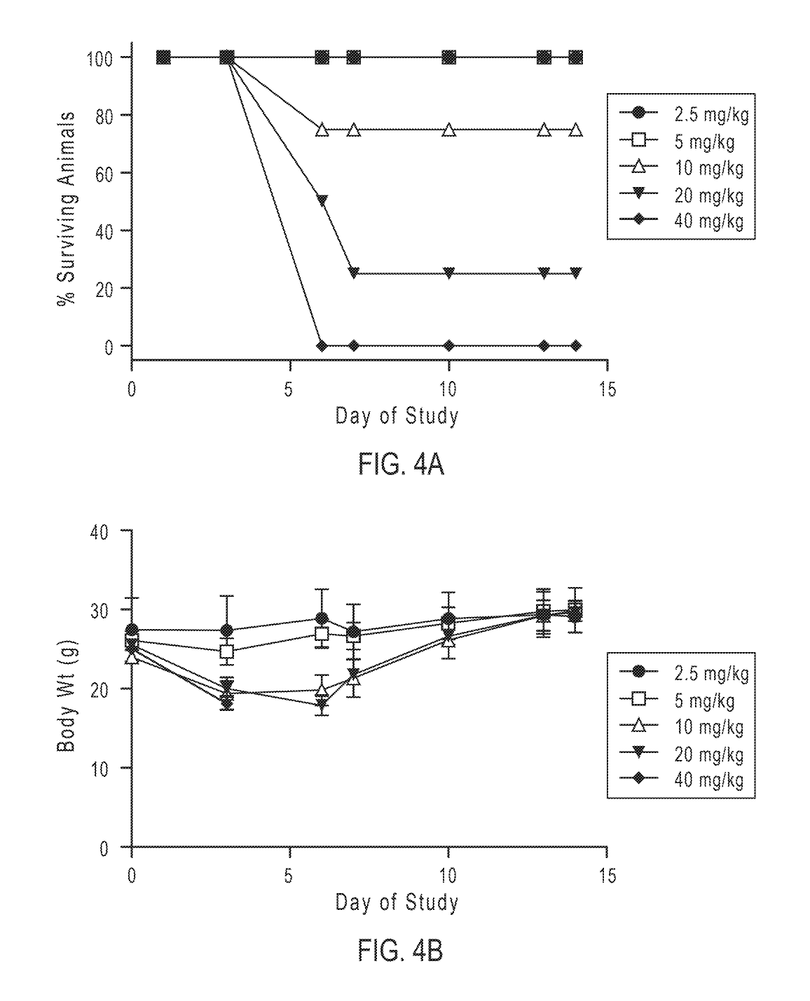

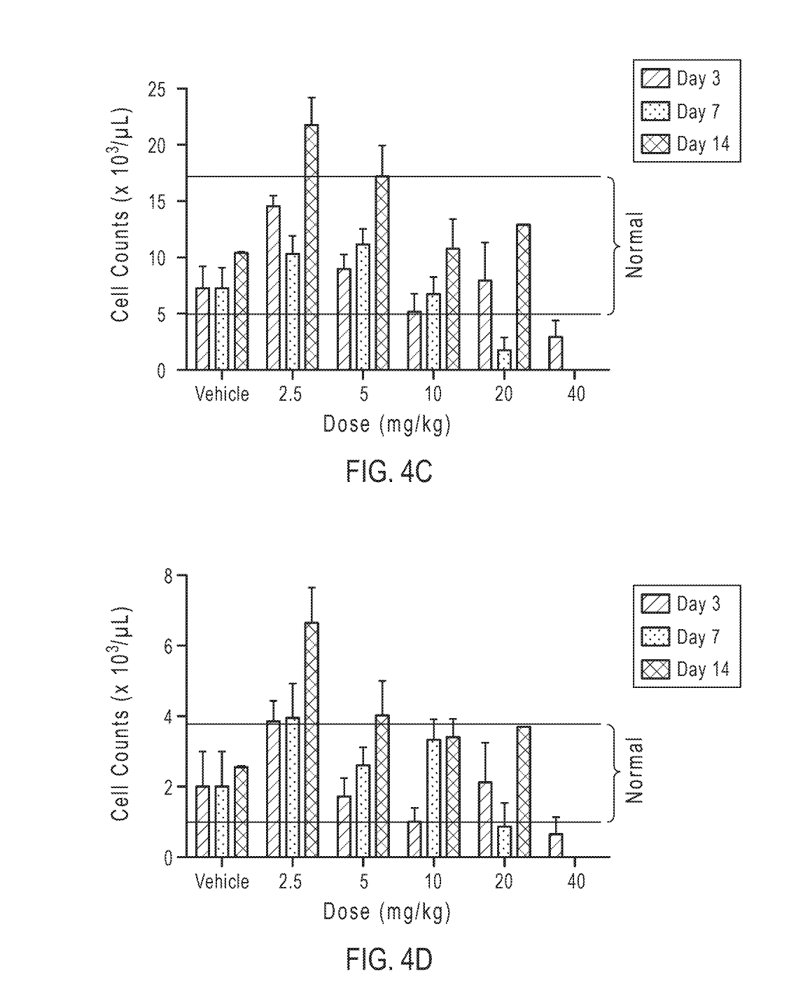

The acute toxicity of compounds provided by the present disclosure was assessed by dosing mice with test compound during a two week period. Compound (5) was administered by intraperitoneal (IP) injection on day 1 of the study and the body weight, white blood cell count, granulocyte count, and general health were monitored over a two week period. Four (4) animals per group were dosed at concentrations of 2.5 mg/kg, 5 mg/kg, 10 mg/kg, 20 mg/kg, and 40 mg/kg of compound (5). Blood samples were analyzed on days 3, 7, and 14. The results are shown in FIGS. 4A-4D.

### Example 30

### Breast Cancer Xenograft Study

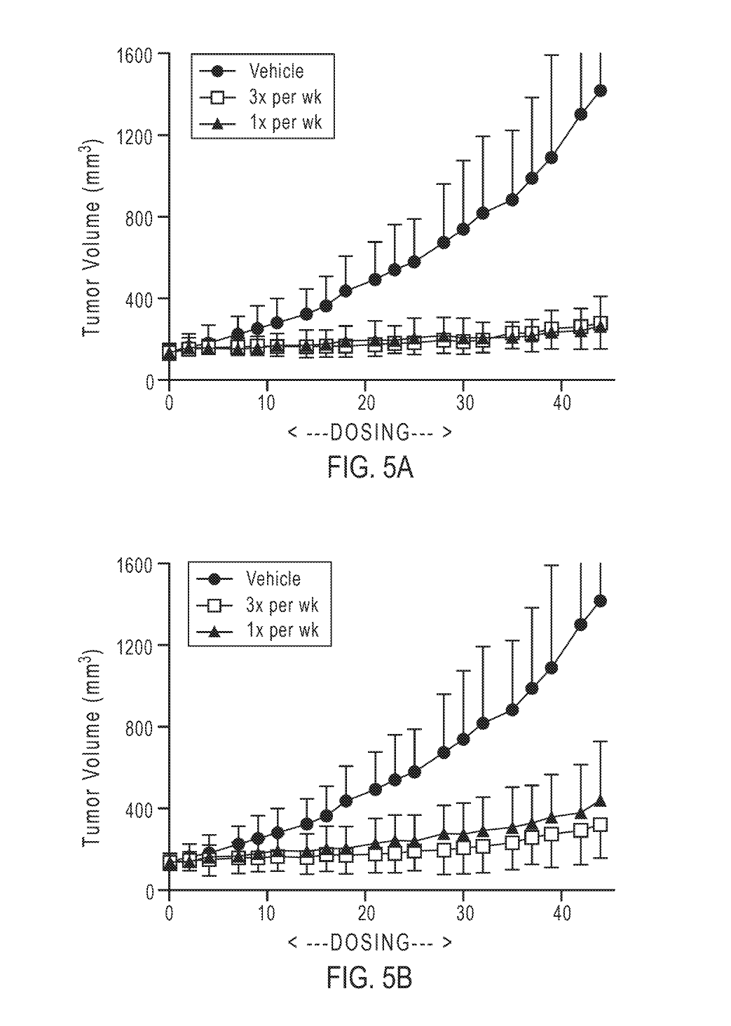

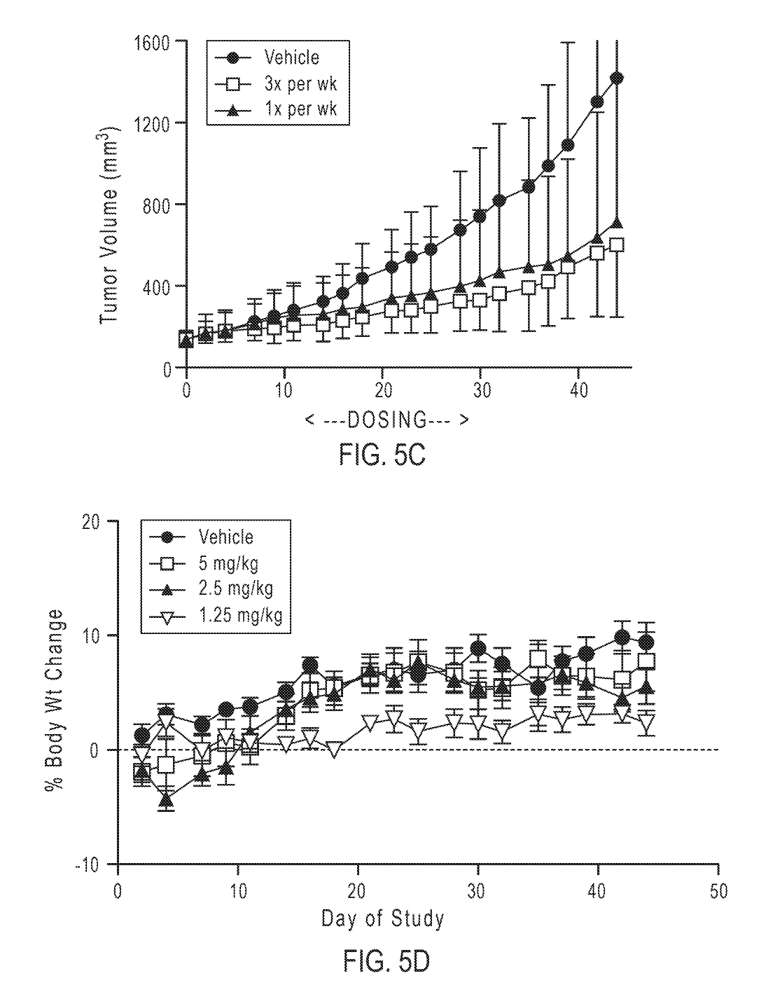

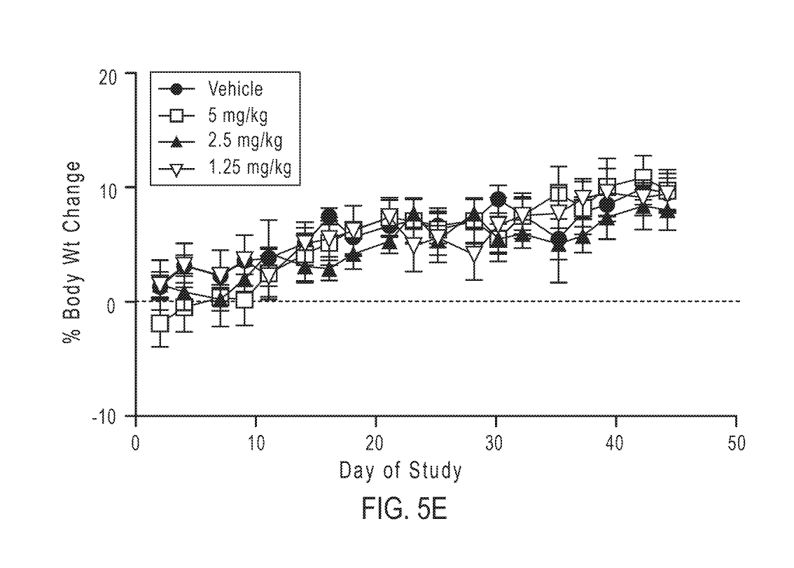

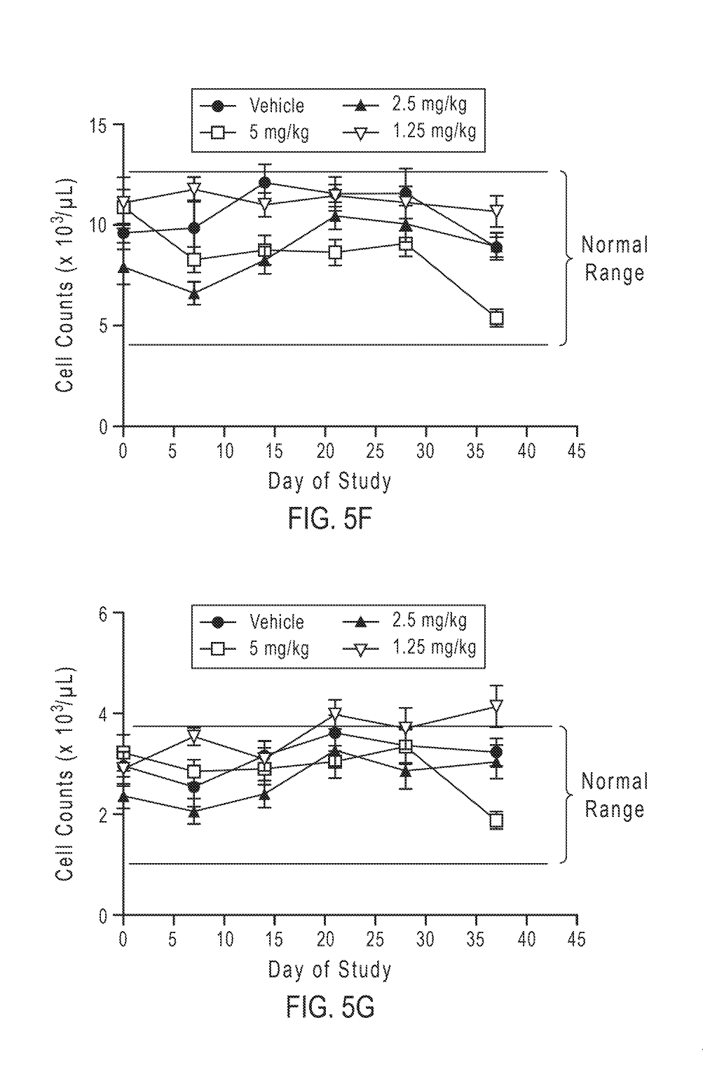

The efficacy of compounds provided by the present disclosure was evaluated on a triple negative breast cancer xenograft model—MDA-MB-231. The tumors were implanted into mice and the study was initiated after the volume of the tumors was 140 mm³. The animals were IP dosed once a week or 3 times per week for 3 weeks with either vehicle or 1.25 mg/kg, 2.5 mg/kg, or 5 mg/kg of compound (5). The tumor volume, body weight, white blood cell count, and granulocyte count were determined during the study and 20 days after the final dose was administered. The results are presented in FIGS. 5A-5G. The growth of the tumors was nearly completely suppressed for the 2.5 mg/kg and 5 mg/kg dosing regimens. Tumor growth remained about 90% suppressed 20 days post-dosing following the 5 mg/kg treatment regimens. The 1.25 mg/kg and 2.5 mg/kg regimens demonstrated a dose response for tumor growth post-treatment. As shown in FIGS. 5D-5E, the animal body weights increased for all dosing regimens; and as shown in FIGS. 5F-5G, the white blood cell count and granulocyte count remained within normal range both during and following treatment with compound (5) for all dosing regiments.

### Example 31

### Prostate Cancer Xenograft Study

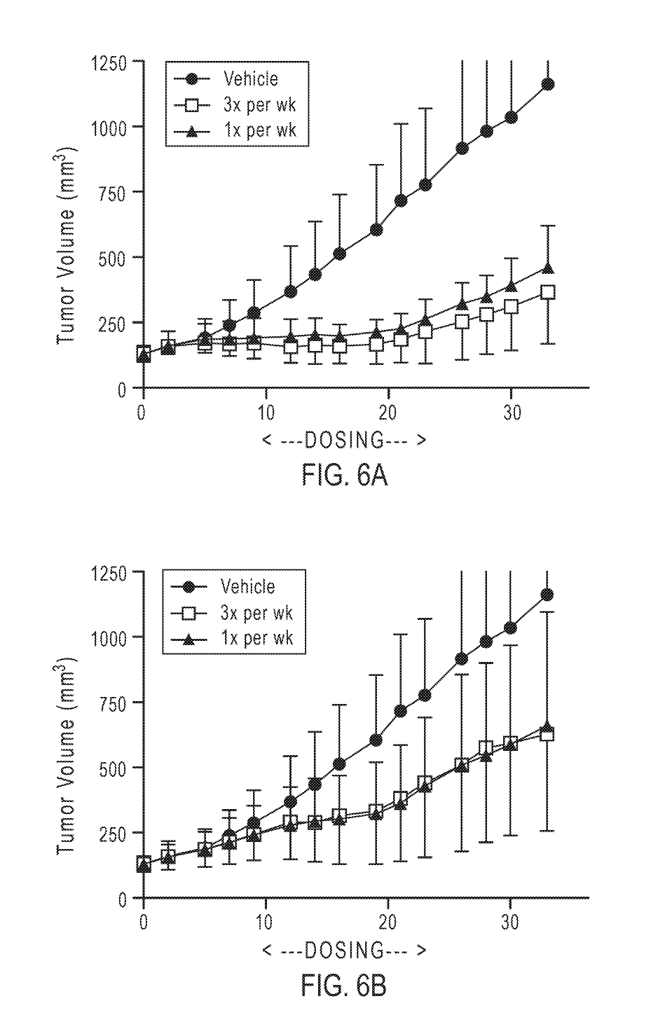

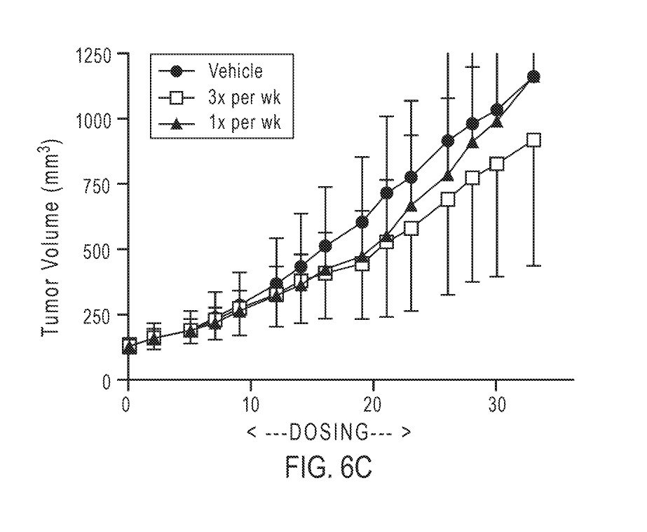

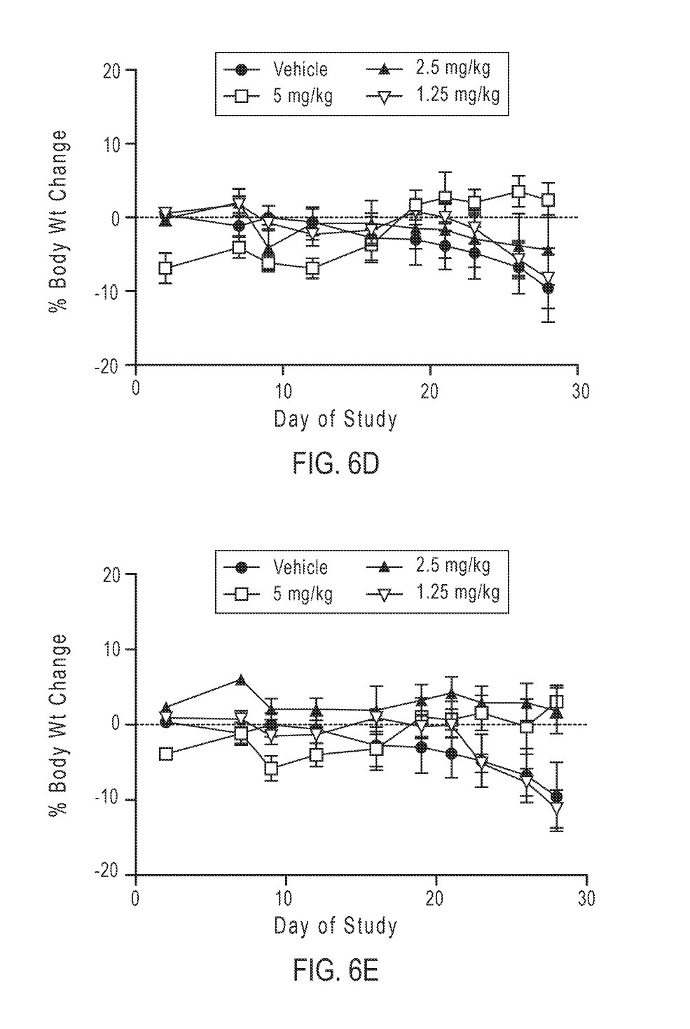

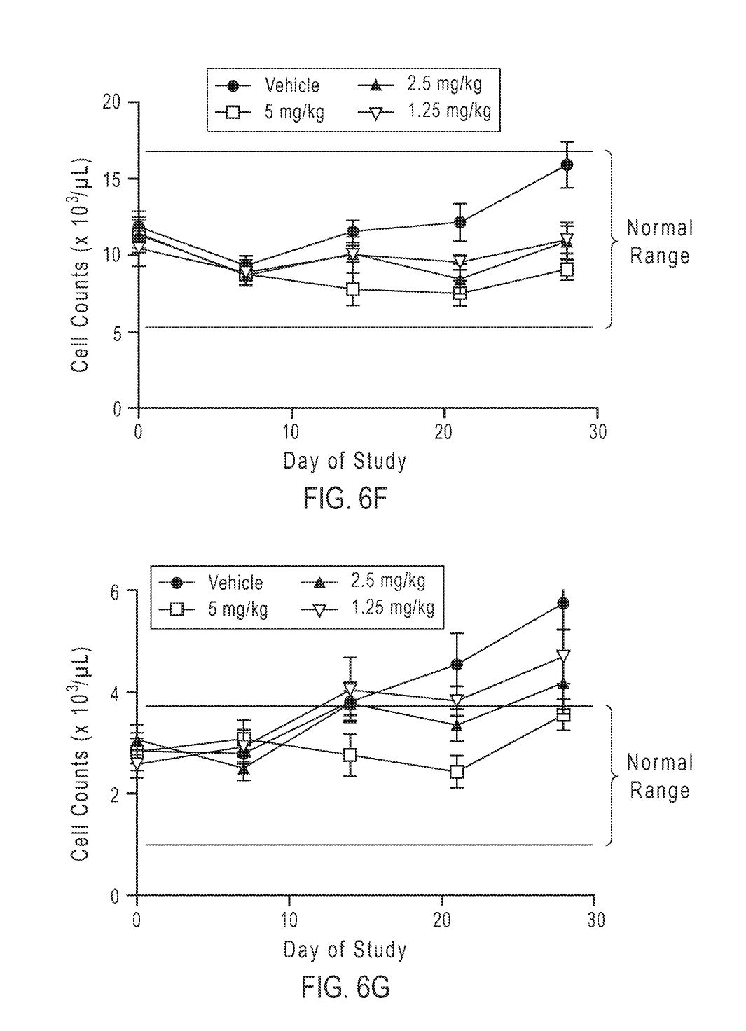

The efficacy of compounds provided by the present disclosure was evaluated on a prostate cancer xenograft model—PC3. PC3 tumors were implanted into mice and the study was initiated after the volume of the tumors was 130 mm³. The animals were IP dosed once a week or 3 times per week for 3 weeks with either vehicle or 1.25 mg/kg, 2.5 mg/kg, or 5 mg/kg of compound (5). The tumor volume, body weight, white blood cell count, and granulocyte count were determined during the study and 10 days after the final dose was administered. The results are presented in FIGS. 6A-6G. The growth of the tumors was nearly completely suppressed for the 5 mg/kg dosing regimen. The 1.25 mg/kg, 2.5 mg/kg, and 5 mg/kg regimens demonstrated a dose response for tumor growth post-treatment. As shown in FIGS. 6D-6E, the animal body weights remained the same increased for all dosing regimens; and as shown in FIGS. 6F-6G, the white blood cell count and granulocyte count remained within normal range both during and following treatment with compound (5) for all dosing regimens.

### Example 32

### Prostate Cancer Xenograft Study—Large Tumor Study

The effect of compounds provided by the present disclosure on the growth of large tumors was evaluated.

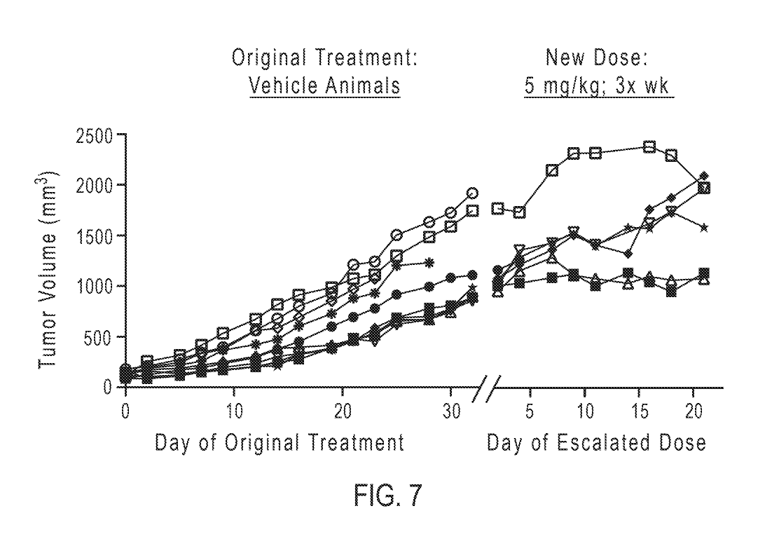

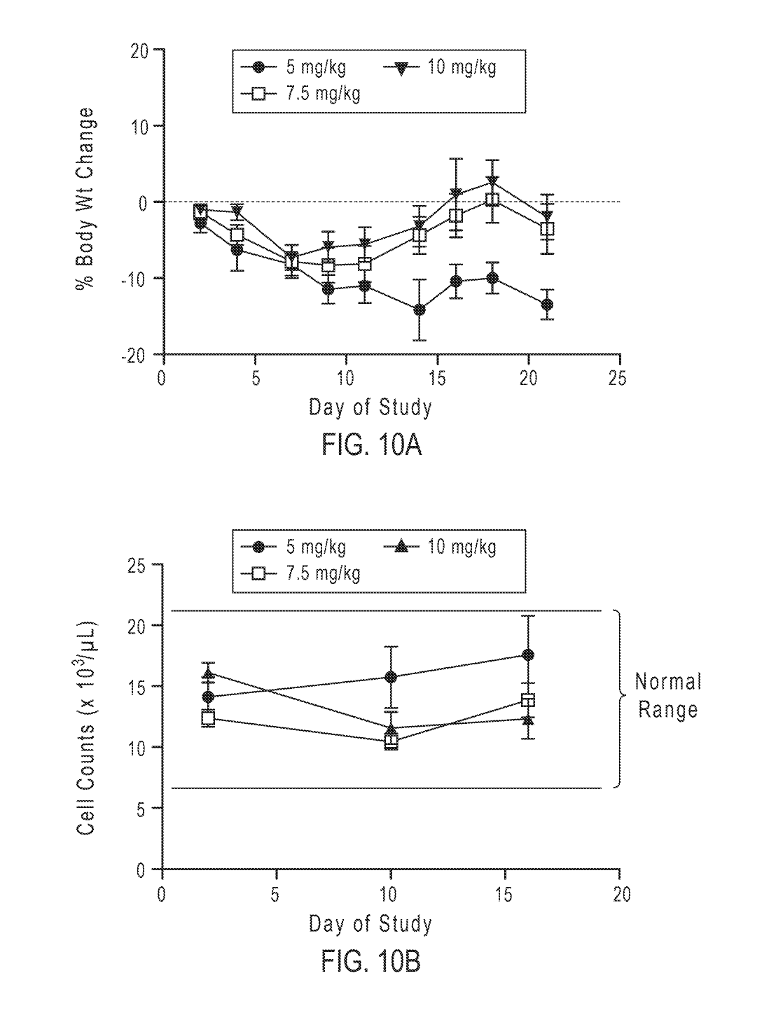

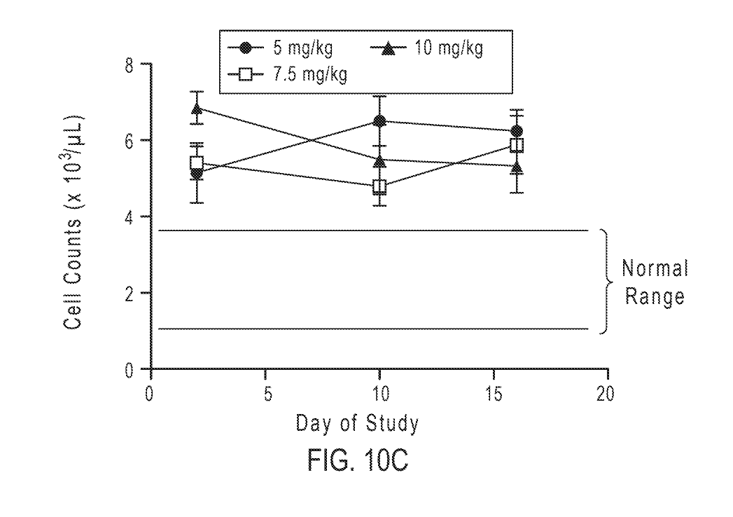

PC3 tumors were implanted into mice and the study was initiated after the volume of the tumors was 500 mm³. Five (5) mg/kg compound (5) was administered by IP injection three times per week for two weeks. The tumor size, body weight, white cell count, and granulocyte count were measured. The results are presented in FIG. 7, and show that the growth rate slowed for some large tumors. As shown in FIGS. 10A-10C, the body weight decreased and the cell counts remained within the normal range during the study.

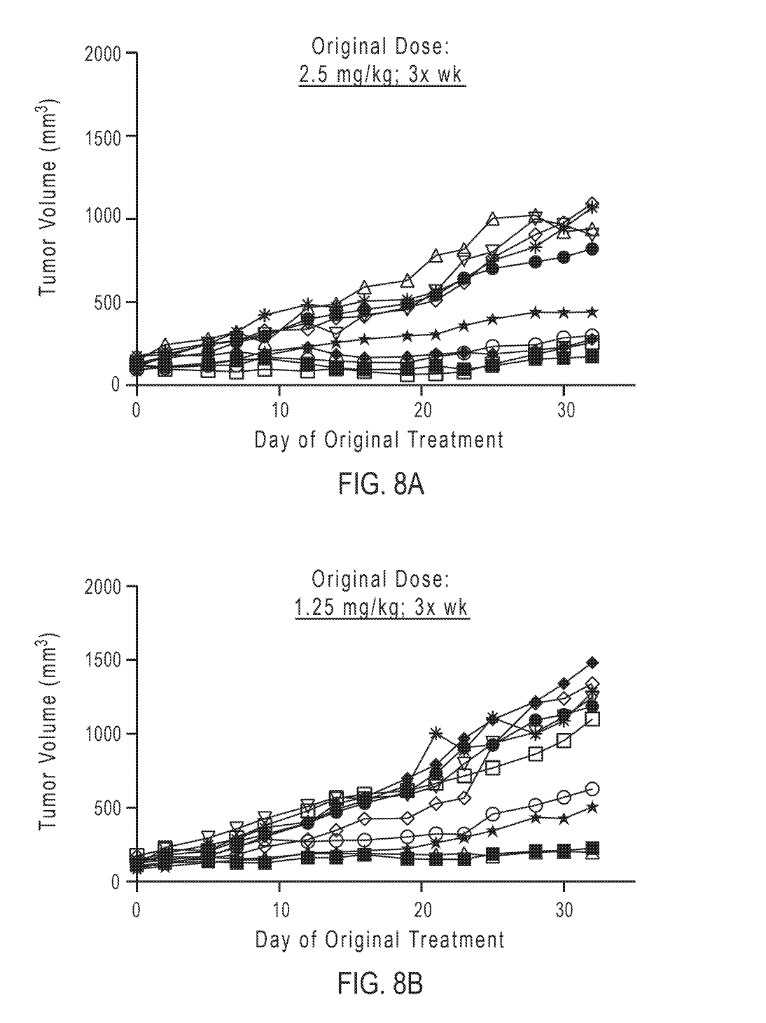

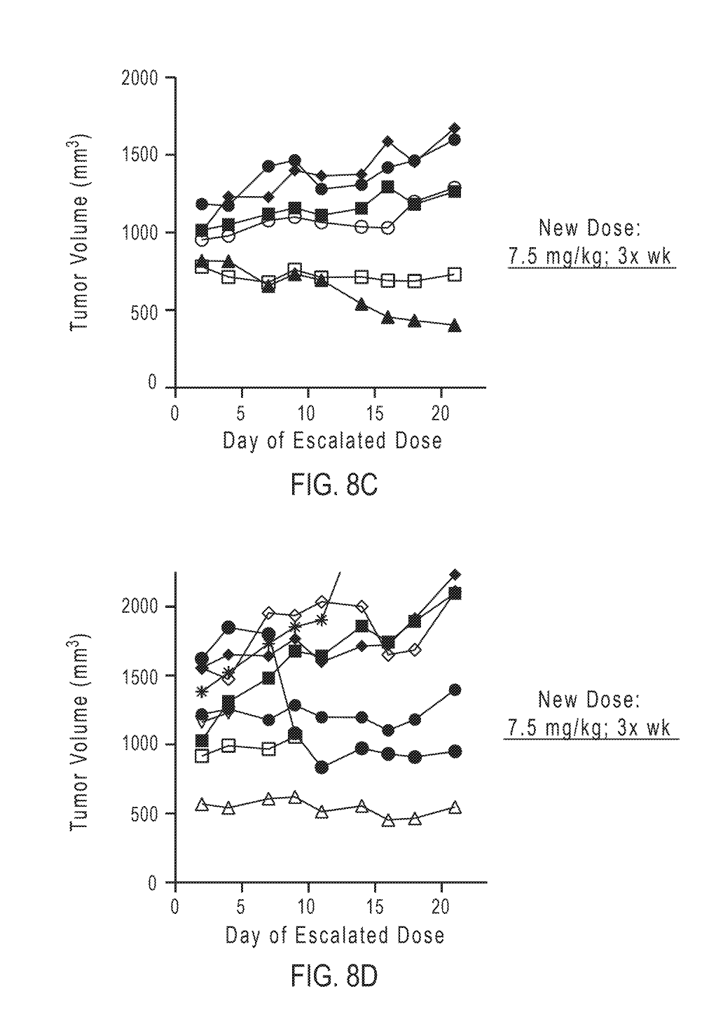

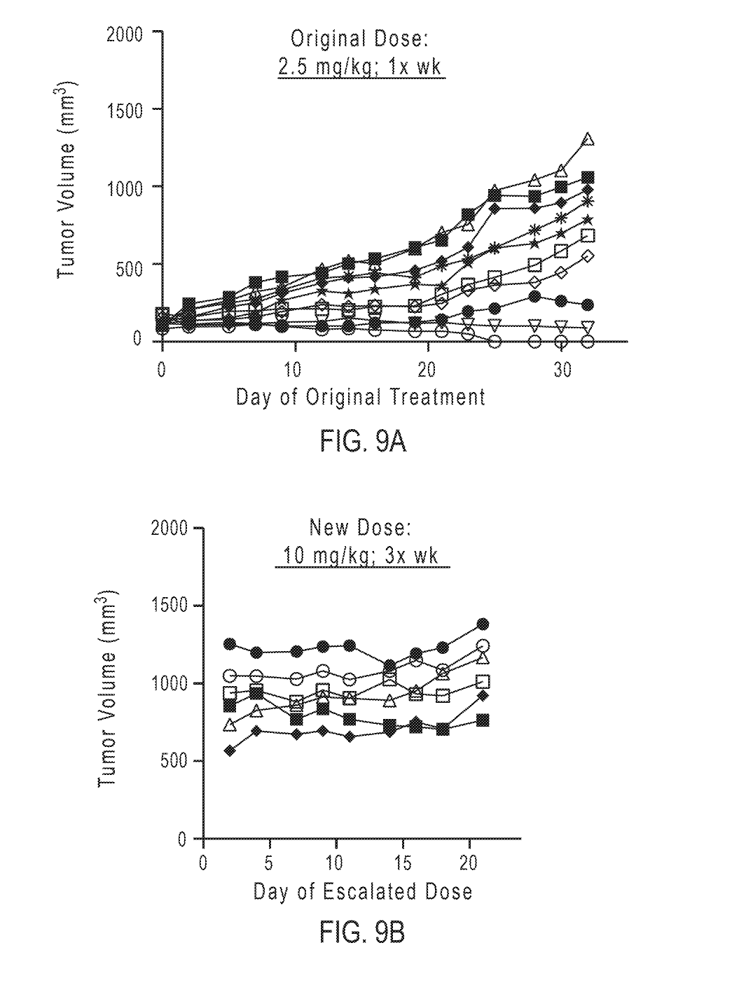

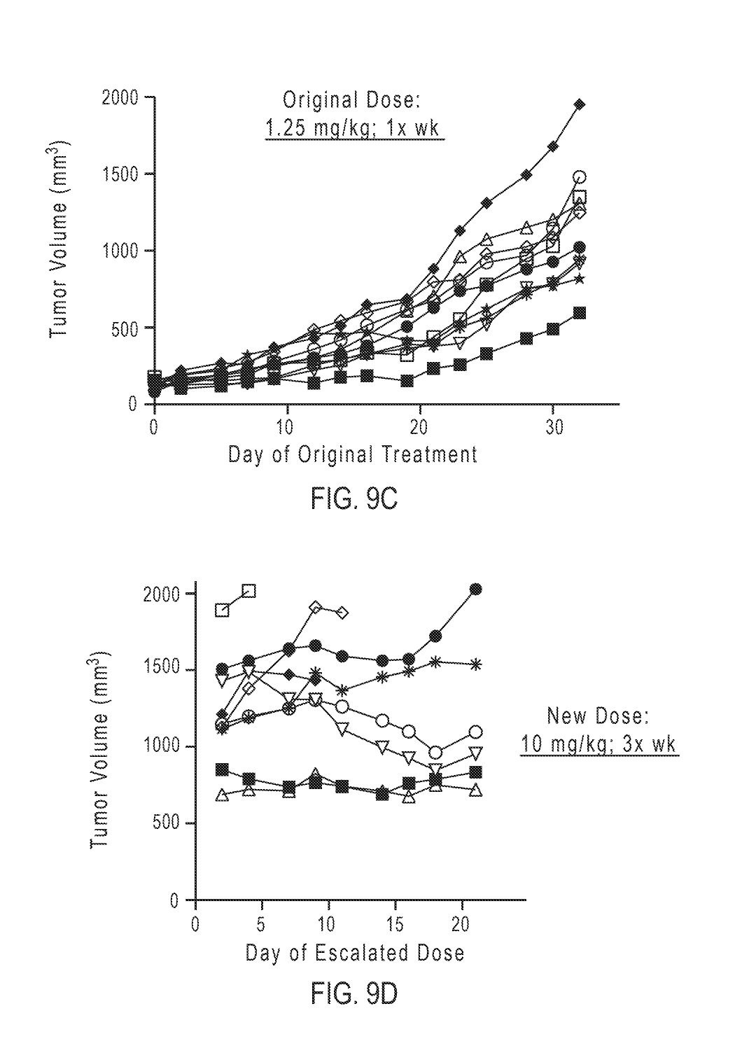

In a counterpart study, mice in which the PC3 tumor size was at least 500 mm³ were administered either 7.5 mg/kg or 10 mg/kg compound (5) three times a week for four weeks. In these studies, the mice were first administered a dose of either 2.5 mg/kg or 1.25 mg/kg, three times per week for three weeks. The tumor size, body weight, white cell count, and granulocyte count were measured. The tumor volume for the 7.5 mg/kg dosing regimen is resented in FIGS. 8A-8D, and for the 10 mg/kg dosing regimen in FIGS. 9A-9D. The results show that the PC3 tumor growth slowed in many of the animals and in some the size of the tumor decreased. As shown in FIGS. 10A-10C, the body weights for the 7.5 mg/kg or 10 mg/kg dosing regimens remained about the same and the cell counts remained within the normal range during the study.

### Example 33

### Prostate Cancer Xenograft Study

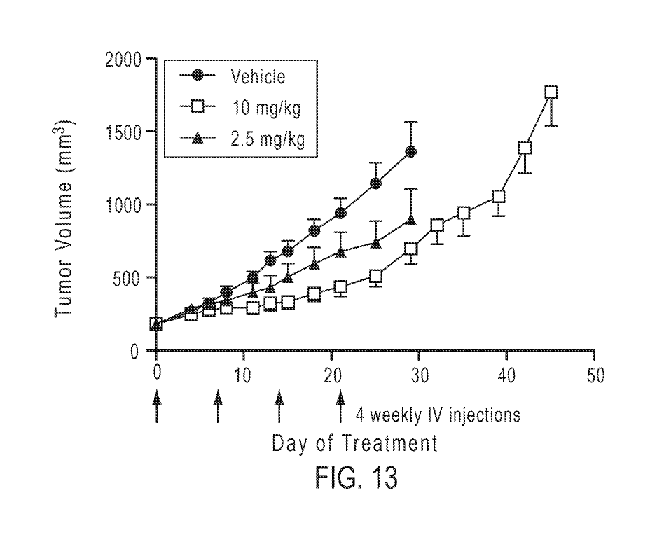

The efficacy of compounds provided by the present disclosure was evaluated on a prostate cancer xenograft model—PC3. Human prostate cancer PC3 cells were implanted into the flank of nude mice and the study was initiated after the volume of the tumors was 150 mm³. The animals were IV dosed once a week for 4 weeks with either vehicle or 2.5 mg/kg or 10 mg/kg of compound (5), compound (7), or compound (9). The tumor volume, body weight, white blood cell count, and granulocyte count were determined during the study and 24 days after the final dose was administered. The results are presented in FIGS. 11-13, respectively. The compounds exhibited dose-dependent tumor growth suppression. The animals maintained weight and myelosuppression was not detected during the study.

### Example 34

### Triple Negative Breast Cancer Study

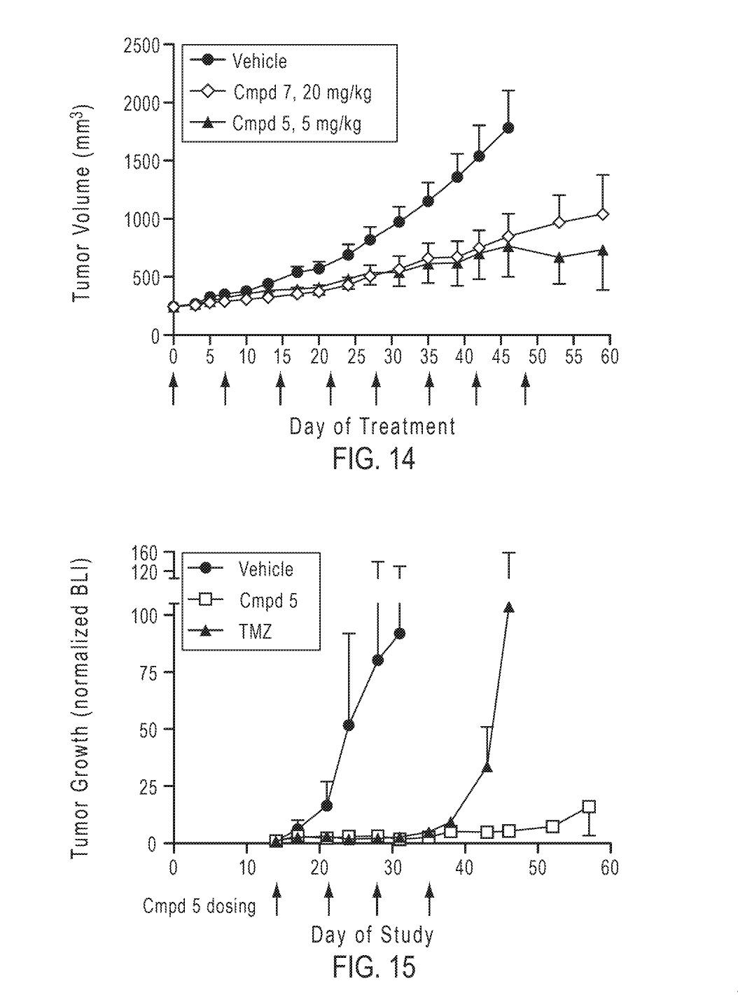

The efficacy of compounds provided by the present disclosure was evaluated on a triple negative breast cancer xenograft model—MDA-MB-231. Human triple negative breast cancer cells were implanted into the flank of nude mice and the study was initiated after the volume of the tumors was 150 mm³. The animals were IV dosed once a week for 8 weeks with either vehicle or 5 mg/kg of compound (5), or 20 mg/kg compound (7). The tumor volume, body weight, white blood cell count, and granulocyte count were determined during the study and 12 days after the final dose was administered. The results are presented in FIG. 14. The compounds exhibited dose-dependent tumor growth suppression. The animals maintained weight and myelosuppression was not detected during the study.

### Example 35

### Orthotopic Glioblastoma Study

The efficacy of compounds provided by the present disclosure was evaluated using an orthotopic luciferase human glioblastoma model—U251 MG. Human glioblastoma cells were intracolonically injected (3×10⁵ cells/3 μL) into athymic mice (female, 4-5 weeks-old, nu/un homozygous). Treatment began when the BLI reached a log-phase growth, at about 14 days after injection. Ten mice were assigned to each arm of the study. The mice were dosed with vehicle (IP, once weekly for 4 weeks), temozolomide (TMZ, 4 mg/kg, OG once daily for 5 days), or compound (5) (10 mg/kg, IP once weekly for 4 weeks). The tumor volume was measured using bioluminescence imaging. Compound (5) crossed the BBB and suppressed glioblastoma tumor growth. The results are shown in FIG. 15.

### Example 36

### Orthotopic Glioblastoma Study

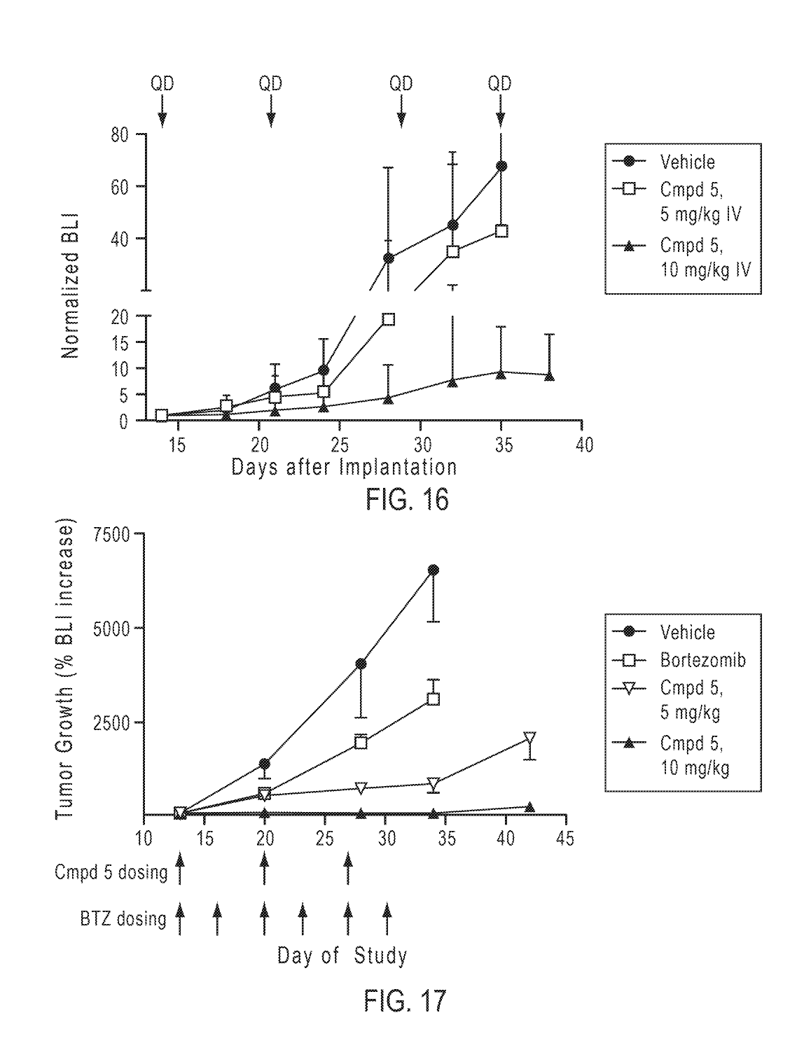

The efficacy of compounds provided by the present disclosure was evaluated using an orthotopic luciferase human glioblastoma model—U251 MG. Human glioblastoma cells were intracolonically injected (3×10⁵ cells/3 μL) into athymic mice (female, 4-5 weeks-old, nu/un homozygous). Treatment began when the BLI reached a log-phase growth, at about 14 days after injection. Ten mice were assigned to each arm of the study. The mice were dosed with vehicle (IP, once weekly for 4 weeks), compound (5) (5 mg/kg, IV once weekly for 4 weeks), or compound (5) (10 mg/kg, IV once weekly for 4 weeks). The tumor volume was measured using bioluminescence imaging. The results are shown in FIG. 16. Compound (5) demonstrated dose-dependent suppression of glioblastoma tumor growth, Compounds (40) and (50) suppressed glioblastoma tumor growth at a dose of 10 mg/kg.

### Example 37

### Orthotopic Multiple Myeloma Study

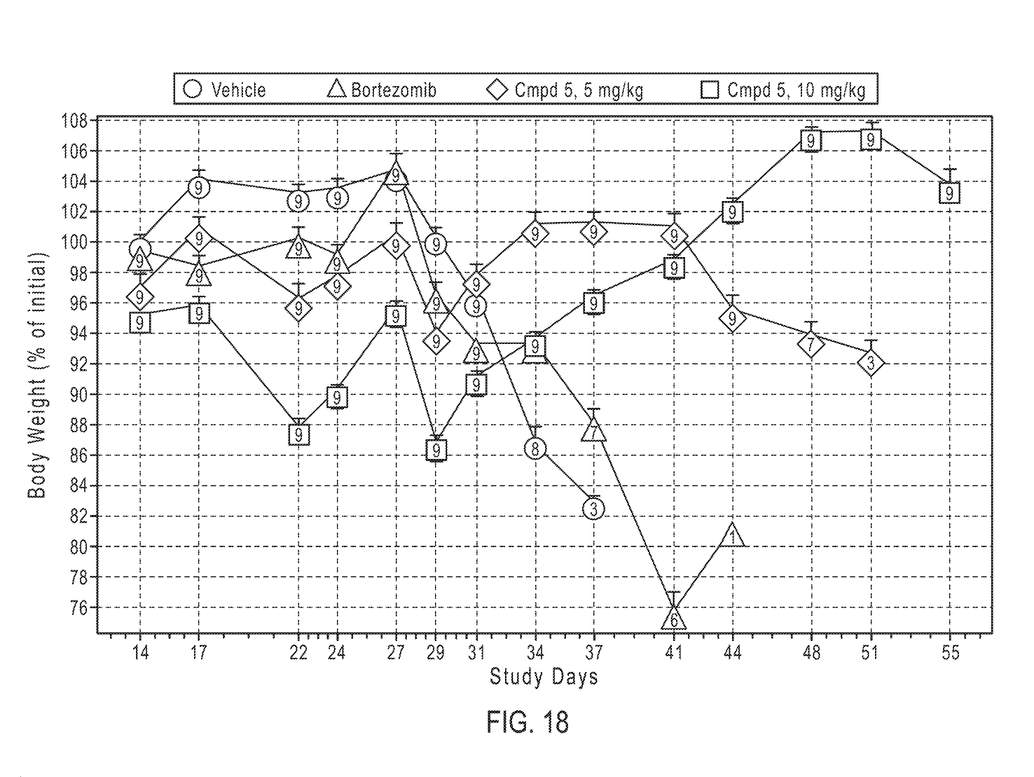

The efficacy of compounds provided by the present disclosure was evaluated using an orthotopic luciferase human multiple myeloma model—U266. Human multiple myeloma cells were IV injected (1×10⁶ cells) into NSG mice (female, 5-6 weeks-old, Jackson Labs). Treatment began when the BLI reached a log-phase growth, at about 12 to 14 days after injection. Nine mice were assigned to each arm of the study. The mice were dosed with vehicle (IP, once weekly for 3 weeks), bortezomid (BTZ, 0.8 mg/kg, IP twice weekly for 3 weeks), compound (5) (5 mg/kg, IV once weekly for 3 weeks), or compound (5) (10 mg/kg, IV once weekly for 3 weeks). The tumor volume was measured using bioluminescence imaging. The results are shown in FIG. 17. Compound (5) demonstrated dose-dependent suppression of glioblastoma tumor growth, Compound (5) exhibited dose-dependent suppression of multiple myeloma tumor growth. The body weight of the mice during the course of the study is shown in FIG. 18.

### Example 38

### Myeloprotection Study

The myeloprotective effects of methotrexate administered prior to treatment with compound (5) was evaluated.

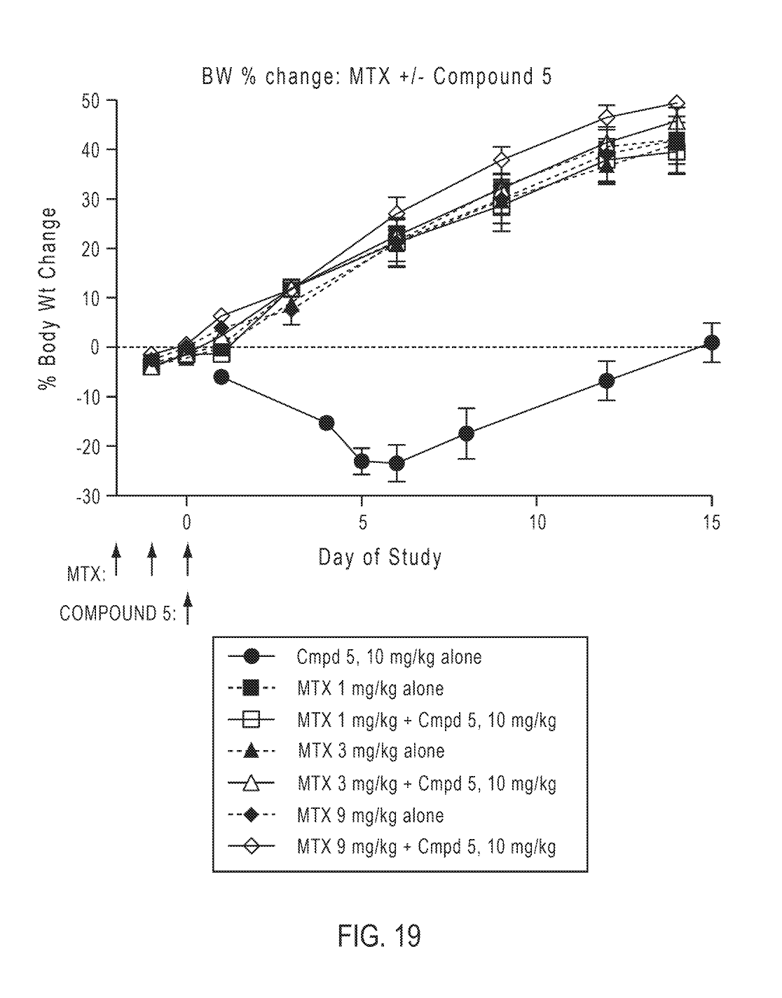

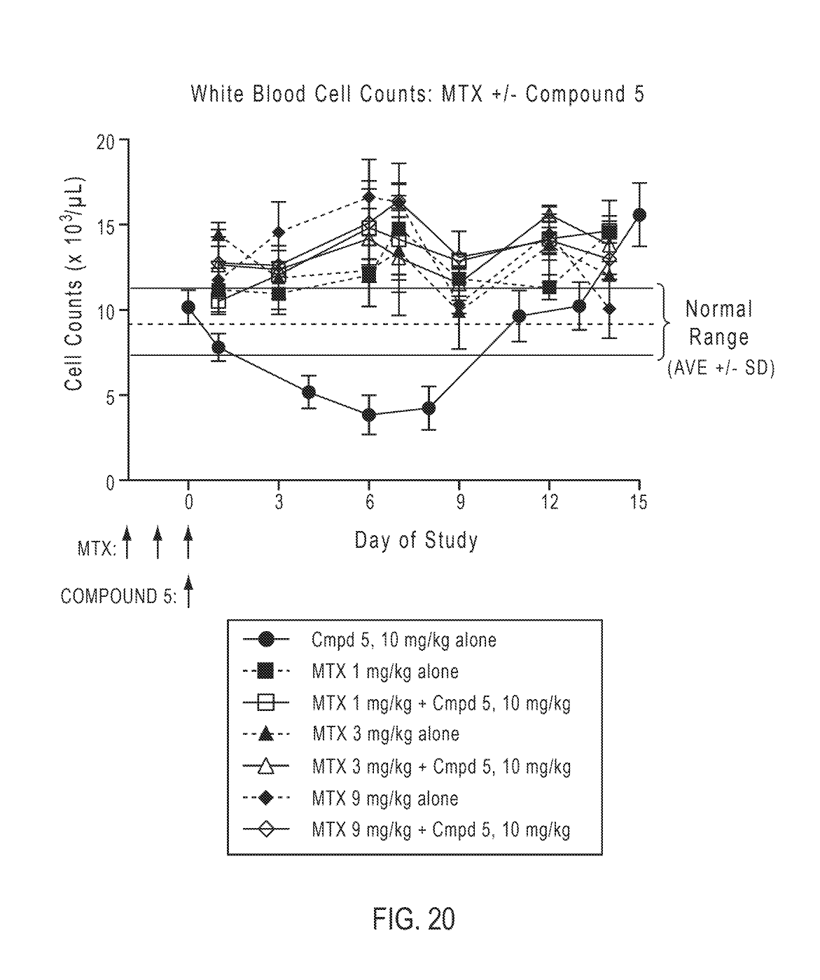

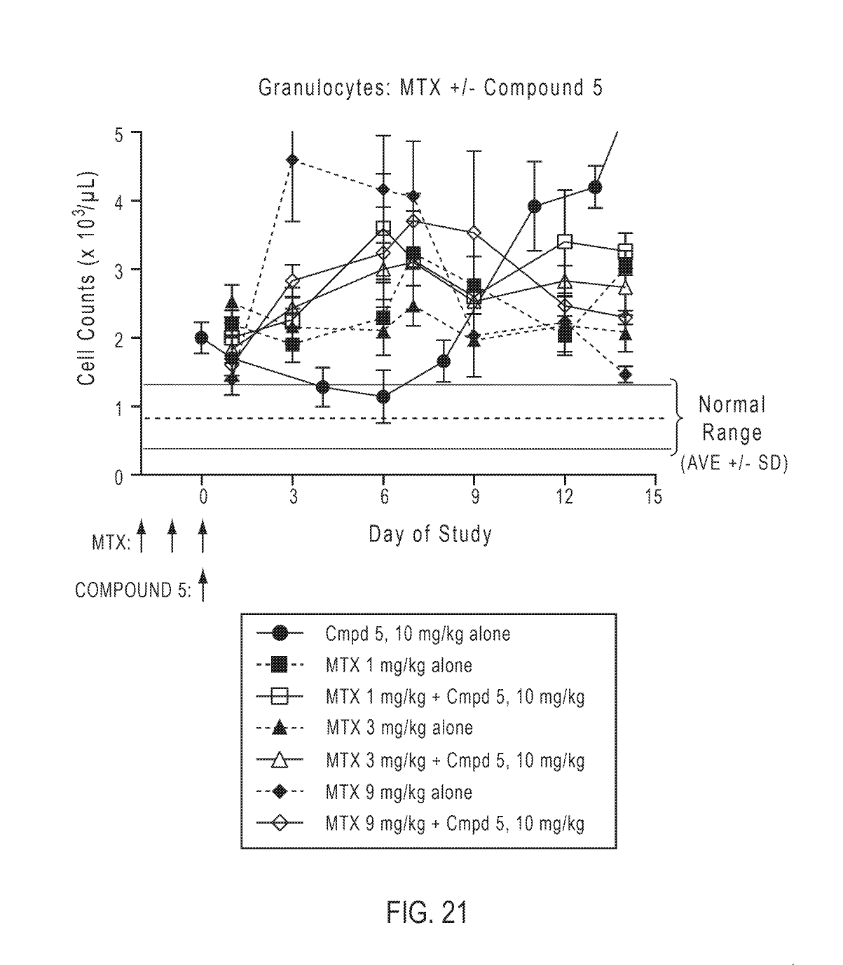

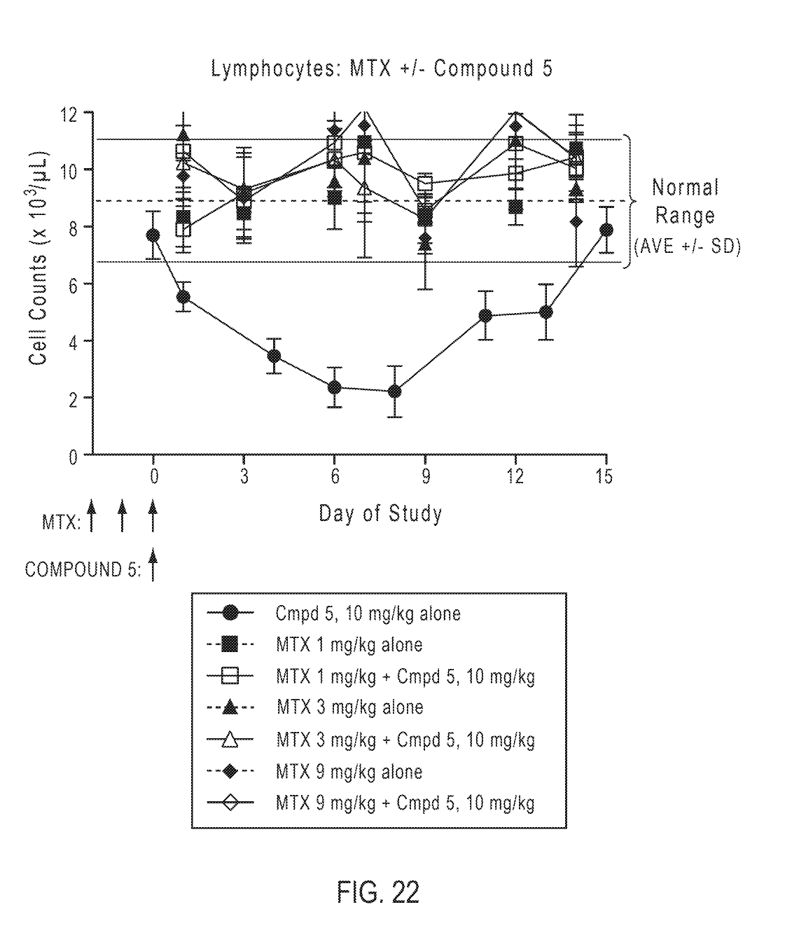

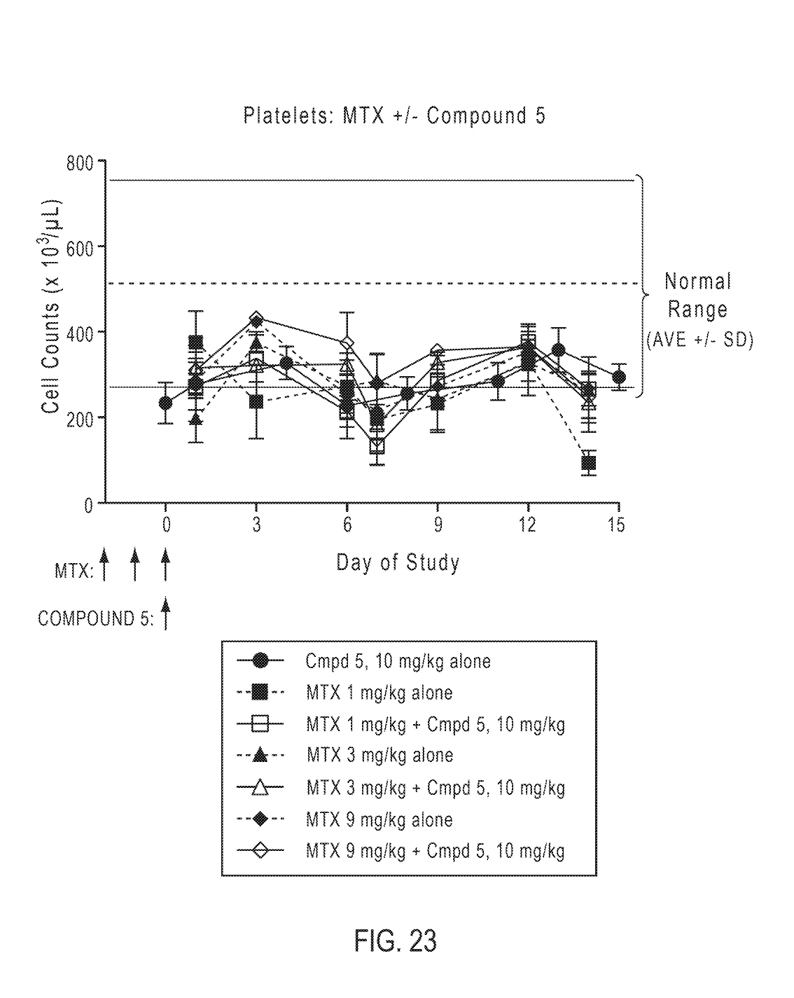

Three (3) Sprague-Dawley rates were used in each arm of the study. Methotrexate (MTX) at a dose of 1 mg/kg, 3 kg/mg, or 9 mg/kg was administered as an aqueous solution as an aqueous solution of disodium methotrexate Na₂MTX) to the rats by oral gavage on day −2, day −1, and day 0 of the study. Compound (5) was administered by IV on day 0 at a dose of 10 mg/kg. Vehicle was administered to other animals. The percent change in body weight of the animals is shown in FIG. 19 up to 15 days after administration of compound (5). The white blood cell count, granulocyte count, lymphocyte count, and platelet count are shown in FIGS. 20-23, respectively.

According to aspects of the invention, a method of reducing the effects of chemotherapy on normal/healthy cells in a patient being treated for cancer or abnormal cell proliferation, comprises: administering to the patient a therapeutically effective amount of a cell cycle inhibitor; and administering to the patient a therapeutically effective amount of a chemotherapeutic compound comprises a compound of Formula (1):

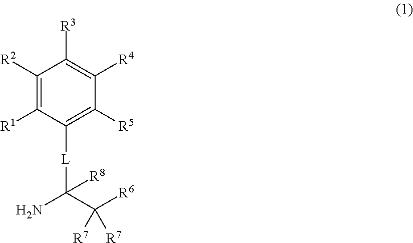

or a pharmaceutically acceptable salt thereof, wherein:

at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅₋₁₀ heteroaryl, C₆₋₁₆ heteroarylalkyl, and substituted C₆₋₁₆ heteroarylalkyl;

one of R¹, R², R³, R⁴, and R⁵ comprises a chemotherapeutic moiety;

each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;

R⁶ is selected from a carboxylic acid (—COOH), a carboxylic acid analog, and a carboxylic acid (bio)isostere;

each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;

R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

According to any of the preceding aspects, the chemotherapeutic moiety is a moiety of Formula (2a):

-A-NQ(-Z—C(R¹¹)₂—C(R¹¹)₂—R⁹)(—C(R¹¹)₂—C(R¹¹)₂—R⁹) (2a)

wherein,
A is selected from a bond (“—”), methylene (—CH₂—), oxygen (—O—), methyleneoxy (—CH₂—O—), carbonyl (—C(═O)—), methylenecarbonyl (—CH₂—C(═O)—), oxycarbonyl (—O—C(═O)—), and methyleneoxycarbonyl (—CH₂—O—C(═O)—);
Z is selected from a bond (“—”) and oxygen (—O—);
Q is selected from —O⁻ (a negatively charged oxygen atom that is bound to a positively charged nitrogen atom) and a free electron pair (:);
each R¹¹ is independently selected from hydrogen and deuterio; and
each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

According to any of the preceding aspects, the chemotherapeutic moiety is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected chloro (—Cl), bromo (—Br), iodo (—I), methylsulfonyloxy (—OSO₂CH₃), and trifluoromethylsulfonyloxy (—OSO₂CF₃).

According to any of the preceding aspects,

R⁶ is selected from —COOH, —S(O)OH, and —P(O)(OH)H, and 1H-tetrazole;

each R⁷ is independently selected from hydrogen, deuterio, fluoro, hydroxyl, and methyl;

R⁸ is selected from hydrogen, methyl, ethyl, propyl, and isopropyl;

each R¹⁰ is independently selected from hydrogen and C₁₋₄ alkyl; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

According to any of the preceding aspects,

at least one of R¹ and R⁵ is independently selected from, halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, substituted C₁₋₄ alkyl, C₁₋₄ alkoxy, substituted C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl;

each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring; and

one of R¹, R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

According to any of the preceding aspects, each of R², R³, and R⁵ is hydrogen.

According to any of the preceding aspects,

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;

one of R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R², R³, R⁴, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is hydrogen, methyl, or ethyl.

According to any of the preceding aspects,

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;

R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of R², R³, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

According to any of the preceding aspects, the compound of Formula (1) is selected from:
3-amino-3-[5-[bis(2-chloroethyl)amino]-2-methyl-phenyl]propanoic acid (1);
3-amino-3-[4-[bis(2-chloroethyl)amino]-2-methyl-phenyl]propanoic acid (2);
3-amino-4-[5-[bis(2-chloroethyl)amino]-2-methyl-phenyl]butanoic acid (3);
3-amino-4-[4-[bis(2-chloroethyl)amino]-2-methyl-phenyl]butanoic acid (4);
(3S)-3-amino-4-[5-[bis(2-chloroethyl)amino]-2-methyl-phenyl]butanoic acid (5);
(3R)-3-amino-4-[5-[bis(2-chloroethyl)amino]-2-methyl-phenyl]butanoic acid (6);
(3S)-3-amino-4-[5-[bis(2-chloroethyl)amino]-2-methoxy-phenyl]butanoic acid (7);
(3S)-3-amino-4-[3-[bis(2-chloroethyl)amino]-2,6-dimethyl-phenyl]butanoic acid (8);
(3S)-3-amino-4-[5-[bis(2-chloroethyl)amino]-2-methyl-phenyl]-3-methyl-butanoic acid (9);
[(2R)-2-amino-3-[5-[bis(2-chloroethyl)amino]-2-methyl-phenyl]propyl]phosphinic acid (10);
(3R)-3-amino-4-[5-(bis(2-methylsulfonyloxyethyl)amino)-2-methyl-phenyl]butanoic acid (11);
(3R)-3-amino-4-[5-(bis(2-bromoethyl)amino)-2-methyl-phenyl]butanoic acid (12);
(3R)-3-amino-4-[5-(2-chloroethyl(2-methylsulfonyloxyethyl)amino)-2-methyl-phenyl]butanoic acid (13);
(3R)-3-amino-4-[5-(2-bromoethyl(2-chloroethyl)amino)-2-methyl-phenyl]butanoic acid (14);
(3R)-3-amino-4-[5-(2-bromoethyl(2-methylsulfonyloxyethyl)amino)-2-methyl-phenyl]butanoic acid (15);
(3S)-3-amino-4-[[5-[bis(2-chloroethyl)amino]-2-methyl-phenyl]amino]-4-oxo-butanoic acid (16);
(3S)-3-amino-4-[2-[bis(2-chloroethyl)amino]phenoxy]butanoic acid (17);
(3R)-3-amino-5-[5-[bis(2-chloroethyl)amino]-2-methyl-phenyl]pentanoic acid (18);
(3R)-3-amino-4-[5-(2-chloroethyl(chloromethyl)carbamoyl)oxy-2-methyl-phenyl]butanoic acid (19);
(3R)-3-amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21);
3-[(2R)-2-amino-4-hydroxy-4-oxo-butyl]-N,N-bis(2-chloroethyl)-4-methyl-benzeneamine oxide (22); and
(3R)-3-amino-4-[2-[bis(2-chloroethyl)carbamoyl]phenyl]butanoic acid (23);
or a pharmaceutically acceptable salt of any of the foregoing.

According to any of the preceding aspects, the cell cycle inhibitor comprises a myelosuppressor, an immunosuppressor, or a combination thereof.

According to any of the preceding aspects, the cell cycle inhibitor is selected from methotrexate or salts thereof, mycophenolic acid or derivatives or salts thereof, leflunomide or salts thereof, or a combination of any of the foregoing.

According to any of the preceding aspects, the therapeutically effective amount of the cell cycle inhibitor is effective in reducing the level of myelosuppression associated with the administration of the chemotherapeutic agent, compared to the level of myelosuppression associated with the administration of the chemotherapeutic agent without the administration of the cell cycle inhibitor.

According to any of the preceding aspects, the method results in a higher therapeutic index for the chemotherapeutic agent compared to a therapeutic index for the chemotherapeutic agent without administering the cell cycle inhibitor.

According to any of the preceding aspects, the cell cycle inhibitor comprises a myelosuppressor.

According to any of the preceding aspects, the cancer comprises brain cancer.

According to any of the preceding aspects, the cell cycle inhibitor is effective in arresting the growth of hematopoietic stem cells, hematopoietic progenitor cells, T-cells, multipotent progenitors, common myeloid progenitors, common lymphoid progenitors, granulocyte-monocyte progenitors, and megakaryocyte-erythroid progenitors, renal epithelial cells, T-cells, and a combination of any of the foregoing.

According to any of the preceding aspects, the cell cycle inhibitor is administered before administering the chemotherapeutic agent.

According to the present invention, methods of treating cancer in a patient, comprise administering to the patient being treated for the cancer, a therapeutically effective amount of a cell cycle inhibitor; and a therapeutically effective amount of a chemotherapeutic agent comprises a compound of Formula (1):

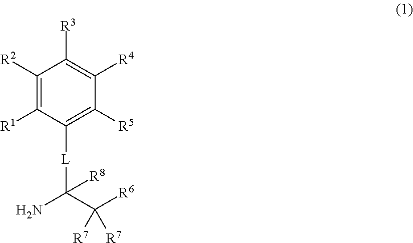

or a pharmaceutically acceptable salt thereof, wherein:

at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅₋₁₀ heteroaryl, C₆₋₁₆ heteroarylalkyl, and substituted C₆₋₁₆ heteroarylalkyl;

one of R¹, R², R³, R⁴, and R⁵ comprises a chemotherapeutic moiety;

each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;

R⁶ is selected from a carboxylic acid (—COOH), a carboxylic acid analog, and a carboxylic acid (bio)isostere;

each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;

R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

According to any of the preceding aspects, the chemotherapeutic moiety is a moiety of Formula (2a):

-A-NQ(-Z—C(R¹¹)₂—C(R¹¹)₂—R⁹)(—C(R¹¹)₂—C(R¹¹)₂—R⁹) (2a)

wherein,

A is selected from a bond (“—”), methylene (—CH₂—), oxygen (—O—), methyleneoxy (—CH₂—O—), carbonyl (—C(═O)—), methylenecarbonyl (—CH₂—C(═O)—), oxycarbonyl (—O—C(═O)—), and methyleneoxycarbonyl (—CH₂—O—C(═O)—);

Z is selected from a bond (“—”) and oxygen (—O—);

Q is selected from —O⁻ (a negatively charged oxygen atom that is bound to a positively charged nitrogen atom) and a free electron pair (:);

each R¹¹ is independently selected from hydrogen and deuterio; and

each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

According to any of the preceding aspects, the chemotherapeutic moiety is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected chloro (—Cl), bromo (—Br), iodo (—I), methylsulfonyloxy (—OSO₂CH₃), and trifluoromethylsulfonyloxy (—OSO₂CF₃).

According to any of the preceding aspects,

R⁶ is selected from —COOH, —S(O)OH, and —P(O)(OH)H, and 1H-tetrazole;

each R⁷ is independently selected from hydrogen, deuterio, fluoro, hydroxyl, and methyl;

R⁸ is selected from hydrogen, methyl, ethyl, propyl, and isopropyl;

each R¹⁰ is independently selected from hydrogen and C₁₋₄ alkyl; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

According to any of the preceding aspects,

at least one of R¹ and R⁵ is independently selected from, halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, substituted C₁₋₄ alkyl, C₁₋₄ alkoxy, substituted C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl;

each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring; and

one of R¹, R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

According to any of the preceding aspects, each of R², R³, and R⁵ is hydrogen.

According to any of the preceding aspects,

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;

one of R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R², R³, R⁴, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is hydrogen, methyl, or ethyl.

According to any of the preceding aspects,

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;

R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of R², R³, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

According to any of the preceding aspects, the compound of Formula (1) is selected from:
3-amino-3-[5-[bis(2-chloroethyl)amino]-2-methyl-phenyl]propanoic acid (1);
3-amino-3-[4-[bis(2-chloroethyl)amino]-2-methyl-phenyl]propanoic acid (2);
3-amino-4-[5-[bis(2-chloroethyl)amino]-2-methyl-phenyl]butanoic acid (3);
3-amino-4-[4-[bis(2-chloroethyl)amino]-2-methyl-phenyl]butanoic acid (4);
(3S)-3-amino-4-[5-[bis(2-chloroethyl)amino]-2-methyl-phenyl]butanoic acid (5);
(3R)-3-amino-4-[5-[bis(2-chloroethyl)amino]-2-methyl-phenyl]butanoic acid (6);
(3S)-3-amino-4-[5-[bis(2-chloroethyl)amino]-2-methoxy-phenyl]butanoic acid (7);
(3S)-3-amino-4-[3-[bis(2-chloroethyl)amino]-2,6-dimethyl-phenyl]butanoic acid (8);
(3S)-3-amino-4-[5-[bis(2-chloroethyl)amino]-2-methyl-phenyl]-3-methyl-butanoic acid (9);
[(2R)-2-amino-3-[5-[bis(2-chloroethyl)amino]-2-methyl-phenyl]propyl]phosphinic acid (10);
(3R)-3-amino-4-[5-(bis(2-methylsulfonyloxyethyl)amino)-2-methyl-phenyl]butanoic acid (11);
(3R)-3-amino-4-[5-(bis(2-bromoethyl)amino)-2-methyl-phenyl]butanoic acid (12);
(3R)-3-amino-4-[5-(2-chloroethyl(2-methylsulfonyloxyethyl)amino)-2-methyl-phenyl]butanoic acid (13);
(3R)-3-amino-4-[5-(2-bromoethyl(2-chloroethyl)amino)-2-methyl-phenyl]butanoic acid (14);
(3R)-3-amino-4-[5-(2-bromoethyl(2-methylsulfonyloxyethyl)amino)-2-methyl-phenyl]butanoic acid (15);
(3S)-3-amino-4-[[5-[bis(2-chloroethyl)amino]-2-methyl-phenyl]amino]-4-oxo-butanoic acid (16);
(3S)-3-amino-4-[2-[bis(2-chloroethyl)amino]phenoxy]butanoic acid (17);
(3R)-3-amino-5-[5-[bis(2-chloroethyl)amino]-2-methyl-phenyl]pentanoic acid (18);
(3R)-3-amino-4-[5-(2-chloroethyl(chloromethyl)carbamoyl)oxy-2-methyl-phenyl]butanoic acid (19);
(3R)-3-amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21);
3-[(2R)-2-amino-4-hydroxy-4-oxo-butyl]-N,N-bis(2-chloroethyl)-4-methyl-benzeneamine oxide (22); and
(3R)-3-amino-4-[2-[bis(2-chloroethyl)carbamoyl]phenyl]butanoic acid (23);
or a pharmaceutically acceptable salt of any of the foregoing.

According to any of the preceding aspects, the cell cycle inhibitor comprises a myelosuppressor, an immunosuppressor, or a combination thereof.

According to any of the preceding aspects, the cell cycle inhibitor is selected from methotrexate or salts thereof, mycophenolic acid or derivatives or salts thereof, leflunomide or salts thereof, or a combination of any of the foregoing.

According to any of the preceding aspects, the therapeutically effective amount of the cell cycle inhibitor is effective in reducing the level of myelosuppression associated with the administration of the chemotherapeutic agent, compared to the level of myelosuppression associated with the administration of the chemotherapeutic agent without the administration of the cell cycle inhibitor.

According to any of the preceding aspects, the method results in a higher therapeutic index for the chemotherapeutic agent compared to a therapeutic index for the chemotherapeutic agent without administering the cell cycle inhibitor.

According to any of the preceding aspects, the cell cycle inhibitor comprises a myelosuppressor.

According to any of the preceding aspects, the cancer comprises brain cancer.

According to any of the preceding aspects, the cell cycle inhibitor is effective in arresting the growth of hematopoietic stem cells, hematopoietic progenitor cells, T-cells, multipotent progenitors, common myeloid progenitors, common lymphoid progenitors, granulocyte-monocyte progenitors, and megakaryocyte-erythroid progenitors, renal epithelial cells, T-cells, and a combination of any of the foregoing.

According to any of the preceding aspects, the cell cycle inhibitor is administered before administering the chemotherapeutic agent.

According to the present invention, methods of promoting recovery from the effects of a chemotherapeutic regimen for treating cancer in a patient comprise administering to the patient: a therapeutically effective amount of a cell cycle inhibitor to inhibit the proliferation of normal, healthy cells; and a therapeutically effective about of a chemotherapeutic agent comprises a compound of Formula (1):

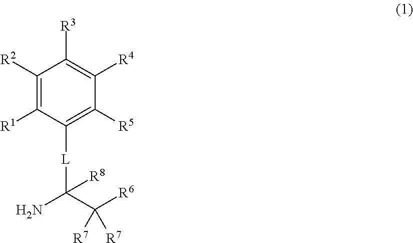

or a pharmaceutically acceptable salt thereof, wherein:

at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅₋₁₀ heteroaryl, C₆₋₁₆ heteroarylalkyl, and substituted C₆₋₁₆ heteroarylalkyl;

one of R¹, R², R³, R⁴, and R⁵ comprises a chemotherapeutic moiety;

each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;

R⁶ is selected from a carboxylic acid (—COOH), a carboxylic acid analog, and a carboxylic acid (bio)isostere;

each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;

R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

According to any of the preceding aspects, the chemotherapeutic moiety is a moiety of Formula (2a):

-A-NQ(-Z—C(R¹¹)₂—C(R¹¹)₂—R⁹)(—C(R¹¹)₂—C(R¹¹)₂—R⁹) (2a)

wherein,

A is selected from a bond (“—”), methylene (—CH₂—), oxygen (—O—), methyleneoxy (—CH₂—O—), carbonyl (—C(═O)—), methylenecarbonyl (—CH₂—C(═O)—), oxycarbonyl (—O—C(═O)—), and methyleneoxycarbonyl (—CH₂—O—C(═O)—);

Z is selected from a bond (“—”) and oxygen (—O—);

Q is selected from —O⁻ (a negatively charged oxygen atom that is bound to a positively charged nitrogen atom) and a free electron pair (:);

each R¹¹ is independently selected from hydrogen and deuterio; and

each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

According to aspects of the invention, a method of reducing the effects of chemotherapy on normal/healthy cells in a patient being treated for cancer or abnormal cell proliferation, comprises: administering to the patient a therapeutically effective amount of a cell cycle inhibitor; and administering to the patient a therapeutically effective amount of a chemotherapeutic compound comprises a compound of Formula (1):

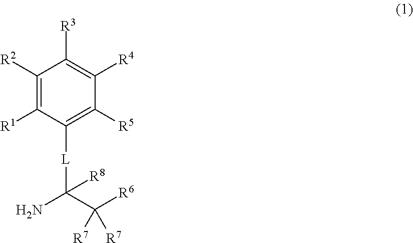

or a pharmaceutically acceptable salt thereof, wherein:

at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅₋₁₀ heteroaryl, C₆₋₁₆ heteroarylalkyl, and substituted C₆₋₁₆ heteroarylalkyl;

one of R¹, R², R³, R⁴, and R⁵ comprises a chemotherapeutic moiety;

each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;

R⁶ is selected from a carboxylic acid (—COOH), a carboxylic acid analog, and a carboxylic acid (bio)isostere;

each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;

R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

According to any of the preceding aspects, the chemotherapeutic moiety is a moiety of Formula (2a):

-A-NQ(-Z—C(R¹¹)₂—C(R¹¹)₂—R⁹)(—C(R¹¹)₂—C(R¹¹)₂—R⁹) (2a)

wherein,

A is selected from a bond (“—”), methylene (—CH₂—), oxygen (—O—), methyleneoxy (—CH₂—O—), carbonyl (—C(═O)—), methylenecarbonyl (—CH₂—C(═O)—), oxycarbonyl (—O—C(═O)—), and methyleneoxycarbonyl (—CH₂—O—C(═O)—);

Z is selected from a bond (“—”) and oxygen (—O—);

Q is selected from —O⁻ (a negatively charged oxygen atom that is bound to a positively charged nitrogen atom) and a free electron pair (:);

each R¹¹ is independently selected from hydrogen and deuterio; and

each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

According to any of the preceding aspects, the chemotherapeutic moiety is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected chloro (—Cl), bromo (—Br), iodo (—I), methylsulfonyloxy (—OSO₂CH₃), and trifluoromethylsulfonyloxy (—OSO₂CF₃).

According to any of the preceding aspects,

R⁶ is selected from —COOH, —S(O)OH, and —P(O)(OH)H, and 1H-tetrazole;

each R⁷ is independently selected from hydrogen, deuterio, fluoro, hydroxyl, and methyl;

R⁸ is selected from hydrogen, methyl, ethyl, propyl, and isopropyl;

each R¹⁰ is independently selected from hydrogen and C₁₋₄ alkyl; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

According to any of the preceding aspects,

at least one of R¹ and R⁵ is independently selected from, halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, substituted C₁₋₄ alkyl, C₁₋₄ alkoxy, substituted C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl;

each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring; and

one of R¹, R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

According to any of the preceding aspects, each of R², R³, and R⁵ is hydrogen.

According to any of the preceding aspects,

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;

one of R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R², R³, R⁴, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is hydrogen, methyl, or ethyl.

According to any of the preceding aspects,

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;

R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of R², R³, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

According to any of the preceding aspects, the compound of Formula (1) is selected from:
3-amino-3-[5-[bis(2-chloroethyl)amino]-2-methyl-phenyl]propanoic acid (1);
3-amino-3-[4-[bis(2-chloroethyl)amino]-2-methyl-phenyl]propanoic acid (2);
3-amino-4-[5-[bis(2-chloroethyl)amino]-2-methyl-phenyl]butanoic acid (3);
3-amino-4-[4-[bis(2-chloroethyl)amino]-2-methyl-phenyl]butanoic acid (4);
(3S)-3-amino-4-[5-[bis(2-chloroethyl)amino]-2-methyl-phenyl]butanoic acid (5);
(3R)-3-amino-4-[5-[bis(2-chloroethyl)amino]-2-methyl-phenyl]butanoic acid (6);
(3S)-3-amino-4-[5-[bis(2-chloroethyl)amino]-2-methoxy-phenyl]butanoic acid (7);
(3S)-3-amino-4-[3-[bis(2-chloroethyl)amino]-2,6-dimethyl-phenyl]butanoic acid (8);
(3S)-3-amino-4-[5-[bis(2-chloroethyl)amino]-2-methyl-phenyl]-3-methyl-butanoic acid (9);
[(2R)-2-amino-3-[5-[bis(2-chloroethyl)amino]-2-methyl-phenyl]propyl]phosphinic acid (10);
(3R)-3-amino-4-[5-(bis(2-methyl sulfonyloxyethyl)amino)-2-methyl-phenyl]butanoic acid (11);
(3R)-3-amino-4-[5-(bis(2-bromoethyl)amino)-2-methyl-phenyl]butanoic acid (12);
(3R)-3-amino-4-[5-(2-chloroethyl(2-methylsulfonyloxyethyl)amino)-2-methyl-phenyl]butanoic acid (13);
(3R)-3-amino-4-[5-(2-bromoethyl(2-chloroethyl)amino)-2-methyl-phenyl]butanoic acid (14);
(3R)-3-amino-4-[5-(2-bromoethyl(2-methylsulfonyloxyethyl)amino)-2-methyl-phenyl]butanoic acid (15);
(3S)-3-amino-4-[[5-[bis(2-chloroethyl)amino]-2-methyl-phenyl]amino]-4-oxo-butanoic acid (16);
(3S)-3-amino-4-[2-[bis(2-chloroethyl)amino]phenoxy]butanoic acid (17);
(3R)-3-amino-5-[5-[bis(2-chloroethyl)amino]-2-methyl-phenyl]pentanoic acid (18);
(3R)-3-amino-4-[5-(2-chloroethyl(chloromethyl)carbamoyl)oxy-2-methyl-phenyl]butanoic acid (19);
(3R)-3-amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21);
3-[(2R)-2-amino-4-hydroxy-4-oxo-butyl]-N,N-bis(2-chloroethyl)-4-methyl-benzeneamine oxide (22); and
(3R)-3-amino-4-[2-[bis(2-chloroethyl)carbamoyl]phenyl]butanoic acid (23);
or a pharmaceutically acceptable salt of any of the foregoing.

According to any of the preceding aspects, the cell cycle inhibitor comprises a myelosuppressor, an immunosuppressor, or a combination thereof.

According to any of the preceding aspects, the cell cycle inhibitor is selected from methotrexate or salts thereof, mycophenolic acid or derivatives or salts thereof, leflunomide or salts thereof, or a combination of any of the foregoing.

According to any of the preceding aspects, the therapeutically effective amount of the cell cycle inhibitor is effective in reducing the level of myelosuppression associated with the administration of the chemotherapeutic agent, compared to the level of myelosuppression associated with the administration of the chemotherapeutic agent without the administration of the cell cycle inhibitor.

According to any of the preceding aspects, the method results in a higher therapeutic index for the chemotherapeutic agent compared to a therapeutic index for the chemotherapeutic agent without administering the cell cycle inhibitor.

According to any of the preceding aspects, the cell cycle inhibitor comprises a myelosuppressor.

According to any of the preceding aspects, the cancer comprises brain cancer.

According to any of the preceding aspects, the cell cycle inhibitor is effective in arresting the growth of hematopoietic stem cells, hematopoietic progenitor cells, T-cells, multipotent progenitors, common myeloid progenitors, common lymphoid progenitors, granulocyte-monocyte progenitors, and megakaryocyte-erythroid progenitors, renal epithelial cells, T-cells, and a combination of any of the foregoing.

According to any of the preceding aspects, the cell cycle inhibitor is administered before administering the chemotherapeutic agent.

According to the present invention, methods of treating cancer in a patient, comprise administering to the patient being treated for the cancer, a therapeutically effective amount of a cell cycle inhibitor; and a therapeutically effective amount of a chemotherapeutic agent comprises a compound of Formula (1):

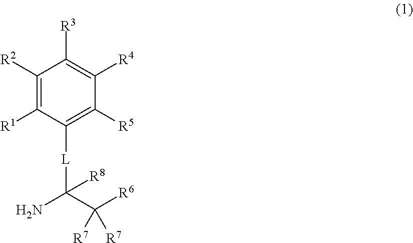

or a pharmaceutically acceptable salt thereof, wherein:

at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅₋₁₀ heteroaryl, C₆₋₁₆ heteroarylalkyl, and substituted C₆₋₁₆ heteroarylalkyl;

one of R¹, R², R, R⁴, and R⁵ comprises a chemotherapeutic moiety;

each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;

R⁶ is selected from a carboxylic acid (—COOH), a carboxylic acid analog, and a carboxylic acid (bio)isostere;

each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;

R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

According to any of the preceding aspects, the chemotherapeutic moiety is a moiety of Formula (2a):

-A-NQ(-Z—C(R¹¹)₂—C(R¹¹)₂—R⁹)(—C(R¹¹)₂—C(R¹¹)₂—R⁹) (2a)

wherein,

A is selected from a bond (“—”), methylene (—CH₂—), oxygen (—O—), methyleneoxy (—CH₂—O—), carbonyl (—C(═O)—), methylenecarbonyl (—CH₂—C(═O)—), oxycarbonyl (—O—C(═O)—), and methyleneoxycarbonyl (—CH₂—O—C(═O)—);

Z is selected from a bond (“—”) and oxygen (—O—);

Q is selected from —O⁻ (a negatively charged oxygen atom that is bound to a positively charged nitrogen atom) and a free electron pair (:);

each R¹¹ is independently selected from hydrogen and deuterio; and

each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

According to any of the preceding aspects, the chemotherapeutic moiety is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected chloro (—Cl), bromo (—Br), iodo (—I), methylsulfonyloxy (—OSO₂CH₃), and trifluoromethylsulfonyloxy (—OSO₂CF₃).

According to any of the preceding aspects,

R⁶ is selected from —COOH, —S(O)OH, and —P(O)(OH)H, and 1H-tetrazole;

each R⁷ is independently selected from hydrogen, deuterio, fluoro, hydroxyl, and methyl;

R⁸ is selected from hydrogen, methyl, ethyl, propyl, and isopropyl;

each R¹⁰ is independently selected from hydrogen and C₁₋₄ alkyl; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

According to any of the preceding aspects,

at least one of R¹ and R⁵ is independently selected from, halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, substituted C₁₋₄ alkyl, C₁₋₄ alkoxy, substituted C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl;

each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring; and

one of R¹, R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃.

According to any of the preceding aspects, each of R², R³, and R⁵ is hydrogen.

According to any of the preceding aspects,

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;

one of R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of the other of R², R³, R⁴, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is hydrogen, methyl, or ethyl.

According to any of the preceding aspects,

R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;

R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;

each of R², R³, and R⁵ is hydrogen;

R⁶ is selected from —COOH, —S(O)OH, —P(O)(OH)H, and 1H-tetrazole;

each R⁷ is independently selected from hydrogen, methyl, hydroxyl, and fluoro;

R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and

L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

According to any of the preceding aspects, the compound of Formula (1) is selected from:
3-amino-3-[5-[bis(2-chloroethyl)amino]-2-methyl-phenyl]propanoic acid (1);
3-amino-3-[4-[bis(2-chloroethyl)amino]-2-methyl-phenyl]propanoic acid (2);
3-amino-4-[5-[bis(2-chloroethyl)amino]-2-methyl-phenyl]butanoic acid (3);
3-amino-4-[4-[bis(2-chloroethyl)amino]-2-methyl-phenyl]butanoic acid (4);
(3S)-3-amino-4-[5-[bis(2-chloroethyl)amino]-2-methyl-phenyl]butanoic acid (5);
(3R)-3-amino-4-[5-[bis(2-chloroethyl)amino]-2-methyl-phenyl]butanoic acid (6);
(3S)-3-amino-4-[5-[bis(2-chloroethyl)amino]-2-methoxy-phenyl]butanoic acid (7);
(3S)-3-amino-4-[3-[bis(2-chloroethyl)amino]-2,6-dimethyl-phenyl]butanoic acid (8);
(3S)-3-amino-4-[5-[bis(2-chloroethyl)amino]-2-methyl-phenyl]-3-methyl-butanoic acid (9);
[(2R)-2-amino-3-[5-[bis(2-chloroethyl)amino]-2-methyl-phenyl]propyl]phosphinic acid (10);
(3R)-3-amino-4-[5-(bis(2-methyl sulfonyloxyethyl)amino)-2-methyl-phenyl]butanoic acid (11);
(3R)-3-amino-4-[5-(bis(2-bromoethyl)amino)-2-methyl-phenyl]butanoic acid (12);
(3R)-3-amino-4-[5-(2-chloroethyl(2-methylsulfonyloxyethyl)amino)-2-methyl-phenyl]butanoic acid (13);
(3R)-3-amino-4-[5-(2-bromoethyl(2-chloroethyl)amino)-2-methyl-phenyl]butanoic acid (14);
(3R)-3-amino-4-[5-(2-bromoethyl(2-methylsulfonyloxyethyl)amino)-2-methyl-phenyl]butanoic acid (15);
(3S)-3-amino-4-[[5-[bis(2-chloroethyl)amino]-2-methyl-phenyl]amino]-4-oxo-butanoic acid (16);
(3S)-3-amino-4-[2-[bis(2-chloroethyl)amino]phenoxy]butanoic acid (17);
(3R)-3-amino-5-[5-[bis(2-chloroethyl)amino]-2-methyl-phenyl]pentanoic acid (18);
(3R)-3-amino-4-[5-(2-chloroethyl(chloromethyl)carbamoyl)oxy-2-methyl-phenyl]butanoic acid (19);
(3R)-3-amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21);
3-[(2R)-2-amino-4-hydroxy-4-oxo-butyl]-N,N-bis(2-chloroethyl)-4-methyl-benzeneamine oxide (22); and
(3R)-3-amino-4-[2-[bis(2-chloroethyl)carbamoyl]phenyl]butanoic acid (23);
or a pharmaceutically acceptable salt of any of the foregoing.

According to any of the preceding aspects, the cell cycle inhibitor comprises a myelosuppressor, an immunosuppressor, or a combination thereof.

According to any of the preceding aspects, the cell cycle inhibitor is selected from methotrexate or salts thereof, mycophenolic acid or derivatives or salts thereof, leflunomide or salts thereof, or a combination of any of the foregoing.

According to any of the preceding aspects, the therapeutically effective amount of the cell cycle inhibitor is effective in reducing the level of myelosuppression associated with the administration of the chemotherapeutic agent, compared to the level of myelosuppression associated with the administration of the chemotherapeutic agent without the administration of the cell cycle inhibitor.

According to any of the preceding aspects, the method results in a higher therapeutic index for the chemotherapeutic agent compared to a therapeutic index for the chemotherapeutic agent without administering the cell cycle inhibitor.

According to any of the preceding aspects, the cell cycle inhibitor comprises a myelosuppressor.

According to any of the preceding aspects, the cancer comprises brain cancer.

According to any of the preceding aspects, the cell cycle inhibitor is effective in arresting the growth of hematopoietic stem cells, hematopoietic progenitor cells, T-cells, multipotent progenitors, common myeloid progenitors, common lymphoid progenitors, granulocyte-monocyte progenitors, and megakaryocyte-erythroid progenitors, renal epithelial cells, T-cells, and a combination of any of the foregoing.

According to any of the preceding aspects, the cell cycle inhibitor is administered before administering the chemotherapeutic agent.

According to the present invention, methods of promoting recovery from the effects of a chemotherapeutic regimen for treating cancer in a patient comprise administering to the patient: a therapeutically effective amount of a cell cycle inhibitor to inhibit the proliferation of normal, healthy cells; and a therapeutically effective about of a chemotherapeutic agent comprises a compound of Formula (1):

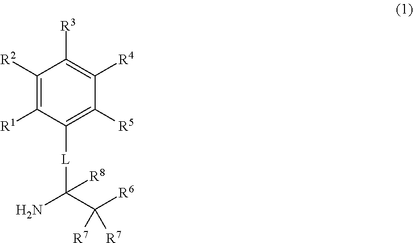

or a pharmaceutically acceptable salt thereof, wherein:

at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅-C₁₀ heteroaryl, C₆₋₁₆ heteroarylalkyl, and substituted C₆₋₁₆ heteroarylalkyl;

one of R¹, R², R³, R⁴, and R⁵ comprises a chemotherapeutic moiety;

each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;

R⁶ is selected from a carboxylic acid (—COOH), a carboxylic acid analog, and a carboxylic acid (bio)isostere;

each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;

R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;

each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and

L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

According to any of the preceding aspects, the chemotherapeutic moiety is a moiety of Formula (2a):

-A-NQ(-Z—C(R¹¹)₂—C(R¹¹)₂—R⁹)(—C(R¹¹)₂—C(R¹¹)₂—R⁹) (2a)

wherein,

A is selected from a bond (“—”), methylene (—CH₂—), oxygen (—O—), methyleneoxy (—CH₂—O—), carbonyl (—C(═O)—), methylenecarbonyl (—CH₂—C(═O)—), oxycarbonyl (—O—C(═O)—), and methyleneoxycarbonyl (—CH₂—O—C(═O)—);

Z is selected from a bond (“—”) and oxygen (—O—);

Q is selected from —O⁻ (a negatively charged oxygen atom that is bound to a positively charged nitrogen atom) and a free electron pair (:);

each R¹¹ is independently selected from hydrogen and deuterio; and

each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

According to any of the preceding aspects, the cell cycle inhibitor is selected from methotrexate or salts thereof, mycophenolic acid or derivatives or salts thereof, leflunomide or salts thereof, or a combination of any of the foregoing.

According to any of the preceding aspects, the method further comprises administering a therapeutically effective amount of a compound effective in stimulating recovery of inhibited normal, healthy cells.

According to any of the preceding aspects, the compound is effective in stimulating the recovery of the hematopoietic cell population.

Finally it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

1. A method of reducing the effects of chemotherapy on normal/healthy cells in a patient being treated for cancer or abnormal cell proliferation, comprising:
administering to the patient a therapeutically effective amount of a cell cycle inhibitor; and
administering to the patient a therapeutically effective amount of a chemotherapeutic compound comprising a compound of Formula (1): 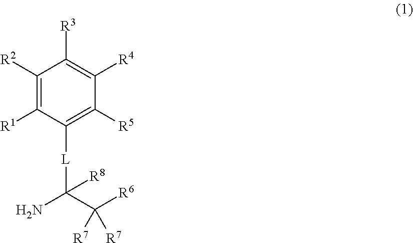
or a pharmaceutically acceptable salt thereof, wherein:
at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅₋₁₀ heteroaryl, C₆₋₁₆ heteroarylalkyl, and substituted C₆₋₁₆ heteroarylalkyl;
one of R¹, R², R³, R⁴, and R⁵ comprises a chemotherapeutic moiety;
each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;
R⁶ is selected from a carboxylic acid (—COOH), a carboxylic acid analog, and a carboxylic acid (bio)isostere;
each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;
R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;
each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and
L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

2. The method of claim 1, wherein the chemotherapeutic moiety is a moiety of Formula (2a):
-A-NQ(-Z—C(R¹¹)₂—C(R¹¹)₂—R⁹)(—C(R¹¹)₂—C(R¹¹)₂—R⁹) (2a)
wherein,
A is selected from a bond (“—”), methylene (—CH₂—), oxygen (—O—), methyleneoxy (—CH₂—O—), carbonyl (—C(═O)—), methylenecarbonyl (—CH₂—C(═O)—), oxycarbonyl (—O—C(═O)—), and methyleneoxycarbonyl (—CH₂—O—C(═O)—);
Z is selected from a bond (“—”) and oxygen (—O—);
Q is selected from —O⁻ (a negatively charged oxygen atom that is bound to a positively charged nitrogen atom) and a free electron pair (:);
each R¹¹ is independently selected from hydrogen and deuterio; and
each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

3. The method of claim 1, wherein the chemotherapeutic moiety is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected chloro (—Cl), bromo (—Br), iodo (—I), methylsulfonyloxy (—OSO₂CH₃), and trifluoromethylsulfonyloxy (—OSO₂CF₃).

4-5. (canceled)

6. The method of claim 1, wherein each of R², R³, and R⁵ is hydrogen.

7. The method of claim 1, wherein,
R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;
one of R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;
each of the other of R², R³, R⁴, and R⁵ is hydrogen;
R⁶ is selected from —COOH, —S(O)OH, —P(O)(OH)H, and 1H-tetrazole;
each R⁷ is independently selected from hydrogen, methyl, hydroxyl, and fluoro;
R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and
L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is hydrogen, methyl, or ethyl.

8. The method of claim 1, wherein,
R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;
R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;
each of R², R³, and R⁵ is hydrogen;
R⁶ is selected from —COOH, —S(O)OH, —P(O)(OH)H, and 1H-tetrazole;
each R⁷ is independently selected from hydrogen, methyl, hydroxyl, and fluoro;
R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and
L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

9. The method of claim 1, wherein the compound of Formula (1) is selected from:
3-amino-3-[5-[bis(2-chloroethyl)amino]-2-methyl-phenyl]propanoic acid (1);
3-amino-3-[4-[bis(2-chloroethyl)amino]-2-methyl-phenyl]propanoic acid (2);
3-amino-4-[5-[bis(2-chloroethyl)amino]-2-methyl-phenyl]butanoic acid (3);
3-amino-4-[4-[bis(2-chloroethyl)amino]-2-methyl-phenyl]butanoic acid (4);
(3S)-3-amino-4-[5-[bis(2-chloroethyl)amino]-2-methyl-phenyl]butanoic acid (5);
(3R)-3-amino-4-[5-[bis(2-chloroethyl)amino]-2-methyl-phenyl]butanoic acid (6);
(3S)-3-amino-4-[5-[bis(2-chloroethyl)amino]-2-methoxy-phenyl]butanoic acid (7);
(3S)-3-amino-4-[3-[bis(2-chloroethyl)amino]-2,6-dimethyl-phenyl]butanoic acid (8);
(3S)-3-amino-4-[5-[bis(2-chloroethyl)amino]-2-methyl-phenyl]-3-methyl-butanoic acid (9);
[(2R)-2-amino-3-[5-[bis(2-chloroethyl)amino]-2-methyl-phenyl]propyl]phosphinic acid (10);
(3R)-3-amino-4-[5-(bis(2-methyl sulfonyloxyethyl)amino)-2-methyl-phenyl]butanoic acid (11);
(3R)-3-amino-4-[5-(bis(2-bromoethyl)amino)-2-methyl-phenyl]butanoic acid (12);
(3R)-3-amino-4-[5-(2-chloroethyl(2-methylsulfonyloxyethyl)amino)-2-methyl-phenyl]butanoic acid (13);
(3R)-3-amino-4-[5-(2-bromoethyl(2-chloroethyl)amino)-2-methyl-phenyl]butanoic acid (14);
(3R)-3-amino-4-[5-(2-bromoethyl(2-methyl sulfonyloxyethyl)amino)-2-methyl-phenyl]butanoic acid (15);
(3S)-3-amino-4-[[5-[bis(2-chloroethyl)amino]-2-methyl-phenyl]amino]-4-oxo-butanoic acid (16);
(3R)-3-amino-5-[5-[bis(2-chloroethyl)amino]-2-methyl-phenyl]pentanoic acid (18);
(3R)-3-amino-4-[5-(2-chloroethyl(chloromethyl)carbamoyl)oxy-2-methyl-phenyl]butanoic acid (19);
(3R)-3-amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21); and
3-[(2R)-2-amino-4-hydroxy-4-oxo-butyl]-N,N-bis(2-chloroethyl)-4-methyl-benzeneamine oxide (22);
or a pharmaceutically acceptable salt of any of the foregoing.

10. The method of claim 1, wherein the cell cycle inhibitor comprises a myelosuppressor, an immunosuppressor, or a combination thereof.

11. The method of claim 1, wherein the cell cycle inhibitor is selected from methotrexate or salts thereof, mycophenolic acid or derivatives or salts thereof, leflunomide or salts thereof, or a combination of any of the foregoing.

12. The method of claim 1, wherein the therapeutically effective amount of the cell cycle inhibitor is effective in reducing the level of myelosuppression associated with the administration of the chemotherapeutic agent, compared to the level of myelosuppression associated with the administration of the chemotherapeutic agent without the administration of the cell cycle inhibitor.

13-14. (canceled)

15. The method of claim 1, wherein the cancer comprises brain cancer.

16. The method of claim 1, wherein the cell cycle inhibitor is effective in arresting the growth of hematopoietic stem cells, hematopoietic progenitor cells, T-cells, multipotent progenitors, common myeloid progenitors, common lymphoid progenitors, granulocyte-monocyte progenitors, and megakaryocyte-erythroid progenitors, renal epithelial cells, T-cells, and a combination of any of the foregoing.

17. The method of claim 1, wherein the cell cycle inhibitor is administered before administering the chemotherapeutic agent.

18. A method of treating cancer in a patient, comprising administering to the patient being treated for the cancer,
a therapeutically effective amount of a cell cycle inhibitor; and
a therapeutically effective amount of a chemotherapeutic agent comprising a compound of Formula (1): 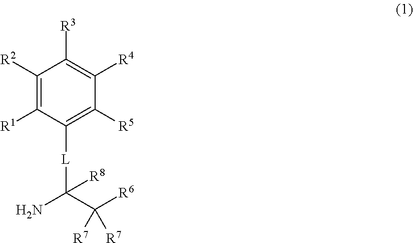
or a pharmaceutically acceptable salt thereof, wherein:
at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅-C₁₀ heteroaryl, C₆₋₁₆ heteroarylalkyl, and substituted C₆₋₁₆ heteroarylalkyl;
one of R¹, R², R³, R⁴, and R⁵ comprises a chemotherapeutic moiety;
each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;
R⁶ is selected from a carboxylic acid (—COOH), a carboxylic acid analog, and a carboxylic acid (bio)isostere;
each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;
R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;
each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and
L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

19. The method of claim 18, wherein the chemotherapeutic moiety is a moiety of Formula (2a):
-A-NQ(-Z—C(R¹¹)₂—C(R¹¹)₂—R⁹)(—C(R¹¹)₂—C(R¹¹)₂—R⁹) (2a)
wherein,
A is selected from a bond (“—”), methylene (—CH₂—), oxygen (—O—), methyleneoxy (—CH₂—O—), carbonyl (—C(═O)—), methylenecarbonyl (—CH₂—C(═O)—), oxycarbonyl (—O—C(═O)—), and methyleneoxycarbonyl (—CH₂—O—C(═O)—);
Z is selected from a bond (“—”) and oxygen (—O—);
Q is selected from —O⁻ (a negatively charged oxygen atom that is bound to a positively charged nitrogen atom) and a free electron pair (:);
each R¹¹ is independently selected from hydrogen and deuterio; and
each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

20. The method of claim 18, wherein the chemotherapeutic moiety is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected chloro (—Cl), bromo (—Br), iodo (—I), methylsulfonyloxy (—OSO₂CH₃), and trifluoromethylsulfonyloxy (—OSO₂CF₃).

21-22. (canceled)

23. The method of claim 18, wherein each of R², R³, and R⁵ is hydrogen.

24. The method of claim 18, wherein,
R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;
one of R², R³, R⁴, and R⁵ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;
each of the other of R², R³, R⁴, and R⁵ is hydrogen;
R⁶ is selected from —COOH, —S(O)OH, —P(O)(OH)H, and 1H-tetrazole;
each R⁷ is independently selected from hydrogen, methyl, hydroxyl, and fluoro;
R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and
L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is hydrogen, methyl, or ethyl.

25. The method of claim 18, wherein,
R¹ is selected from halogen, —N(R¹⁰)₂, —N⁺(—O⁻)(R¹⁰)₂, —N(R¹⁰)(OR¹⁰), —NO₂, —NO, —CN, —COOR¹⁰, —CON(R¹⁰)₂, —OH, C₁₋₄ alkyl, C₁₋₄ alkoxy, C₁₋₄ alkylsulfanyl, C₁₋₄ alkylsulfinyl, C₁₋₄ alkylsulfonyl, C₁₋₄ heteroalkyl, C₁₋₄ heteroalkoxy, C₁₋₄ fluoroalkyl, C₁₋₄ fluoroalkoxy, C₃₋₅ cycloalkyl, C₃₋₅ cycloalkyloxy, and C₄₋₈ cycloalkylalkyl; wherein each R¹⁰ is independently selected from hydrogen, deuterio, C₁₋₄ alkyl, and C₁₋₄ alkoxy, or two geminal R¹⁰ together with the nitrogen to which they are bonded form a 3- to 6-membered heterocyclic ring;
R⁴ is selected from —N(—CH₂—CH₂—R⁹)₂, —CH₂—N(—CH₂—CH₂—R⁹)₂, —N⁺(—O⁻)(—CH₂—CH₂—R⁹)₂, —CH₂—N⁺(—O⁻)(—CH₂—CH₂—R⁹)₂, —N(—O—CH₂—CH₂—R⁹)(—CH₂—CH₂—R⁹), —CH₂—N(—O—CH₂—CH₂—R⁹)(—CH₂—CH₂—R⁹), —O—N(—CH₂—CH₂—R⁹)₂, —CH₂—O—N(—CH₂—CH₂—R⁹)₂, —CO—N(—CH₂—CH₂—R⁹)₂, —CH₂—CO—N(—CH₂—CH₂—R⁹)₂, —O—CO—N(—CH₂—CH₂—R⁹)₂, and —CH₂—O—CO—N(—CH₂—CH₂—R⁹)₂, wherein each R⁹ is independently selected from —Cl, —Br, —I, —OSO₂CH₃, and —OSO₂CF₃;
each of R², R³, and R⁵ is hydrogen;
R⁶ is selected from —COOH, —S(O)OH, —P(O)(OH)H, and 1H-tetrazole;
each R⁷ is independently selected from hydrogen, methyl, hydroxyl, and fluoro;
R⁸ is selected from hydrogen, methyl, ethyl, propyl, isopropyl, cyclopropyl, butyl, isobutyl, cyclobutyl, tert-butyl, hydroxyl, C₁₋₄ alkoxy, C₁₋₄ fluoroalkyl, and C₁₋₄ fluoroalkoxy; and
L is selected from a bond “—”, —CH₂—, —C(OH)H—, —CHCH₃—, —C(CH₃)₂—, —CF₂—, —O—, —SO₂—, —NR¹⁷—, —CO—, —CH₂—CH₂—, —CH₂—CHCH₃—, —CHCH₃—CH₂—, —CH₂—CHOH—, —CHOH—CH₂—, —CH₂—CF₂—, —CF₂—CH₂—, —CO—NR¹⁷—, —NR¹⁷—CO—, —CH₂—NR¹⁷—, —NR¹⁷—CH₂—, —CH₂—O—, —O—CH₂—, —CH₂—S—, —S—CH₂—, —CH₂—SO₂—, —SO₂—CH₂—, —CH₂—CO—, and —CO—CH₂—, wherein R¹⁷ is selected from hydrogen, methyl, and ethyl.

26. The method of claim 18, wherein the compound of Formula (1) is selected from:
3-amino-3-[5-[bis(2-chloroethyl)amino]-2-methyl-phenyl]propanoic acid (1);
3-amino-3-[4-[bis(2-chloroethyl)amino]-2-methyl-phenyl]propanoic acid (2);
3-amino-4-[5-[bis(2-chloroethyl)amino]-2-methyl-phenyl]butanoic acid (3);
3-amino-4-[4-[bis(2-chloroethyl)amino]-2-methyl-phenyl]butanoic acid (4);
(3S)-3-amino-4-[5-[bis(2-chloroethyl)amino]-2-methyl-phenyl]butanoic acid (5);
(3R)-3-amino-4-[5-[bis(2-chloroethyl)amino]-2-methyl-phenyl]butanoic acid (6);
(3S)-3-amino-4-[5-[bis(2-chloroethyl)amino]-2-methoxy-phenyl]butanoic acid (7);
(3S)-3-amino-4-[3-[bis(2-chloroethyl)amino]-2,6-dimethyl-phenyl]butanoic acid (8);
(3S)-3-amino-4-[5-[bis(2-chloroethyl)amino]-2-methyl-phenyl]-3-methyl-butanoic acid (9);
[(2R)-2-amino-3-[5-[bis(2-chloroethyl)amino]-2-methyl-phenyl]propyl]phosphinic acid (10);
(3R)-3-amino-4-[5-(bis(2-methyl sulfonyloxyethyl)amino)-2-methyl-phenyl]butanoic acid (11);
(3R)-3-amino-4-[5-(bis(2-bromoethyl)amino)-2-methyl-phenyl]butanoic acid (12);
(3R)-3-amino-4-[5-(2-chloroethyl(2-methylsulfonyloxyethyl)amino)-2-methyl-phenyl]butanoic acid (13);
(3R)-3-amino-4-[5-(2-bromoethyl(2-chloroethyl)amino)-2-methyl-phenyl]butanoic acid (14);
(3R)-3-amino-4-[5-(2-bromoethyl(2-methyl sulfonyloxyethyl)amino)-2-methyl-phenyl]butanoic acid (15);
(3S)-3-amino-4-[[5-[bis(2-chloroethyl)amino]-2-methyl-phenyl]amino]-4-oxo-butanoic acid (16);
(3R)-3-amino-5-[5-[bis(2-chloroethyl)amino]-2-methyl-phenyl]pentanoic acid (18);
(3R)-3-amino-4-[5-(2-chloroethyl(chloromethyl)carbamoyl)oxy-2-methyl-phenyl]butanoic acid (19);
(3R)-3-amino-4-[5-[bis(2-chloroethyl)carbamoyloxymethyl]-2-nitro-phenyl]butanoic acid (20);
(3R)-3-amino-4-[5-(2-chloroethoxy(2-chloroethyl)amino)-2-methyl-phenyl]butanoic acid (21); and
3-[(2R)-2-amino-4-hydroxy-4-oxo-butyl]-N,N-bis(2-chloroethyl)-4-methyl-benzeneamine oxide (22);
or a pharmaceutically acceptable salt of any of the foregoing.

27. The method of claim 18, wherein the cell cycle inhibitor comprises a myelosuppressor, an immunosuppressor, or a combination thereof.

28. The method of claim 18, wherein the cell cycle inhibitor is selected from methotrexate or salts thereof, mycophenolic acid or derivatives or salts thereof, leflunomide or salts thereof, or a combination of any of the foregoing.

29. The method of claim 18, wherein the therapeutically effective amount of the cell cycle inhibitor is effective in reducing the level of myelosuppression associated with the administration of the chemotherapeutic agent, compared to the level of myelosuppression associated with the administration of the chemotherapeutic agent without the administration of the cell cycle inhibitor.

30. The method of claim 18, wherein the method results in a higher therapeutic index for the chemotherapeutic agent compared to a therapeutic index for the chemotherapeutic agent without administering the cell cycle inhibitor.

31. (canceled)

32. The method of claim 18, wherein the cancer comprises brain cancer.

33. The method of claim 18, wherein the cell cycle inhibitor is effective in arresting the growth of hematopoietic stem cells, hematopoietic progenitor cells, T-cells, multipotent progenitors, common myeloid progenitors, common lymphoid progenitors, granulocyte-monocyte progenitors, and megakaryocyte-erythroid progenitors, renal epithelial cells, T-cells, and a combination of any of the foregoing.

34. The method of claim 18, wherein the cell cycle inhibitor is administered before administering the chemotherapeutic agent.

35. A method of promoting recovery from the effects of a chemotherapeutic regimen for treating cancer in a patient comprising administering to the patient:
a therapeutically effective amount of a cell cycle inhibitor to inhibit the proliferation of normal, healthy cells; and
a therapeutically effective about of a chemotherapeutic agent comprising a compound of Formula (1): 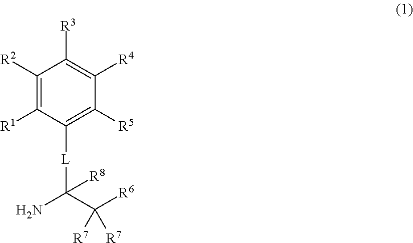
or a pharmaceutically acceptable salt thereof, wherein:
at least one of R¹ and R⁵ is independently selected from halogen, —N(R¹⁰)₂, —N⁺(—O⁻)(R¹⁰)₂, —N(OR¹⁰)(R¹⁰), —NO₂, —NO, —N(R¹⁰)(S(═O)R¹⁰), —N(R¹⁰)(S(═O)₂R¹⁰), —N(R¹⁰)(C(O)R¹⁰), —N(R¹⁰)(C(O)OR¹⁰), —N(R¹⁰)(C(O)N(R¹⁰)₂, —CN, —COOR¹⁰, —CON(R¹⁰)₂, —OH, —SH, C₁₋₄ alkylsulfanyl, C₁₋₄ alkylsulfinyl, C₁₋₄ alkylsulfonyl, —S(O)N(R¹⁰)₂, —S(O)₂N(R¹⁰)₂, C₁₋₄ fluoroalkyl, C₁₋₄ fluoroalkoxy, C₁₋₆ alkyl, substituted C₁₋₆ alkyl, C₁₋₆ alkoxy, substituted C₁₋₆ alkoxy, C₃₋₆ cycloalkyl, substituted C₃₋₆ cycloalkyl, C₃₋₆ cycloalkyloxy, substituted C₃₋₆ cycloalkyloxy, C₄₋₁₂ cycloalkylalkyl, substituted C₄₋₁₂ cycloalkylalkyl, C₆₋₁₀ aryl, substituted C₆₋₁₀ aryl, C₇₋₁₆ arylalkyl, substituted C₇₋₁₆ arylalkyl, C₁₋₆ heteroalkyl, substituted C₁₋₆ heteroalkyl, C₁₋₆ heteroalkoxy, substituted C₁₋₆ heteroalkoxy, C₃₋₆ heterocycloalkyl, substituted C₃₋₆ heterocycloalkyl, C₄₋₁₂ heterocycloalkylalkyl, substituted C₄₋₁₂ heterocycloalkylalkyl, C₅₋₁₀ heteroaryl, substituted C₅-C₁₀ heteroaryl, C₆₋₁₆ heteroarylalkyl, and substituted C₆₋₁₆ heteroarylalkyl;
one of R¹, R², R³, R⁴, and R⁵ comprises a chemotherapeutic moiety;
each of the other of R¹, R², R³, R⁴, and R⁵ is independently selected from hydrogen, deuterio, halogen, —OH, —N(R¹⁰)₂, —NO₂, —NO, —CN, —COOR¹⁰, —CON(R¹⁰)₂, C₁₋₄ alkylsulfanyl, C₁₋₄ alkylsulfinyl, C₁₋₄ alkylsulfonyl, C₁₋₆ alkyl, substituted C₁₋₆ alkyl, C₃₋₆ cycloalkyl, substituted C₃₋₆ cycloalkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₄₋₈ cycloalkylalkyl, and C₄₋₈ cycloalkylheteroalkyl;
R⁶ is selected from a carboxylic acid (—COOH), a carboxylic acid analog, and a carboxylic acid (bio)isostere;
each R⁷ is independently selected from hydrogen, deuterio, halogen, hydroxyl, C₁₋₆ alkyl, C₃₋₆ cycloalkyl, benzyl, and phenyl; or two R⁷ together with the carbon to which they are bonded form a ring selected from a C₃₋₆ cycloalkyl ring and a C₃₋₆ heterocycloalkyl ring;
R⁸ is selected from hydrogen, deuterio, halogen, C₁₋₆ alkyl, substituted C₁₋₆ alkyl, C₁₋₆ heteroalkyl, substituted C₁₋₆ heteroalkyl, C₁₋₆ alkoxy, substituted C₁₋₆ alkoxy, C₁₋₆ heteroalkoxy, substituted C₁₋₆ heteroalkoxy, C₃₋₆ cycloalkyl, substituted C₃₋₆ cycloalkyl, C₃₋₆ cycloalkyloxy, substituted C₃₋₆ cycloalkyloxy, —OH, —COOR¹⁰, C₁₋₄ fluoroalkyl, C₁₋₄ fluoroalkoxy, C₃₋₆ cycloalkyl, and phenyl;
each R¹⁰ is independently selected from hydrogen, deuterio, C₁₋₄ alkyl and C₁₋₄ alkoxy, or two geminal R¹⁰ together with the nitrogen to which they are bonded form a 3- to 6-membered heterocyclic ring; and
L is —(X)ₐ—, wherein,
each X is independently selected from a bond (“—”), —C(R¹⁶)₂—, wherein each R¹⁶ is independently selected from hydrogen, deuterio, halogen, hydroxyl, C₁₋₄ alkyl and C₁₋₄ alkoxy, or two R¹⁶ together with the carbon to which they are bonded form a C₃₋₆ cycloalkyl ring or a C₃₋₆ heterocycloalkyl ring, —O—, —S—, —SO—, —SO₂—, —CO—, and —N(R¹⁷)—, wherein R¹⁷ is selected from hydrogen and C₁₋₄ alkyl; and
a is selected from 0, 1, 2, 3, and 4.

36. The method of claim 35, wherein the chemotherapeutic moiety is a moiety of Formula (2a):
-A-NQ(-Z—C(R¹¹)₂—C(R¹¹)₂—R⁹)(—C(R¹¹)₂—C(R¹¹)₂—R⁹) (2a)
wherein,
A is selected from a bond (“—”), methylene (—CH₂—), oxygen (—O—), methyleneoxy (—CH₂—O—), carbonyl (—C(═O)—), methylenecarbonyl (—CH₂—C(═O)—), oxycarbonyl (—O—C(═O)—), and methyleneoxycarbonyl (—CH₂—O—C(═O)—);
Z is selected from a bond (“—”) and oxygen (—O—);
Q is selected from —O⁻ (a negatively charged oxygen atom that is bound to a positively charged nitrogen atom) and a free electron pair (:);
each R¹¹ is independently selected from hydrogen and deuterio; and
each R⁹ is independently selected from fluoro (—F), chloro (—Cl), bromo (—Br), iodo (—I), alkyl sulfonate (—OSO₂R⁴⁰, wherein R⁴⁰ is selected from C₁₋₄ alkyl), C₁₋₄ (per)fluoroalklyl sulfonate (—OSO₂R⁴⁰, wherein R⁴⁰ is selected from C₁₋₄ (per)fluoroalkyl), and (substituted) aryl sulfonate (—OSO₂R⁴⁰, wherein R⁴⁰ is selected from C₆₋₁₀ aryl).

37. The method of claim 35, wherein the cell cycle inhibitor is selected from methotrexate or salts thereof, mycophenolic acid or derivatives or salts thereof, leflunomide or salts thereof, or a combination of any of the foregoing.

38-39. (canceled)
